(19) United States
(12) Patent Application Publication
(10) Pub. No.: US 2007/0198198 A1
(43) Pub. Date: Aug. 23, 2007

(54) Methods and apparatuses for diagnosing AML and MDS

(75) Inventors: Michael E. Burczynski, Swampscott, MA (US); Andrew J. Dorner, Lexington, MA (US); Natalie C. Twine, Goffstown, NH (US); William L. Trepicchio, Andover, MA (US); Jennifer Stover, Topsfield, MA (US)

Correspondence Address: KIRKPATRICK & LOCKHART PRESTON GATES ELLIS LLP, STATE STREET FINANCIAL CENTER, ONE LINCOLN STREET, BOSTON, MA, 02111-2950, US

(73) Assignee: Wyeth, Madison, NJ (US)

(21) Appl. No.: 11/789,104

(22) Filed: Apr. 23, 2007

Related U.S. Application Data
(62) Division of application No. 10/834,114, filed on Apr. 29, 2004.
(60) Provisional application No. 60/466,055, filed on Apr. 29, 2003.

(51) Int. Cl. C12Q 1/68; G06F 19/00

(52) U.S. Cl. 702/20; 435/6

(57) ABSTRACT

Methods, systems and equipment for diagnosing or monitoring the progression or treatment of AML or MDS. This invention identifies a plurality of AML or MDS disease genes which are differentially expressed in bone marrow cells of AML or MDS patients as compared to disease-free humans. These AML or MDS disease genes can be used as molecular markers for detecting the presence or absence of AML or MDS. These genes can also be used for the early identification of MDS patients who eventually progress to AML.

### CROSS-REFERENCE TO RELATED APPLICATIONS

The present application claims priority from and incorporates by reference the entire disclosure of U.S. Provisional Patent Application Ser. No. 60/466,055, filed Apr. 29, 2003.

### TECHNICAL FIELD

This invention relates to methods, systems and equipment for diagnosing AML and MDS.

### BACKGROUND

Myelodysplastic syndromes (MDS) are a heterogeneous group of clonal disorders of bone marrow cell precursors characterized by variable clinical courses and outcomes. Approximately 30 percent of patients with MDS eventually progress to acute myelogenous leukemia (AML) and a clinical diagnostic assay especially suited to early identification of this subset of patients would help focus therapeutic options in these individuals.

A number of indices have been identified as important prognostic factors in MDS, including cytogenetic assessment, quantitation of blast percentages, and morphologic assessment of cell lines. Different risk classification systems have been developed to predict the overall survival of MDS patients and the progression from MDS to AML. Examples of these classification systems include the French-American-British (FAB) classification, the International Prognostic Scoring System (IPSS), the Bournemouth score, the Sanz score, and the Lille score. The French-American-British (FAB) classification system categorizes patients into one of five categories on the basis of observed cell morphologies and percentage of myeloblasts in the bone marrow and associates a median expected survival time with each category. The International Prognostic Scoring System (IPSS) incorporates assessment of cytogenetics, the number of cell lines involved, and the percentage of blasts in the bone marrow in patients and assigns a risk and median survival time to an overall IPSS score.

Recent expression profiling studies have revealed differences in AC133 surface-marker positive hematopoeitic stem cell fractions from patients with MDS versus AML (Miyazato et al., BLOOD, 98: 422-427 (2001)). Similar results have recently been observed in transcriptional profiles of CD34⁺ cells purified from bone marrow of patients with myelodysplastic syndromes, which are radically altered from the transcriptional profiles of CD34⁺ cells from normal individuals (Hofmann et al., BLOOD, 100: 3553-3560 (2002)). These studies, however, involved positive selection of specific cell subtypes, which is laborious and time-consuming.

### SUMMARY OF THE INVENTION

The present invention identifies numerous AML or MDS disease genes which are differentially expressed in bone marrow mononuclear cells (BMMCs) of AML or MDS patients as compared to BMMCs of disease-free humans. These disease genes can be used as molecular markers for diagnosing or monitoring the progression or treatment of AML or MDS. These genes can also be used for the early identification of MDS patients who eventually progress to AML.

In one aspect, the present invention provides methods useful for diagnosing or monitoring the progression or treatment of AML or MDS. The methods include comparing an expression profile of at least one gene in a bone marrow sample of a patient of interest to a reference expression profile, where the gene is differentially expressed in BMMCs of patients who have AML or MDS as compared to BMMCs of disease-free humans. In many embodiments, the gene is an AML or MDS disease gene selected from Tables 1 and 3.

Any number of AML or MDS disease genes can be employed. In one embodiment, the AML or MDS disease gene(s) is selected from those that have p values of no more than 0.005, 0.001, 0.0005, 0.0001, or less. In another embodiment, the AML or MDS disease gene(s) is selected from those that are significantly correlated with the class distinction between AML or MDS patients and disease-free humans. For instance, the AML or MDS disease gene(s) can be selected from those above the 1%, 5%, or 10% significance level in a permutation test.

In yet another embodiment, the AML or MDS disease genes are selected to include at least one gene upregulated in BMMCs of disease-free humans, at least one gene upregulated in BMMCs of AML patients, and at least one gene upregulated in BMMCs of MDS patients. In one example, the AML or MDS disease genes include the 91 genes depicted in Table 7a.

In many embodiments, the reference expression profile is an average expression profile of one or more AML or MDS genes in bone marrow samples of disease-free humans or patients of a known disease class. The reference expression profile and the expression profile of the patient of interest can be prepared using the same or comparable method. The expression profiles can also be prepared using different methods. Suitable methods for preparing a gene expression profile include, but are not limited to, quantitative RT-PCR, Northern Blot, in situ hybridization, slot-blotting, nuclease protection assay, nucleic acid arrays, immunoassays (such as ELISA, RIA, FACS, or Western Blot), two-dimensional gel electrophoresis, mass spectroscopy, and protein arrays.

In many embodiments, the bone marrow samples used in the present invention are whole bone marrow samples or samples containing enriched BMMCs or bone marrow leukocytes. The patient of interest may have AML, MDS which eventually progresses to AML, or MDS which does not progress to AML. The patient of interest may also be free from AML or MDS.

In one embodiment, the expression profile of the patient of interest is compared to at least two reference expression profiles. Each of the reference expression profiles is an average expression profile of one or more AML or MDS genes in bone marrow samples of disease-free humans or patients of a known disease class.

In another embodiment, the expression profile of the patient of interest is compared to at least three reference expression profiles. The first reference expression profile is an average expression profile of one or more AML or MDS genes in bone marrow samples of disease-free humans. The second reference expression profile is an average expression profile of the AML or MDS gene(s) in bone marrow samples of patients having AML. The third reference expression profile is an average expression profile of the AML or MDS gene(s) in bone marrow samples of patients having MDS.

Comparison of expression profiles can be performed manually or electronically. In one embodiment, the expression profile of the patient of interest is compared to two or more reference expression profiles by using a weighted voting algorithm.

The present invention also features methods for detecting early progression from MDS to AML. In one embodiment, the methods include assigning a class membership to an MDS patient. Where the bone marrow expression profile of the MDS patient is substantially similar to that of AML patients (e.g., resulting in an AML class membership), or the prediction confidence score is relatively low (e.g., below 0.1, 0.05, 0.01, or less), a positive prediction can be made that the MDS patient is likely to develop AML.

In another aspect, the present invention provides other methods that are useful for diagnosing or monitoring the progression or treatment of AML or MDS. The methods include comparing an expression profile of one or more genes in a bone marrow sample of a patient of interest to a reference expression profile, where the gene(s) is selected from Tables 8b and 9b.

In still another aspect, the methods of the present invention include comparing an expression profile of one or more genes in a bone marrow sample of a patient of interest to a reference expression profile, wherein the gene(s) is selected from Table 10b.

In addition to the genes listed in Tables 1, 3, 8b, 9b, and 10b, the present invention contemplate detection of the expression profiles of other genes that can hybridize under stringent or nucleic acid array hybridization conditions to the qualifiers selected from Tables 1, 3, 8b, 9b, and 10b. These genes may include hypothetical or putative genes which are supported by mRNA or EST data.

In a further aspect, the present invention features diagnostic kits or apparatuses. In one embodiment, the kits or apparatuses of the present invention include one or more polynucleotides, each of which is capable of hybridizing under stringent or nucleic acid array hybridization conditions to an RNA transcript, or the complement thereof, of a gene selected from Tables 1, 3, 8b, 9b, and 10b. In another embodiment, the kits or apparatuses of the present invention include one or more antibodies, each of which specifically recognizes a polypeptide product of a gene selected from Tables 1, 3, 8b, 9b, and 10b.

Moreover, the present invention features electronic systems for carrying out the methods of the present invention. In one embodiment, a system of the present invention includes (1) an input device through which an expression profile of at least one AML or MDS disease gene in a bone marrow sample of a patient of interest is inputted to the system; (2) a storage medium which includes one or more reference expression profiles of the AML or MDS disease gene; and (3) a processor which executes a program to compare the expression profile of the patient of interest to the reference expression profile(s).

Other features, objects, and advantages of the present invention are apparent in the detailed description that follows. It should be understood, however, that the detailed description, while indicating preferred embodiments of the invention, are given by way of illustration only, not limitation. Various changes and modifications within the scope of the invention will become apparent to those skilled in the art from the detailed description.

### BRIEF DESCRIPTION OF DRAWINGS

The patent or application file contains at least one drawing executed in color. Copies of this patent or patent application publication with color drawing(s) will be provided by the Office upon request and payment of the necessary fee. The drawing is provided for illustration, not limitation.

FIG. 1 shows a dendrogram which groups the expression profiles of the disease-free humans, AML patients, and MDS patients into three respective clusters.

FIG. 2 illustrates relative expression levels of groups of genes that are upregulated in disease-free humans, AML patients, and MDS patients, respectively.

FIG. 3 depicts the individual prediction confidence scores for an untrained test set of disease-free, AML and MDS samples, and the samples from the MDS patients who eventually progressed to AML.

### DETAILED DESCRIPTION

Numerous AML or MDS disease genes are identified by the present invention. These genes are differentially expressed in bone marrow cells of patients who have AML or MDS compared to bone marrow cells of disease-free humans. These genes can be used as molecular markers for diagnosing or monitoring the progression or treatment of AML or MDS. These genes can also be used for the detection of early stages of progression from MDS to AML. In many embodiments, the methods of the present invention do not require positive selection of specific cell subtypes (such as CD34⁺), thereby allowing for rapid diagnosis of AML or MDS.

### A. GENERAL METHODS FOR IDENTIFYING AML OR MDS DISEASE GENES

The availability of the human genome sequence, together with new developments in technology, such as DNA microarrays and computational biology, allows systemic gene expression studies for various diseases. This invention employs the systematic gene expression analysis technique to identify genes that are differentially expressed in BMMCs of AML or MDS patients versus disease-free patients. In many embodiments, polynucleotide arrays, such as cDNA or oligonucleotide arrays, are used for detecting and/or comparing gene expression profiles. Polynucleotide arrays allow quantitative detection of expression profiles of a large number of genes at one time. Suitable polynucleotide arrays for this purpose include, but are not limited to, Genechip® microarrays from Affymetrix (Santa Clara, Calif.) and cDNA microarrays from Agilent Technologies (Palo Alto, Calif.).

Polynucleotides to be hybridized to microarrays can be labeled with one or more labeling moieties to allow for detection of hybridized polynucleotide complexes. The labeling moieties can include compositions that are detectable by spectroscopic, photochemical, biochemical, bioelectronic, immunochemical, electrical, optical or chemical means. Exemplary labeling moieties include radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like. The polynucleotides to be hybridized to the microarrays can be either DNA or RNA.

Hybridization reactions can be performed in absolute or differential hybridization formats. In the absolute hybridization format, polynucleotides derived from one sample, such as BMMCs from an AML or MDS patient or a disease-free human, are hybridized to the probes in a microarray. Signals detected after the formation of hybridization complexes correlate to the polynucleotide levels in the sample. In the differential hybridization format, polynucleotides derived from two biological samples, such as one from an AML or MDS patient and the other from a disease-free human, are labeled with different labeling moieties. A mixture of these differently labeled polynucleotides is added to a microarray. The microarray is then examined under conditions in which the emissions from the two different labels are individually detectable. In one embodiment, the fluorophores Cy3 and Cy5 (Amersham Pharmacia Biotech, Piscataway N.J.) are used as the labeling moieties for the differential hybridization format.

Signals gathered from microarrays can be analyzed using commercially available software, such as those provide by Affymetrix or Agilent Technologies. Controls, such as for scan sensitivity, probe labeling and cDNA quantitation, can be included in the hybridization experiments. In many embodiments, the microarray expression signals are scaled and/or normalized before being further analyzed. For instance, the expression signals for each gene can be normalized to take into account variations in hybridization intensities when more than one array is used under similar test conditions. Signals for individual polynucleotide complex hybridization can also be normalized using the intensities derived from internal normalization controls contained on each array. In addition, genes with relatively consistent expression levels across the samples can be used to normalize the expression levels of other genes. In one embodiment, the expression levels are normalized across the samples such that the mean is zero and the standard deviation is one. In another embodiment, the expression data detected by the microarray are subject to a variation filter which excludes genes showing minimal or insignificant variation across the samples.

The gene expression profiles in AML or MDS BMMCs can be compared to the corresponding gene expression profiles in disease-free BMMCs. Genes that are differentially expressed in AML or MDS BMMCs compared to disease-free BMMCs are identified. By “differentially expressed,” it means that the average expression level of a gene in AML or MDS BMMCs has a statistically significant difference from that of disease-free BMMCs. In one embodiment, the average expression level of an AML (or MDS) disease gene in AML (or MDS) BMMCs is substantially higher or lower than that in disease-free BMMCs. In another embodiment, the average expression level of an AML (or MDS) disease gene in AML (or MDS) BMMCs is at least 1, 2, 3, 4, 5, 10, 20, or more folds higher or lower than that in disease-free BMMCs. In yet another embodiment, the p-value of a Student's t-test (e.g., two-tailed distribution, two sample unequal variance) for the difference in the average expression levels of an AML or MDS disease gene in AML or MDS BMMCs versus disease-free BMMCs is no more than 0.05, 0.01, 0.005, 0.001, 0.0005, 0.0001, or less.

In one embodiment, AML or MDS disease genes are identified by using clustering algorithms based on the microarray gene expression data. A clustering analysis can be either unsupervised or supervised. Examples of unsupervised cluster algorithms include, but are not limited to, self-organized maps (SOMs), principle component analysis, average linkage clustering, and hierarchical clustering. Examples of supervised cluster algorithms include, but are not limited to, nearest-neighbors test, support vector machines, and SPLASH. Under a supervised cluster analysis, the disease status of each sample is already known. Two-class or multi-class correlation metrics can be used.

In one example, a permutation test-based neighborhood analysis is used to analyze the microarray gene expression data for the identification and selection of AML or MDS disease genes. The algorithm for the neighborhood analysis is described in Golub et al., SCIENCE, 286: 531-537 (1999), and Slonim et al., PROCS. OF THE FOURTH ANNUAL INTERNATIONAL CONFERENCE ON COMPUTATIONAL MOLECULAR BIOLOGY, Tokyo, Japan, April 8-11, p 263-272 (2000), both of which are incorporated herein by reference.

Under one form of the neighborhood analysis, the expression profile of each gene is represented by an expression vector g=(e₁, e₂, e₃, . . . , eₙ), where eᵢ corresponds to the expression level of gene “g” in the ith sample. A class distinction is represented by an idealized expression pattern c=(c₁, c₂, c₃, . . . , cₙ), where cᵢ=1 or −1, depending on whether the ith sample is isolated from class 0 or class 1. Class 0 may consist of patients with a particular disease or diseases such as AML or MDS, and class 1 may represent disease-free humans.

The correlation of gene “g” to the class distinction can be calculated using a signal-to-noise score:

$$P ( g , c ) = \frac{x 0 ( g ) - x 1 ( g )}{sd 0 ( g ) + sd 1 ( g )}$$

where x0(g) and x1(g) represent the means of the log of the expression level of gene “g” in class 0 and class 1, respectively, and sd0(g) and sd1(g) represent the standard deviation of the log of the expression of gene “g” in class 0 and class 1, respectively. A higher absolute value of a signal-to-noise score indicates that the gene is more highly expressed in one class than in the other. An unusually high density of genes within the neighborhoods of the class distinction, as compared to random patterns, suggests that many genes have expression patterns that are significantly correlated with the class distinction.

AML or MDS disease genes can be selected based on the neighborhood analysis. In one embodiment, the selected AML or MDS disease genes have top absolute P(g,c) values. In another embodiment, the selected AML (or MDS) disease genes include genes that are highly expressed in AML (or MDS) BMMCs, as well as genes that are highly expressed in disease-free BMMCs.

In still another embodiment, the selected AML or MDS disease genes are limited to those shown to be significantly correlated to the class distinction under a permutation test (e.g., above the 1%, 2%, 5%, or 10% significance level). As used herein, x % significance level means that x % of random neighborhoods contain as many genes as the real neighborhood around the class distinction.

The above-described methods can be readily adapted to the identification of genes whose expression profiles in bone marrow cells are correlated with different stages of disease progression, or different clinical responses to a therapeutic treatment. For instance, BMMC gene expression profiles of MDS patients who eventually progress to AML can be compared to BMMC gene expression profiles of MDS patients who do not progress to AML. Genes that are differentially expressed in these two classes of patients may be identified and used as molecular markers for the prediction of progression from MDS to AML. For another instance, AML or MDS patients can be grouped based on their different responses to a therapeutic treatment. The global gene expression analysis is employed to search for genes which are differentially expressed in one group of patients as compared to another group of patients. The genes thus identified can be used for the prognosis or prediction of clinical outcome of an AML or MDS patient of interest.

### B. IDENTIFICATION OF AML OR MDS DISEASE GENES

In one embodiment, HG-U95Av2 or HG-U95A genechips (manufactured by Affymetrix, Inc.) were used for the identification of AML or MDS disease genes. See Examples 1-4, infra. RNA transcripts were isolated from BMMCs of AML or MDS patients and disease-free humans. cRNA was prepared from the RNA transcripts using protocols according to the Affymetrix's Expression Analysis Technical Manuals and then hybridized to the genechip. Hybridization signals were collected for each oligonucleotide probe on a genechip. Signals for the oligonucleotide probes of the same qualifier were averaged. Qualifiers that produced different hybridization signals for AML or MDS samples relative to disease-free samples were identified.

Table 1 lists examples of qualifiers on HG-U95Av2 or HG-U95A genechips that showed different hybridization signals for AML samples compared to disease-free samples. Each qualifier represents multiple oligonucleotide probes, and each of these oligonucleotide probes is stably attached to a different respective region on the genechip. Each qualifier in Table 1 corresponds to at least one AML disease gene which is differentially expressed in AML BMMCs compared to disease-free BMMCs. At least one oligonucleotide probe of the qualifier can hybridize under nucleic acid array hybridization conditions to an RNA transcript of the corresponding AML disease gene.

Table 1 illustrates the ratio of the average expression level of each AML disease gene in AML BMMCs over that in disease-free BMMCs (“AML/Disease-Free”), and the ratio of the average expression level of each AML disease gene in MDS BMMCs over that in disease-free BMMCs (“MDS/Disease-Free”). Table 1 also provides the p-value of a Student's t-test (two-tailed distribution, two sample unequal variance) for the difference between the average expression levels of each AML disease gene in AML BMMCs versus disease-free BMMCs (“p value (AML vs Disease-Free)”). The p-value suggests the statistical significance of the difference observed between the average expression levels. Lesser p-values indicate more statistical significance for the observed difference.

TABLE 1
Expression Profiles of AML Disease Genes in AML and Disease-Free BMMCs

| | | | | p value |
|---|---|---|---|---|
| Qualifier | Gene Name | AML/Disease-Free | MDS/Disease-Free | (AML vs Disease-Free) |
| 1065_at | FLT3 | 11.743421 | 1.9983553 | 9.673E−05 |
| 41071_at | SPINK2 | 8.6161525 | 3.1442831 | 0.0001253 |
| 32609_at | H2AFO | 5.8846154 | 3.9109312 | 0.000259 |
| 39610_at | HOXB2 | 5.7894737 | 2.4736842 | 0.0002488 |
| 32755_at | ACTA2 | 5.5263158 | 1.25387 | 2.747E−06 |
| 38487_at | STAB1 | 4.8185118 | 2.831216 | 0.0001913 |
| 41654_at | ADA | 4.5526316 | 2.0263158 | 1.869E−05 |
| 41138_at | MIC2 | 4.5394737 | 2.2039474 | 4.257E−05 |
| 39317_at | CMAH | 4.4473684 | 1.5526316 | 0.0001253 |
| 39070_at | SNL | 4.2005958 | 2.1896723 | 0.0001608 |
| 39421_at | RUNX1 | 4.1447368 | 1.1447368 | 1.518E−05 |
| 36536_at | SCHIP1 | 4.0866873 | 1.625387 | 0.0005349 |
| 34397_at | OA48-18 | 3.869969 | 2.1362229 | 1.169E−09 |
| 38717_at | DKFZP586A0522 | 3.8504155 | 1.0110803 | 1.246E−07 |
| 33777_at | TBXAS1 | 3.8259109 | 0.6983806 | 7.678E−06 |
| 255_s_at | INHA | 3.7151703 | 1.9504644 | 0.0003908 |
| 286_at | H2AFO | 3.6978947 | 2.7789474 | 0.000229 |
| 39175_at | PFKP | 3.6947368 | 1.4842105 | 3.788E−07 |
| 37532_at | ACADM | 3.6786114 | 1.9148936 | 9.482E−07 |
| 33352_at | UNK_X57985 | 3.6064593 | 2.4222488 | 0.0001612 |
| 39710_at | P311 | 3.6049461 | 0.8560558 | 5.794E−07 |
| 39002_at | TCAP | 3.4210526 | 1.3684211 | 0.0001773 |
| 39971_at | LYL1 | 3.4144737 | 2.5263158 | 2.037E−07 |
| 40274_at | DBP | 3.3697047 | 1.3863928 | 2.634E−06 |
| 32251_at | FLJ21174 | 3.365651 | 1.7867036 | 0.000215 |
| 34862_at | LOC51097 | 3.3552632 | 1.0432331 | 6.139E−05 |
| 1475_s_at | MYB | 3.3436533 | 2.1826625 | 0.0004177 |
| 36785_at | HSPB1 | 3.3133971 | 1.9617225 | 7.08E−05 |
| 36215_at | PRKACB | 3.245614 | 1.1695906 | 0.0006812 |
| 943_at | RUNX1 | 3.2409972 | 1.1634349 | 0.0001006 |
| 40365_at | GNA15 | 3.2311449 | 1.6603364 | 4.756E−05 |
| 33412_at | LGALS1 | 3.2003664 | 0.9718521 | 6.642E−05 |
| 32543_at | CALR | 3.1500782 | 1.1021365 | 1.068E−06 |
| 33986_r_at | HSPCB | 3.1015038 | 2.3571429 | 3.752E−06 |
| 32245_at | M6A | 3.0892449 | 1.6475973 | 1.216E−07 |
| 35731_at | ITGA4 | 3.0747922 | 1.4127424 | 0.0009317 |
| 37033_s_at | GPX1 | 3.0237975 | 2.7631579 | 3.878E−11 |
| 33131_at | SOX4 | 2.9802632 | 1.5197368 | 1.185E−05 |
| 1750_at | UNK_AD000092 | 2.9736842 | 1.1578947 | 2.761E−05 |
| 1011_s_at | YWHAE | 2.9256966 | 1.25387 | 1.834E−08 |
| 36465_at | IRF5 | 2.8865132 | 1.2335526 | 2.027E−06 |
| 35576_f_at | UNK_AL009179 | 2.8654971 | 2.3684211 | 8.747E−06 |
| 1751_g_at | UNK_AD000092 | 2.854251 | 1.0931174 | 1.605E−05 |
| 36347_f_at | H2BFD | 2.852292 | 2.2665535 | 2.19E−05 |
| 2042_s_at | MYB | 2.7909563 | 1.5122313 | 2.394E−06 |
| 36943_r_at | PLAGL1 | 2.7758913 | 1.2478778 | 5.83E−05 |
| 630_at | DCTD | 2.7631579 | 1.268797 | 8.157E−07 |
| 38826_at | 37501 | 2.737691 | 1.3752122 | 0.0001572 |
| 39023_at | IDH1 | 2.7236842 | 1.0065789 | 0.0002949 |
| 2025_s_at | APEX | 2.7174515 | 1.0886427 | 1.592E−05 |
| 478_g_at | IRF5 | 2.7150193 | 0.9242619 | 2.67E−06 |
| 2067_f_at | BAX | 2.6913876 | 1.8301435 | 2.306E−06 |
| 948_s_at | PPID | 2.673445 | 1.291866 | 9.863E−12 |
| 36597_at | NOLC1 | 2.6702786 | 1.3467492 | 7.715E−07 |
| 32246_g_at | M6A | 2.6644737 | 1.5296053 | 7.567E−06 |
| 39372_at | FADS1 | 2.6430206 | 1.0640732 | 1.098E−05 |
| 34378_at | ADFP | 2.6315789 | 2.2368421 | 0.0002101 |
| 41213_at | PRDX1 | 2.6245801 | 1.5285554 | 4.563E−07 |
| 1470_at | POLD2 | 2.6210526 | 1.2315789 | 0.0001583 |
| 38454_g_at | ICAM2 | 2.6177285 | 1.4958449 | 1.348E−05 |
| 35796_at | PTK9L | 2.593985 | 1.0902256 | 1.312E−05 |
| 40133_s_at | GRHPR | 2.5887393 | 1.0832313 | 4.383E−05 |
| 31665_s_at | CDA02 | 2.5837321 | 1.507177 | 1.016E−05 |
| 40485_at | HSA249128 | 2.5730994 | 1.1695906 | 4.453E−05 |
| 1997_s_at | BAX | 2.5657895 | 1.5789474 | 0.0001042 |
| 37348_s_at | TRIP7 | 2.5614035 | 1.4298246 | 1.61E−05 |
| 41108_at | PGPL | 2.5589837 | 1.2522686 | 6.48E−08 |
| 31522_f_at | H2BFG | 2.556391 | 2.1804511 | 5.38E−05 |
| 39061_at | BST2 | 2.556391 | 1.0526316 | 4.816E−06 |
| 39968_at | LTC4S | 2.5554017 | 1.8282548 | 0.0002229 |
| 38745_at | LIPA | 2.5531915 | 0.9574468 | 0.0003633 |
| 32139_at | ZNF185 | 2.5263158 | 1.3157895 | 2.883E−07 |
| 32696_at | PBX3 | 2.5263158 | 1 | 0.0002592 |
| 32096_at | UNK_AC005546 | 2.5164474 | 1.3322368 | 0.0005233 |
| 34651_at | COMT | 2.4947368 | 0.9789474 | 6.264E−09 |
| 40634_at | NAP1L1 | 2.4860022 | 1.075028 | 2.186E−05 |
| 32051_at | MGC2840 | 2.4722992 | 0.9972299 | 7.406E−09 |
| 39691_at | SH3GLB1 | 2.4671053 | 1.2582237 | 2.013E−06 |
| 40854_at | UQCRC2 | 2.4657534 | 0.9516943 | 1.301E−11 |
| 631_g_at | DCTD | 2.4586466 | 1.443609 | 8.701E−09 |
| 32825_at | HRMT1L2 | 2.4552632 | 1.2 | 3.639E−08 |
| 33415_at | NME2 | 2.4548311 | 1.1390416 | 2.008E−07 |
| 40184_at | CSNK1A1 | 2.4493927 | 0.8906883 | 4.093E−05 |
| 38811_at | ATIC | 2.4473684 | 1.1842105 | 1.892E−06 |
| 32550_r_at | CEBPA | 2.4409237 | 2.0139635 | 0.0001037 |
| 1161_at | HSPCB | 2.4308111 | 1.3797792 | 6.021E−08 |
| 35255_at | RANBP7 | 2.424812 | 1.0432331 | 5.319E−07 |
| 38671_at | KIAA0620 | 2.424812 | 1.3815789 | 0.0006179 |
| 1519_at | ETS2 | 2.4177632 | 1.8009868 | 0.0007594 |
| 38352_at | PPIH | 2.4148607 | 1.5789474 | 0.0009522 |
| 37016_at | ECHS1 | 2.4043062 | 1.2559809 | 4.536E−05 |
| 40698_at | CLECSF2 | 2.4022556 | 1.9962406 | 1.053E−05 |
| 34345_at | C20ORF14 | 2.3987854 | 1.0020243 | 6.468E−06 |
| 40877_s_at | MN7 | 2.3982125 | 1.9513406 | 0.0002734 |
| 1920_s_at | CCNG1 | 2.3947368 | 1.3157895 | 0.0002743 |
| 39672_at | PTPN7 | 2.3923445 | 1.4593301 | 5.175E−05 |
| 36626_at | HSD17B4 | 2.3684211 | 0.9064327 | 2.756E−06 |
| 41379_at | KIAA0594 | 2.3684211 | 1.6015038 | 8.257E−08 |
| 39091_at | JWA | 2.3684211 | 1.0441426 | 8.997E−06 |
| 36624_at | IMPDH2 | 2.36195 | 1.0903796 | 1.121E−05 |
| 1474_s_at | MYB | 2.3464912 | 1.4912281 | 6.46E−06 |
| 32062_at | KIAA0014 | 2.3440043 | 1.3510581 | 1.989E−06 |
| 38642_at | ALCAM | 2.3402256 | 0.9586466 | 0.0004905 |
| 31523_f_at | H2BFH | 2.3402256 | 2.3120301 | 0.0005074 |
| 35305_at | XPNPEPL | 2.3391813 | 1.3450292 | 3.516E−06 |
| 34470_at | TFEC | 2.3355263 | 1.1842105 | 6.589E−07 |
| 32232_at | NDUFB5 | 2.3335913 | 1.1609907 | 1.461E−10 |
| 31528_f_at | H2BFE | 2.3299101 | 2.4261874 | 1.61E−05 |
| 38780_at | AKR1A1 | 2.3120301 | 1.0230934 | 1.01E−06 |
| 40774_at | CCT3 | 2.3089983 | 1.2478778 | 1.119E−06 |
| 37147_at | SCGF | 2.3054569 | 1.2035841 | 0.000612 |
| 38376_at | ACADVL | 2.2941176 | 1.1981424 | 1.71E−09 |
| 32221_at | MRPS18B | 2.2932331 | 1.0526316 | 4.902E−09 |
| 38213_at | UNK_U78027 | 2.2894737 | 1.1315789 | 3.033E−06 |
| 32819_at | H2B/S | 2.2894737 | 2.1541353 | 0.0001574 |
| 39638_at | TFAP4 | 2.2781955 | 1.2406015 | 7.211E−07 |
| 1456_s_at | IFI16 | 2.2781955 | 1.443609 | 0.0005642 |
| 32241_at | TARDBP | 2.2768879 | 1.7505721 | 1.322E−08 |
| 38416_at | CCT6A | 2.2672065 | 1.0931174 | 1.449E−07 |
| 674_g_at | MTHFD1 | 2.2645429 | 1.1634349 | 1.728E−06 |
| 39377_at | MRPS27 | 2.2556391 | 1.1729323 | 1.803E−07 |
| 32260_at | PEA15 | 2.2556391 | 1.6165414 | 0.0004353 |
| 41375_at | LSM2 | 2.2437673 | 1.0803324 | 3.948E−08 |
| 1527_s_at | CG018 | 2.2421053 | 1.2631579 | 0.0001287 |
| 41749_at | C21ORF33 | 2.2389991 | 1.1647972 | 3.534E−09 |
| 39056_at | PAICS | 2.2368421 | 1.0394737 | 5.427E−05 |
| 31524_f_at | H2BFK | 2.2336329 | 1.8485237 | 7.188E−05 |
| 41163_at | P24B | 2.2330827 | 0.8120301 | 0.0003901 |
| 31801_at | UNK_AI808712 | 2.2291022 | 1.2074303 | 5.754E−09 |
| 33173_g_at | FLJ10849 | 2.2291022 | 1.625387 | 7.299E−07 |
| 37692_at | DBI | 2.2291022 | 0.8823529 | 0.0001216 |
| 37306_at | KIAA0068 | 2.2291022 | 1.25387 | 0.0002182 |
| 38695_at | NDUFS4 | 2.2285143 | 1.2091939 | 1.848E−09 |
| 40976_at | KATNB1 | 2.2248804 | 1.291866 | 0.0002678 |
| 37386_i_at | KDELR1 | 2.2208559 | 1.4977865 | 3.425E−09 |
| 39580_at | KIAA0649 | 2.2105263 | 1.3684211 | 0.0003004 |
| 35741_at | PIP5K2B | 2.2105263 | 1.2631579 | 1.269E−06 |
| 37927_at | CHC1 | 2.2105263 | 1.0105263 | 4.298E−05 |
| 40467_at | SDHD | 2.2050817 | 1.0889292 | 0.0001754 |
| 36955_at | C5ORF8 | 2.2009569 | 0.7894737 | 0.0002307 |
| 40789_at | AK2 | 2.1983806 | 0.8137652 | 0.0005989 |
| 38704_at | MACF1 | 2.1944692 | 0.9500446 | 9.013E−05 |
| 31863_at | KIAA0179 | 2.1901528 | 1.1460102 | 3.782E−05 |
| 39471_at | M11S1 | 2.1870555 | 1.1308677 | 9.412E−07 |
| 32184_at | LMO2 | 2.1868421 | 1.2078947 | 3.656E−07 |
| 39516_at | POP5 | 2.1842105 | 1.1842105 | 4.881E−10 |
| 40127_at | PMX1 | 2.1804511 | 1.4849624 | 0.0006778 |
| 38075_at | SYPL | 2.1779952 | 0.932293 | 2.59E−07 |
| 34889_at | ATP6A1 | 2.1750806 | 1.047261 | 5.196E−05 |
| 40441_g_at | PAI-RBP1 | 2.1745152 | 0.9833795 | 1.426E−05 |
| 36928_at | ZNF146 | 2.1743979 | 1.2778769 | 7.443E−05 |
| 36673_at | MPI | 2.1710526 | 1.2582237 | 7.142E−07 |
| 39799_at | FABP5 | 2.1710526 | 1.507177 | 1.489E−05 |
| 38473_at | TARS | 2.1659919 | 1.4979757 | 5.793E−07 |
| 35184_at | KIAA0546 | 2.1654135 | 1.3984962 | 9.042E−06 |
| 32803_at | CNIL | 2.1649485 | 1.3266413 | 2.116E−08 |
| 33836_at | NPIP | 2.1606648 | 1.4750693 | 6.794E−05 |
| 36023_at | PRH1 | 2.1594427 | 1.3931889 | 1.09E−08 |
| 36458_at | KIAA1018 | 2.15311 | 1.4712919 | 3.703E−05 |
| 36833_at | UNK_U78027 | 2.1513158 | 0.9868421 | 1.041E−05 |
| 1499_at | FNTA | 2.150913 | 1.2083781 | 2.732E−10 |
| 38732_at | CLNS1A | 2.1403509 | 0.8684211 | 1.971E−07 |
| 41535_at | CDK2AP1 | 2.1365477 | 1.2256165 | 1.059E−06 |
| 39818_at | RCL | 2.1362229 | 1.1842105 | 0.0003654 |
| 40576_f_at | HNRPDL | 2.1337127 | 1.1522048 | 2.224E−06 |
| 263_g_at | AMD1 | 2.1332587 | 1.3605823 | 4.314E−05 |
| 40842_at | SNRPA | 2.1315789 | 1.1785714 | 8.509E−07 |
| 37726_at | MRPL3 | 2.1291866 | 1.1244019 | 9.984E−07 |
| 33230_at | NMP200 | 2.1281465 | 1.1498856 | 7.867E−05 |
| 34610_at | GNB2L1 | 2.122807 | 1.6491228 | 3.564E−05 |
| 1196_at | CHC1 | 2.1217105 | 0.7401316 | 3.7E−05 |
| 38399_at | UNK_AL034428 | 2.121116 | 1.379201 | 1.992E−12 |
| 38375_at | ESD | 2.120563 | 1.0556916 | 1.25E−09 |
| 38011_at | RMP | 2.1172249 | 1.0944976 | 1.43E−07 |
| 39464_at | HSPA8 | 2.1172249 | 1.3636364 | 0.0001692 |
| 41664_at | TIMM44 | 2.1146617 | 1.3533835 | 3.919E−05 |
| 38612_at | TSPAN-3 | 2.1130031 | 1.1842105 | 1.787E−08 |
| 40979_at | C14ORF3 | 2.1106337 | 1.5950591 | 1.739E−08 |
| 34302_at | EIF3S4 | 2.1101365 | 1.0964912 | 1.417E−08 |
| 1009_at | HINT1 | 2.1092204 | 1.2999604 | 4.663E−07 |
| 35771_at | DEAF1 | 2.1052632 | 1.4254386 | 8.593E−06 |
| 37700_at | BLMH | 2.1052632 | 1.3421053 | 9.506E−06 |
| 41282_s_at | PEX10 | 2.1052632 | 1.3596491 | 2.53E−05 |
| 1735_g_at | UNK_M60556 | 2.1052632 | 1.7894737 | 0.0003621 |
| 35814_at | GA17 | 2.1040218 | 1.1655909 | 5.368E−12 |
| 41812_s_at | KIAA0906 | 2.1 | 1.2 | 0.0003867 |
| 1846_at | LGALS8 | 2.098338 | 1.4542936 | 0.0005103 |
| 37768_at | MPG | 2.0921053 | 0.9868421 | 0.0001115 |
| 41357_at | ATP5B | 2.0910384 | 1.5576102 | 7.479E−07 |
| 40099_at | ARHGEF2 | 2.0897833 | 1.2848297 | 2.735E−05 |
| 41133_at | G3BP | 2.0882852 | 0.8658744 | 2.226E−05 |
| 35801_at | ITPA | 2.0864662 | 1.2030075 | 1.448E−09 |
| 39779_at | TARBP1 | 2.075188 | 1.5338346 | 1.677E−05 |
| 195_s_at | CASP4 | 2.0732907 | 1.2395475 | 1.606E−05 |
| 31838_at | HSU79274 | 2.0723684 | 1.0361842 | 1.159E−06 |
| 35355_at | DDX30 | 2.0676692 | 1.6541353 | 0.000258 |
| 40788_at | AK2 | 2.0614035 | 1.2938596 | 0.0008134 |
| 39418_at | DKFZP564M182 | 2.0594966 | 1.2307944 | 1.128E−07 |
| 38763_at | SORD | 2.0594966 | 1.201373 | 1.086E−05 |
| 40721_g_at | UNK_AL022398 | 2.0567867 | 1.101108 | 8.949E−05 |
| 37774_at | 37501 | 2.0526316 | 1.3947368 | 0.0001028 |
| 39785_at | KIAA0092 | 2.0467836 | 1.2573099 | 1.259E−05 |
| 38072_at | UNK_AL031432 | 2.0467836 | 1.3011696 | 5.933E−05 |
| 33414_at | PM5 | 2.0433437 | 1.1145511 | 3.371E−07 |
| 33944_at | APLP2 | 2.0391787 | 1.1859806 | 2.089E−06 |
| 37497_at | HHEX | 2.0379437 | 1.0648715 | 1.248E−05 |
| 2062_at | IGFBP7 | 2.0300752 | 0.99087 | 0.0001227 |
| 40516_at | AHR | 2.0300752 | 1.5037594 | 0.0004152 |
| 39693_at | MGC5508 | 2.0263158 | 0.7368421 | 8.008E−05 |
| 33866_at | TPM4 | 2.0230263 | 1.3322368 | 1.39E−06 |
| 512_at | NR1H3 | 2.0230263 | 1.1842105 | 0.0005192 |
| 33984_at | HSPCB | 2.0218641 | 1.2594387 | 1.376E−08 |
| 37229_at | ATR | 2.0210526 | 1.2315789 | 4.241E−06 |
| 38768_at | HADHSC | 2.0195838 | 0.8996328 | 9.592E−06 |
| 1521_at | NME1 | 2.018797 | 0.9022556 | 0.0001755 |
| 351_f_at | UNK_D28423 | 2.0168067 | 1.084697 | 2.697E−05 |
| 39507_at | OGT | 2.0158406 | 1.3413388 | 2.692E−05 |
| 41448_at | UNK_AC004080 | 2.0131579 | 1.6578947 | 0.0001027 |
| 1151_at | RPL22 | 2.0118846 | 1.4282683 | 2.996E−08 |
| 36608_at | MDH1 | 2.0110803 | 1.101108 | 4.011E−06 |
| 32853_at | TOMM70A | 2.0095694 | 1.291866 | 6.838E−05 |
| 35818_at | HCS | 2.0086609 | 1.419054 | 4.671E−06 |
| 39062_at | UNK_AL008726 | 2.0081758 | 0.8124681 | 0.0001906 |
| 33873_at | TCFL1 | 2.0065789 | 1.7434211 | 2.327E−08 |
| 37722_s_at | DHPS | 2.0065789 | 1.0526316 | 1.013E−06 |
| 39390_at | NUP133 | 2 | 1.3157895 | 5.535E−07 |
| 34839_at | KIAA1104 | 2 | 0.9736842 | 8.728E−05 |
| 34223_at | CSF3R | 0.4988038 | 0.7787081 | 2.841E−05 |
| 33466_at | LOC90355 | 0.498615 | 0.6855956 | 5.363E−05 |
| 752_s_at | DNAJB1 | 0.4978663 | 0.7539118 | 4.145E−05 |
| 1352_at | IL8RA | 0.4977117 | 0.7551487 | 0.0007667 |
| 37002_at | BLVRB | 0.4963004 | 1.0596119 | 0.0003982 |
| 38578_at | TNFRSF7 | 0.4962406 | 1.037594 | 1.609E−06 |
| 32493_at | TEF | 0.495716 | 1.2851897 | 0.0008307 |
| 38740_at | ZFP36L1 | 0.4925776 | 1.585695 | 3.316E−07 |
| 676_g_at | IFITM1 | 0.4925646 | 0.8968177 | 0.0001586 |
| 33333_at | PIP3-E | 0.4910141 | 0.9820282 | 1.557E−06 |
| 34652_at | NPAS1 | 0.4904306 | 1.3157895 | 5.16E−05 |
| 596_s_at | CSF3R | 0.4878352 | 0.8862959 | 1.99E−06 |
| 1794_at | CCND3 | 0.4871221 | 0.8902576 | 9.989E−10 |
| 37294_at | BTG1 | 0.4867432 | 0.8132173 | 1.285E−07 |
| 148_at | ELL2 | 0.4849624 | 1.037594 | 4.484E−06 |
| 40227_at | ESDN | 0.4824561 | 1.4285714 | 0.0002084 |
| 39301_at | CAPN3 | 0.4817128 | 0.9901873 | 0.0001876 |
| 32747_at | ALDH2 | 0.4814727 | 0.6655653 | 1.82E−05 |
| 36136_at | PIG11 | 0.4805492 | 0.9153318 | 1.009E−06 |
| 1427_g_at | SLA | 0.4798762 | 0.8049536 | 7.266E−05 |
| 41107_at | SNPH | 0.4778393 | 0.7894737 | 4.666E−07 |
| 936_s_at | PPP1R2 | 0.4778393 | 1.932133 | 0.0004586 |
| 37025_at | PIG7 | 0.4776815 | 0.9553631 | 1.017E−08 |
| 38735_at | KIAA0513 | 0.4776648 | 0.7894737 | 2.144E−06 |
| 31621_s_at | ELN | 0.4773562 | 0.9822521 | 5.257E−07 |
| 34435_at | AQP9 | 0.4773562 | 1.119951 | 7.24E−05 |
| 36640_at | MYL2 | 0.4768108 | 0.8363731 | 8.979E−08 |
| 358_at | P2Y10 | 0.4766634 | 0.8043694 | 4.144E−05 |
| 1305_s_at | CYP4F3 | 0.4766634 | 0.6703078 | 0.0001544 |
| 32775_r_at | PLSCR1 | 0.4748714 | 0.718243 | 2.895E−05 |
| 36280_at | GZMK | 0.4736842 | 1.1210526 | 0.000479 |
| 38065_at | HMG2 | 0.4727871 | 0.6064593 | 1.165E−06 |
| 33080_s_at | RAP1GA1 | 0.4703247 | 1.8669013 | 6.463E−07 |
| 106_at | RUNX3 | 0.46875 | 1.3199013 | 8.181E−05 |
| 1096_g_at | CD19 | 0.4685494 | 0.8472401 | 4.213E−08 |
| 39245_at | UNK_U72507 | 0.4681763 | 0.7894737 | 0.0002339 |
| 32140_at | SORL1 | 0.465532 | 0.5020023 | 0.000443 |
| 40667_at | CD6 | 0.4636591 | 0.9774436 | 2.156E−05 |
| 31410_at | TACI | 0.4633867 | 1.0469108 | 0.0001213 |
| 1426_at | SLA | 0.4618421 | 0.7776316 | 3.35E−06 |
| 32193_at | PLXNC1 | 0.4612655 | 0.505618 | 9.079E−05 |
| 39330_s_at | ACTN1 | 0.4608819 | 0.3840683 | 1.167E−06 |
| 35012_at | MNDA | 0.4605263 | 0.4425837 | 3.865E−05 |
| 38646_s_at | REG1A | 0.4570637 | 0.900277 | 7.389E−06 |
| 34949_at | KIAA1048 | 0.4554656 | 1.0931174 | 0.0001806 |
| 35739_at | MTMR3 | 0.4539474 | 0.8289474 | 7.272E−06 |
| 32434_at | MARCKS | 0.4530892 | 1.2768879 | 6.69E−06 |
| 33813_at | TNFRSF1B | 0.4518072 | 1.062936 | 1.603E−05 |
| 37285_at | ALAS2 | 0.4512862 | 1.9426375 | 0.0007351 |
| 39609_at | SIM2 | 0.4511278 | 1.0230934 | 3.587E−05 |
| 39829_at | ARL7 | 0.4511278 | 1.6917293 | 5.719E−06 |
| 40098_at | EHD1 | 0.4497002 | 0.8394404 | 4.185E−08 |
| 35911_r_at | UNK_AJ003147 | 0.4495614 | 0.9247076 | 5.089E−06 |
| 41641_at | C4.4A | 0.4485646 | 0.9868421 | 0.0006562 |
| 36781_at | SERPINA1 | 0.4475091 | 0.8865747 | 4.251E−05 |
| 38081_at | LTA4H | 0.4466299 | 0.5317578 | 1.843E−06 |
| 31525_s_at | UNK_J00153 | 0.4453922 | 1.3547222 | 1.317E−05 |
| 37721_at | DHPS | 0.4428755 | 1.8164313 | 4.969E−06 |
| 40570_at | FOXO1A | 0.4421053 | 0.8368421 | 1.155E−05 |
| 1913_at | CCNG2 | 0.4417293 | 0.4793233 | 1.201E−05 |
| 40260_g_at | RBM9 | 0.4411765 | 0.5340557 | 9.363E−07 |
| 291_s_at | TACSTD2 | 0.4385965 | 0.7368421 | 3.142E−06 |
| 1478_at | ITK | 0.4385965 | 1.1842105 | 4.849E−07 |
| 39351_at | CD59 | 0.4375396 | 0.741915 | 2.96E−07 |
| 40932_at | DKFZP667O2416 | 0.4362881 | 0.6752078 | 1.735E−07 |
| 35785_at | GABARAPL1 | 0.4361733 | 1.2343705 | 1.032E−05 |
| 38976_at | CORO1A | 0.4358145 | 0.3161024 | 8.823E−08 |
| 41627_at | SDF2 | 0.4355717 | 0.4355717 | 0.0009381 |
| 37625_at | IRF4 | 0.4342105 | 0.5328947 | 9.737E−06 |
| 35520_at | CLDN9 | 0.4342105 | 0.6789474 | 0.0004536 |
| 38225_at | KCNH2 | 0.4325883 | 0.7137707 | 3E−07 |
| 41038_at | NCF2 | 0.4293629 | 0.5771006 | 0.000186 |
| 33963_at | AZU1 | 0.4282155 | 0.538773 | 1.147E−06 |
| 37536_at | CD83 | 0.4274498 | 1.1035185 | 0.0004906 |
| 39929_at | KIAA0922 | 0.4251012 | 0.6072874 | 2.17E−06 |
| 296_at | FKBP1A | 0.4245909 | 1.227333 | 0.0003299 |
| 110_at | CSPG4 | 0.4243421 | 1.2631579 | 5.212E−07 |
| 39331_at | TUBB | 0.4241948 | 1.4316575 | 0.0005476 |
| 39733_at | HERPUD1 | 0.4215636 | 0.6208482 | 6.916E−09 |
| 35601_at | UNK_L00022 | 0.4210526 | 1.9684211 | 8.47E−06 |
| 37405_at | SELENBP1 | 0.420913 | 1.8072037 | 0.0001048 |
| 1389_at | MME | 0.4203691 | 0.7484621 | 1.258E−05 |
| 36155_at | KIAA0275 | 0.4202037 | 1.3688455 | 1.826E−05 |
| 32675_at | BST1 | 0.4184211 | 0.4223684 | 4.188E−07 |
| 32067_at | CREM | 0.4179567 | 1.1764706 | 9.959E−06 |
| 31496_g_at | SCYC2 | 0.4155125 | 0.5193906 | 0.0002606 |
| 39729_at | PRDX2 | 0.4145258 | 1.0769338 | 0.0002097 |
| 37061_at | CHIT1 | 0.4139254 | 0.7894737 | 2.886E−07 |
| 1104_s_at | HSPA1A | 0.4131443 | 0.388601 | 0.0004659 |
| 32673_at | BTN2A1 | 0.4102167 | 0.8049536 | 0.0001146 |
| 32649_at | TCF7 | 0.4093567 | 0.9502924 | 0.0005313 |
| 33752_at | NS1-BP | 0.4088346 | 0.7683271 | 3.875E−07 |
| 33979_at | RNASE3 | 0.4056905 | 0.3334776 | 1.97E−06 |
| 39908_at | TAF6L | 0.4050164 | 2.3992599 | 1.464E−05 |
| 39221_at | LILRB2 | 0.4024768 | 0.4334365 | 1.282E−05 |
| 32254_at | VAMP2 | 0.4024165 | 0.5375794 | 7.274E−08 |
| 31930_f_at | RHCE | 0.4023769 | 1.0135823 | 0.0002026 |
| 40739_at | CA4 | 0.4004577 | 0.6636156 | 4.719E−07 |
| 38906_at | SPTA1 | 0.4004577 | 1.0183066 | 2.177E−05 |
| 1105_s_at | TRB@ | 0.3996101 | 1.2207602 | 2.25E−05 |
| 33757_f_at | PSG11 | 0.3984962 | 0.3834586 | 1.474E−05 |
| 31692_at | HSPA1B | 0.3947368 | 0.4251012 | 4.523E−05 |
| 38417_at | AMPD2 | 0.3947368 | 0.7002288 | 6.51E−07 |
| 32066_g_at | CREM | 0.3947368 | 1.0696095 | 4.351E−05 |
| 1117_at | CDA | 0.3922542 | 0.5561072 | 9.035E−08 |
| 41409_at | ICB-1 | 0.3897402 | 0.5996003 | 1.947E−05 |
| 1353_g_at | IL8RA | 0.3896104 | 0.6254272 | 0.0003481 |
| 35530_f_at | IGL@ | 0.3854123 | 0.7801492 | 0.0005962 |
| 1780_at | FGR | 0.3851559 | 0.5806081 | 1.006E−09 |
| 33758_f_at | PSG11 | 0.3832715 | 0.4455121 | 1.086E−07 |
| 31931_f_at | RHCE | 0.3824013 | 0.9436678 | 0.0001234 |
| 40699_at | CD8A | 0.3822715 | 1.0387812 | 2.068E−05 |
| 37024_at | PIG7 | 0.3803828 | 0.8660287 | 1.557E−07 |
| 33439_at | TCF8 | 0.3802953 | 0.5680359 | 1.298E−06 |
| 1106_s_at | TRA@ | 0.3739612 | 0.9903047 | 1.78E−07 |
| 38894_g_at | UNK_AL008637 | 0.3726469 | 0.386093 | 2.328E−06 |
| 37105_at | CTSG | 0.3722783 | 0.4390194 | 1.507E−06 |
| 35763_at | KIAA0540 | 0.3692699 | 0.5857385 | 0.0001956 |
| 36338_at | UNK_W28504 | 0.367823 | 0.7446172 | 1.687E−05 |
| 35674_at | PADI2 | 0.3651316 | 0.6019737 | 0.000164 |
| 32606_at | BASP1 | 0.3636901 | 0.6031934 | 4.229E−05 |
| 35367_at | LGALS3 | 0.3630735 | 0.9414579 | 1.074E−05 |
| 35379_at | COL9A1 | 0.3625134 | 1.0875403 | 5.216E−05 |
| 404_at | IL4R | 0.3581118 | 0.7080846 | 1.229E−06 |
| 36459_at | ENPP4 | 0.354901 | 0.6084017 | 4.977E−07 |
| 32901_s_at | NPM1P14 | 0.3541022 | 0.5688854 | 6.017E−05 |
| 37623_at | NR4A2 | 0.353902 | 0.4355717 | 0.0004838 |
| 34965_at | CST7 | 0.3510002 | 0.8271204 | 1.339E−06 |
| 35714_at | PDXK | 0.3446998 | 0.3891772 | 5.449E−06 |
| 33238_at | UNK_U23852 | 0.3444976 | 0.8325359 | 2.384E−05 |
| 675_at | IFITM1 | 0.3407009 | 1.1539871 | 8.102E−05 |
| 1150_at | PTPRE | 0.3391028 | 0.8742494 | 4.312E−08 |
| 595_at | TNFAIP3 | 0.3383459 | 1.0352805 | 0.0002947 |
| 38895_i_at | NCF4 | 0.3383459 | 0.3233083 | 4E−05 |
| 40876_at | GYG | 0.3354416 | 0.4506438 | 4.617E−06 |
| 33309_at | UNK_AA521060 | 0.3340081 | 0.4402834 | 6.027E−05 |
| 649_s_at | CXCR4 | 0.3329106 | 0.5545339 | 1.837E−06 |
| 32916_at | PTPRE | 0.3320216 | 0.7894737 | 3.836E−06 |
| 33143_s_at | SLC16A3 | 0.3282548 | 0.3739612 | 0.0001067 |
| 40215_at | UGCG | 0.3277061 | 1.4597815 | 0.0005417 |
| 37420_i_at | UNK_AL022723 | 0.3264826 | 0.8713787 | 1.805E−08 |
| 34832_s_at | KIAA0763 | 0.3222342 | 0.8485499 | 1.626E−08 |
| 36488_at | EGFL5 | 0.3217478 | 0.387289 | 6.435E−06 |
| 39706_at | CPNE3 | 0.3207237 | 0.4111842 | 2.066E−05 |
| 35566_f_at | IGHM | 0.3202847 | 0.6152838 | 2.81E−05 |
| 35013_at | LBP | 0.3192407 | 0.7635893 | 1.464E−07 |
| 40419_at | EPB72 | 0.3188259 | 0.7507591 | 2.708E−11 |
| 38138_at | S100A11 | 0.3174173 | 0.3947368 | 0.0003843 |
| 35095_r_at | LILRA3 | 0.3095975 | 0.5417957 | 5.255E−05 |
| 37579_at | PIR121 | 0.3082707 | 0.5075188 | 6.97E−11 |
| 679_at | CTSG | 0.301199 | 0.4037656 | 1.471E−07 |
| 1797_at | CDKN2D | 0.3007519 | 0.647452 | 1.703E−08 |
| 330_s_at | TUBA1 | 0.2960526 | 0.6217105 | 9.294E−09 |
| 33371_s_at | RAB31 | 0.2955466 | 0.5303644 | 1.314E−06 |
| 2002_s_at | BCL2A1 | 0.2944862 | 0.8897243 | 0.0001119 |
| 32793_at | TRB@ | 0.2914165 | 1.0014129 | 5.826E−07 |
| 38017_at | CD79A | 0.2882206 | 0.8521303 | 0.000402 |
| 36674_at | SCYA4 | 0.2858439 | 0.4083485 | 0.0006351 |
| 41694_at | BN51T | 0.2835126 | 0.6062809 | 3.325E−11 |
| 32607_at | BASP1 | 0.2834008 | 0.5237854 | 1.318E−08 |
| 34509_at | MGAM | 0.2809991 | 0.5352364 | 0.0003162 |
| 40171_at | FRAT2 | 0.2808195 | 0.2331332 | 1.22E−05 |
| 38194_s_at | IGKC | 0.2790896 | 0.4314367 | 0.0003358 |
| 41096_at | S100A8 | 0.2769991 | 0.6864532 | 2.61E−10 |
| 36479_at | GAS11 | 0.2766532 | 0.5398111 | 7.537E−10 |
| 35449_at | KLRB1 | 0.2763158 | 0.9769737 | 1.3E−06 |
| 37701_at | RGS2 | 0.2729811 | 0.6312687 | 1.769E−05 |
| 36983_f_at | HP | 0.2722323 | 0.3266788 | 0.0005412 |
| 36979_at | SLC2A3 | 0.2683363 | 0.7456925 | 1.597E−07 |
| 40159_r_at | NCF1 | 0.2677108 | 0.1639046 | 5.219E−06 |
| 32794_g_at | TRB@ | 0.2617506 | 0.9330144 | 8.238E−05 |
| 34498_at | VNN2 | 0.2535302 | 0.6931964 | 8.91E−06 |
| 34105_f_at | IGHG3 | 0.2529206 | 0.3703481 | 0.0003575 |
| 41166_at | IGHM | 0.2494619 | 0.5693602 | 1.851E−06 |
| 34095_f_at | UNK_U80114 | 0.2467105 | 0.3700658 | 2.808E−05 |
| 189_s_at | PLAUR | 0.24344 | 0.5278325 | 5.635E−06 |
| 2090_i_at | UNK_H12458 | 0.2432028 | 0.6211025 | 4.197E−11 |
| 39872_at | UNK_AL031588 | 0.242915 | 0.652834 | 1.371E−06 |
| 37145_at | GNLY | 0.2421053 | 1.0210526 | 0.0002205 |
| 35536_at | ECE2 | 0.2416062 | 0.6703721 | 0.0002898 |
| 37864_s_at | IGHG3 | 0.2407991 | 0.4318942 | 0.0007698 |
| 307_at | ALOX5 | 0.2404488 | 0.5850922 | 9.063E−10 |
| 40729_s_at | UNK_Y14768 | 0.2395033 | 1.0245417 | 3.21E−10 |
| 37121_at | NKG7 | 0.2378331 | 0.5582471 | 4.201E−08 |
| 38868_at | FCAR | 0.2356638 | 0.5302435 | 8.868E−06 |
| 36591_at | TUBA1 | 0.2329033 | 0.6593218 | 7.198E−11 |
| 31315_at | IGL@ | 0.2306904 | 0.6049214 | 5.861E−05 |
| 39128_r_at | PPP2R4 | 0.2269737 | 0.375 | 6.867E−06 |
| 41827_f_at | UNK_AI932613 | 0.2254155 | 0.3407202 | 1.059E−05 |
| 41471_at | S100A9 | 0.224093 | 0.5379747 | 2.65E−09 |
| 35094_f_at | LILRA3 | 0.2208647 | 0.5028195 | 8.558E−07 |
| 38968_at | SH3BP5 | 0.2195578 | 0.4905014 | 8.275E−15 |
| 33849_at | PBEF | 0.2166463 | 0.995104 | 3.814E−06 |
| 37078_at | CD3Z | 0.2129501 | 1.2309557 | 4.499E−06 |
| 32451_at | MS4A3 | 0.2128146 | 0.3130435 | 3.608E−10 |
| 37099_at | ALOX5AP | 0.2114456 | 0.478051 | 1.677E−11 |
| 37200_at | FCGR3B | 0.2111383 | 0.624235 | 0.0007861 |
| 35966_at | QPCT | 0.2105263 | 0.7157895 | 2.151E−07 |
| 37975_at | CYBB | 0.2101261 | 0.1801081 | 0.0002892 |
| 33093_at | IL18RAP | 0.2083333 | 0.2302632 | 0.0001605 |
| 35315_at | ORM1 | 0.2030075 | 0.3338346 | 8.943E−08 |
| 33273_f_at | IGL@ | 0.1959686 | 0.3879379 | 3.492E−05 |
| 33304_at | ISG20 | 0.1958384 | 0.8873929 | 2.359E−08 |
| 33499_s_at | IGHM | 0.1900166 | 0.420751 | 7.758E−05 |
| 37096_at | ELA2 | 0.1859842 | 0.4215984 | 2.613E−15 |
| 33274_f_at | IGL@ | 0.1847086 | 0.3951519 | 2.818E−05 |
| 33500_i_at | UNK_S71043 | 0.1835471 | 0.4319623 | 7.656E−05 |
| 33501_r_at | UNK_S71043 | 0.1823727 | 0.426546 | 6.172E−05 |
| 41164_at | IGHM | 0.1821862 | 0.4932879 | 1.374E−07 |
| 31495_at | SCYC2 | 0.1790559 | 0.3743896 | 1.193E−09 |
| 32275_at | SLPI | 0.1789801 | 0.583524 | 8.607E−10 |
| 31506_s_at | DEFA3 | 0.1770106 | 0.6866596 | 1.16E−10 |
| 37233_at | OLR1 | 0.1762218 | 0.331297 | 1.281E−07 |
| 37066_at | PRTN3 | 0.1741486 | 0.370227 | 2.901E−11 |
| 32529_at | CKAP4 | 0.1726089 | 0.5885682 | 6.187E−10 |
| 38533_s_at | ITGAM | 0.1658255 | 0.2487383 | 8.608E−09 |
| 41165_g_at | IGHM | 0.1609045 | 0.5093379 | 3.789E−09 |
| 39318_at | TCL1A | 0.1475279 | 0.1874003 | 1.723E−05 |
| 36197_at | CHI3L1 | 0.1468788 | 0.6884945 | 2.386E−07 |
| 988_at | CEACAM1 | 0.1389918 | 0.3169014 | 1.905E−06 |
| 31477_at | TFF3 | 0.1324278 | 0.3056027 | 2.883E−08 |
| 37054_at | BPI | 0.1295953 | 0.4641629 | 9.024E−08 |
| 35919_at | TCN1 | 0.1240602 | 0.2180451 | 3.345E−06 |
| 37897_s_at | UNK_AI985964 | 0.1160991 | 0.1764706 | 3.779E−08 |
| 36372_at | HK3 | 0.1148325 | 0.1399522 | 2.374E−07 |
| 266_s_at | CD24 | 0.1084773 | 0.4379269 | 5.846E−08 |
| 36447_at | FCN1 | 0.1046544 | 0.4530343 | 2.343E−09 |
| 1962_at | ARG1 | 0.1009792 | 0.4406365 | 5.456E−08 |
| 36984_f_at | HPR | 0.098472 | 0.2937182 | 9.495E−07 |
| 34319_at | S100P | 0.0958522 | 0.5001743 | 2.392E−09 |
| 36105_at | CEACAM6 | 0.0914953 | 0.4234925 | 4.785E−09 |
| 38326_at | G0S2 | 0.0886728 | 0.6621854 | 5.064E−05 |
| 38615_at | GW112 | 0.0868799 | 0.1775371 | 3.114E−08 |
| 31792_at | ANXA3 | 0.0858726 | 0.4127424 | 2.711E−07 |
| 31793_at | DEFA3 | 0.0775822 | 0.5472095 | 8.209E−10 |
| 33530_at | CEACAM8 | 0.0758328 | 0.2911438 | 1.378E−09 |
| 34546_at | DEFA4 | 0.071914 | 0.4445235 | 2.286E−13 |
| 681_at | MMP8 | 0.0711638 | 0.4203113 | 1.616E−08 |
| 36464_at | SGP28 | 0.0584795 | 0.1776878 | 2.94E−07 |
| 31381_at | PGLYRP | 0.0553506 | 0.1340066 | 2.603E−07 |
| 31859_at | MMP9 | 0.039135 | 0.1550349 | 6.851E−07 |
| 38879_at | S100A12 | 0.0390829 | 0.2344971 | 4.845E−10 |
| 37149_s_at | UNK_U95626 | 0.0319286 | 0.3698401 | 6.402E−11 |
| 36710_at | CAMP | 0.0292477 | 0.207509 | 7.084E−10 |
| 32821_at | LCN2 | 0.0229556 | 0.1896334 | 1.893E−09 |

Table 2 provides the cytogenetic band, gene title, and Unigene and Entrez accession numbers for each AML disease gene depicted in Table 1. The Entrez nucleotide sequence database collects sequences from a variety of sources, such as GenBank, RefSeq and PDB. The database is publicly accessible. The oligonucleotide probes of each qualifier may be derived from the sequence of the Entrez accession number that corresponds to the qualifier.

TABLE 2
Examples of AML Disease Genes

| | Cytogenetic | | Unigene | Entrez Accession |
|---|---|---|---|---|
| Gene Name | Band | Gene Title | No. | No |
| FLT3 | 13q12 | fms-related tyrosine kinase 3 | Hs.385 | U02687 |
| SPINK2 | 4q11 | serine protease inhibitor, Kazal type, 2 (acrosin- | Hs.98243 | X57655 |
| | | trypsin inhibitor) | | |
| H2AFO | 1q21.3 | H2A histone family, member O | Hs.795 | AI885852 |
| HOXB2 | 17q21-q22 | homeo box B2 | Hs.2733 | X16665 |
| ACTA2 | 10q23.3 | actin, alpha 2, smooth muscle, aorta | Hs.195851 | X13839 |
| STAB1 | 3p21.31 | KIAA0246 protein | Hs.301989 | D87433 |
| ADA | 20q12-q13.11 | adenosine deaminase | Hs.1217 | X02994 |
| MIC2 | Xp22.32, | antigen identified by monoclonal antibodies | Hs.177543 | M16279 |
| | Yp11.3 | 12E7, F21 and O13 | | |
| CMAH | 6p22-p23 | cytidine monophosphate-N-acetylneuraminic | Hs.24697 | D86324 |
| | | acid hydroxylase (CMP-N-acetylneuraminate | | |
| | | monooxygenase) | | |
| SNL | 7p22 | singed (Drosophila)-like (sea urchin fascin | Hs.118400 | U03057 |
| | | homolog like) | | |
| RUNX1 | 21q22.3 | runt-related transcription factor 1 (acute | Hs.129914 | D43969 |
| | | myeloid leukemia 1; aml1 oncogene) | | |
| SCHIP1 | 3q25.32 | schwannomin interacting protein 1 | Hs.61490 | AF070614 |
| OA48-18 | 17, 17q21 | acid-inducible phosphoprotein | Hs.278670 | AF069250 |
| DKFZP586A0522 | 12q11 | DKFZP586A0522 protein | Hs.288771 | AL050159 |
| TBXAS1 | 7q34-q35 | thromboxane A synthase 1 (platelet, cytochrome | Hs.2001 | D34625 |
| | | P450, subfamily V) | | |
| INHA | 2q33-q36 | inhibin, alpha | Hs.1734 | M13981 |
| H2AFO | 1q21.3 | H2A histone family, member O | Hs.795 | L19779 |
| PFKP | 10p15.3-p15.2 | phosphofructokinase, platelet | Hs.99910 | D25328 |
| ACADM | 1p31 | acyl-Coenzyme A dehydrogenase, C-4 to C-12 | Hs.79158 | M91432 |
| | | straight chain | | |
| UNK_X57985 | 1q21-q23 | H2B histone family, member Q | Hs.2178 | X57985 |
| P311 | 5q21.3 | P311 protein | Hs.142827 | U30521 |
| TCAP | 17q12 | titin-cap (telethonin) | Hs.343603 | AJ010063 |
| LYL1 | 19p13.2 | lymphoblastic leukemia derived sequence 1 | Hs.46446 | M22637 |
| DBP | 19q13.3 | D site of albumin promoter (albumin D-box) | Hs.155402 | U48213 |
| | | binding protein | | |
| FLJ21174 | Xq22.1, Xq22.1-q22.3 | AA149307: zl25h05.s1 | Hs.194329 | AA149307 |
| | | Soares_pregnant_uterus_NbHPU Homo sapiens | | |
| | | cDNA clone IMAGE: 503001 3′, mRNA | | |
| | | sequence. | | |
| LOC51097 | 1q44 | ESTs, Highly similar to CGI-49 protein | Hs.238126 | AA005018 |
| | | [H. sapiens] | | |
| MYB | 6q22-q23 | v-myb avian myeloblastosis viral oncogene | Hs.1334 | U22376 |
| | | homolog | | |
| HSPB1 | 7p12.3 | heat shock 27 kD protein 1 | Hs.76067 | Z23090 |
| PRKACB | 1p36.1 | protein kinase, cAMP-dependent, catalytic, beta | Hs.87773 | M34181 |
| RUNX1 | 21q22.3 | runt-related transcription factor 1 (acute | Hs.129914 | D43968 |
| | | myeloid leukemia 1; aml1 oncogene) | | |
| GNA15 | 19p13.3 | guanine nucleotide binding protein (G protein), | Hs.73797 | M63904 |
| | | alpha 15 (Gq class) | | |
| LGALS1 | 22q13.1 | lectin, galactoside-binding, soluble, 1 (galectin | Hs.227751 | AI535946 |
| | | 1) | | |
| CALR | 13q14.3, | calreticulin | Hs.16488 | M84739 |
| | 19p13.3-p13.2 | | | |
| HSPCB | 6p12 | heat shock 90 kD protein 1, beta | Hs.74335 | W28616 |
| M6A | 14q11.1 | Homo sapiens m6A methyltransferase (MT- | Hs.268149 | AF014837 |
| | | A70) gene, complete cds | | |
| ITGA4 | 2q31-q32 | integrin, alpha 4 (antigen CD49D, alpha 4 | Hs.40034 | X16983 |
| | | subunit of VLA-4 receptor) | | |
| GPX1 | 3p21.3 | glutathione peroxidase 1 | Hs.76686 | X13710 |
| SOX4 | 17p11.2, 6p22.3 | SRY (sex determining region Y)-box 4 | Hs.83484 | X70683 |
| UNK_AD000092 | 19p13.2 | phenylalanine-tRNA synthetase-like | Hs.23111 | AD000092 |
| YWHAE | 17p13.3 | tyrosine 3-monooxygenase/tryptophan 5- | Hs.79474 | U54778 |
| | | monooxygenase activation protein, epsilon | | |
| | | polypeptide | | |
| IRF5 | 7q32 | interferon regulatory factor 5 | Hs.334450 | U51127 |
| UNK_AL009179 | 6p21.3, 6p22-p21.3 | H2B histone family, member C | Hs.137594, | AL009179 |
| | | | Hs.151506, | |
| | | | Hs.154576, | |
| | | | Hs.180779, | |
| | | | Hs.182138, | |
| | | | Hs.182140, | |
| | | | Hs.352109, | |
| | | | Hs.356901 | |
| UNK_AD000092 | 19p13.2 | phenylalanine-tRNA synthetase-like | Hs.23111 | AD000092 |
| H2BFD | 6p21.3, 6p22-p21.3 | H2B histone family, member D | Hs.154576 | AA873858 |
| MYB | 6q22-q23 | v-myb avian myeloblastosis viral oncogene | Hs.1334 | M15024 |
| | | homolog | | |
| PLAGL1 | 6q24-q25 | pleomorphic adenoma gene-like 1 | Hs.75825 | U81992 |
| DCTD | 4q35.1 | dCMP deaminase | Hs.76894 | L39874 |
| 37501 | Xq24 | KIAA0128 protein; septin 2 | Hs.90998 | D50918 |
| IDH1 | 2q33.3 | isocitrate dehydrogenase 1 (NADP+), soluble | Hs.11223 | AF020038 |
| APEX | 14q11.2-q12 | APEX nuclease (multifunctional DNA repair | Hs.73722 | M80261 |
| | | enzyme) | | |
| IRF5 | 7q32 | interferon regulatory factor 5 | Hs.334450 | U51127 |
| BAX | 19q13.3-q13.4 | BCL2-associated X protein | Hs.159428 | L22475 |
| PPID | 4q31.3 | peptidylprolyl isomerase D (cyclophilin D) | Hs.143482 | D63861 |
| NOLC1 | 10q24.32 | nucleolar phosphoprotein p130 | Hs.75337 | D21262 |
| M6A | 14q11.1 | Homo sapiens m6A methyltransferase (MT- | Hs.268149 | AF014837 |
| | | A70) gene, complete cds | | |
| FADS1 | 11q12.2-q13.1 | Homo sapiens clone 23716 mRNA sequence | Hs.132898 | W26480 |
| ADFP | 9p21.2 | adipose differentiation-related protein; | Hs.3416 | X97324 |
| | | adipophilin | | |
| PRDX1 | 1p34.1 | proliferation-associated gene A (natural killer- | Hs.180909 | X67951 |
| | | enhancing factor A) | | |
| POLD2 | 7p15.1 | polymerase (DNA directed), delta 2, regulatory | Hs.74598 | U21090 |
| | | subunit (50 kD) | | |
| ICAM2 | 17q23-q25 | intercellular adhesion molecule 2 | Hs.347326 | X15606 |
| PTK9L | 3p21.1 | protein tyrosine kinase 9-like (A6-related | Hs.6780 | Y17169 |
| | | protein) | | |
| GRHPR | 9q12 | ESTs, Weakly similar to 3-phosphoglycerate | Hs.155742 | W28944 |
| | | dehydrogenase [H. sapiens] | | |
| CDA02 | 3q25.1 | EST, Weakly similar to cDNA EST | Hs.332404 | W27675 |
| | | EMBL: D71941 comes from this gene | | |
| | | [C. elegans] | | |
| HSA249128 | 11p11.2 | AA176780: zp32a10.s1 Stratagene | Hs.14512 | AA176780 |
| | | neuroepithelium (#937231) Homo sapiens | | |
| | | cDNA clone IMAGE: 611130 3′ similar to | | |
| | | contains Alu repetitive element;, mRNA | | |
| | | sequence. | | |
| BAX | 19q13.3-q13.4 | BCL2-associated X protein | Hs.159428 | U19599 |
| TRIP7 | 6q15 | thyroid hormone receptor interactor 7 | Hs.77558 | AA845349 |
| PGPL | Xp22.33 | Homo sapiens mRNA for putative GTP-binding | Hs.372587 | Y14391 |
| | | protein | | |
| H2BFG | 6p21.3 | H2B histone family, member G | Hs.182137 | Z80779 |
| BST2 | 19p13.2 | bone marrow stromal cell antigen 2 | Hs.118110 | D28137 |
| LTC4S | 5q35 | leukotriene C4 synthase | Hs.456 | U50136 |
| LIPA | 10q23.2-q23.3 | lipase A, lysosomal acid, cholesterol esterase | Hs.85226 | X76488 |
| | | (Wolman disease) | | |
| ZNF185 | Xq28 | zinc finger protein 185 (LIM domain) | Hs.16622 | Y09538 |
| PBX3 | 9q33-q34 | pre-B-cell leukemia transcription factor 3 | Hs.294101 | X59841 |
| UNK_AC005546 | 19p13.13 | lymphoblastic leukemia derived sequence 1 | Hs.158947 | AC005546 |
| COMT | 22q11.21 | catechol-O-methyltransferase | Hs.240013 | M58525 |
| NAP1L1 | 12q14.1 | nucleosome assembly protein 1-like 1 | Hs.302649 | M86667 |
| MGC2840 | 11pter-p15.5 | Homo sapiens mRNA for putative | Hs.155356 | AJ224875 |
| | | glucosyltransferase, partial cds | | |
| SH3GLB1 | 1p22 | Chromosome 1 specific transcript KIAA0491 | Hs.136309 | AB007960 |
| UQCRC2 | 16p12 | ubiquinol-cytochrome c reductase core protein | Hs.173554 | J04973 |
| | | II | | |
| DCTD | 4q35.1 | dCMP deaminase | Hs.76894 | L39874 |
| HRMT1L2 | 19q13.3 | HMT1 (hnRNP methyltransferase, S. cerevisiae)- | Hs.20521 | Y10805 |
| | | like 2 | | |
| NME2 | 17q21.3 | non-metastatic cells 2, protein (NM23B) | Hs.275163 | X58965 |
| | | expressed in | | |
| CSNK1A1 | 13q13, 5 | casein kinase 1, alpha 1 | Hs.283738 | L37042 |
| ATIC | 2q35 | 5-aminoimidazole-4-carboxamide | Hs.90280 | D82348 |
| | | ribonucleotide formyltransferase/IMP | | |
| | | cyclohydrolase | | |
| CEBPA | 19q13.1 | CCAAT/enhancer binding protein (C/EBP), | Hs.76171 | Y11525 |
| | | alpha | | |
| HSPCB | 6p12 | heat shock 90 kD protein 1, beta | Hs.74335 | J04988 |
| RANBP7 | 11p15.3 | RAN binding protein 7 | Hs.5151 | AF098799 |
| KIAA0620 | 3q22.1 | KIAA0620 protein | Hs.301685 | AB014520 |
| ETS2 | 21q22.2 | v-ets avian erythroblastosis virus E26 oncogene | Hs.85146 | J04102 |
| | | homolog 2 | | |
| PPIH | 11 | cyclophilin | Hs.9880 | AF016371 |
| ECHS1 | 10q26.2-q26.3 | enoyl Coenzyme A hydratase, short chain, 1, | Hs.76394 | D13900 |
| | | mitochondrial | | |
| CLECSF2 | 12p13-p12 | C-type (calcium dependent, carbohydrate- | Hs.85201 | X96719 |
| | | recognition domain) lectin, superfamily member | | |
| | | 2 (activation-induced) | | |
| C20ORF14 | 20q13.33 | putative mitochondrial outer membrane protein | Hs.31334 | AF026031 |
| | | import receptor | | |
| MN7 | 15q11-q13 | Homo sapiens D15F37 pseudogene, S3 allele, | Hs.286132 | AF041080 |
| | | mRNA sequence | | |
| CCNG1 | 5q32-q34 | cyclin G1 | Hs.79101 | X77794 |
| PTPN7 | 1q32.1 | protein tyrosine phosphatase, non-receptor type 7 | Hs.35 | M64322 |
| HSD17B4 | 5q21 | hydroxysteroid (17-beta) dehydrogenase 4 | Hs.75441 | X87176 |
| KIAA0594 | 9q21.12 | KIAA0594 protein | Hs.103283 | AB011166 |
| JWA | 3p14 | vitamin A responsive; cytoskeleton related | Hs.92384 | AF070523 |
| IMPDH2 | 3p21.2 | IMP (inosine monophosphate) dehydrogenase 2 | Hs.75432 | L33842 |
| MYB | 6q22-q23 | v-myb avian myeloblastosis viral oncogene | Hs.1334 | U22376 |
| | | homolog | | |
| KIAA0014 | 8q24.3 | KIAA0014 gene product | Hs.155650 | D25216 |
| ALCAM | 3q13.1 | activated leucocyte cell adhesion molecule | Hs.10247 | Y10183 |
| H2BFH | 21q22.3, 6p21.3, | H2B histone family, member H | Hs.137594, | Z80780 |
| | 6p21.31, | | Hs.151506, | |
| | 6p21.33, 6p22-p21.3 | | Hs.154576, | |
| | | | Hs.180779, | |
| | | | Hs.182137, | |
| | | | Hs.182138, | |
| | | | Hs.247817, | |
| | | | Hs.285735, | |
| | | | Hs.352109, | |
| | | | Hs.356901, | |
| | | | Hs.367748 | |
| XPNPEPL | 10q25.3 | X-prolyl aminopeptidase (aminopeptidase P)- | Hs.284202 | X95762 |
| | | like | | |
| TFEC | 7q21.2-q21.3 | transcription factor EC | Hs.113274 | D43945 |
| NDUFB5 | 3q27.1 | NADH dehydrogenase (ubiquinone) 1 beta | Hs.19236 | AF047181 |
| | | subcomplex, 5 (16 kD, SGDH) | | |
| H2BFE | 6p22-p21.3 | H2B histone family, member E | Hs.182432 | Z83738 |
| AKR1A1 | 1p33-p32 | aldo-keto reductase family 1, member A1 | Hs.89529 | J04794 |
| | | (aldehyde reductase) | | |
| CCT3 | 1q23 | chaperonin containing TCP1, subunit 3 | Hs.1708 | X74801 |
| | | (gamma) | | |
| SCGF | 19q13.3 | stem cell growth factor; lymphocyte secreted C- | Hs.105927 | AF020044 |
| | | type lectin | | |
| ACADVL | 17p13-p11 | acyl-Coenzyme A dehydrogenase, very long | Hs.82208 | L46590 |
| | | chain | | |
| MRPS18B | 6p21.3 | Homo sapiens mRNA; cDNA | Hs.274417 | AL050361 |
| | | DKFZp564H0223 (from clone | | |
| | | DKFZp564H0223) | | |
| UNK_U78027 | Xq21.33-q22 | Human BTK region clone ftp-3 mRNA | Hs.159494 | U78027 |
| H2B/S | 6p21.33 | Homo sapiens mRNA for for histone H2B, | Hs.247817 | AJ223352 |
| | | clone pjG4-5-14 | | |
| TFAP4 | 16p13 | transcription factor AP-4 (activating enhancer- | Hs.3005 | S73885 |
| | | binding protein 4) | | |
| IF116 | 1q22 | interferon, gamma-inducible protein 16 | Hs.155530 | M63838 |
| TARDBP | 1p36.22 | TAR DNA binding protein | Hs.193989 | AL050265 |
| CCT6A | 7p14.1 | chaperonin containing TCP1, subunit 6A (zeta | Hs.82916 | L27706 |
| | | 1) | | |
| MTHFD1 | 14q24 | methylenetetrahydrofolate dehydrogenase | Hs.172665 | J04031 |
| | | (NADP+ dependent), methenyltetrahydrofolate | | |
| | | cyclohydrolase, formyltetrahydrofolate | | |
| | | synthetase | | |
| MRPS27 | 5q13.1 | KIAA0264 protein | Hs.122669 | D87453 |
| PEA15 | 1q21.1 | phosphoprotein enriched in astrocytes 15 | Hs.194673 | X86809 |
| LSM2 | 6p21.3 | Homo sapiens mRNA for G7b protein (G7b | Hs.103106 | AJ245416 |
| | | gene, located in the class III region of the major | | |
| | | histocompatibility complex | | |
| CG018 | 13q12-q13 | Novel human gene mapping to chomosome 13 | Hs.22174 | U50527 |
| C21ORF33 | 21q22.3 | ESI (zebrafish) protein, human homolog of | Hs.182423 | U53003 |
| PAICS | 4pter-q21 | multifunctional polypeptide similar to SAICAR | Hs.117950 | X53793 |
| | | synthetase and AIR carboxylase | | |
| H2BFK | 6p21.3 | H2B histone family, member K | Hs.182140 | Z80782 |
| P24B | 15q24-q25 | integral type I protein | Hs.179516 | AL109672 |
| UNK_AI808712 | | Homo sapiens mRNA; cDNA DKFZp586L141 | | AI808712 |
| | | (from clone DKFZp586L141) | | |
| FLJ10849 | 4q13.3 | T75292: yc89b05.r1 Soares infant brain 1NIB | Hs.8768 | T75292 |
| | | Homo sapiens cDNA clone IMAGE: 23231 5′, | | |
| | | mRNA sequence. | | |
| DBI | 2q12-q21 | diazepam binding inhibitor (GABA receptor | Hs.78888 | AI557240 |
| | | modulator, acyl-Coenzyme A binding protein) | | |
| KIAA0068 | 15q11 | KIAA0068 protein | Hs.77257 | D38549 |
| NDUFS4 | 5q11.1 | NADH dehydrogenase (ubiquinone) Fe—S | Hs.10758 | AA203303 |
| | | protein 4 (18 kD) (NADH-coenzyme Q | | |
| | | reductase) | | |
| KATNB1 | 16q13 | katanin p80 (WD40-containing) subunit B 1 | Hs.275675 | AF052432 |
| KDELR1 | 19q13.3 | KDEL (Lys-Asp-Glu-Leu) endoplasmic | Hs.78040 | X55885 |
| | | reticulum protein retention receptor 1 | | |
| KIAA0649 | 9q34.3 | KIAA0649 gene product | Hs.26163 | AB014549 |
| PIP5K2B | 17q12 | phosphatidylinositol-4-phosphate 5-kinase, type | Hs.6335 | U85245 |
| | | II, beta | | |
| CHC1 | 1p36.1 | chromosome condensation 1 | Hs.84746 | X12654 |
| SDHD | 11q23 | succinate dehydrogenase complex, subunit D, | Hs.168289 | AB006202 |
| | | integral membrane protein | | |
| C5ORF8 | 5q35.3 | endoplasmic reticulum glycoprotein | Hs.75864 | U10362 |
| AK2 | 1p34 | adenylate kinase 2 | Hs.171811 | U54645 |
| MACF1 | 1p32-p31 | actin binding protein; macrophin (microfilament | Hs.108258 | AB007934 |
| | | and actin filament cross-linker protein) | | |
| KIAA0179 | 21q22.3 | KIAA0179 protein | Hs.152629 | D80001 |
| M11S1 | 11p13 | membrane component, chromosome 11, surface | Hs.278672 | Z48042 |
| | | marker 1 | | |
| LMO2 | 11p13 | LIM domain only 2 (rhombotin-like 1) | Hs.184585 | X61118 |
| POP5 | 12q24.23 | ESTs, Highly similar to HSPC004 [H. sapiens] | Hs.279913 | AI827793 |
| PMX1 | 10q24.31, 1q24 | paired mesoderm homeo box 1 | Hs.155606 | M95929 |
| SYPL | 7q11.23 | synaptophysin-like protein | Hs.80919 | X68194 |
| ATP6A1 | 3q13.31 | ESTs, Moderately similar to alternatively | Hs.281866 | AA056747 |
| | | spliced product using exon 13A [H. sapiens] | | |
| PAI-RBP1 | 1p31-p22 | DKFZP564M2423 protein | Hs.165998 | AL080119 |
| ZNF146 | 19q13.1 | zinc finger protein 146 | Hs.301819 | X70394 |
| MPI | 15q22-qter | mannose phosphate isomerase | Hs.75694 | X76057 |
| FABP5 | 8q21.13 | fatty acid binding protein 5 (psoriasis- | Hs.153179 | M94856 |
| | | associated) | | |
| TARS | 5p13-cen | threonyl-tRNA synthetase | Hs.84131 | M63180 |
| KIAA0546 | 12q13.3 | KIAA0546 protein | Hs.26764 | AB011118 |
| CNIL | 14q22.1 | cornichon-like | Hs.201673 | AF104398 |
| NPIP | | nuclear pore complex interacting protein | | AC002045 |
| PRH1 | 12p13.2 | AI864120: wg64a06.x1 | Hs.278469 | AI864120 |
| | | Soares_NSF_F8_9W_OT_PA_P_S1 Homo | | |
| | | sapiens cDNA clone IMAGE: 2369842 3′, | | |
| | | mRNA sequence. | | |
| KIAA1018 | 15q12 | KIAA1018 protein | Hs.5400 | AB023235 |
| UNK_U78027 | Xq22 | Human BTK region clone ftp-3 mRNA | Hs.69089 | U78027 |
| FNTA | 8p22-q11 | farnesyltransferase, CAAX box, alpha | Hs.356463 | L10413 |
| CLNS1A | 11q13.5-q14 | chloride channel, nucleotide-sensitive, 1A | Hs.84974 | X91788 |
| CDK2AP1 | 12q24.31 | deleted in oral cancer (mouse, homolog) 1 | Hs.3436 | AF006484 |
| RCL | 6p12.3 | putative c-Myc-responsive | Hs.109752 | W94101 |
| HNRPDL | 4q13-q21 | heterogeneous nuclear ribonucleoprotein D-like | Hs.170311 | D89678 |
| AMD1 | 6q21-q22 | S-adenosylmethionine decarboxylase 1 | Hs.262476 | M21154 |
| SNRPA | 19q13.1 | small nuclear ribonucleoprotein polypeptide A | Hs.173255 | M60784 |
| MRPL3 | 3q21-q23 | ribosomal protein, mitochondrial, L3 | Hs.79086 | X06323 |
| NMP200 | 11q12.2 | nuclear matrix protein NMP200 related to | Hs.173980 | AJ131186 |
| | | splicing factor PRP19 | | |
| GNB2L1 | 5q35.3 | guanine nucleotide binding protein (G protein), | Hs.5662 | W25845 |
| | | beta polypeptide 2-like 1 | | |
| CHC1 | 1p36.1 | chromosome condensation 1 | Hs.84746 | D00591 |
| UNK_AL034428 | 20p12.2-p11.22 | small nuclear ribonucleoprotein polypeptide B″ | Hs.82575 | AL034428 |
| ESD | 13q14.1-q14.2 | esterase D/formylglutathione hydrolase | Hs.82193 | AF112219 |
| RMP | 19q12 | RPB5-mediating protein | Hs.7943 | AB006572 |
| HSPA8 | 11q23.3-q25 | heat shock 70 kD protein 10 (HSC71) | Hs.180414 | W28493 |
| TIMM44 | 19p13.3-p13.2 | translocase of inner mitochondrial membrane 44 | Hs.123178 | AF026030 |
| | | (yeast) homolog | | |
| TSPAN-3 | 15q23 | tetraspan 3 | Hs.100090 | M69023 |
| C14ORF3 | 14q23.3-31 | chromosome 14 open reading frame 3 | Hs.204041 | AJ243310 |
| EIF3S4 | 19p13.2 | eukaryotic translation initiation factor 3, subunit | Hs.28081 | U96074 |
| | | 4 (delta, 44 kD) | | |
| HINT1 | 5q31.2 | histidine triad nucleotide-binding protein | Hs.256697 | U51004 |
| DEAF1 | 11p15.5 | suppressin (nuclear deformed epidermal | Hs.6574 | AF049460 |
| | | autoregulatory factor-1 (DEAF-1)-related) | | |
| BLMH | 17q11.2 | bleomycin hydrolase | Hs.78943 | X92106 |
| PEX10 | 1p36.32 | peroxisome biogenesis factor 10 | Hs.247220 | AA194159 |
| UNK_M60556 | 14q24 | transforming growth factor, beta 3 | Hs.2025 | M60556 |
| GA17 | X | dendritic cell protein | Hs.69469 | AF064603 |
| KIAA0906 | 3p25.1 | KIAA0906 protein | Hs.56966 | AB020713 |
| LGALS8 | 1q42-q43 | lectin, galactoside-binding, soluble, 8 (galectin | Hs.4082 | L78132 |
| | | 8) | | |
| MPG | 16p13.3 | N-methylpurine-DNA glycosylase | Hs.79396 | M74905 |
| ATP5B | 12p13-qter | ATP synthase, H+ transporting, mitochondrial | Hs.25 | W27997 |
| | | F1 complex, beta polypeptide | | |
| ARHGEF2 | 1q21-q22 | guanine nucleotide regulatory factor | Hs.337774 | AB014551 |
| G3BP | 5q33.1 | Ras-GTPase-activating protein SH3-domain- | Hs.220689 | U32519 |
| | | binding protein | | |
| ITPA | 20p | Homo sapiens putative oncogene protein | Hs.6817 | AF026816 |
| | | mRNA, partial cds | | |
| TARBP1 | 1q42.3 | TAR (HIV) RNA-binding protein 1 | Hs.151518 | U38847 |
| CASP4 | 11q22.2-q22.3 | caspase 4, apoptosis-related cysteine protease | Hs.74122 | U28014 |
| HSU79274 | 12q24.11 | protein predicted by clone 23733 | Hs.150555 | U79274 |
| DDX30 | 3p21.31 | KIAA0890 protein | Hs.323462 | AB020697 |
| AK2 | 1p34 | adenylate kinase 2 | Hs.171811 | U84371 |
| DKFZP564M182 | 16p13.3 | DKFZP564M182 protein | Hs.85963 | AJ007398 |
| SORD | 15q15.3 | sorbitol dehydrogenase | Hs.878 | L29254 |
| UNK_AL022398 | 1q32.3-q41 | AL022398: Homo sapiens DNA sequence from | Hs.261373 | AL022398 |
| | | PAC 434O14 on chromosome 1q32.3.-41. | | |
| | | Contains the HSD11B1 gene for | | |
| | | Hydroxysteroid (11-beta) Dehydrogenase 1, the | | |
| | | ADORA2BP adenosine A2b receptor LIKE | | |
| | | pseudogene, the IRF6 gene for Interferon | | |
| | | Regulatory Factor 6 and two novel genes. | | |
| | | Contains ESTs and GSSs, complete sequence. | | |
| 37501 | Xq24 | AI819942: wj88e02.x1 NCI_CGAP_Lym12 | Hs.90998 | AI819942 |
| | | Homo sapiens cDNA clone IMAGE: 2409914 3′ | | |
| | | similar to SW: GBB5_HUMAN O14775 | | |
| | | GUANINE NUCLEOTIDE-BINDING | | |
| | | PROTEIN BETA SUBUNIT 5;, mRNA | | |
| | | sequence. | | |
| KIAA0092 | 11q21 | KIAA0092 gene product | Hs.151791 | D42054 |
| UNK_AL031432 | 1p36.13-p35.1 | Human DNA sequence from clone 465N24 on | Hs.8084 | AL031432 |
| | | chromosome 1p35.1-36.13. Contains two novel | | |
| | | genes, ESTs, GSSs and CpG islands | | |
| PM5 | 16p13.11 | pM5 protein | Hs.227823 | X57398 |
| APLP2 | 11q24 | amyloid beta (A4) precursor-like protein 2 | Hs.279518 | S60099 |
| HHEX | 10q24.1 | hematopoietically expressed homeobox | Hs.118651 | L16499 |
| IGFBP7 | 4q12 | insulin-like growth factor binding protein 7 | Hs.119206 | L19182 |
| AHR | 7p15 | aryl hydrocarbon receptor | Hs.170087 | L19872 |
| MGC5508 | 11q13.1 | Homo sapiens clone 25036 mRNA sequence | Hs.13662 | N53547 |
| TPM4 | 19p13.1 | tropomyosin 4 | Hs.250641 | X05276 |
| NR1H3 | 11q11 | nuclear receptor subfamily 1, group H, member 3 | Hs.370969 | U22662 |
| HSPCB | 6p12 | heat shock 90 kD protein 1, beta | Hs.74335 | M16660 |
| ATR | 3q22-q24 | ataxia telangiectasia and Rad3 related | Hs.77613 | U49844 |
| HADHSC | 4q22-q26 | L-3-hydroxyacyl-Coenzyme A dehydrogenase, | Hs.8110 | X96752 |
| | | short chain | | |
| NME1 | 17q21.3 | non-metastatic cells 1, protein (NM23A) | Hs.118638 | X17620 |
| | | expressed in | | |
| UNK_D28423 | | D28423: Human mRNA for pre-mRNA splicing | | D28423 |
| | | factor SRp20, 5′UTR (sequence from the 5′cap | | |
| | | to the start codon). | | |
| OGT | Xq13 | O-linked N-acetylglucosamine (GlcNAc) | Hs.100293 | AL050366 |
| | | transferase (UDP-N- | | |
| | | acetylglucosamine: polypeptide-N- | | |
| | | acetylglucosaminyl transferase) | | |
| UNK_AC004080 | | Homo sapiens homeobox protein (HOX-1.3) | | AC004080 |
| | | gene, complete cds | | |
| RPL22 | | ribosomal protein L22 | | X59357 |
| MDH1 | 2p16 | malate dehydrogenase 1, NAD (soluble) | Hs.75375 | D55654 |
| TOMM70A | 3q12.3 | translocase of outer mitochondrial membrane 70 | Hs.21198 | AB018262 |
| | | (yeast) homolog A | | |
| HCS | 7p21.2, Xq22.1 | cytochrome c-1 | Hs.169248 | D00265 |
| UNK_AL008726 | 20q13.1 | protective protein for beta-galactosidase | Hs.118126 | AL008726 |
| | | (galactosialidosis) | | |
| TCFL1 | 1q21 | transcription factor-like 1 | Hs.2430 | D43642 |
| DHPS | 19p13.11-p13.12 | deoxyhypusine synthase | Hs.79064 | U26266 |
| NUP133 | 1q42.13 | Homo sapiens clone 23770 mRNA sequence | Hs.12457 | AF052123 |
| KIAA1104 | 10p15.2 | KIAA1104 protein | Hs.260116 | AB029027 |
| CSF3R | 1p35-p34.3 | colony stimulating factor 3 receptor | Hs.2175 | M59818 |
| | | (granulocyte) | | |
| LOC90355 | 5q15 | Homo sapiens clone 23860 mRNA sequence | Hs.25925 | AF038182 |
| DNAJB1 | 19p13.2 | heat shock 40 kD protein 1 | Hs.82646 | D85429 |
| IL8RA | 2q35 | interleukin 8 receptor, alpha | Hs.194778 | U11870 |
| BLVRB | 19q13.1-q13.2 | biliverdin reductase B (flavin reductase | Hs.76289 | D32143 |
| | | (NADPH)) | | |
| TNFRSF7 | 12p13 | tumor necrosis factor receptor superfamily, | Hs.180841 | M63928 |
| | | member 7 | | |
| TEF | 22q13.2 | thyrotrophic embryonic factor | Hs.121481 | U44059 |
| ZFP36L1 | 14q22-q24 | butyrate response factor 1 (EGF-response factor | Hs.85155 | X79067 |
| | | 1) | | |
| IFITM1 | 11, 11p15.5, | interferon induced transmembrane protein 1 (9-27) | Hs.146360, | J04164 |
| | 8q13.1 | | Hs.174195 | |
| PIP3-E | 6q25.2 | KIAA0403 protein | Hs.185140 | AB007863 |
| NPAS1 | 19q13.2-q13.3 | neuronal PAS domain protein 1 | Hs.79564 | U77968 |
| CSF3R | 1p35-p34.3 | colony stimulating factor 3 receptor | Hs.2175 | M59820 |
| | | (granulocyte) | | |
| CCND3 | 6p21 | cyclin D3 | Hs.83173 | M92287 |
| BTG1 | 12q22 | B-cell translocation gene 1, anti-proliferative | Hs.77054 | X61123 |
| ELL2 | 5q21.2 | ELL-RELATED RNA POLYMERASE II, | Hs.98124 | U88629 |
| | | ELONGATION FACTOR | | |
| ESDN | 3q12.2-q12.3 | Human mRNA for unknown product, partial cds | Hs.173374 | D29810 |
| CAPN3 | 15q15.1-q21.1 | calpain, large polypeptide L3 | Hs.40300 | X85030 |
| ALDH2 | 12q24.2 | aldehyde dehydrogenase 2, mitochondrial | Hs.195432 | X05409 |
| PIG11 | 11q11 | p53-induced protein | Hs.96908 | AF010315 |
| SLA | 8q24 | Src-like-adapter | Hs.75367 | D89077 |
| SNPH | 20p13 | KIAA0374 gene product; syntaphilin | Hs.323833 | AB002372 |
| PPPIR2 | | protein phosphatase 1, regulatory (inhibitor) | | U68111 |
| | | subunit 2 | | |
| PIG7 | 16p13.3-p12 | LPS-induced TNF-alpha factor | Hs.76507 | AL120815 |
| KIAA0513 | 16q23.3 | KIAA0513 gene product | Hs.301658 | AB011085 |
| ELN | 7q11.23 | elastin (supravalvular aortic stenosis, Williams- | Hs.9295 | M36860 |
| | | Beuren syndrome) | | |
| AQP9 | 15q22.1-22.2 | aquaporin 9 | Hs.104624 | AB008775 |
| MYL2 | 12q23-q24.3 | myosin, light polypeptide 2, regulatory, cardiac, | Hs.75535 | X66141 |
| | | slow | | |
| P2Y10 | Xq21.1 | putative purinergic receptor | Hs.296433 | AF000545 |
| CYP4F3 | 19p13.2, 19pter-p13.11 | cytochrome P450, subfamily IVF, polypeptide 3 | Hs.101, | D12620 |
| | | (leukotriene B4 omega hydroxylase) | Hs.106242 | |
| PLSCR1 | 3q23 | phospholipid scramblase 1 | Hs.198282 | AB006746 |
| GZMK | 5q11-q12 | granzyme K (serine protease, granzyme 3; | Hs.3066 | U26174 |
| | | tryptase II) | | |
| HMG2 | 4q31 | high-mobility group (nonhistone chromosomal) | Hs.80684 | X62534 |
| | | protein 2 | | |
| RAP1GA1 | 1p36.1-p35 | KIAA0474 gene product | Hs.75151 | AB007943 |
| RUNX3 | 1p36 | runt-related transcription factor 3 | Hs.170019 | Z35278 |
| CD19 | 16p11.2 | CD19 antigen | Hs.96023 | M28170 |
| UNK_U72507 | | Human 40871 mRNA partial sequence | Hs.234216 | U72507 |
| SORL1 | 11q23.2-q24.2 | sortilin-related receptor, L(DLR class) A | Hs.278571 | Y08110 |
| | | repeats-containing | | |
| CD6 | 11q13 | CD6 antigen | Hs.81226 | X60992 |
| TACI | 17p11.2 | transmembrane activator and CAML interactor | Hs.158341 | AF023614 |
| SLA | 8q24 | Src-like-adapter | Hs.75367 | D89077 |
| PLXNC1 | 12q23.3 | plexin C1 | Hs.286229 | AF030339 |
| ACTN1 | 14q24 | actinin, alpha 1 | Hs.119000 | M95178 |
| MNDA | 1q22 | myeloid cell nuclear differentiation antigen | Hs.153837 | M81750 |
| REG1A | 2p12 | regenerating islet-derived 1 alpha (pancreatic | Hs.1032 | AI763065 |
| | | stone protein, pancreatic thread protein) | | |
| KIAA1048 | 2p24.3-p14 | KIAA1048 protein | Hs.135941 | AB028971 |
| MTMR3 | 22q12.2 | FYVE (Fab1 YGLO23 Vsp27 EEA1 domain) | Hs.63302 | AB002369 |
| | | dual-specificity protein phosphatase | | |
| MARCKS | 6q22.2 | myristoylated alanine-rich protein kinase C | Hs.75607 | D10522 |
| | | substrate (MARCKS, 80K-L) | | |
| TNFRSF1B | 1p36.3-p36.2 | tumor necrosis factor receptor superfamily, | Hs.256278 | AI813532 |
| | | member 1B | | |
| ALAS2 | Xp11.21 | aminolevulinate, delta-, synthase 2 | Hs.323383 | X60364 |
| | | (sideroblastic/hypochromic anemia) | | |
| SIM2 | 21q22.13 | single-minded (Drosophila) homolog 2 | Hs.27311 | U80457 |
| ARL7 | 2q37.2 | ADP-ribosylation factor-like 7 | Hs.111554 | AB016811 |
| EHD1 | 11q13 | EH domain containing 1 | Hs.155119 | AF001434 |
| UNK_J003147 | 16p13.3 | matrix metalloproteinase-like 1 | Hs.198265, | AJ003147 |
| | | | Hs.290222 | |
| C4.4A | 19q13.32 | GPI-anchored metastasis-associated protein | Hs.11950 | AJ223603 |
| | | homolog | | |
| SERPINA1 | 14q32.1 | protease inhibitor 1 (anti-elastase), alpha-1- | Hs.297681 | X01683 |
| | | antitrypsin | | |
| LTA4H | 12q22 | leukotriene A4 hydrolase | Hs.81118 | J03459 |
| UNK_J00153 | 16p13.3 | hemoglobin, alpha 2 | Hs.272572, | J00153 |
| | | | Hs.347939 | |
| DHPS | 19p13.11-p13.12 | deoxyhypusine synthase | Hs.79064 | U79262 |
| FOXO1A | 13q14.1 | forkhead box O1A (rhabdomyosarcoma) | Hs.170133 | AF032885 |
| CCNG2 | 4q13.3 | cyclin G2 | Hs.79069 | U47414 |
| RBM9 | 22q13.1 | RNA binding motif protein 9 | Hs.5011 | AL009266 |
| TACSTD2 | 1p32-p31 | membrane component, chromosome 1, surface | Hs.23582 | J04152 |
| | | marker 1 (40 kD glycoprotein, identified by | | |
| | | monoclonal antibody GA733) | | |
| ITK | 5q31-q32 | IL2-inducible T-cell kinase | Hs.211576 | L10717 |
| CD59 | 11p13 | CD59 antigen p18-20 (antigen identified by | Hs.278573 | M84349 |
| | | monoclonal antibodies 16.3A5, EJ16, EJ30, | | |
| | | EL32 and G344) | | |
| DKFZP667O2416 | 1p35.3 | H18080: ym38h10.s1 Soares infant brain 1NIB | Hs.19066 | H18080 |
| | | Homo sapiens cDNA clone IMAGE: 50768 3′ | | |
| | | similar to contains Alu repetitive | | |
| | | element; contains LTR5 repetitive element;, | | |
| | | mRNA sequence. | | |
| GABARAPL1 | 12p13.1 | ESTs, Moderately similar to MM46 [H. sapiens] | Hs.336429 | W28281 |
| CORO1A | 16q13 | coronin, actin-binding protein, 1A | Hs.109606 | D44497 |
| SDF2 | 17q11.2 | stromal cell-derived factor 2 | Hs.118684 | D50645 |
| IRF4 | 6p25-p23 | interferon regulatory factor 4 | Hs.82132 | U52682 |
| CLDN9 | 16p13.3 | claudin 9 | Hs.296949 | AI701514 |
| KCNH2 | 7q35-q36 | Homo sapiens HERG-USO (HERG) mRNA, | Hs.188021 | AF052728 |
| | | alternatively spliced, partial cds | | |
| NCF2 | 1q25 | neutrophil cytosolic factor 2 (65 kD, chronic | Hs.949 | M32011 |
| | | granulomatous disease, autosomal 2) | | |
| AZU1 | 19p13.3 | azurocidin 1 (cationic antimicrobial protein 37) | Hs.72885 | M96326 |
| CD83 | 6p23 | CD83 antigen (activated B lymphocytes, | Hs.79197 | Z11697 |
| | | immunoglobulin superfamily) | | |
| KIAA0922 | 4q31.23 | KIAA0922 protein | Hs.37892 | AB023139 |
| FKBP1A | | Tubulin, Beta | | AF141349 |
| CSPG4 | 15 | chondroitin sulfate proteoglycan 4 (melanoma- | Hs.9004 | X96753 |
| | | associated) | | |
| TUBB | 6p21.3 | tubulin, beta polypeptide | Hs.336780 | X79535 |
| HERPUD1 | 16q12.2-q13 | KIAA0025 gene product; MMS-inducible gene | Hs.146393 | AF055001 |
| UNK_L00022 | | Human Ig active epsilon1 5′ UT, V-D-J region | | L00022 |
| | | subgroup VH-I, gene | | |
| SELENBP1 | 1q21-q22 | selenium binding protein 1 | Hs.334841 | U29091 |
| MME | 3q25.1-q25.2 | membrane metallo-endopeptidase (neutral | Hs.1298 | J03779 |
| | | endopeptidase, enkephalinase, CALLA, CD10) | | |
| KIAA0275 | 10pter-q25.3 | KIAA0275 gene product | Hs.74583 | D87465 |
| BST1 | 4p15 | bone marrow stromal cell antigen 1 | Hs.169998 | D21878 |
| CREM | 10p12.1-p11.1 | cAMP responsive element modulator | Hs.351252 | S68271 |
| SCYC2 | 1q23, 1q23-q25 | small inducible cytokine subfamily C, member 2 | Hs.174228, | D63789 |
| | | | Hs.3195 | |
| PRDX2 | 13q12 | thioredoxin-dependent peroxide reductase 1 | Hs.146354 | L19185 |
| | | (thiol-specific antioxidant 1, natural killer- | | |
| | | enhancing factor B) | | |
| CHIT1 | 1q31-q32 | chitinase 1 (chitotriosidase) | Hs.91093 | U29615 |
| HSPA1A | 6p21.3 | heat shock 70 kD protein 1 | Hs.274402, | M11717 |
| | | | Hs.8997 | |
| BTN2A1 | 6p22.1 | butyrophilin, subfamily 2, member A1 | Hs.169963 | U90543 |
| TCF7 | 5q31.1 | transcription factor 7 (T-cell specific, HMG- | Hs.169294 | X59871 |
| | | box) | | |
| NS1-BP | 1q25.1-q31.1 | NS1-binding protein | Hs.197298 | AB020657 |
| RNASE3 | 14q24-q31 | ribonuclease, RNase A family, 3 (eosinophil | Hs.73839 | X55990 |
| | | cationic protein) | | |
| TAF6L | 11q13.1 | PCAF associated factor 65 alpha | Hs.131846 | AF069735 |
| LILRB2 | 19q13.4 | leukocyte immunoglobulin-like receptor, | Hs.22405 | AF004231 |
| | | subfamily B (with TM and ITIM domains), | | |
| | | member 2 | | |
| VAMP2 | 17p13.1 | Homo sapiens mRNA; cDNA DKFZp586L1323 | Hs.194534 | AL050223 |
| | | (from clone DKFZp586L1323) | | |
| RHCE | 1p36.11 | Rhesus blood group, D antigen | Hs.278994 | X63096 |
| CA4 | 17q23 | carbonic anhydrase IV | Hs.89485 | M83670 |
| SPTA1 | 1q21 | spectrin, alpha, erythrocytic 1 (elliptocytosis 2) | Hs.1985 | M61877 |
| TRB@ | 7q34 | T cell receptor beta locus | Hs.303157 | M12886 |
| PSG11 | 19q13.2 | pregnancy specific beta-1-glycoprotein 11 | Hs.334408 | M69245 |
| HSPA1B | 6p21.3 | heat shock 70 kD protein 1 | Hs.274402, | M59830 |
| | | | Hs.8997 | |
| AMPD2 | | adenosine monophosphate deaminase 2 | | M91029 |
| | | (isoform L) | | |
| CREM | 10p12.1-p11.1 | cAMP responsive element modulator | Hs.351252 | S68134 |
| CDA | 1p36.2-p35 | cytidine deaminase | Hs.72924 | L27943 |
| ICB-1 | 1p35.3 | basement membrane-induced gene | Hs.10649 | AF044896 |
| IL8RA | 2q35 | interleukin 8 receptor, alpha | Hs.194778 | U11870 |
| IGL@ | 22q11.1-q11.2 | immunoglobulin lambda locus | Hs.181125 | X92997 |
| FGR | 1p36.2-p36.1 | Gardner-Rasheed feline sarcoma viral (v-fgr) | Hs.1422 | M19722 |
| | | oncogene homolog | | |
| PSG11 | 19q13.2 | pregnancy specific beta-1-glycoprotein 11 | Hs.334408 | U25988 |
| RHCE | 1p36.11 | Rhesus blood group, D antigen | Hs.278994 | AI632247 |
| CD8A | 2p12 | CD8 antigen, alpha polypeptide (p32) | Hs.85258 | M12824 |
| PIG7 | 16p13.3-p12 | LPS-induced TNF-alpha factor | Hs.76507 | AF010312 |
| TCF8 | 10p11.2 | transcription factor 8 (represses interleukin 2 | Hs.232068 | D15050 |
| | | expression) | | |
| TRA@ | 14q11.2 | T cell receptor alpha locus | Hs.74647 | M12959 |
| UNK_AL008637 | 22q13.1 | neutrophil cytosolic factor 4 (40 kD) | Hs.196352 | AL008637 |
| CTSG | 14q11.2 | cathepsin G | Hs.100764 | M16117 |
| KIAA0540 | 3p21.31 | Homo sapiens mRNA for KIAA0540 protein, | Hs.64742 | AB011112 |
| | | partial cds | | |
| UNK_W28504 | | W28504: 48e7 Human retina cDNA randomly | Hs.348515 | W28504 |
| | | primed sublibrary Homo sapiens cDNA, mRNA | | |
| | | sequence. | | |
| PADI2 | 1p35.2-p35.1 | peptidyl arginine deiminase, type II | Hs.33455 | AB023211 |
| BASP1 | 5p15.1-p14 | brain acid-soluble protein 1 | Hs.79516 | AA135683 |
| LGALS3 | 14q21-q22 | lectin, galactoside-binding, soluble, 3 (galectin | Hs.621 | AB006780 |
| | | 3) | | |
| COL9A1 | 6q12-q14 | collagen, type IX, alpha 1 | Hs.154850 | X54412 |
| IL4R | 16p11.2-12.1 | interleukin 4 receptor | Hs.75545 | X52425 |
| ENPP4 | 6p12.3 | KIAA0879 protein | Hs.54037 | AB020686 |
| NPM1P14 | 7q22-q31 | nucleophosmin 1 (nucleolar phosphoprotein | Hs.7879 | AC005192 |
| | | B23, numatrin) pseudogene 14 | | |
| NR4A2 | 2q22-q23 | nuclear receptor subfamily 4, group A, member 2 | Hs.82120 | X75918 |
| CST7 | 20p11.21 | cystatin F (leukocystatin) | Hs.143212 | AF031824 |
| PDXK | 21q22.3 | pyridoxal (pyridoxine, vitamin B6) kinase | Hs.38041 | U89606 |
| UNK_U23852 | 1p34.3 | U23852: Human T-lymphocyte specific protein | Hs.1765 | U23852 |
| | | tyrosine kinase p56lck (lck) abberant mRNA, | | |
| | | complete cds. | | |
| IFITM1 | 11 | interferon induced transmembrane protein 1 (9-27) | Hs.146360 | J04164 |
| PTPRE | | protein tyrosine phosphatase, receptor type, | | X54134 |
| | | epsilon polypeptide | | |
| TNFAIP3 | 6q23.1-q25.3 | tumor necrosis factor, alpha-induced protein 3 | Hs.211600 | M59465 |
| NCF4 | 22q13.1 | neutrophil cytosolic factor 4 (40 kD) | Hs.196352 | X77094 |
| GYG | 3q24-q25.1 | glycogenin | Hs.174071 | U31525 |
| UNK_AA521060 | | Homo sapiens clone 23551 mRNA sequence | Hs.184019 | AA521060 |
| CXCR4 | 2q21 | chemokine (C—X—C motif), receptor 4 (fusin) | Hs.89414 | L06797 |
| PTPRE | 10q26 | protein tyrosine phosphatase, receptor type, | Hs.31137 | X54134 |
| | | epsilon polypeptide | | |
| SLC16A3 | 22q12.3-q13.2 | solute carrier family 16 (monocarboxylic acid | Hs.85838 | U81800 |
| | | transporters), member 3 | | |
| UGCG | 9q31 | UDP-glucose ceramide glucosyltransferase | Hs.152601 | D50840 |
| UNK_AL022723 | 6p21.3 | Human DNA sequence from clone 377H14 on | Hs.110309 | AL022723 |
| | | chromosome 6p21.32-22.1. Contains the HLA- | | |
| | | G gene for major histocompatibility complex, | | |
| | | class I, G (HLA 6.0) two MHC class I | | |
| | | pseudogenes, an RPL7A (60S Ribosomal | | |
| | | Protein L7A) pseudogene, a gene for a novel | | |
| | | MHC class 1 protein, an interferon-inducible | | |
| | | protein 1-8U pseudogene, an RPL23A (60S | | |
| | | Ribosomal Protein L23A) pseudogene, an | | |
| | | HCGIX pseudogene, an MICB or . . . | | |
| KIAA0763 | 3p25.1 | KIAA0763 gene product | Hs.4764 | AB018306 |
| EGFL5 | 9q32-q33.3 | EGF-like-domain, multiple 5 | Hs.5599 | AB011542 |
| CPNE3 | 8q21.2 | copine III | Hs.14158 | AB014536 |
| IGHM | | Human rearranged immunoglobulin heavy chain | | AF015128 |
| | | mRNA, partial cds | | |
| LBP | 20q11.23-q12 | AF013512: Homo sapiens lipopolysaccharide | Hs.154078 | AF013512 |
| | | binding protein (LBP) exon 15, complete | | |
| | | sequence and complete cds. | | |
| EPB72 | 9q34.1 | erythrocyte membrane protein band 7.2 | Hs.160483 | X85116 |
| | | (stomatin) | | |
| S100A11 | 1q21, 7q22-q31.1 | S100 calcium-binding protein A11 (calgizzarin) | Hs.256290 | D38583 |
| LILRA3 | 19q13.4 | leukocyte immunoglobulin-like receptor, | Hs.113277 | AF025527 |
| | | subfamily A (without TM domain), member 3 | | |
| PIR121 | 5q34 | p53 inducible protein | Hs.258503 | L47738 |
| CTSG | 14q11.2 | cathepsin G | Hs.100764 | J04990 |
| CDKN2D | 19p13 | cyclin-dependent kinase inhibitor 2D (p19, | Hs.29656 | U40343 |
| | | inhibits CDK4) | | |
| TUBA1 | | tubulin, alpha 1 (testis specific) | | X06956 |
| RAB31 | 18p11.3 | RAB31, member RAS oncogene family | Hs.223025 | U59877 |
| BCL2A1 | 15q24.3 | BCL2-related protein A1 | Hs.227817 | U27467 |
| TRB@ | 7q34 | T cell receptor beta locus | Hs.303157 | X00437 |
| CD79A | 19q13.2 | CD79A antigen (immunoglobulin-associated | Hs.79630 | U05259 |
| | | alpha) | | |
| SCYA4 | 17q12 | small inducible cytokine A4 (homologous to | Hs.75703 | J04130 |
| | | mouse Mip-1b) | | |
| BN51T | 8q21 | BN51 (BHK21) temperature sensitivity | Hs.1276 | M17754 |
| | | complementing | | |
| BASP1 | 5p15.1-p14 | brain acid-soluble protein 1 | Hs.79516 | AF039656 |
| MGAM | 7q32.3 | maltase-glucoamylase (alpha-glucosidase) | Hs.122785 | AF016833 |
| FRAT2 | 10q23-q24.1 | GSK-3 binding protein FRAT2 | Hs.140720 | AF062739 |
| IGKC | 2p12 | immunoglobulin kappa variable 1D-8 | Hs.156110 | M63438 |
| S100A8 | 1q21 | S100 calcium-binding protein A8 (calgranulin | Hs.100000 | AI126134 |
| | | A) | | |
| GAS11 | 16q24.3 | growth arrest specific 11 | Hs.54877 | AF050078 |
| KLRB1 | 12p13 | killer cell lectin-like receptor subfamily B, | Hs.169824 | U11276 |
| | | member 1 | | |
| RGS2 | 1q31 | regulator of G-protein signalling 2, 24 kD | Hs.78944 | L13463 |
| HP | 16q22.1 | haptoglobin | Hs.75990 | X00442 |
| SLC2A3 | 12p13.3 | solute carrier family 2 (facilitated glucose | Hs.7594 | M20681 |
| | | transporter), member 3 | | |
| NCF1 | 7q11.23 | neutrophil cytosolic factor 1 (47 kD, chronic | Hs.1583 | M55067 |
| | | granulomatous disease, autosomal 1) | | |
| TRB@ | 7q34 | T cell receptor beta locus | Hs.303157 | X00437 |
| VNN2 | 6q23-q24 | Vanin 2 | Hs.121102 | D89974 |
| IGHG3 | 14q32.33 | Homo sapiens isolate RP immunoglobulin | Hs.300697 | AI147237 |
| | | heavy chain FW2-JH region gene, partial cds | | |
| IGHM | 14q32.33 | immunoglobulin heavy constant mu | Hs.153261 | X58529 |
| UNK_U80114 | | Human immunoglobulin heavy chain variable | | U80114 |
| | | region (V4-31) gene, partial cds | | |
| PLAUR | 19q13 | plasminogen activator, urokinase receptor | Hs.179657 | U09937 |
| UNK_H12458 | | yj12d03.s1 Soares placenta Nb2HP Homo | | H12458 |
| | | sapiens cDNA clone IMAGE: 148517 3′ similar | | |
| | | to SP: WNT6_MOUSE P22727 WNT-6 | | |
| | | PROTEIN;, mRNA sequence. | | |
| UNK_AL031588 | 22q13.2-q13.3 | Human DNA sequence from clone 1163J1 on | Hs.122552 | AL031588 |
| | | chromosome 22q13.2-13.33. Contains the 3′ | | |
| | | part of a gene for anovel KIAA0279 LIKE | | |
| | | EGF-like domain containing protein (similar to | | |
| | | mouse Celsr1, rat MEGF2), a novel gene for a | | |
| | | protein similar to C. elegans B0035.16 and | | |
| | | bacterial tRNA (5-Methylaminomethyl-2- | | |
| | | thiouridylate)-Methyltransferases, and the 3′ | | |
| | | part of a novel gene for a protein similar to | | |
| | | mouse B99 . . . | | |
| GNLY | 2p12-q11 | granulysin | Hs.105806 | M85276 |
| ECE2 | | KIAA0604 gene product | Hs.129801 | AB011176 |
| IGHG3 | 14q32.33 | immunoglobulin heavy constant gamma 3 (G3m | Hs.300697 | Y14737 |
| | | marker) | | |
| ALOX5 | 10q11.2 | arachidonate 5-lipoxygenase | Hs.89499 | J03600 |
| UNK_Y14768 | 6p21.3 | Homo sapiens DNA, cosmid clones TN62 and | Hs.890 | Y14768 |
| | | TN82 | | |
| NKG7 | 19q13.41 | natural killer cell group 7 sequence | Hs.10306 | S69115 |
| FCAR | 19q13.2-q13.4 | Fc fragment of IgA, receptor for | Hs.193122 | U43774 |
| TUBA1 | 2q36.2 | tubulin, alpha 1 (testis specific) | Hs.75318 | X06956 |
| IGL@ | 22q11.1-q11.2 | Human immunoglobulin (mAb59) light chain V | Hs.181125 | D84143 |
| | | region mRNA, partial sequence | | |
| PPP2R4 | 9q34 | protein phosphatase 2A, regulatory subunit B′ | Hs.236963 | X73478 |
| | | (PR 53) | | |
| UNK_AI932613 | | Human rearranged immunoglobulin lambda | Hs.350074 | AI932613 |
| | | light chain mRNA | | |
| S100A9 | 1q21 | S100 calcium-binding protein A9 (calgranulin | Hs.112405 | W72424 |
| | | B) | | |
| LILRA3 | 19q13.4 | leukocyte immunoglobulin-like receptor, | Hs.113277 | AF025527 |
| | | subfamily A (without TM domain), member 3 | | |
| SH3BP5 | 3p24.3 | SH3-domain binding protein 5 (BTK- | Hs.109150 | AB005047 |
| | | associated) | | |
| PBEF | 7q11.23 | pre-B-cell colony-enhancing factor | Hs.239138 | U02020 |
| CD3Z | 1q22-q23 | CD3Z antigen, zeta polypeptide (TiT3 complex) | Hs.97087 | J04132 |
| MS4A3 | 11q12-q13.1 | membrane-spanning 4-domains, subfamily A, | Hs.99960 | L35848 |
| | | member 3 (hematopoietic cell-specific) | | |
| ALOX5AP | 13q12 | arachidonate 5-lipoxygenase-activating protein | Hs.100194 | AI806222 |
| FCGR3B | 1q23 | Fc fragment of IgG, low affinity IIIa, receptor | Hs.176663 | J04162 |
| | | for (CD16) | | |
| QPCT | 2p22.3 | glutaminyl-peptide cyclotransferase (glutaminyl | Hs.79033 | X71125 |
| | | cyclase) | | |
| CYBB | Xp21.1 | cytochrome b-245, beta polypeptide (chronic | Hs.88974 | X04011 |
| | | granulomatous disease) | | |
| IL18RAP | 2p24.3-p24.1 | interleukin 18 receptor accessory protein | Hs.158315 | AF077346 |
| ORM1 | 9q31-q32, 9q32 | orosomucoid 1 | Hs.572 | X02544 |
| IGL@ | 22q11.1-q11.2, | immunoglobulin lambda locus | Hs.8997 | X57809 |
| | 6p21.3 | | | |
| ISG20 | 15q26 | interferon stimulated gene (20 kD) | Hs.183487 | U88964 |
| IGHM | | immunoglobulin heavy constant alpha 1 | Hs.293441 | AF067420 |
| ELA2 | 19p13.3 | elastase 2, neutrophil | Hs.99863 | M34379 |
| IGL@ | 22q11.1-q11.2 | immunoglobulin lambda locus | Hs.181125 | M18645 |
| UNK_S71043 | | immunoglobulin heavy constant alpha 1 | | S71043 |
| UNK_S71043 | | immunoglobulin heavy constant alpha 1 | | S71043 |
| IGHM | 14q32.33 | immunoglobulin heavy constant mu | Hs.153261 | X67301 |
| SCYC2 | 1q23, 1q23-q25 | small inducible cytokine subfamily C, member 2 | Hs.174228, | D63789 |
| | | | Hs.3195 | |
| SLPI | 20q12 | secretory leukocyte protease inhibitor | Hs.251754 | X04470 |
| | | (antileukoproteinase) | | |
| DEFA3 | 8p23.2-p23.1, | defensin, alpha 3, neutrophil-specific | Hs.274463, | L12691 |
| | 8pter-p23.3 | | Hs.294176 | |
| OLR1 | 12p13.2-p12.3 | oxidised low density lipoprotein (lectin-like) | Hs.77729 | AF079167 |
| | | receptor 1 | | |
| PRTN3 | 19p13.3 | proteinase 3 (serine proteinase, neutrophil, | Hs.928 | X55668 |
| | | Wegener granulomatosis autoantigen) | | |
| CKAP4 | 12q23.3 | transmembrane protein (63 kD), endoplasmic | Hs.74368 | X69910 |
| | | reticulum/Golgi intermediate compartment | | |
| ITGAM | 16p11.2 | integrin, alpha M (complement component | Hs.172631 | J03925 |
| | | receptor 3, alpha; also known as CD11b (p170), | | |
| | | macrophage antigen alpha polypeptide) | | |
| IGHM | 14q32.33 | immunoglobulin heavy constant mu | Hs.153261 | X67301 |
| TCL1A | 14q32.1 | T-cell leukemia/lymphoma 1A | Hs.2484 | X82240 |
| CHI3L1 | 1q31.1 | chitinase 3-like 1 (cartilage glycoprotein-39) | Hs.75184 | Y08374 |
| CEACAM1 | 19q13.2 | carcinoembryonic antigen-related cell adhesion | Hs.50964 | X16354 |
| | | molecule 1 (biliary glycoprotein) | | |
| TFF3 | 21q22.3 | trefoil factor 3 (intestinal) | Hs.352107 | L08044 |
| BPI | 20q11.23-q12 | bactericidal/permeability-increasing protein | Hs.89535 | J04739 |
| TCN1 | 11q11-q12 | transcobalamin I (vitamin B12 binding protein, | Hs.2012 | J05068 |
| | | R binder family) | | |
| UNK_AI985964 | 21q22.3 | trefoil factor 3 (intestinal) | Hs.82961 | AI985964 |
| HK3 | 5q35.2 | hexokinase 3 (white cell) | Hs.159237 | U51333 |
| CD24 | 6q21 | CD24 antigen (small cell lung carcinoma cluster | Hs.286124 | L33930 |
| | | 4 antigen) | | |
| FCN1 | 9q34 | ficolin (collagen/fibrinogen domain-containing) 1 | Hs.252136 | S80990 |
| ARG1 | 6q23 | arginase, liver | Hs.332405 | M14502 |
| HPR | 16q22.1 | haptoglobin-related protein | Hs.328822 | X89214 |
| S100P | 4p16 | S100 calcium-binding protein P | Hs.2962 | AA131149 |
| CEACAM6 | 19q13.2 | carcinoembryonic antigen-related cell adhesion | Hs.73848 | M18728 |
| | | molecule 6 (non-specific cross reacting antigen) | | |
| G0S2 | 1q32.2-q41 | putative lymphocyte G0/G1 switch gene | Hs.95910 | M69199 |
| GW112 | 13q14.2 | differentially expressed in hematopoietic | Hs.273321 | AF097021 |
| | | lineages | | |
| ANXA3 | 4q13-q22 | annexin A3 | Hs.1378 | M20560 |
| DEFA3 | 8p23.2-p23.1, | defensin, alpha 1, myeloid-related sequence | Hs.274463 | AL036554 |
| | 8pter-p23.3 | | | |
| CEACAM8 | 19q13.2 | carcinoembryonic antigen-related cell adhesion | Hs.41 | M33326 |
| | | molecule 8 | | |
| DEFA4 | 8p23 | defensin, alpha 4, corticostatin | Hs.2582 | AI250799 |
| MMP8 | 11q22.3 | matrix metalloproteinase 8 (neutrophil | Hs.73862 | J05556 |
| | | collagenase) | | |
| SGP28 | 6p12.2 | specific granule protein (28 kDa); cysteine-rich | Hs.54431 | X94323 |
| | | secretory protein-3 | | |
| PGLYRP | 19q13.2-q13.3 | peptidoglycan recognition protein | Hs.137583 | AF076483 |
| MMP9 | 20q11.2-q13.1 | matrix metalloproteinase 9 (gelatinase B, 92 kD | Hs.151738 | J05070 |
| | | gelatinase, 92 kD type IV collagenase) | | |
| S100A12 | 1q21 | S100 calcium-binding protein A12 (calgranulin | Hs.19413 | D83664 |
| | | C) | | |
| UNK_U95626 | 3q21-q23 | U95626: Homo sapiens ccr2b (ccr2), ccr2a | Hs.105938 | U95626 |
| | | (ccr2), ccr5 (ccr5) and ccr6 (ccr6) genes, | | |
| | | complete cds, and lactoferrin (lactoferrin) gene, | | |
| | | partial cds, complete sequence. | | |
| CAMP | 3p21.3 | cathelicidin antimicrobial peptide | Hs.51120 | Z38026 |
| LCN2 | 9q34 | lipocalin 2 (oncogene 24p3) | Hs.204238 | AI762213 |

Table 3 lists examples of qualifiers on HG-U95Av2 or HG-U95A genechips that showed different hybridization signals for MDS samples compared to disease-free samples. Each qualifier in Table 3 corresponds to at least one MDS disease gene. At least one oligonucleotide of the qualifier can hybridize under nucleic acid array hybridization conditions to an RNA transcript of the corresponding MDS disease gene.

Table 3 also demonstrates the ratio of the average expression level of each MDS disease gene in MDS BMMCs over that in disease-free BMMCs (“MDS/Disease-Free”), and the ratio of the average expression level of the MDS disease gene in AML BMMCs over that in disease-free BMMCs (“AML/Disease-Free”). In addition, Table 3 provides the p-value of a Student's t-test (two-tailed distribution, two sample unequal variance) for the difference between the average expression levels of each MDS disease gene in MDS BMMCs versus disease-free BMMCs (“p value (MDS vs Disease-Free)”). Table 4 provides the cytogenetic band, gene title, and Unigene and Entrez accession numbers for each MDS disease gene of Table 3.

TABLE 3
Expression Profiles of MDS Disease Genes in MDS and Disease-Free BMMCs

| | | | | p value |
|---|---|---|---|---|
| | | AML/ | MDS/ | (MDS vs |
| Qualifier | Gene Name | Disease-Free | Disease-Free | Disease-Free) |
| 36710_at | CAMP | 0.0292477 | 0.207509 | 7.644E−10 |
| 38976_at | CORO1A | 0.4358145 | 0.3161024 | 1.081E−09 |
| 32821_at | LCN2 | 0.0229556 | 0.1896334 | 1.903E−09 |
| 38879_at | S100A12 | 0.0390829 | 0.2344971 | 2.496E−09 |
| 33530_at | CEACAM8 | 0.0758328 | 0.2911438 | 1.292E−08 |
| 41184_s_at | UNK_X87344 | 1.0809717 | 0.437247 | 2.071E−08 |
| 31495_at | SCYC2 | 0.1790559 | 0.3743896 | 2.09E−08 |
| 37897_s_at | UNK_AI985964 | 0.1160991 | 0.1764706 | 2.19E−08 |
| 38533_s_at | ITGAM | 0.1658255 | 0.2487383 | 2.681E−08 |
| 38615_at | GW112 | 0.0868799 | 0.1775371 | 8.208E−08 |
| 31477_at | TFF3 | 0.1324278 | 0.3056027 | 1.669E−07 |
| 39330_s_at | ACTN1 | 0.4608819 | 0.3840683 | 2.433E−07 |
| 36372_at | HK3 | 0.1148325 | 0.1399522 | 2.688E−07 |
| 31381_at | PGLYRP | 0.0553506 | 0.1340066 | 3.474E−07 |
| 33758_f_at | PSG11 | 0.3832715 | 0.4455121 | 4.069E−07 |
| 32675_at | BST1 | 0.4184211 | 0.4223684 | 4.131E−07 |
| 40159_r_at | NCF1 | 0.2677108 | 0.1639046 | 4.157E−07 |
| 37149_s_at | UNK_U95626 | 0.0319286 | 0.3698401 | 5.56E−07 |
| 38968_at | SH3BP5 | 0.2195578 | 0.4905014 | 5.932E−07 |
| 40685_at | ALDH3B1 | 0.6466965 | 0.4871221 | 6.901E−07 |
| 679_at | CTSG | 0.301199 | 0.4037656 | 7.835E−07 |
| 36139_at | C6ORF5 | 1.6197822 | 2.0553539 | 8.107E−07 |
| 35315_at | ORM1 | 0.2030075 | 0.3338346 | 8.467E−07 |
| 36464_at | SGP28 | 0.0584795 | 0.1776878 | 8.819E−07 |
| 37233_at | OLR1 | 0.1762218 | 0.331297 | 9.295E−07 |
| 37105_at | CTSG | 0.3722783 | 0.4390194 | 1.596E−06 |
| 32612_at | GSN | 0.510014 | 0.3388449 | 1.868E−06 |
| 31859_at | MMP9 | 0.039135 | 0.1550349 | 2.146E−06 |
| 32451_at | MS4A3 | 0.2128146 | 0.3130435 | 2.186E−06 |
| 37099_at | ALOX5AP | 0.2114456 | 0.478051 | 2.25E−06 |
| 37215_at | UNK_AF046798 | 0.5595568 | 0.3434903 | 5.235E−06 |
| 38894_g_at | UNK_AL008637 | 0.3726469 | 0.386093 | 5.362E−06 |
| 40171_at | FRAT2 | 0.2808195 | 0.2331332 | 6.504E−06 |
| 33979_at | RNASE3 | 0.4056905 | 0.3334776 | 7.078E−06 |
| 39329_at | ACTN1 | 0.6416573 | 0.4837626 | 1.137E−05 |
| 37967_at | LY117 | 0.5693992 | 0.3737774 | 1.145E−05 |
| 35919_at | TCN1 | 0.1240602 | 0.2180451 | 1.154E−05 |
| 33757_f_at | PSG11 | 0.3984962 | 0.3834586 | 1.159E−05 |
| 36984_f_at | HPR | 0.098472 | 0.2937182 | 1.376E−05 |
| 36488_at | EGFL5 | 0.3217478 | 0.387289 | 1.431E−05 |
| 37066_at | PRTN3 | 0.1741486 | 0.370227 | 1.461E−05 |
| 32941_at | ICSBP1 | 1.3694952 | 0.4994629 | 1.649E−05 |
| 681_at | MMP8 | 0.0711638 | 0.4203113 | 1.776E−05 |
| 988_at | CEACAM1 | 0.1389918 | 0.3169014 | 2.153E−05 |
| 36447_at | FCN1 | 0.1046544 | 0.4530343 | 2.337E−05 |
| 39318_at | TCL1A | 0.1475279 | 0.1874003 | 2.721E−05 |
| 1913_at | CCNG2 | 0.4417293 | 0.4793233 | 2.958E−05 |
| 40013_at | CLIC2 | 1.4210526 | 2.0526316 | 3.349E−05 |
| 38895_i_at | NCF4 | 0.3383459 | 0.3233083 | 3.469E−05 |
| 36105_at | CEACAM6 | 0.0914953 | 0.4234925 | 3.611E−05 |
| 266_s_at | CD24 | 0.1084773 | 0.4379269 | 4.092E−05 |
| 36184_at | PLOD | 0.5619982 | 0.3746655 | 5.093E−05 |
| 1962_at | ARG1 | 0.1009792 | 0.4406365 | 5.453E−05 |
| 39221_at | LILRB2 | 0.4024768 | 0.4334365 | 5.457E−05 |
| 41138_at | MIC2 | 4.5394737 | 2.2039474 | 5.931E−05 |
| 32550_r_at | CEBPA | 2.4409237 | 2.0139635 | 7.178E−05 |
| 1825_at | IQGAP1 | 0.6206023 | 0.371517 | 7.791E−05 |
| 31792_at | ANXA3 | 0.0858726 | 0.4127424 | 7.965E−05 |
| 39128_r_at | PPP2R4 | 0.2269737 | 0.375 | 8.498E−05 |
| 33583_r_at | RBMS3 | 1.1348684 | 2.3684211 | 9.47E−05 |
| 39706_at | CPNE3 | 0.3207237 | 0.4111842 | 9.837E−05 |
| 37096_at | ELA2 | 0.1859842 | 0.4215984 | 0.0001034 |
| 35012_at | MNDA | 0.4605263 | 0.4425837 | 0.0001109 |
| 36617_at | ID1 | 2.8462604 | 4.7368421 | 0.0001128 |
| 32909_at | AQP5 | 1.3663968 | 2.7024291 | 0.000117 |
| 40876_at | GYG | 0.3354416 | 0.4506438 | 0.0001247 |
| AFFX- | 18SRNA5_Hs_AFFX | 0.9932088 | 3.6417657 | 0.0001356 |
| HUMRGE/ | | | | |
| M10098_5_at | | | | |
| 34768_at | TXNDC | 1.4605263 | 0.4657895 | 0.0001386 |
| 35629_at | UNK_AL022238 | 0.5986842 | 0.4934211 | 0.0001429 |
| 34095_f_at | UNK_U80114 | 0.2467105 | 0.3700658 | 0.0001457 |
| 40172_g_at | FRAT2 | 0.5413534 | 0.4511278 | 0.0001621 |
| 37975_at | CYBB | 0.2101261 | 0.1801081 | 0.0001624 |
| 39383_at | ADCY6 | 1.3550668 | 2.2623723 | 0.0001688 |
| 41827_f_at | UNK_AI932613 | 0.2254155 | 0.3407202 | 0.000181 |
| 36713_at | DKFZP434C091 | 0.7437071 | 2.3569794 | 0.0001863 |
| 35714_at | PDXK | 0.3446998 | 0.3891772 | 0.0001918 |
| 33093_at | IL18RAP | 0.2083333 | 0.2302632 | 0.000209 |
| 37054_at | BPI | 0.1295953 | 0.4641629 | 0.0002365 |
| 34546_at | DEFA4 | 0.071914 | 0.4445235 | 0.0002622 |
| 33309_at | UNK_AA521060 | 0.3340081 | 0.4402834 | 0.0002988 |
| 33352_at | UNK_X57985 | 3.6064593 | 2.4222488 | 0.0003344 |
| 39436_at | BNIP3L | 0.6537829 | 2.0106908 | 0.0003659 |
| 31528_f_at | H2BFE | 2.3299101 | 2.4261874 | 0.0004022 |
| 33143_s_at | SLC16A3 | 0.3282548 | 0.3739612 | 0.0004079 |
| 34892_at | TNFRSF10B | 1.9736842 | 2.5119617 | 0.0004095 |
| 38585_at | UNK_M91036 | 3.4927558 | 8.3116499 | 0.0004101 |
| 1257_s_at | QSCN6 | 1.1403509 | 3.7231969 | 0.0004178 |
| 34597_at | PPYR1 | 0.7655502 | 2.1052632 | 0.0004199 |
| 39315_at | ANGPT1 | 3.6090226 | 2.1428571 | 0.000431 |
| 34320_at | PTRF | 2.2156197 | 2.4448217 | 0.0004344 |
| 34105_f_at | IGHG3 | 0.2529206 | 0.3703481 | 0.0005086 |
| 41198_at | GRN | 0.7404381 | 0.3653155 | 0.0005659 |
| 38487_at | STAB1 | 4.8185118 | 2.831216 | 0.0006067 |
| 37194_at | GATA2 | 2.7379619 | 2.3852184 | 0.0006456 |
| 41249_at | UNK_AL031282 | 0.4605263 | 0.4093567 | 0.0007797 |
| 38747_at | CD34 | 2.5725953 | 2.0145191 | 0.0008012 |
| 1531_at | UNK_U50535 | 1.2947368 | 2.0526316 | 0.000849 |
| 38514_at | IGLL1 | 1.754386 | 0.3333333 | 0.000868 |

TABLE 4
Examples of MDS Disease Genes

| | Cytogenetic | | Unigene | Entrez |
|---|---|---|---|---|
| Gene Name | Band | Gene Title | No. | Accession No |
| CAMP | 3p21.3 | cathelicidin antimicrobial peptide | Hs.51120 | Z38026 |
| CORO1A | 16q13 | coronin, actin-binding protein, 1A | Hs.109606 | D44497 |
| LCN2 | 9q34 | lipocalin 2 (oncogene 24p3) | Hs.204238 | AI762213 |
| S100A12 | 1q21 | S100 calcium-binding protein A12 | Hs.19413 | D83664 |
| | | (calgranulin C) | | |
| CEACAM8 | 19q13.2 | carcinoembryonic antigen-related | Hs.41 | M33326 |
| | | cell adhesion molecule 8 | | |
| UNK_X87344 | 6p21.3 | H. sapiens DMA, DMB, HLA-Z1, | Hs.180062 | X87344 |
| | | IPP2, LMP2, TAP1, LMP7, TAP2, | | |
| | | DOB, DQB2 and RING8, 9, 13 and | | |
| | | 14 genes | | |
| SCYC2 | 1q23, 1q23-q25 | small inducible cytokine subfamily | Hs.174228, | D63789 |
| | | C, member 2 | Hs.3195 | |
| UNK_AI985964 | 21q22.3 | trefoil factor 3 (intestinal) | Hs.82961 | AI985964 |
| ITGAM | 16p11.2 | integrin, alpha M (complement | Hs.172631 | J03925 |
| | | component receptor 3, alpha; also | | |
| | | known as CD11b (p170), | | |
| | | macrophage antigen alpha | | |
| | | polypeptide) | | |
| GW112 | 13q14.2 | differentially expressed in | Hs.273321 | AF097021 |
| | | hematopoietic lineages | | |
| TFF3 | 21q22.3 | trefoil factor 3 (intestinal) | Hs.352107 | L08044 |
| ACTN1 | 14q24 | actinin, alpha 1 | Hs.119000 | M95178 |
| HK3 | 5q35.2 | hexokinase 3 (white cell) | Hs.159237 | U51333 |
| PGLYRP | 19q13.2-q13.3 | peptidoglycan recognition protein | Hs.137583 | AF076483 |
| PSG11 | 19q13.2 | pregnancy specific beta-1- | Hs.334408 | U25988 |
| | | glycoprotein 11 | | |
| BST1 | 4p15 | bone marrow stromal cell antigen 1 | Hs.169998 | D21878 |
| NCF1 | 7q11.23 | neutrophil cytosolic factor 1 (47 kD, | Hs.1583 | M55067 |
| | | chronic granulomatous disease, | | |
| | | autosomal 1) | | |
| UNK_U95626 | 3q21-q23 | U95626: Homo sapiens ccr2b | Hs.105938 | U95626 |
| | | (ccr2), ccr2a (ccr2), ccr5 (ccr5) and | | |
| | | ccr6 (ccr6) genes, complete cds, and | | |
| | | lactoferrin (lactoferrin) gene, partial | | |
| | | cds, complete sequence. | | |
| SH3BP5 | 3p24.3 | SH3-domain binding protein 5 | Hs.109150 | AB005047 |
| | | (BTK-associated) | | |
| ALDH3B1 | 11q13 | aldehyde dehydrogenase 7 | Hs.83155 | U10868 |
| CTSG | 14q11.2 | cathepsin G | Hs.100764 | J04990 |
| C6ORF5 | 6q21 | DKFZP586G0522 protein | Hs.7446 | AL050289 |
| ORM1 | 9q31-q32, | orosomucoid 1 | Hs.572 | X02544 |
| | 9q32 | | | |
| SGP28 | 6p12.2 | specific granule protein (28 kDa); | Hs.54431 | X94323 |
| | | cysteine-rich secretory protein-3 | | |
| OLR1 | 12p13.2-p12.3 | oxidised low density lipoprotein | Hs.77729 | AF079167 |
| | | (lectin-like) receptor 1 | | |
| CTSG | 14q11.2 | cathepsin G | Hs.100764 | M16117 |
| GSN | 9q33 | gelsolin (amyloidosis, Finnish type) | Hs.290070 | X04412 |
| MMP9 | 20q11.2-q13.1 | matrix metalloproteinase 9 | Hs.151738 | J05070 |
| | | (gelatinase B, 92 kD gelatinase, | | |
| | | 92 kD type IV collagenase) | | |
| MS4A3 | 11q12-q13.1 | membrane-spanning 4-domains, | Hs.99960 | L35848 |
| | | subfamily A, member 3 | | |
| | | (hematopoietic cell-specific) | | |
| ALOX5AP | 13q12 | arachidonate 5-lipoxygenase- | Hs.100194 | AI806222 |
| | | activating protein | | |
| UNK_AF046798 | 14q21-q22 | phosphorylase, glycogen; liver | Hs.771 | AF046798 |
| | | (Hers disease, glycogen storage | | |
| | | disease type VI) | | |
| UNK_AL008637 | 22q13.1 | neutrophil cytosolic factor 4 (40 kD) | Hs.196352 | AL008637 |
| FRAT2 | 10q23-q24.1 | GSK-3 binding protein FRAT2 | Hs.140720 | AF062739 |
| RNASE3 | 14q24-q31 | ribonuclease, RNase A family, 3 | Hs.73839 | X55990 |
| | | (eosinophil cationic protein) | | |
| ACTN1 | 14q24 | actinin, alpha 1 | Hs.119000 | X15804 |
| LY117 | 6p21.3 | DNA segment on chromosome 6 | Hs.88411 | AF000424 |
| | | (unique) 49 expressed sequence | | |
| TCN1 | 11q11-q12 | transcobalamin I (vitamin B12 | Hs.2012 | J05068 |
| | | binding protein, R binder family) | | |
| PSG11 | 19q13.2 | pregnancy specific beta-1- | Hs.334408 | M69245 |
| | | glycoprotein 11 | | |
| HPR | 16q22.1 | haptoglobin-related protein | Hs.328822 | X89214 |
| EGFL5 | 9q32-q33.3 | EGF-like-domain, multiple 5 | Hs.5599 | AB011542 |
| PRTN3 | 19p13.3 | proteinase 3 (serine proteinase, | Hs.928 | X55668 |
| | | neutrophil, Wegener granulomatosis | | |
| | | autoantigen) | | |
| ICSBP1 | 16q24.1 | interferon consensus sequence | Hs.14453 | M91196 |
| | | binding protein 1 | | |
| MMP8 | 11q22.3 | matrix metalloproteinase 8 | Hs.73862 | J05556 |
| | | (neutrophil collagenase) | | |
| CEACAM1 | 19q13.2 | carcinoembryonic antigen-related | Hs.50964 | X16354 |
| | | cell adhesion molecule 1 (biliary | | |
| | | glycoprotein) | | |
| FCN1 | 9q34 | ficolin (collagen/fibrinogen domain- | Hs.252136 | S80990 |
| | | containing) 1 | | |
| TCL1A | 14q32.1 | T-cell leukemia/lymphoma 1A | Hs.2484 | X82240 |
| CCNG2 | 4q13.3 | cyclin G2 | Hs.79069 | U47414 |
| CLIC2 | Xq28 | chloride intracellular channel 2 | Hs.54570 | Y12696 |
| NCF4 | 22q13.1 | neutrophil cytosolic factor 4 (40 kD) | Hs.196352 | X77094 |
| CEACAM6 | 19q13.2 | carcinoembryonic antigen-related | Hs.73848 | M18728 |
| | | cell adhesion molecule 6 (non- | | |
| | | specific cross reacting antigen) | | |
| CD24 | 6q21 | CD24 antigen (small cell lung | Hs.286124 | L33930 |
| | | carcinoma cluster 4 antigen) | | |
| PLOD | 1p36.3-p36.2 | procollagen-lysine, 2-oxoglutarate | Hs.75093 | L06419 |
| | | 5-dioxygenase (lysine hydroxylase, | | |
| | | Ehlers-Danlos syndrome type VI) | | |
| ARG1 | 6q23 | arginase, liver | Hs.332405 | M14502 |
| LILRB2 | 19q13.4 | leukocyte immunoglobulin-like | Hs.22405 | AF004231 |
| | | receptor, subfamily B (with TM and | | |
| | | ITIM domains), member 2 | | |
| MIC2 | Xp22.32, | antigen identified by monoclonal | Hs.177543 | M16279 |
| | Yp11.3 | antibodies 12E7, F21 and O13 | | |
| CEBPA | 19q13.1 | CCAAT/enhancer binding protein | Hs.76171 | Y11525 |
| | | (C/EBP), alpha | | |
| IQGAP1 | 15q26.1 | IQ motif containing GTPase | Hs.1742 | L33075 |
| | | activating protein 1 | | |
| ANXA3 | 4q13-q22 | annexin A3 | Hs.1378 | M20560 |
| PPP2R4 | 9q34 | protein phosphatase 2A, regulatory | Hs.236963 | X73478 |
| | | subunit B′ (PR 53) | | |
| RBMS3 | 3p24-p23 | RNA binding motif, single stranded | Hs.158446 | AA523313 |
| | | interacting protein 3 | | |
| CPNE3 | 8q21.2 | copine III | Hs.14158 | AB014536 |
| ELA2 | 19p13.3 | elastase 2, neutrophil | Hs.99863 | M34379 |
| MNDA | 1q22 | myeloid cell nuclear differentiation | Hs.153837 | M81750 |
| | | antigen | | |
| ID1 | 20q11 | inhibitor of DNA binding 1, | Hs.75424 | X77956 |
| | | dominant negative helix-loop-helix | | |
| | | protein | | |
| AQP5 | 12q13 | aquaporin 5 | Hs.298023 | U46569 |
| GYG | 3q24-q25.1 | glycogenin | Hs.174071 | U31525 |
| 18SRNA5_Hs_AFFX | | 18SRNA5 control sequence (H. sapiens) | | M10098 |
| | | [AFFX] | | |
| TXNDC | 14q21.3 | DKFZP564E1962 protein | Hs.24766 | AL080080 |
| UNK_AL022238 | | Human DNA sequence from clone | | AL022238 |
| | | 1042K10 on chromosome 22q13.1-13.2. | | |
| | | Contains the ADSL gene for | | |
| | | Adenylosuccinate lyase (EC 4.3.2.2, | | |
| | | Adenylosuccinase, ASL) and 4 | | |
| | | novel genes (one with probable | | |
| | | rabGAP domains and Src homology | | |
| | | domain 3). Contains ESTs, STSs, | | |
| | | GSSs and a putative CpG island | | |
| UNK_U80114 | | Human immunoglobulin heavy | | U80114 |
| | | chain variable region (V4-31) gene, | | |
| | | partial cds | | |
| FRAT2 | 10q23-q24.1 | GSK-3 binding protein FRAT2 | Hs.140720 | AF062739 |
| CYBB | Xp21.1 | cytochrome b-245, beta polypeptide | Hs.88974 | X04011 |
| | | (chronic granulomatous disease) | | |
| ADCY6 | 12q12-q13 | KIAA0422 protein | Hs.12373 | AB007882 |
| UNK_AI932613 | | Human rearranged immunoglobulin | Hs.350074 | AI932613 |
| | | lambda light chain mRNA | | |
| DKFZP434C091 | 1q44 | DKFZP434C091 protein | Hs.51692 | AL080170 |
| PDXK | 21q22.3 | pyridoxal (pyridoxine, vitamin B6) | Hs.38041 | U89606 |
| | | kinase | | |
| IL18RAP | 2p24.3-p24.1 | interleukin 18 receptor accessory | Hs.158315 | AF077346 |
| | | protein | | |
| BPI | 20q11.23-q12 | bactericidal/permeability-increasing | Hs.89535 | J04739 |
| | | protein | | |
| DEFA4 | 8p23 | defensin, alpha 4, corticostatin | Hs.2582 | AI250799 |
| UNK_AA521060 | | Homo sapiens clone 23551 mRNA | Hs.184019 | AA521060 |
| | | sequence | | |
| UNK_X57985 | 1q21-q23 | H2B histone family, member Q | Hs.2178 | X57985 |
| BNIP3L | 8p21 | BCL2/adenovirus E1B 19 kD- | Hs.132955 | AF079221 |
| | | interacting protein 3-like | | |
| H2BFE | 6p22-p21.3 | H2B histone family, member E | Hs.182432 | Z83738 |
| SLC16A3 | 22q12.3-q13.2 | solute carrier family 16 | Hs.85838 | U81800 |
| | | (monocarboxylic acid transporters), | | |
| | | member 3 | | |
| TNFRSF10B | 8p22-p21 | tumor necrosis factor receptor | Hs.51233 | AF016266 |
| | | superfamily, member 10b | | |
| UNK_M91036 | 11p15.5 | hemoglobin, gamma G | Hs.266959, | M91036 |
| | | | Hs.283108 | |
| QSCN6 | 1q24 | quiescin Q6 | Hs.77266 | L42379 |
| PPYR1 | 10q11.2 | pancreatic polypeptide receptor 1 | Hs.54426 | U42387 |
| ANGPT1 | 8q22.3-q23 | angiopoietin 1 | Hs.2463 | D13628 |
| PTRF | 17q21.2 | Homo sapiens mRNA; cDNA | Hs.29759 | AL050224 |
| | | DKFZp586L2123 (from clone | | |
| | | DKFZp586L2123) | | |
| IGHG3 | 14q32.33 | Homo sapiens isolate RP | Hs.300697 | AI147237 |
| | | immunoglobulin heavy chain FW2- | | |
| | | JH region gene, partial cds | | |
| GRN | 17q21.32 | granulin | Hs.180577 | AF055008 |
| STAB1 | 3p21.31 | KIAA0246 protein | Hs.301989 | D87433 |
| GATA2 | 3q21, 3q22.1 | GATA-binding protein 2 | Hs.367725 | M68891 |
| UNK_AL031282 | 1p36.33-p36.21 | Human DNA sequence from clone | Hs.220324 | AL031282 |
| | | 283E3 on chromosome 1p36.21-36.33. | | |
| | | Contains the alternatively | | |
| | | spliced gene for Matrix | | |
| | | Metalloproteinase in the Female | | |
| | | Reproductive tract MIFR1, -2, | | |
| | | MMP21/22A, -B and -C, a novel | | |
| | | gene, the alternatively spliced | | |
| | | CDC2L2 gene for Cell Division | | |
| | | Cycle 2-Like 2 (PITSLRE, | | |
| | | p58/GTA, Galactosyltransferase | | |
| | | Associated Protein Kinase) beta 1, | | |
| | | beta 2-1, beta 2-2 and alpha 2-4, a . . . | | |
| CD34 | 1q32 | CD34 antigen | Hs.367690 | M81945 |
| UNK_U50535 | | Human BRCA2 region, mRNA | Hs.110630 | U50535 |
| | | sequence CG006 | | |
| IGLL1 | 22q11.23 | immunoglobulin lambda-like | Hs.348935 | M27749 |
| | | polypeptide 3 | | |

The AML and MDS disease genes listed in Tables 1-4 were identified based on HG-U95Av2 and HG-U95A genechip annotation provided by Affymetrix. AML or MDS disease genes can also be identified based on the corresponding Unigene or Entrez accession numbers. In addition, AML or MDS disease genes can be determined by BLAST searching the oligonucleotide probes or target sequences of the corresponding qualifiers against a human genome sequence database. Human genome sequence databases suitable for this purpose include, but are not limited to, the Entrez human genome database at the National Center for Biotechnology Information (NCBI). The NCBI provides publicly accessible BLAST programs, such as “blastn,” for searching its sequence database. In one embodiment, the query sequence for the BLAST search is an unambiguous segment (i.e., without “n” residues) of the target sequence of a qualifier. Gene or genes that have substantial sequence identity with the unambiguous segment are identified. These genes may produce different hybridization signals on the qualifier for AML or MDS samples compared to disease-free samples.

The oligonucleotide probe sequences as well as the target sequence of each qualifier on HG-U95Av2 or HG-U95A genechips may be obtained from Affymetrix or from the sequence files maintained at Affymetrix website “www.affymetrix.com/support/technical/byproduct.affx?product=hgu95sequence.” The oligonucleotide probe sequences can be found in the sequence files “HG_U95Av2 Probe Sequences, FASTA” and “HG_U95A Probe Sequences, FASTA,” and the target sequences may be found in “HG_U95Av2 Target Sequences, FASTA” and “HG_U95A Target Sequences, FASTA.” All of these sequence files are incorporated herein by reference in their entireties.

The above-described methods can be readily adapted to the identification of disease genes associated with other blood or bone marrow diseases. These disease genes are differentially expressed in bone marrow or blood cells of patients who have the blood or bone marrow diseases as compared to disease-free humans. Blood or bone marrow diseases that are amenable to the present invention include, but are not limited to, acute lymphocytic leukemia, chronic lymphocytic leukemia, chronic myelogenous leukemia, Hodgkin's disease, non-Hodgkin's disease, and other types of leukemia and lymphoma.

### C. DIAGNOSIS AND MONITORING THE TREATMENT OR PROGRESSION OF AML AND MDS

The disease genes of the present invention can be used for the detection or diagnosis of AML or MDS. The disease genes of the present invention can also be used to monitor the treatment or progression of AML or MDS. The disease genes of the present invention can be used independently or in combination with other clinical criteria. In many embodiments, the methods of the present invention include comparing the expression profile of one or more AML or MDS disease genes in a bone marrow sample of a patient of interest to a reference expression profile of the same gene or genes. The difference or similarity in the expression profiles is suggestive of AML, MDS, or disease-free status of the patient of interest.

Numerous methods can be used for determining the expression profile of AML or MDS disease genes in a bone marrow sample. In many embodiments, the expression profile is determined by measuring the levels of RNA transcripts of the disease genes. Methods suitable for this purpose include, but are not limited to, RT-PCT, Northern Blot, in situ hybridization, slot-blotting, nuclease protection assay, and polynucleotide arrays. In many other embodiments, the expression profile is determined by detecting the levels of polypeptides encoded by the disease genes. Methods suitable for this purpose include, but are not limited to, immunoassays such as ELISA (enzyme-linked immunosorbent assay), RIA (radioimmunoassay), FACS (fluorescence-activated cell sorter), Western Blot, dot blot, immunohistochemistry, or antibody-based radioimaging. Other methods, such as high-throughput protein sequencing, two-dimensional SDS-polyacrylamide gel electrophoresis, or mass spectrometry, can also be used.

Examples of bone marrow samples suitable for the present invention include, but are not limited to, whole bone marrow samples, or bone marrow samples containing enriched or purified BMMCs or bone marrow leukocytes. Any method known in the art (e.g., aspiration or biopsy) may be used to collect bone marrow samples. Bone marrow samples containing enriched or purified BMMCs or bone marrow leukocytes can be prepared by Ficoll gradients or CPTs (cell purification tubes). By “enriched,” it means that the cell percentage of BMMCs or bone marrow leukocytes in the sample is higher than that in the original whole bone marrow. In many instances, the enriched or purified BMMCs are un-fractionated.

In one embodiment, quantitative RT-PCR (such as TaqMan, ABI) is used for detecting and comparing the expression profiles of AML or MDS disease genes in bone marrow samples. Quantitative RT-PCR involves reverse transcription (RT) of RNA to cDNA followed by relative quantitative PCR.

In PCR, the number of molecules of the amplified target DNA increases by a factor approaching two with every cycle of the reaction until some reagent becomes limiting. Thereafter, the rate of amplification becomes increasingly diminished until there is not an increase in the amplified target between cycles. If a graph is plotted on which the cycle number is on the X axis and the log of the concentration of the amplified target DNA is on the Y axis, a curved line of characteristic shape can be formed by connecting the plotted points. Beginning with the first cycle, the slope of the line is positive and constant. This is said to be the linear portion of the curve. After some reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR is proportional to the starting concentration of the target before the PCR is begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction.

The final concentration of the target DNA in the plateau portion of the curve is determined by the availability of reagents in the reaction mix and is independent of the original concentration of target DNA. Therefore, the sampling and quantifying of the amplified PCR products can be carried out when the PCR reactions are in the linear portion of their curves. In addition, relative concentrations of the amplifiable cDNAs can be normalized to some independent standard, which may be based on either internally existing RNA species or externally introduced RNA species. The abundance of a particular mRNA species may also be determined relative to the average abundance of all mRNA species in the sample.

In one example, the PCR amplification utilizes internal PCR standards that are approximately as abundant as the target. This strategy is effective if the products of the PCR amplifications are sampled during their linear phases. If the products are sampled when the reactions are approaching the plateau phase, then the less abundant product may become relatively over-represented. Comparisons of relative abundances made for many different RNA samples, such as is the case when examining RNA samples for differential expression, may become distorted in such a way as to make differences in relative abundances of RNAs appear less than they actually are. This can be improved if the internal standard is much more abundant than the target. If the internal standard is more abundant than the target, then direct linear comparisons may be made between RNA samples.

A problem inherent in clinical samples is that they are of variable quantity and/or quality. This problem can be overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

In another example, the relative quantitative RT-PCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs. While empirical determination of the linear range of the amplification curve and normalization of cDNA preparations are tedious and time-consuming processes, the resulting RT-PCR assays may, in certain cases, be superior to those derived from a relative quantitative RT-PCR with an internal standard.

In another embodiment, nucleic acid arrays (including bead arrays) are used for detecting and comparing the expression patterns of AML or MDS disease genes in bone marrow samples. Construction of nucleic acid arrays is well known in the art. A nucleic acid array of the present invention can comprise at least 2, 5, 10, 15, 20, 25, 30, 35, 40, 45, 50, 60, 70, 80, 90, 100, 150, 200, 250 or more different polynucleotide probes, each different probe capable of hybridizing to a different respective AML or MDS disease gene. Multiple probes for the same gene can be used on the same array. Probes for other disease genes can also be included in a nucleic acid array of the present invention. The probe density on a nucleic acid array of the present invention can be in any range. For instance, the density can be at least or no greater than 5, 10, 25, 50, 100, 200, 300, 400, 500, 1000, or more probes/cm².

In yet another embodiment, nuclease protection assays are used to quantify RNAs derived from bone marrow samples. There are many different versions of nuclease protection assays. The common characteristic of these nuclease protection assays is that they involve hybridization of an antisense nucleic acid with the RNA to be quantified. The resulting hybrid double-stranded molecule is then digested with a nuclease which digests single-stranded nucleic acids more efficiently than double-stranded molecules. The amount of antisense nucleic acid that survives digestion is a measure of the amount of the target RNA species to be quantified. Examples of nuclease protection assays include, but are not limited to, the RNase protection assay manufactured by Ambion, Inc. (Austin, Tex.).

In a further embodiment, immunoassays, such as ELISA, are used to detect and compare the expression profiles of AML or MDS disease genes. In an exemplifying ELISA, antibodies capable of binding to the target proteins are immobilized onto a selected surface exhibiting protein affinity, such as wells in a polystyrene or polyvinylchloride microtiter plate. Then, samples to be tested are added to the wells. After binding and washing to remove non-specifically bound immunocomplexes, the bound antigen(s) can be detected. Detection can be achieved by the addition of a second antibody which is specific for the target proteins and is linked to a detectable label. Detection may also be achieved by the addition of a second antibody, followed by the addition of a third antibody that has binding affinity for the second antibody, with the third antibody being linked to a detectable label. Before being added to the microtiter plate, cells in the samples can be lysed and/or extracted to separate the target proteins from potentially interfering substances.

In another exemplifying ELISA, the samples suspected of containing the target proteins are immobilized onto the well surface and then contacted with the antibodies of the invention. After binding and washing to remove non-specifically bound immunocomplexes, the bound antigen is detected. Where the initial antibodies are linked to a detectable label, the immunocomplexes can be detected directly. The immunocomplexes can also be detected using a second antibody that has binding affinity for the first antibody, with the second antibody being linked to a detectable label.

Another exemplary ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Examples of these nonspecific protein include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can also be used. After binding of a protein or antibody to the well, coating with a non-reactive material to reduce background, and washing to remove unbound material, the immobilizing surface is contacted with the control and/or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 4° C. overnight. Detection of the immunocomplex then requires a labeled secondary binding ligand or antibody, or a secondary binding ligand or antibody in conjunction with a labeled tertiary antibody or third binding ligand.

Following all incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. In one embodiment, the label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl)-benzthiazoline-6-sulfonic acid (ABTS) and H₂O₂, in the case of peroxidase as the enzyme label. Quantitation can be achieved by measuring the degree of color generation, e.g., using a spectrophotometer.

Another immunoassay format suitable for the present invention is RIA (radioimmunoassay). An exemplary RIA is based on the competition between radiolabeled-polypeptides and unlabeled polypeptides for binding to a limited quantity of antibodies. Suitable radiolabels include, but are not limited to, I¹²⁵. In one embodiment, a fixed concentration of I¹²⁵-labeled polypeptide is incubated with a series of dilution of an antibody specific to the polypeptide. When the unlabeled polypeptide is added to the system, the amount of the I¹²⁵-polypeptide that binds to the antibody is decreased. A standard curve can therefore be constructed to represent the amount of antibody-bound I¹²⁵ polypeptide as a function of the concentration of the unlabeled polypeptide. From this standard curve, the concentration of the polypeptide in unknown samples can be determined. Any RIA protocol known in the art may be used in the present invention.

Suitable antibodies for the present invention include, but are not limited to, polyclonal antibodies, monoclonal antibodies, chimeric antibodies, humanized antibodies, single chain antibodies, Fab fragments, or fragments produced by a Fab expression library. Neutralizing antibodies (i.e., those which inhibit dimer formation) can also be used. Methods for preparing antibodies are well known in the art. In many embodiments, the antibodies of the present invention can bind to the respective AML or MDS disease gene products or other desired antigens with a binding affinity constant Kₐ of at least 10⁶ M⁻¹, 10⁷ M⁻¹, or more.

The antibodies of this invention can be labeled with one or more detectable moieties to allow for detection of antibody-antigen complexes. The detectable moieties can include compositions detectable by spectroscopic, enzymatic, photochemical, biochemical, bioelectronic, immunochemical, electrical, optical or chemical means. Exemplary detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

In still another embodiment, the expression profiles of AML or MDS disease genes are determined by measuring the biological activities of the polypeptides encoded by the disease genes. If a biological activity of a polypeptide is known, suitable in vitro assays can be developed to evaluate such an activity, thereby allowing the determination the amount of the polypeptide in a sample of interest.

The expression profile of AML or MDS disease genes in a sample of interest is compared to a reference expression profile. In many embodiments, the reference expression profile is an average expression profile of the AML or MDS disease genes in reference bone marrow samples. The reference bone marrow samples can be prepared from disease-free humans, or patients with known disease status. In many instances, the reference bone marrow samples are prepared by using the same or comparable method as is the sample of interest. In many other instances, the reference expression profile is obtained by using the same or comparable methodology as is the expression profile to be compared.

The similarity or difference between expression profiles can be determined by comparing each component in an expression profile to the corresponding component in another expression profile. An expression profile can be constructed based on, for example, the absolute or relative expression values of AML or MDS disease genes, the ratios between expression values of different AML or MDS disease genes, or other measures that are indicative of expression levels or patterns.

The similarity or difference between two corresponding components can be evaluated based on fold changes, absolute differences, or other suitable means. In one example, a component in an expression profile is a mean value, and the corresponding component in another expression profile falls within the standard deviation of the mean value. In such a case, the former expression profile may be considered similar to the latter expression profile with respect to that component. Other criteria, such as a multiple or fraction of the standard deviation or a certain degree of percentage increase or decrease (e.g., less than 10% change), may be used to measure similarity.

One or more AML or MDS disease genes can be used for the comparison of expression profiles. In many embodiments, at least 2, 3, 4, 6, 8, 10, 12, 14, 16, 18, 20, 30, 40, 50, 60, 70, 80, 90, 100, 150, 200, or more AML or MDS disease genes are used for diagnosing or monitoring the progression or treatment of AML or MDS. In one example, at least 50% (e.g., at least 60%, 70%, 80%, 90%, or more) of the components in an expression profile are similar to the corresponding components in another expression profile. Under these circumstances, the former expression profile may be considered similar to the latter expression profile. Different components in an expression profile may have different weights in the comparison. In certain cases, lower similarity requirements, such as less than 50% of the components, can be used to determine the similarities between expression profiles.

The AML or MDS disease genes, as well as the similarity criteria, can be selected such that the accuracy of diagnosis or prediction (the ratio of correct calls over the total of correct and incorrect calls) is relatively high. In many embodiments, the accuracy of diagnosis or prediction is at least 50%, 60%, 70%, 80%, 90%, or more. AML or MDS disease genes with diagnosis or prediction accuracy of less than 50% can also be used, provided that the diagnosis or prediction is statistically significant. In many cases, the gene expression-based methods are combined with other clinical tests to improve the accuracy of AML or MDS diagnosis.

Any AML or MDS disease gene can be used in diagnosing or monitoring the progression or treatment of AML or MDS. In one embodiment, the AML (or MDS) disease genes are selected to have p-value of no greater than 0.05, 0.01, 0.005, 0.001, 0.0005, 0.0001, or less. In another embodiment, the AML (or MDS) disease genes are selected to have significant correlations with the class distinction between AML samples (or MDS samples) and disease-free samples. For instance, the disease genes can be chosen from those above the 1%, 5%, or 10% significance level under the permutation test. The selected disease genes can include both AML and MDS disease genes.

In yet another embodiment, the selected AML (or MDS) disease genes include at least two groups of genes. The first group includes upregulated AML (or MDS) disease genes which have AML/Disease-Free ratios (or MDS/Disease-Free ratios) of at least 2, 3, 4, 5, 10, or more. The second group includes downregulated AML (or MDS) disease genes which have AML/Disease-Free ratios (MDS/Disease-Free ratios) of no greater than 0.5, 0.333, 0.25, 0.2, 0.1, or less. Each group may include at least 1, 2, 4, 6, 8, 10, 12, 14, 16, 18, 20, or more AML (or MDS) disease genes.

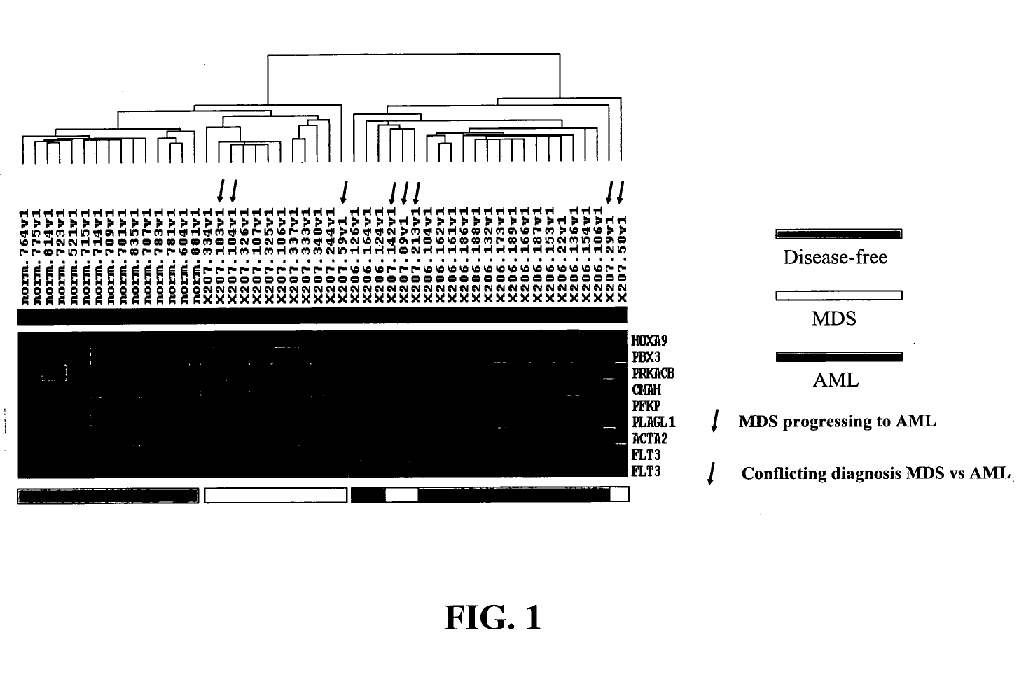

In a further embodiment, the gene set used in the present invention does not consist of genes selected from those described in Miyazato et al., supra, Tables 2 and 3 of Hofmann et al., supra, and Tables 3 and 4 and FIG. 1 of Larramendy et al., HAEMATOLOGICA, 87: 569-577 (2002), and nucleophosmin (NPM)/B23/numatrin. Miyazato et al., Hofmann et al., and Larramendy et al. are incorporated herein by reference.

In still another embodiment, the AML or MDS disease genes are selected from Tables 1, 3, 8b, and 9b. In one example, the selected AML disease genes include at least one gene shared by both Tables 1 and 8b, and the selected MDS disease genes include at least one gene shared by both Tables 3 and 9b. Examples of AML disease genes that are listed in both Tables 1 and 8b include, but are not limited to, FLT3, SPINK2, KIAA0246 (STAB1), HOXB2, ACTA2, MIC2, H2AFO, PFKP, RUNX1, CMAH, ADA, SCHIP-1, OA48-18, MYB, TBXAS1, H2BFQ, BAX, RUNX1, SNL, UNK_AF014837 (M6A), ITGA4, UNK_AA149307 (FLJ21174), ACADM, DBP, H2BFC, LYL1, DKFZP586A0522, DCTD, ETS2, H2BFG, BAX, PRKACB, HSPCB, LYL1, H2BFD, UNK_U78027, MYB, H2AFO, KIAA0128, UNK_AA005018 (LOC51097), HSPB1, KIAA0620, SOX4, UNK_AJ223352 (H2B/S), APEX, P311, CSNK1A1, UNK_N53547 (MGC5508), POLD2, UNK_AB007960 (SH3GLB1), GNA15, H2BFH, ATIC, NAP1L1, CCNG1, NPIP, UNK_AA176780 (HSA249128), PLAGL1, PAGA (PRDX1), GPX1, COMT, FARSL, JWA, LGALS1, IFI16, KIAA0906, RANBP7, MYB, IDH1, HSPCB, DCTD, FARSL, ADFP, UNK_T75292 (FLJ10849), CALR, PPID, CCT3, C14ORF3, PTPN7, UNK_Y14391 (PGPL), UNK_AA056747 (ATP6A1), LIPA, ICAM2, BST2, TARDBP, P130 (NOLC1), H2BFE, SPN (DEAF1), AMD1, HRMT1L2, UNK_AI808712, UQCRC2, PIP5K2B, ADE2H1 (PAICS), IRF5, ACF7 (MACF1), GP36B (C5ORF8), TFAP4, ATP5B, LTC4S, H2BFK, M11S1, UNK_AF041080 (MN7), FABP5, CLECSF2, RPML3 (MRPL3), KIAA0594, NME2, CCT6A, UNK_AF026816 (ITPA), AKR1A1, CHC1, ACADVL, SNRPA, CNIL, UNK_D28423, ALCAM, UNK_AI819942, DBI, NDUFB5, UNK_AL031432, SNRPB2, P24B, UNK_AJ245416 (LSM2), RMP, OGT, CYC1 (HCS), UNK_W28944 (GRHPR), FNTA, DOC1 (CDK2AP1), NDUFS4, RPL22, LMO2, KIAA0546, NME1, IMPDH2, PBX3, SDHD, UNK_AJ224875 (MGC2840), TOMM70A, HINT, DKFZP564M2423 (PAI-RBP1), IRF5, TCF8, MNDA, CD83, KIAA0474 (RAP1GA1), LGALS3, PLXNC1, ALDH2, NS1-BP, S100A11, TRA@, UNK_W28281(GABARAPL1), KIAA0403 (PIP3-E), KIAA0513, CORO1A, NCF2, BST1, CXCR4, IL4R, TRB@, BTN2A1, PSG11, HSPA1B, PTPRE, CD8A, NCF4, PTPRE, HSPA1A, MYL2, UGCG, GYG, EGFL5, CA4, ELN, CSPG4, AMPD2, NCF4, KIAA0879 (ENPP4), NPM1P14, CST7, PDXK, MMPL1, FGR, HBA2, EPB72, UNK_U80114, IGL@, AZU1, TUBA1, UNK_D29810 (ESDN), CD19, C4.4A, SIM2, COL9A1, SH3BP5, RNASE3, NR4A2, UNK_AF015128 (IGHM), PIR121, UNK_AL050223 (VAMP2), RGS2, PDI2, MME, CDKN2D, KIAA0763, IGHA1 (IGHM), PSG11, SLC16A3, CPNE3, SLC2A3, CHIT1, BCL2A1, CDA, FCAR, CD3Z, UNK_AL022723, IGHA1, IGHA1, TRB@, UNK_U72507, TRB@, NCF1, IL8RA, KLRB1, IGKV1D-8 (IGKC), UNK_AI147237 (IGHG3), UNK_Y14768, CYBB, BN51T, FRAT2, ISG20, RAB31, QPCT, TUBA1, MGAM, PLAUR, IGHM, HP, IGHG3, IGHM, S100A8, BASP1, UNK_D84143 (IGL@), IGHM, PBEF, UNK_AF013512 (LBP), PPP2R4, ALOX5, ALOX5AP, CD79A, SCYA4, VNN2, UNK_W28504, BPI, CTSG, BASP1, UNK_AL031588, UNK_AI932613, LILRA3, UNK_H12458, PRTN3, NKG7, FCGR3A, KIAA0604 (ECE2), S100A9, P63 (CKAP4), ORM1, MS4A3, SLPI, IGL@, CTSG, CHI3L1, HK3, IGL@, GNLY, ELA2, DEFA3, FCN1, GAS11, CEACAM1, IL18RAP, ITGAM, S100P, MMP8, TFF3, OLR1, TCN1, CD24, ARG1, SCYC2, DEFA4, ANXA3, HPR, CEACAM6, TFF3, DEFA1, G0S2, CEACAM8, TCL1A, PGLYRP, GW112, UNK_U95626, MMP9, SGP28, S100A12, CAMP, and LCN2. Examples of MDS disease genes that are listed in both Tables 3 and 9b include, but are not limited to, HBG2, ID1, KIAA0246 (STAB1), 18SRNA5_Hs_AFFX, TNFRSF10B, H2BFQ, GATA2, QSCN6, H2BFE, DKFZP434C091, MIC2, UNK_AL050224 (PTRF), ANGPT1, PSG11, SLC16A3, MNDA, CPNE3, GRN, BPI, ANXA3, FCN1, D6S49E, PYGL, CEACAM1, CD24, UNK_AI147237, PPP2R4, IQGAP1, OLR1, CEACAM6, PDXK, NCF4, NCF4, GSN, UNK_AI932613, RNASE3, ITGAM, ORM1, PSG11, CTSG, ACTN1, IGLL3, NCF1, CTSG, TCN1, UNK_U95626, CORO1A, HPR, IL18RAP, FRAT2, MS4A3, GW112, SCYC2, CEACAM8, PRTN3, ELA2, CYBB, DEFA4, TFF3, SGP28, HK3, PGLYRP, TFF3, S100A12, CAMP, MMP9, TCL1A, and LCN2.

In another example, the selected AML disease genes include at least one gene which is in Table 8b but not Table 1, and the selected MDS disease genes include at least one gene which is in Table 9b but not Table 3. Examples of such AML disease genes include, but are not limited to, LGALS3BP, HOXA9, MT1A, FLT3, ITM2A, PROML1, DDX21, UNK_W28186, CCNA1, SPARC, TPS1, H2AFA, MN1, DF, DRAP1, BMI1, MRC1, TSC22, MEST, RNASE6, UNK_AL050224, ANGPT1, HSU37012, KRT18, FOXC1, CLIM1, UNK_AI743507, ID1, 121, MYC, TIMP1, GSTM4, LGALS2, UNK_D87002, HBG2, KIAA0125, TEGT, MOX2, GRO2, UNK_AF010313, ADA, CLU, PGDS, ETFB, LOC51035, CD34, SSBP2, UNK_U51712, PPP1R8, NFE2, CPA3, STIP1, EDN1, SNRPC, CALR, TNFRSF10B, GATA2, IGFBP2, CD34, ID1, TRIP7, TIF1B, C1NH, POLR2E, CCR2, TFP1, MTA1, GATA2, UNK_AL035494, ST3GALV1, AMD1, CAPN4, IARS, GNAI1, CTSW, MYB, MAFF, MT1F, UNK_AF063002, CDC4L, UNK_U79260, SFRS7, KIAA0015, FCER1A, AMD1, D123, UNK_AI816034, UNK_W25874, CAMK2G, HSF2, H1F2, D6S81E, ZYX, P23, TACTILE, SMARCA2, KIAA1097, TARS, AKR1C3, F13A1, NRGN, HOXB5, PSMA4, TRIP6, CCT8, OS4, CDK4, EIF4G1, UNK_AF052159, PDNP2, HOMER-3, UNK_U34994, UNK_AL049432, UNK_U79291, HNRPR, PHKB, MYB, PQBP1, AARS, GP110, ADPRT, CSNK1G2, ITGA7, SPC18, UBE2N, UNK_AB007916, H2BFR, ARHB, SFPQ, UNK_W26056, KIAA0233, NDUFV2, CLIC4L, TNP1, ODF1, DHCR7, UNK_AA846749, IER3, CD3E, KIAA0796, GIPR, DAPK2, GADD45B, LPO, NRG2, MSX1, HSF4, PMS2L11, RABGGTA, UNK_X90579, GRM4, ADTAB, UNK_AB029343, UNK_AA586695, UPK1A, SIAT4C, CEACAM3, TNFAIP3, PRG1, GDF1, UNK_AA883101, UNK_L27065, KIAA0751, PTGDS, TFF3, UNK_AF090102, LRP3, SEC14L1, HBB, UNK_L40385, TNNT1, TBCD, UNK_AL050065, UNK_H08175, GCL, MPP2, RHOK, UNK_W26214, MTHFR, KIAA1080, UNK_AJ224442, UNK_W29012, PRF1, UNK_U92818, UNK_X61755, 28SRNA3_Hs_AFFX, UNK_AI687419, UNK_X14675, ACVR1B, UP, GJB1, KRTHA5, CSH1, CYCL, UNK_AF035314, UNK_X72475, RB1, KIAA0061, UNK_M96936, TNXA, SLC22A6, HUMRTVLH3, GFPT2, UNK_W28907, UNK_AI817548, SMARCA4, RSN, CHN2, KIAA0895, UNK_AA151971, FETUB, FECH, PTPRN, GZMB, KIAA0320, FCGR3B, MUC3, KIAA0168, UNK_AF070633, UNK_M14087, CYP4F2, IGHD, and ABL1. Examples of such MDS disease genes include, but are not limited to, UNK_N55205, DDX21, HOXB2, FBN2, UNK_W28186, FBN2, UNK_W28186, PF4, HOXA9, EDN1, H2AFO, SPINK2, ID1, OA48-18, HYPA, BMI1, ETS2, PPBP, CPA3, CDC42, RHAG, H1F2, PPBP, HSPCB, H2BFG, H2BFC, UNK_AF041080, H2BFH, TSC22, SNL, FLT3, PPM1A, UNK_AF010313, TEGT, LYL1, PEA15, SOX4, UNK_AF070569, H2AFO, NFE2, UNK_AJ223352, DKFZP434N093, PAI2, ADFP, ACADM, UNK_AF041081, PROML1, ITM2A, H2BFD, CLU, CLECSF2, UNK_U51712, 18SRNAM_Hs_AFFXMAFF, UNK_W27675, NRIP1, TRIP6, PPM1A, UNK_S62138, ATP5B, TPD52L2, UNK_S62138, UBE2E3, NP, BTG3, KIAA0907, ITGAX, TSSC3, KIAA1096, UNK_AL049265, H2AFL, GPX1, UNK_AC004381, SOS1, KRAS2, PMP22, AMD1, GNA15, BACH1, IARS, C140RF3, HSPB1, GNB2L1, IDS, UNK_Z24724, H4FG, CD9, TARDBP, UNK_AL035494, ITGB1, KIAA1097, TYROBP, UNK_L40385, IGHA1, BCAT1, BACTIN5_Hs_AFFX, IL8RA, BN51T, CAPG, CSPG2, BTN2A1, IGHA1, KIAA0604, MCC, CYBA, NR4A2, PTPRN, UNK_AF013512, UNK_U72507, ECGF1, D5S346, GNLY, RHOK, UNK_X72475, UNK_AL031588, LILRA3, FGL2, IGKV1D-8, SDF2, UNK_X14675, IGL@, UNK_W28504, IGL@, UNK_AI126004, S100A9, CDA, MPO, DEFA3, RSN, FGL2, AZU1, F11, IGHG3, SCYA4, NKG7, OPHN1, D6S2245E, SLPI, KIAA1080, HP, ACVR1B, UNK_H08175, 28SRNA3_Hs_AFFX, KIAA0061, UNK_Z97632, DEFA1, NR2C1, UNK_M96936, DGCR6, KIAA0483, KIAA0372, UNK_W26214, UNK_AF035314, CYP4F2, SLC22A6, ATPASEP, UNK_W28907, POU1F1, CCNT2, KIAA0895, CHN2, KIAA0320, UNK_W27838, POU1F1, MUC3, FECH, UNK_AL096744, FETUB, SMARCA4, BRD1, UNK_AF070633, UNK_J04178, KIAA0168, UNK_M14087, and ABL1.

In addition to the genes depicted in Tables 1, 3, 8b, and 9b, the present invention contemplates detection of the expression profiles of other genes that can hybridize under stringent or nucleic acid array hybridization conditions to the qualifiers selected from Tables 1, 3, 8b, and 9b. These genes may include hypothetical or putative genes that are supported by EST or mRNA data. As used herein, a gene can hybridize to a qualifier if an RNA transcript of the gene can hybridize to at least one oligonucleotide probe of the qualifier. In many instances, an RNA transcript of the gene can hybridize under stringent or nucleic acid array hybridization conditions to at least 50%, 60%, 70%, 80%, 90% or 100% of the oligonucleotide probes of the qualifier.

“Stringent conditions” are at least as stringent as, for example, conditions G-L shown in Table 5. “Highly stringent conditions” are at least as stringent as conditions A-F shown in Table 5. As used in Table 5, hybridization is carried out under the hybridization conditions (Hybridization Temperature and Buffer) for about four hours, followed by two 20-minute washes under the corresponding wash conditions (Wash Temp. and Buffer).

TABLE 5
Stringency Conditions

| Stringency | Poly-nucleotide | Hybrid | Hybridization | Wash Temp. |
|---|---|---|---|---|
| Condition | Hybrid | Length (bp)¹ | Temperature and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| ¹The hybrid length is that anticipated for the hybridized region(s) of the hybridizing polynucleotides. When hybridizing a polynucleotide to a target polynucleotide | | | | |
| # of unknown sequence, the hybrid length is assumed to be that of the hybridizing polynucleotide. When polynucleotides of known sequence are hybridized, the hybrid length | | | | |
| # can be determined by aligning the sequences of the polynucleotides and identifying the region or regions of optimal sequence complementarity. | | | | |
| ^{H}SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers. | | | | |
| T_{B}* − T_{R}*: The hybridization temperature for hybrids anticipated to be less than 50 base pairs in length should be 5-10° C. less than the melting temperature (Tₘ) of the hybrid, | | | | |
| # where Tₘ is determined according to the following equations. For hybrids less than 18 base pairs in length, Tₘ(° C.) = 2(# of A + T bases) + 4(# of G + C bases). For hybrids between | | | | |
| # 18 and 49 base pairs in length, Tₘ(° C.) = 81.5 + 16.6(log₁₀Na⁺) + 0.41(% G + C) − (600/N), where N is the number of bases in the hybrid, and Na⁺ is the molar concentration of sodium | | | | |
| # ions in the hybridization buffer (Na⁺ for 1xSSC = 0.165M). | | | | |

In one embodiment, the selected AML or MDS disease genes include at least one gene capable of hybridizing under stringent or nucleic acid array hybridization conditions to a qualifier commonly shared by Tables 1 and 8b, or a qualifier commonly shared by Tables 3 and 9b. Examples of qualifiers listed in both Table 1 and 8b include 1065_at, 41071_at, 38487_at, 39610_at, 32755_at, 41138_at, 32609_at, 39175_at, 39421_at, 39317_at, 41654_at, 36536_at, 34397_at, 1475_s_at, 33777_at, 33352_at, 1997_s_at, 943_at, 39070_at, 32245_at, 35731_at, 32251_at, 37532_at, 40274_at, 35576_f_at, 39971_at, 38717_at, 630_at, 1519_at, 31522_f_at, 2067_f_at, 36215_at, 33986_r_at, 32096_at, 36347_f_at, 38213_at, 2042_s_at, 286_at, 38826_at, 34862_at, 36785_at, 38671_at, 33131_at, 32819_at, 2025_s_at, 39710_at, 40184_at, 39693_at, 1470_at, 39691_at, 40365_at, 31523_f_at, 38811_at, 40634_at, 1920_s_at, 33836_at, 40485_at, 36943_r_at, 41213_at, 37033_s_at, 34651_at, 1751_g_at, 39091_at, 33412_at, 1456_s_at, 41812_s_at, 35255_at, 1474_s_at, 39023_at, 1161_at, 631_g_at, 1750_at, 34378_at, 33173_g_at, 32543_at, 948_s_at, 40774_at, 40979_at, 39672_at, 41108_at, 34889_at, 38745_at, 38454_g_at, 39061_at, 32241_at, 36597_at, 31528_f_at, 35771_at, 263_g_at, 32825_at, 31801_at, 40854_at, 35741_at, 39056_at, 478_g_at, 38704_at, 36955_at, 39638_at, 41357_at, 39968_at, 31524_f_at, 39471_at, 40877_s_at, 39799_at, 40698_at, 37726_at, 41379_at, 33415_at, 38416_at, 35801_at, 38780_at, 1196_at, 38376_at, 40842_at, 32803_at, 351_f_at, 38642_at, 37774_at, 37692_at, 32232_at, 38072_at, 38399_at, 41163_at, 41375_at, 38011_at, 39507_at, 35818_at, 40133_s_at, 1499_at, 41535 at, 38695_at, 1151_at, 32184_at, 35184_at, 1521_at, 36624_at, 32696_at, 40467_at, 32051_at, 32853_at, 1009_at, 40441_g_at, 36465_at, 33439_at, 35012_at, 37536_at, 33080_s_at, 35367_at, 32193_at, 32747_at, 33752_at, 38138_at, 1106_s_at, 35785_at, 33333_at, 38735_at, 38976_at, 41038_at, 32675_at, 649_s_at, 404_at, 1105_s_at, 32673_at, 33758_f_at, 31692_at, 32916_at, 40699_at, 38895_i_at, 1150_at, 1104_s_at, 36640_at, 40215_at, 40876_at, 36488_at, 40739_at, 31621_s_at, 110_at, 38417_at, 38894_g_at, 36459_at, 32901_s_at, 34965_at, 35714_at, 35911_r_at, 1780_at, 31525_s_at, 40419_at, 34095_f_at, 35530_f_at, 33963_at, 330_s_at, 40227_at, 1096_g_at, 41641_at, 39609_at, 35379_at, 38968_at, 33979_at, 37623_at, 35566_f_at, 37579_at, 32254_at, 37701_at, 35674_at, 1389_at, 1797_at, 34832_s_at, 33499_s_at, 33757 f_at, 33143_s_at, 39706_at, 36979_at, 37061_at, 2002_s_at, 1117_at, 38868_at, 37078_at, 37420_i_at, 33501_r_at, 33500_i_at, 32793_at, 39245_at, 32794_g_at, 40159_r_at, 1353_g_at, 35449_at, 38194_s_at, 34105_f_at, 40729_s_at, 37975_at, 41694_at, 40171_at, 33304_at, 33371_s_at, 35966_at, 36591_at, 34509_at, 189_s_at, 41164_at, 36983_f_at, 37864_s_at, 41165_g_at, 41096_at, 32606_at, 31315_at, 41166_at, 33849_at, 35013_at, 39128_r_at, 307_at, 37099_at, 38017_at, 36674_at, 34498_at, 36338_at, 37054_at, 37105_at, 32607_at, 39872_at, 41827_f_at, 35094_f_at, 2090_i_at, 37066_at, 37121_at, 37200_at, 35536_at, 41471_at, 32529_at, 35315_at, 32451_at, 32275_at, 33273_f_at, 679_at, 36197_at, 36372_at, 33274_f_at, 37145_at, 37096_at, 31506_s_at, 36447_at, 36479_at, 988_at, 33093_at, 38533_s_at, 34319_at, 681_at, 37897_s_at, 37233_at, 35919_at, 266_s_at, 1962_at, 31495_at, 34546_at, 31792_at, 36984_f_at, 36105_at, 31477_at, 31793_at, 38326_at, 33530_at, 39318_at, 31381_at, 38615_at, 37149_s_at, 31859_at, 36464_at, 38879_at, 36710_at, and 32821_at. Examples of qualifiers listed in both Table 3 and 9b include 38585_at, 36617_at, 38487_at, AFFX-HUMRGE/M10098_{—}5_at, 34892_at, 33352_at, 37194_at, 1257_s_at, 31528_f_at, 36713_at, 41138_at, 34320_at, 39315_at, 33758_f_at, 33143_s_at, 35012_at, 39706_at, 41198_at, 37054_at, 31792_at, 36447_at, 37967_at, 37215_at, 988_at, 266_s_at, 34105_f_at, 39128_r_at, 1825_at, 37233_at, 36105_at, 35714_at, 38894_g_at, 38895_i_at, 32612_at, 41827_f_at, 33979_at, 38533_s_at, 35315_at, 33757_f_at, 37105_at, 39330_s_at, 38514_at, 40159_r_at, 679_at, 35919_at, 37149_s_at, 38976_at, 36984_f_at, 33093_at, 40171_at, 32451_at, 38615_at, 31495_at, 33530_at, 37066_at, 37096_at, 37975_at, 34546_at, 31477_at, 36464_at, 36372_at, 31381_at, 37897_s_at, 38879_at, 36710_at, 31859_at, 39318_at, and 32821_at.

In another embodiment, the selected AML or MDS disease genes include at least one gene capable of hybridizing under stringent or nucleic acid array hybridization conditions to a qualifier which is shown in Table 8b but not in Table 1, or a qualifier which is shown in Table 9b but not in Table 3. Examples of qualifiers listed in Table 8b but not in Table 1 include 7754_at, 37809_at, 31623_f_at, 34583_at, 40775_at, 41470_at, 40490_at, 41188_at, 1914_at, 671_at, 32905_s_at, 35127_at, 37283 at, 40282_s_at, 39077_at, 41562_at, 36908_at, 39032_at, 37749_at, 34660_at, 34320_at, 39315_at, 33132_at, 35766_at, 41027_at, 36937_s_at, 40610_at, 36617_at, 37724_at, 1693_s_at, 39054_at, 37456_at, 754_s_at, 38585_at, 33528_at, 33989_f_at, 37716_at, 37187_at, 38097_at, 907_at, 36780_at, 35523_at, 36881_at, 37376_at, 38747_at, 32668_at, 39698_at, 37705_at 37179_at, 36749_at, 207_at, 1520_s_at, 38675_at, 1752_at, 34892_at, 203_at, 40422_at, 538_at, 36618_g_at, 37348_s_at, 33425_at, 39775_at, 41332_at, 39936_at, 40767_at, 1643_g_at, 37194_at, 40916_at, 39298_at, 262_at, 36138_at, 40827_at, 33809_at, 40718_at, 1472_g_at, 36711_at, 31622_f_at, 32542_at, 35371_at, 37242_at, 32165_at, 37384_at, 34023_at, 36684_at, 38123_at, 41322_s_at, 35182_f_at, 31670_s_at, 32087_at, 37018_at, 35292_at, 36958_at, 32548_at, 34961_at, 40962_s_at, 33219_at, 38473_at, 37399_at, 38052_at, 33925_at, 34251_at, 1449_at, 39341_at, 39767_at, 41202_s_at, 1942_s_at, 32844_at, 35342_at, 41123_s_at, 38233_at, 2012_s_at, 35848_at, 38443_at, 39792_at, 37392_at, 1476_s_at, 34325_at, 36185_at, 38808_at, 1287_at, 41725_at, 36892_at, 39139_at, 1660_at, 41243_at, 153_f_at, 1826_at, 40638 at, 34099_f_at, 37281_at, 34893_at, 33891_at, 39639_s_at, 36375_at, 39059_at, 37924_g_at, 1237_at, 36277_at, 38113_at, 35590_s_at, 34912_at, 39822_s_at, 34161_at, 35091_at, 214_at, 721_g_at, 179_at, 100_g_at, 38229_at, 35485_at, 32228_at, 37425_g_at, 34060_g_at, 36378_at, 36916_at, 32469_at, 595_at, 32227_at, 888_s_at, 39815_at, 1894_f_at, 38162_at, 38406_f_at, 37898_r_at, 39527_at, 31815_r_at, 36207_at, 31687_f_at, 2077_at, 36114_r_at, 39399_at, 34112_r_at, 41840_r_at, 37556_at, 34655_at, 31562_at, 31357_at, 32897_at, 40278_at, 40089_at, 32525_r_at, 32904_at, 32407_f_at, 416_s_at, AFFX-M27830_{—}3_at, 32815_at, 1339_s_at, 34415_at, 37351_at, 39598_at, 34627_at, 725_i_at, 35955_at, 33021_at, 31586_f_at, 1937_at, 38513_at, 31578_at, 38508_s_at, 36237_at, 34702_f_at, 39640_at, 37434_at, 32162_r_at, 32579_at, 34350_at, 33244_at, 36548_at, 34703_f_at, 32620_at, 33914_r_at, 916_at, 37137_at, 39765_at, 31499_s_at, 732_f_at, 31666_f_at, 36071_at, 31574_i_at, 1350_at, 37467_at, and 2041_i_at. Example of qualifiers listed in Table 9b but not in Table 3 include 35920_at, 40490_at, 39610_at, 38012_at, 41188_at, 38012_at, 41188_at, 1115_at, 37809_at, 1520_s_at, 32609_at, 41071_at, 36618_g_at, 34397_at, 37508_f_at, 41562_at 1519_at 39209_r_at, 36749_at, 39736_at, 32663_at, 37018_at, 39208_i_at, 33986_r_at, 31522_f_at, 35576_f_at, 40877_s_at, 31523_f_at, 39032_at, 39070_at, 1065_at, 36501_at, 38097_at, 33989_f_at, 39971_at, 32260_at, 33131_at, 35224_at, 286_at, 37179_at, 32819_at, 35672_at, 37185_at, 34378_at, 37532_at, 40878_f_at, 41470_at, 40775_at, 36347_f_at, 36780_at, 40698_at, 39698_at, AFFX-HUMRGE/M10098_M_at, 31665_s_at, 40088_at, 39341_at, 857_at, 1842_at, 41357_at, 40076_at, 39420_at, 34850_at, 430_at, 37218_at, 33885_at, 36709_at, 31888_s_at, 32508_at, 35842_at, 34308_at, 37033_s_at, 40617_at, 32857_at, 1940_at, 38653_at, 262_at, 40365 at, 31895_at, 40827_at, 40979_at, 36785_at, 34610_at, 40815_g_at, 34857_at, 39969_at, 39389_at, 32241_at, 40916_at, 32808_at, 33219_at, 38363_at, 2077_at, 33501_r_at, 38201_at, AFFX-HSAC07/X00351_{—}5_at, 1353_g_at, 41694_at, 38391_at, 38112_g_at, 32673_at, 33500_i_at, 35536_at, 1832_at, 35807_at, 37623_at, 916_at, 35013_at, 39245_at, 36879_at, 1252_at, 37145_at, 31562_at, 31586_f_at, 39872_at, 35094_f_at, 39591_s_at, 38194_s_at, 41627_at, 1339_s_at, 33274_f_at, 36338_at, 33273_f_at, 33150_at, 41471_at, 1117_at 33284_at, 31506_s_at, 34350_at, 39593_at, 33963_at, 35591_at, 37864_s_at, 36674_at, 37121_at, 39413_at, 41440_at, 32275_at, 40278_at, 36983_f_at, 34415_at, 41840_r_at, AFFX-M27830_{—}3_at, 38513_at 38249_at, 31793_at, 1407_g_at, 31578_at, 40234_at, 35762_at, 40517_at, 31357_at, 33021_at, 1350_at, 36237_at, 38273_at, 37434_at, 34013_f_at, 32054_at, 36548_at, 33244_at, 39765_at, 33742_f_at, 34014_f_at, 732_f_at, 33914_r_at, 38908_s_at, 32620_at, 32579_at, 39894_f_at, 36071_at, 35418_at, 31666_f_at, 31574_i_at, and 2041_i_at.

In many embodiments, pattern recognition or comparison programs, such as the k-nearest-neighbors algorithm or the weighted voting algorithm, are employed for the comparison of expression profiles. In addition, the serial analysis of gene expression (SAGE) technology, the GEMTOOLS gene expression analysis program (Incyte Pharmaceuticals), the GeneCalling and Quantitative Expression Analysis technology (Curagen), or other suitable methods, programs or systems can be used to compare expression profiles.

The AML or MDS disease genes of the present invention can be used not only for diagnosing or monitoring the treatment or progression of AML or MDS, but also for predicting the progression from MDS to AML. As discussed below, more than 70% MDS patients who were determined to be AML using the gene expression-based analysis of the present invention eventually progressed to AML. Therefore, the AML or MDS disease genes of the present invention can be used as early indicators of AML progression in patients with MDS.

### D. DIAGNOSIS AND MONITORING THE TREATMENT OR PROGRESSION OF AML AND MDS USING WEIGHTED VOTING ALGORITHM

Algorithms, such as the weighted voting program, can be used for diagnosing or monitoring the treatment or progression of AML or MDS. The weighted voting algorithm is described in Golub et al., supra, and Slonim et al., supra, and can assign a patient of interest to one of two or more classes (e.g., AML versus disease-free, MDS versus disease-free, or AML versus MDS versus disease-free). Softwares capable of performing the weighted voting algorithm include, but are not limited to, the GeneCluster 2 software provided by MIT Center for Genome Research at Whitehead Institute.

Under one form of the algorithm, a patient of interest can be assigned to one of two classes (class 0 and class 1). In one example, class 0 includes disease-free humans, and class 1 includes MDS patients. In another example, class 0 includes disease-free humans, and class 1 includes AML patients. A set of MDS (or AML) disease genes can be selected to form a class predictor (classifier). Each gene in the class predictor casts a weighted vote for one of the two classes (class 0 and class 1). The vote of gene “g” can be defined as v_{g}=a_{g}(x_{g}−b_{g}), wherein a_{g} equals to P(g,c) and reflects the correlation between the expression level of gene “g” and the class distinction between class 0 and class 1. b_{g} equals to [x0(g)+x1(g)]/2, which is the average of the mean logs of the expression levels of gene “g” in class 0 and class 1. x_{g} represents the normalized log of the expression level of gene “g” in the sample of interest. A positive v_{g} indicates a vote for class 0, and a negative v_{g} indicates a vote for class 1. V0 denotes the sum of all positive votes, and V1 denotes the absolute value of the sum of all negative votes. A prediction strength PS is defined as PS=(V0−V1)/(V0+V1).

Cross-validation can be used to evaluate the accuracy of a class predictor created under the weighted voting algorithm. In one embodiment, cross-validation includes withholding a sample which has been used in the neighborhood analysis for the identification of the disease genes. A class predictor is created based on the remaining samples, and then used to predict the class of the sample withheld. This process is repeated for each sample that has been used in the neighborhood analysis.

Class predictors with different MDS (or AML) disease genes can be evaluated by cross-validation. The best class predictor with the most accurate predication can be identified.

In one embodiment, a positive predication that a test sample belongs to class 0 or class 1 is made if the absolute value of PS for the test sample is no less than 0.3. Other PS threshold, such as no less than 0.1, 0.2, 0.4 or 0.5, may also be used to determine a sample's class membership.

In another embodiment, the AML (or MDS) disease genes in a class predictor are significantly correlated with the class distinction in neighborhood analysis. For instance, the disease genes can be selected from those above the 1%, 5%, or 10% significance level in neighborhood analysis. See Golub et al., supra, and Slonim et al., supra.

In yet another embodiment, a class predictor of the present invention includes top upregulated AML or MDS disease gene or genes, and/or top down-regulated AML or MDS disease gene or genes. A class predictor can include both AML and MDS disease genes. Two-class or multi-class correlation metrics can be used for the prediction of disease status.

In still another embodiment, a class predictor of the present invention includes n MDS (or AML) disease genes. A half of these MDS (or AML) disease genes have top P(g,c) scores, and the other half has top −P(g,c) scores. The number n is the only free parameter in defining the class predictor.

In a further embodiment, a class predictor of the present invention comprises or consists of at least 2, 4, 6, 8, 10, 12, 14, 16, 18, 20, 40, or more AML (or MDS) disease genes. The AML (or MDS) disease genes can include at least two groups of genes. The first group includes disease gene or genes having AML/Disease-Free ratios (or MDS/Disease-Free ratios) of at least 1.5, 2, 3, 4, 5, 10, or more. The second group includes disease gene or genes having AML/Disease-Free ratios (or MDS/Disease-Free ratios) of no greater than 0.667, 0.5, 0.333, 0.25, 0.2, 0.1, or less. In still another embodiment, each disease gene in a class predictor has a p-value of no greater than 0.05, 0.01, 0.005, 0.001, 0.0005, 0.0001 or less.

In many embodiments, a confidence threshold is established to optimize the accuracy of prediction and minimize the incidence of both false positive and false negative results. Average confidence scores collected for the accumulating pool of correctly diagnosed patients and correctly non-diagnosed disease-free individuals can be calculated, and a confidence threshold for a particular predictive gene set can be selected.

### E. OTHER APPLICATIONS

A clinical challenge concerning AML, MDS and other blood or bone marrow diseases is the highly variable response of patients to a therapy. The basic concept of pharmacogenomics is to understand a patient's genotype in relation to available treatment options and then individualize the most appropriate option for the patient. Different classes of patients can be created based on their different responses to a given therapy. Genes differentially expressed in one response class compared to another class may be identified using the global gene expression analysis. These genes are molecular markers for predicting whether a patient of interest will be more or less responsive to the therapy. For patients predicted to have a favorable outcome, efforts to minimized toxicity of the therapy may be considered, whereas for those predicted not to respond to the therapy, treatment with other therapies or experimental regimes can be explored.

In one embodiment, patients are grouped into at least two classes (class 0 and class 1). Class 0 includes patients who die within a specified period of time (such as one year) after initiation of a treatment. Class 1 includes patient who survive beyond the specified period of time after initiation of the treatment. Genes that are differentially expressed in class 0 compared to class 1 can be identified. These genes are prognostic markers of patient clinical outcome. Other clinical outcome criteria, such as time to progression, complete response, partial response, stable disease, or progressive disease, can also be used to group the patients and identify the respective prognosis genes.

The disease genes of the present invention can be used to monitor the progression or treatment of AML or MDS. For instance, the return of a disease gene to the normal expression level is indicative of the effectiveness of a treatment of the disease. The disease genes of the present invention can also be used to identify or test drugs for the treatment of AML or MDS. The ability of a drug candidate to reduce or abolish the abnormal expression of AML or MDS disease genes is suggestive of the effectiveness of the drug candidate in treating AML or MDS. Methods for screening or evaluating drug candidates are well known in the art. These methods can be carried out either in animal models or during human clinical trials.

The present invention contemplates expression vectors encoding AML or MDS disease genes. These AML or MDS disease genes may be under-expressed in AML or MDS tumor cells. By introducing the expression vectors into the patients in need thereof, abnormal expression of these genes may be corrected. Suitable expression vectors and gene delivery techniques are well known in the art.

In addition, this invention contemplates expression vectors encoding sequences that are antisense to AML and MDS disease genes. The AML or MDS disease genes may be over-expressed in AML or MDS tumor cells. By introducing the antisense expression vectors, abnormal expression of these disease genes can be corrected.

Expression of an AML or MDS disease gene can also be inhibited using RNA interference (“RNAi”). RNAi is a technique used in post transcriptional gene silencing (“PTGS”), in which the targeted gene activity is specifically abolished. RNA; resembles in many aspects PTGS in plants and has been detected in many invertebrates including trypanosome, hydra, planaria, nematode and fruit fly (Drosophila melanogaster). It may be involved in the modulation of transposable element mobilization and antiviral state formation. RNAi in mammalian systems is disclosed in PCT application WO00/63364. In one embodiment, dsRNA of at least about 21 nucleotides is introduced into cells to silence the expression of the target gene.

Antibodies against the polypeptides encoded by AML or MDS disease genes can be administered to patients in need thereof. In one embodiment, the antibodies can substantially reduce or inhibit the activity of a disease gene. For instance, the antibodies can reduce the activity of the disease gene by at least about 25%, 50%, 75%, 90%, or more.

A pharmaceutical composition comprising an antibody or an expression vector of the present invention can be prepared. The pharmaceutical composition can be formulated to be compatible with its intended route of administration. Examples of routes of administration include, but are not limited to, parenteral, intravenous, intradermal, subcutaneous, oral, inhalational, transdermal, topical, transmucosal, and rectal administration. Preparation of pharmaceutical compositions is well known in the art.

The present invention further features kits or apparatuses for diagnosing or monitoring the progression or treatment of AML or MDS. In one embodiment, the kits or apparatuses include one or more polynucleotides, each of which is capable of hybridizing under stringent conditions to a gene selected from Tables 1, 3, 8b, 9b, and 10b. The polynucleotides can be labeled with fluorescent, radioactive, or other detectable moieties. Any number of polynucleotides can be included in a kit. For instance, at least 2, 3, 4, 5, 10, 15, 20, or more polynucleotides can be included in a kit or apparatus, and each polynucleotide is capable of hybridizing under stringent conditions to a different respective gene selected from Tables 1, 3, 8b, 9b, and 10b. In one example, the polynucleotides are included in vials, tubes, bottles or other containing means. In another example, the polynucleotides are stably attached to one or more substrate supports. Nucleic acid hybridization can be directly carried out on the substrate supports.

In another embodiment, the kits or apparatuses include one or more antibodies specific for the polypeptides encoded by the genes selected from Tables 1, 3, 8b, 9b, and 10b. The antibodies can be labeled or unlabeled. Any number of antibodies can be included in a kit or apparatus. For instance, at least 2, 3, 4, 5, 10, 15, 20, or more antibodies can be included in a kit or apparatus, and each antibody can specifically recognize a different respective AML or MDS disease gene product. In one example, the kit or apparatus also includes other immunodetection reagents (such as secondary antibodies, controls or enzyme substrates). In another example, the antibodies in a kit of the present invention are included in one or more containers. In yet another example, the antibodies in an apparatus of the present invention are stably attached to one or more substrate supports. Suitable substrate supports include, but are not limited to, films, membranes, column matrices, or microtiter plate wells. Immunoassays can be performed directly on the substrate supports.

Furthermore, the present invention features systems capable of comparing an expression profile of interest to at least one reference expression profile. In many embodiments, the reference expression profiles are stored in a database. The comparison between the expression profile of interest and the reference expression profile(s) can be carried out electronically, such as by using a computer system. The computer system typically comprises a processor coupled to a memory which stores data representing the expression profiles to be compared. In one embodiment, the memory is readable as well as rewritable. The expression profiles can be retrieved or modified. The computer system includes one or more programs capable of causing the processor to compare the expression profiles. In one embodiment, the computer system includes a program capable of executing a weighted voting algorithm. In another embodiment, the computer system is coupled to a polynucleotide array from which hybridization signals can be directly fed into the computer system.

It should be understood that the above-described embodiments and the following examples are given by way of illustration, not limitation. Various changes and modifications within the scope of the present invention will become apparent to those skilled in the art from the present description.

### F. EXAMPLES

### Example 1

### Isolation of RNA and Preparation of Labeled Microarray Targets

BMMCs were isolated from bone marrow aspirates taken from 15 disease-free volunteers, 17 patients with MDS, and 18 patients with AML. Informed consents for the pharmacogenomic portions of these clinical studies were received and the project was approved by the local Institutional Review Boards at the participating clinical sites. MDS patients were primarily of Caucasian descent and had a mean age of 66 years (range of 52-84 years). AML patients were exclusively of Caucasian descent and had a mean age of 45 years (range of 19-65 years). Disease-free volunteers were exclusively of Caucasian descent with a mean age of 23 years (range of 18-32 years).

At screening, bone marrow aspirates from each patient were obtained for pharmacogenomic assessment and histopathologically examined by two independent pathologists. Each bone marrow sample was examined initially by an on-site pathologist and secondly by a single centralized pathologist who screened all samples in the present study and classified the aspirates accordingly. Inclusion criteria for AML patients included blasts in excess of 20% in the bone marrow, morphologic diagnosis of AML according to the FAB classification system and flow cytometry analysis indicating CD33⁺ status. Inclusion criteria for MDS patients included morphologic diagnosis of MDS and FAB classification as refractory anemia, refractory anemia with ringed sideroblasts, refractory anemia with excess blasts, or refractory anemia with excess blasts in transformation (where disease stability had been demonstrated for a minimum of 2 months).

BMMCs from individuals were isolated from whole bone marrow aspirates. All disease-free and diseased bone marrow samples were shipped or stored overnight prior to processing. Total RNA was isolated from BMMC pellets using the RNeasy mini kit (Qiagen, Valencia, Calif.). Labeled target for oligonucleotide arrays was prepared using a modification of the procedure described in Lockhart et al., NATURE BIOTECHNOLOGY, 14: 1675-80 (1996). Labeled probes were hybridized to oligonucleotide arrays comprised of over 12,600 human sequences (HgU95Av2 or HG-U95A, Affymetrix) according to the Affymetrix GeneChip Analysis Suite User Guide.

### Example 2

### Hybridization to Affymetrix Microarrays and Detection of Fluorescence

2 μg total RNA is converted to cDNA by priming with an oligo-dT primer containing a T7 DNA polymerase promoter at the 5′ end. The cDNA is used as the template for in vitro transcription using a T7 DNA polymerase kit (Ambion, Woodlands, Tex.) and biotinylated CTP and UTP (Enzo). Labeled cRNA can be fragmented in 40 mM Tris-acetate pH 8.0, 100 mM KOAc, 30 mM MgOAc for 35 minutes at 94° C. in a final volume of 40 μl.

Individual diseased and disease-free samples are hybridized to HgU95Av2 or HG-U95A genechips (Affymetrix). No samples are pooled. 10 μg of labeled target can be diluted in 1×MES buffer with 100 μg/ml herring sperm DNA and 50 μg/ml acetylated BSA. To normalize arrays to each other and to estimate the sensitivity of the oligonucleotide arrays, in vitro synthesized transcripts of 11 bacterial genes can be included in each hybridization reaction as described in Hill et al., SCIENCE, 290: 809-812 (2000). The abundance of these transcripts can range from 1:300,000 (3 ppm) to 1:1000 (1000 ppm) stated in terms of the number of control transcripts per total transcripts. As determined by the signal response from these control transcripts, the sensitivity of detection of the arrays can range between about 1:300,000 and 1:100,000 copies/million.

Labeled probes are denatured at 99° C. for 5 minutes and then 45° C. for 5 minutes and hybridized to oligonucleotide arrays comprised of over 12,500 human genes (HG-U95Av2 or HgU95A, Affymetrix). Arrays can be hybridized for 16 hours at 45° C. The hybridization buffer can include 100 mM MES, 1 M [Na⁺], 20 mM EDTA, and 0.01% Tween 20. After hybridization, the cartridges can be washed extensively with wash buffer 6×SSPET (e.g., three times at room temperature for at least 10 minutes each time). These hybridization and washing conditions are collectively referred to as “nucleic acid array hybridization conditions.” The washed cartridges can be subsequently stained with phycoerythrin coupled to streptavidin.

12×MES stock contains 1.22 M MES and 0.89 M [Na⁺]. For 1000 ml, the stock can be prepared by mixing 70.4 g MES free acid monohydrate, 193.3 g MES sodium salt and 800 ml of molecular biology grade water, and adjusting volume to 1000 ml. The pH should be between 6.5 and 6.7. 2× hybridization buffer can be prepared by mixing 8.3 ml of 12×MES stock, 17.7 ml of 5 M NaCl, 4.0 ml of 0.5 M EDTA, 0.1 ml of 10% Tween 20 and 19.9 ml of water. 6×SSPET contains 0.9 M NaCl, 60 mM NaH₂PO₄, 6 mM EDTA, pH 7.4, and 0.005% Triton X-100. In some cases, the wash buffer can be replaced with a more stringent wash buffer. 1000 ml stringent wash buffer can be prepared by mixing 83.3 ml of 12×MES stock, 5.2 ml of 5 M NaCl, 1.0 ml of 10% Tween 20 and 910.5 ml of water.

### Example 3

### Gene Expression Data Analysis

Data analysis and absent/present call determination was performed on raw fluorescent intensity values using GENECHIP 3.2 software (Affymetrix). The “average difference” values for each transcript were normalized to “frequency” values using the scaled frequency normalization method in which the average differences for 11 control cRNAs with known abundance spiked into each hybridization solution were used to generate a global calibration curve. See Hill et al., GENOME BIOL., 2(12):research0055.1-0055.13 (2001), which is incorporated herein by reference. This calibration was then used to convert average difference values for all transcripts to frequency estimates, stated in units of parts per million ranging from 1:300,000 (3 parts per million (ppm)) to 1:1000 (1000 ppm).

GENECHIP 3.2 software uses algorithms to calculate the likelihood as to whether a gene is “absent” or “present” as well as a specific hybridization intensity value or “average difference” for each transcript represented on the array. The algorithms used in these calculations are described in the Affymetrix GeneChip Analysis Suite User Guide.

Specific transcripts can be evaluated further if they meet the following criteria. First, genes that are designated “absent” by the GENECHIP 3.2 software in all samples are excluded from the analysis. Second, in comparisons of transcript levels between arrays, a gene is required to be present in at least one of the arrays. Third, for comparisons of transcript levels between groups, a Student's t-test is applied to identify a subset of transcripts that had a significant difference (p<0.05) in frequency values. In certain cases, a fourth criteria, which requires that average fold changes in frequency values across the statistically significant subset of genes be 2-fold or greater, can also be used.

Unsupervised hierarchical clustering of genes and/or arrays on the basis of similarity of their expression profiles was performed using the procedure described in Eisen et al., PROC. NAT. ACAD. SCI., U.S.A., 95: 14863-14868 (1998). Nearest-neighbor prediction analysis and supervised cluster analysis were performed using metrics illustrated in Golub et al., supra. Computer programs for nearest-neighbor prediction analysis and supervised cluster analysis can be obtained from www-genome.wi.mit.edu/cancer/software/genecluster2/gc2.html. For hierarchical clustering and nearest-neighbor prediction analysis, data were log transformed and normalized to have a mean value of zero and a variance of one. A Student's t-test was used to compare disease-free, AML and MDS BMMC expression profiles. A p value of no more than 0.05 (e.g., no more than 0.01, 0.001, or less) may be used to indicate statistical significance.

Expression profiles in various tissues can also be accessed and downloaded from the BioExpress database (GeneLogic, Gaithersburg Md.). GeneLogic GX2000 software based analysis tools including fold change analysis and electronic northerns can be utilized to calculate fold changes and distribution of expression values. Expression profiles for different samples can be exported using the expression analysis tool for further analysis in the hierarchical clustering package (Eisen et al., supra).

A k-nearest-neighbor's approach was used to perform a neighborhood analysis of real and randomly permuted data using a correlation metric (P(g,c)=μ1−μ2/σ1+σ2), where g is the expression vector of a gene, c is the class vector, ∥1 and σ1 define the mean expression level and standard deviation of the gene in class 1, respectively, and μ2 and σ2 define the mean expression level and standard deviation of the gene in class 2, respectively. The measures of correlation for the most statistically significant upregulated genes of the true defined classes (AML versus disease-free, or MDS versus disease-free) can be compared to the most statistically significant measures of correlation observed in randomly permuted class distinctions. The top 1%, 5% and median distance measurements of 100 randomly permuted classes compared to the observed distance measurements for AML (or MDS) and disease-free classes can be plotted to show the statistical verification of the AML (or MDS) disease genes identified by this invention.

### Example 4

### Identification of AML and MDS Disease Genes

Expression profiling analysis of the disease-free BMMC RNA samples, MDS BMMC RNA samples and AML BMMC RNA samples revealed that of the over 12,000 genes on HG-U95Av2 or HgU95A chips, at least 2,768 genes met an initial criteria for further analysis (i.e., at least 1 present call, and at least 1 frequency >10 ppm). Tables 1 and 2 list examples of the identified AML disease genes, and Tables 3 and 4 list examples of the identified MDS disease genes.

An initial unsupervised cluster analysis approach, which hierarchically clusters samples and genes based on a correlation coefficient (Eisen et al., supra), was performed using the 2,768 genes passing the initial filtering criteria (FIG. 1). The dendrogram in FIG. 1 describes sample relationships and groups the disease-free BMMCs, AML BMMCs, and a subset of MDS BMMCs into three respective clusters. Another subset of MDS-diagnosed patient BMMCs clustered with the AML samples, indicating that BMMC profiles from these MDS patients were molecularly more similar to BMMC profiles in AML patients. Evaluation of these MDS patients revealed that they included MDS patients who had conflicting diagnoses and MDS patients who ultimately progressed to AML. This observation indicated that BMMCs of MDS patients destined to progress to AML possessed patterns of expression in at least certain genes that were more similar to patterns of expression observed in patients with AML. Each sample in FIG. 1 has a sample ID starting with “norm.”, “X207.”, or “X206,” respectively. HOXA9, PBX3, PRKACB, CMAH, PFKP, PLAGL1, ACTA2, and FLT3 denote the respective genes in the unsupervised cluster analysis.

### Example 5

### Classification of AML Versus Disease-Free, MDS Versus Disease-Free, and AML Versus MDS Using BMMC Gene Expression Profiles

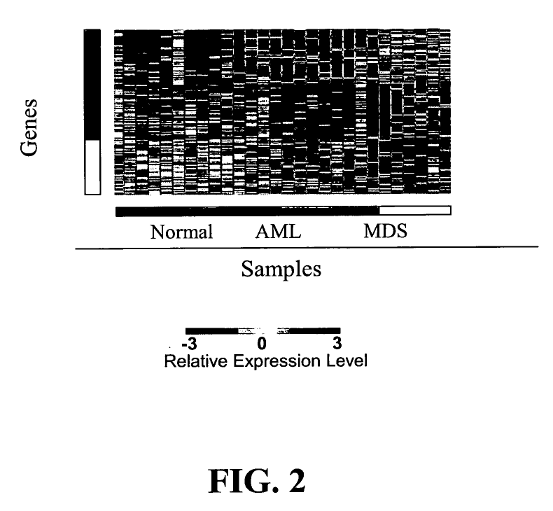

A supervised approach was employed to identify transcripts whose expression levels were most highly correlated with BMMCs from disease-free, AML or MDS patients. To initially build and subsequently train the classifiers, 70% of the disease-free bone marrow expression patterns (n=10 out of 15), AML bone marrow expression pattern (n=12 out of 18) and MDS bone marrow expression patterns (n=6 out of 9 MDS patients who did not have conflicting diagnosis or progress to AML) were randomly selected and used as the training set. The remaining 30% samples were used as the test set. Genecluster's default correlation metric (Golub et al., supra) was used to identify genes with expression levels most highly correlated with the classification vector characteristic of the training set. All 2,768 genes meeting the initial filter criteria were screened using this approach. Predictor sets containing different numbers of genes were then evaluated by “leave one out cross validation” (LOOCV) to identify the predictor set with the highest accuracy for classification of the samples in the training set. Classifier sets containing top genes upregulated in AML BMMCs, top genes upregulated in MDS BMMCs, and top genes upregulated in disease-free BMMCs were prepared. Upregulation can be determined by fold changes. FIG. 2 depicts the relative expression levels of a 93-gene classifier set which includes 31 top genes upregulated in AML BMMCs, 31 top genes upregulated in MDS BMMCs, and 31 top genes upregulated in disease-free BMMCs. The 93-gene classifier set was found to yield 100% prediction accuracy by LOOCV on the training set. The prediction accuracy of other classifier sets thus-prepared was shown in Table 6.

TABLE 6
Prediction Accuracy of Exemplary Classifiers

| Number of Genes | Prediction Accuracy (%) | Prediction Accuracy (%) |
|---|---|---|
| in the Classifier | (Training Set) | (Test Set) |
| 2 | 82 | 79 |
| 3 | 93 | 79 |
| 4 | 93 | 86 |
| 5 | 93 | 93 |
| 6 | 86 | 100 |
| 7 | 96 | 100 |
| 8 | 93 | 100 |
| 9 | 96 | 100 |
| 10 | 96 | 100 |
| 11 | 96 | 100 |
| 12 | 100 | 100 |
| 13 | 100 | 100 |
| 14 | 96 | 100 |
| 15 | 96 | 100 |
| 16 | 97 | 100 |
| 17 | 96 | 100 |
| 18 | 96 | 100 |
| 19 | 96 | 100 |
| 20 | 96 | 100 |
| 21 | 96 | 100 |
| 22 | 96 | 100 |
| 23 | 96 | 100 |
| 24 | 96 | 100 |
| 25 | 96 | 100 |
| 26 | 96 | 100 |
| 27 | 96 | 100 |
| 28 | 96 | 100 |
| 29 | 96 | 100 |
| 30 | 96 | 100 |
| 31 | 96 | 100 |
| 32 | 96 | 100 |
| 33 | 96 | 100 |
| 34 | 100 | 100 |
| 35 | 100 | 100 |
| 36 | 100 | 100 |
| 37 | 100 | 100 |
| 38 | 100 | 100 |
| 39 | 100 | 100 |
| 40 | 100 | 100 |
| 41 | 100 | 100 |
| 42 | 100 | 100 |
| 43 | 100 | 100 |
| 44 | 100 | 100 |
| 45 | 100 | 100 |
| 46 | 100 | 100 |
| 47 | 100 | 100 |
| 48 | 100 | 100 |
| 49 | 100 | 100 |
| 50 | 100 | 100 |

The 93-gene classifier set is depicted in Tables 7a and 7b. The class within which each gene is upregulated is indicated (“Class Predicted”). Table 7b provides the cytogenetic band, the Unigene accession number, and the Entrez accession number for each of the 93 genes.

TABLE 7a
An Exemplary 93-Gene Classifier

| Gene Name | Class Predicted | Gene Title | Qualifier |
|---|---|---|---|
| DEFA4 | Disease-free | defensin, alpha 4, corticostatin | 34546_at |
| TFF3 | Disease-free | trefoil factor 3 (intestinal) | 37897_s_at |
| GW112 | Disease-free | differentially expressed in hematopoietic | 38615_at |
| | | lineages | |
| LCN2 | Disease-free | Lipocalin 2 (oncogene 24p3) | 32821_at |
| HK3 | Disease-free | hexokinase 3 (white cell) | 36372_at |
| CAMP | Disease-free | cathelicidin antimicrobial peptide | 36710_at |
| ELA2 | Disease-free | elastase 2, neutrophil | 37096_at |
| CTSG | Disease-free | cathepsin G | 679_at |
| IGHM | Disease-free | immunoglobulin heavy constant mu | 41165_g_at |
| S100A12 | Disease-free | S100 calcium-binding protein A12 | 38879_at |
| | | (calgranulin C) | |
| SH3BP5 | Disease-free | SH3-domain binding protein 5 (BTK- | 38968_at |
| | | associated) | |
| MS4A3 | Disease-free | membrane-spanning 4-domains, subfamily | 32451_at |
| | | A, member 3 (hematopoietic cell-specific) | |
| SGP28 | Disease-free | specific granule protein (28 kDa); cysteine- | 36464_at |
| | | rich secretory protein-3 | |
| CEACAM8 | Disease-free | carcinoembryonic antigen-related cell | 33530_at |
| | | adhesion molecule 8 | |
| ITGAM | Disease-free | integrin, alpha M (complement component | 38533_s_at |
| | | receptor 3, alpha; also known as CD11b | |
| | | (p170), macrophage antigen alpha | |
| | | polypeptide) | |
| SLPI | Disease-free | secretory leukocyte protease inhibitor | 32275_at |
| | | (antileukoproteinase) | |
| TFF3 | Disease-free | trefoil factor 3 (intestinal) | 31477_at |
| PIR121 | Disease-free | p53 inducible protein | 37579_at |
| GSN | Disease-free | gelsolin (amyloidosis, Finnish type) | 32612_at |
| UNK_U95626 | Disease-free | Cluster Incl U95626: Homo sapiens ccr2b | 37149_s_at |
| | | (ccr2), ccr2a (ccr2), ccr5 (ccr5) and ccr6 | |
| | | (ccr6) genes, complete cds, and lactoferrin | |
| | | (lactoferrin) gene, partial cds, complete | |
| | | sequence. | |
| ALOX5AP | Disease-free | arachidonate 5-lipoxygenase-activating | 37099_at |
| | | protein | |
| BPI | Disease-free | bactericidal/permeability-increasing protein | 37054_at |
| PRTN3 | Disease-free | proteinase 3 (serine proteinase, neutrophil, | 37066_at |
| | | Wegener granulomatosis autoantigen) | |
| PGLYRP | Disease-free | peptidoglycan recognition protein | 31381_at |
| CTSG | Disease-free | cathepsin G | 37105_at |
| AZU1 | Disease-free | azurocidin 1 (cationic antimicrobial protein | 33963_at |
| | | 37) | |
| CEACAM6 | Disease-free | carcinoembryonic antigen-related cell | 36105_at |
| | | adhesion molecule 6 (non-specific cross | |
| | | reacting antigen) | |
| TCN1 | Disease-free | transcobalamin I (vitamin B12 binding | 35919_at |
| | | protein, R binder family) | |
| DEFA1 | Disease-free | defensin, alpha 1, myeloid-related sequence | 31793_at |
| NCF4 | Disease-free | neutrophil cytosolic factor 4 (40 kD) | 38895_i_at |
| S100P | Disease-free | S100 calcium-binding protein P | 34319_at |
| PPID | AML | peptidylprolyl isomerase D (cyclophilin D) | 948_s_at |
| HSPCB | AML | heat shock 90 kD protein 1, beta | 33984_at |
| HSPCB | AML | heat shock 90 kD protein 1, beta | 1161_at |
| UQCRC2 | AML | ubiquinol-cytochrome c reductase core | 40854_at |
| | | protein II | |
| APEX | AML | APEX nuclease (multifunctional DNA | 2025_s_at |
| | | repair enzyme) | |
| CCT8 | AML | chaperonin containing TCP1, subunit 8 | 39767_at |
| | | (theta) | |
| NDUFS4 | AML | NADH dehydrogenase (ubiquinone) Fe—S | 38695_at |
| | | protein 4 (18 kD) (NADH-coenzyme Q | |
| | | reductase) | |
| FARSL | AML | phenylalanine-tRNA synthetase-like | 1750_at |
| KARS | AML | lysyl-tRNA synthetase | 34336_at |
| NDUFB5 | AML | NADH dehydrogenase (ubiquinone) 1 beta | 32232_at |
| | | subcomplex, 5 (16 kD, SGDH) | |
| CCT3 | AML | chaperonin containing TCP1, subunit 3 | 40774_at |
| | | (gamma) | |
| CCT2 | AML | chaperonin containing TCP1, subunit 2 | 35759_at |
| | | (beta) | |
| ESD | AML | esterase D/formylglutathione hydrolase | 38375_at |
| NPM1 | AML | nucleophosmin (nucleolar phosphoprotein | 38542_at |
| | | B23, numatrin) | |
| HRMT1L2 | AML | HMT1 (hnRNP methyltransferase, S. cerevisiae)- | 32825_at |
| | | like 2 | |
| OA48-18 | AML | acid-inducible phosphoprotein | 34397_at |
| SET | AML | SET translocation (myeloid leukemia- | 40189_at |
| | | associated) | |
| FNTA | AML | farnesyltransferase, CAAX box, alpha | 1499_at |
| EIF3S7 | AML | eukaryotic translation initiation factor 3, | 35298_at |
| | | subunit 7 (zeta, 66/67 kD) | |
| SNRPE | AML | small nuclear ribonucleoprotein | 38679_g_at |
| | | polypeptide E | |
| UNK_U47077 | AML | Human DNA-dependent protein kinase | 40129_at |
| | | catalytic subunit (DNA-PKcs) mRNA, | |
| | | complete cds | |
| ADE2H1 | AML | multifunctional polypeptide similar to | 39056_at |
| | | SAICAR synthetase and AIR carboxylase | |
| LDHB | AML | lactate dehydrogenase B | 33819_at |
| HADHB | AML | hydroxyacyl-Coenzyme A | 39741_at |
| | | dehydrogenase/3-ketoacyl-Coenzyme A | |
| | | thiolase/enoyl-Coenzyme A hydratase | |
| | | (trifunctional protein), beta subunit | |
| GA17 | AML | dendritic cell protein | 35814_at |
| RAN | AML | RAN, member RAS oncogene family | 1839_at |
| ACADM | AML | acyl-Coenzyme A dehydrogenase, C-4 to | 37532_at |
| | | C-12 straight chain | |
| NAP1L1 | AML | nucleosome assembly protein 1-like 1 | 571_at |
| ADA | AML | adenosine deaminase | 41654_at |
| ATP5A1 | AML | ATP synthase, H+ transporting, | 40096_at |
| | | mitochondrial F1 complex, alpha subunit, | |
| | | isoform 1, cardiac muscle | |
| EIF3S3 | AML | eukaryotic translation initiation factor 3, | 35327_at |
| | | subunit 3 (gamma, 40 kD) | |
| GPR35 | MDS | G protein-coupled receptor 35 | 31700_at |
| FTH1 | MDS | ferritin, heavy polypeptide 1 | 33943_at |
| TNFRSF1B | MDS | tumor necrosis factor receptor superfamily, | 1583_at |
| | | member 1B | |
| USP12 | MDS | ubiquitin specific protease 12 | 34803_at |
| MGAT1 | MDS | mannosyl (alpha-1,3-)-glycoprotein beta- | 39778_at |
| | | 1,2-N-acetylglucosaminyltransferase | |
| MMPL1 | MDS | matrix metalloproteinase-like 1 | 35910_f_at |
| UNK_AA725102 | MDS | Cluster Incl AA725102: ai08h05.s1 | 32835_at |
| | | Soares_parathyroid_tumor_NbHPA Homo | |
| | | sapiens cDNA clone 1342233 3′ similar to | |
| | | gb: D38081 THROMBOXANE A2 | |
| | | RECEPTOR (HUMAN);, mRNA | |
| | | sequence. | |
| KIAA0077 | MDS | KIAA0077 protein | 36978_at |
| PHF1 | MDS | PHD finger protein 1 | 40446_at |
| MADD | MDS | MAP-kinase activating death domain | 38398_at |
| VDUP1 | MDS | upregulated by 1,25-dihydroxyvitamin D-3 | 31508_at |
| MYO1B | MDS | myosin IB | 32811_at |
| UNK_AL096714 | MDS | Homo sapiens mRNA; cDNA | 38710_at |
| | | DKFZp564E242 (from clone | |
| | | DKFZp564E242) | |
| SGSH | MDS | N-sulfoglucosamine sulfohydrolase | 35626_at |
| | | (sulfamidase) | |
| SAT | MDS | spermidine/spermine N1-acetyltransferase | 34304_s_at |
| SAT | MDS | spermidine/spermine N1-acetyltransferase | 1173_g_at |
| DKFZP586G0522 | MDS | DKFZP586G0522 protein | 36139_at |
| IFIT1 | MDS | interferon-induced protein 56 | 32814_at |
| KRTHB6 | MDS | keratin, hair, basic, 6 (monilethrix) | 32329_at |
| H6PD | MDS | hexose-6-phosphate dehydrogenase | 34066_at |
| | | (glucose 1-dehydrogenase) | |
| CEACAM3 | MDS | carcinoembryonic antigen-related cell | 32469_at |
| | | adhesion molecule 3 | |
| MAPKAPK2 | MDS | mitogen-activated protein kinase-activated | 36179_at |
| | | protein kinase 2 | |
| PPP1R10 | MDS | protein phosphatase 1, regulatory subunit | 38672_at |
| | | 10 | |
| KIAA0230 | MDS | p53-responsive gene 2 | 39327_at |
| UP | MDS | uridine phosphorylase | 37351_at |
| BNIP3L | MDS | BCL2/adenovirus E1B 19 kD-interacting | 39436_at |
| | | protein 3-like | |
| RELA | MDS | v-rel avian reticuloendotheliosis viral | 1271_g_at |
| | | oncogene homolog A (nuclear factor of | |
| | | kappa light polypeptide gene enhancer in | |
| | | B-cells 3 (p65)) | |
| RELA | MDS | v-rel avian reticuloendotheliosis viral | 1295_at |
| | | oncogene homolog A (nuclear factor of | |
| | | kappa light polypeptide gene enhancer in | |
| | | B-cells 3 (p65)) | |
| DKFZP434C091 | MDS | DKFZP434C091 protein | 36713_at |
| KIAA1030 | MDS | KIAA1030 protein | 34089_at |
| STXBP1 | MDS | syntaxin-binding protein 1 | 33942_s_at |

TABLE 7b
An Exemplary 93-Gene Classifier

| | | Unigene | |
|---|---|---|---|
| | Cytogenetic | Accession | |
| Gene Name | Band | No. | Entrez Accession No |
| DEFA4 | 8p23 | Hs.2582 | AI250799 |
| TFF3 | 21q22.3 | Hs.82961 | AI985964 |
| GW112 | 13q14.2 | Hs.273321 | AF097021 |
| LCN2 | 9q34 | Hs.204238 | AI762213 |
| HK3 | 5q35.2 | Hs.159237 | U51333 |
| CAMP | 3p21.3 | Hs.51120 | Z38026 |
| ELA2 | 19p13.3 | Hs.99863 | M34379 |
| CTSG | 14q11.2 | Hs.100764 | J04990 |
| IGHM | 14q32.33 | Hs.153261 | X67301 |
| S100A12 | 1q21 | Hs.19413 | D83664 |
| SH3BP5 | 3p24.3 | Hs.109150 | AB005047 |
| MS4A3 | 11q12-q13.1 | Hs.99960 | L35848 |
| SGP28 | 6p12.2 | Hs.54431 | X94323 |
| CEACAM8 | 19q13.2 | Hs.41 | M33326 |
| ITGAM | 16p11.2 | Hs.172631 | J03925 |
| SLPI | 20q12 | Hs.251754 | X04470 |
| TFF3 | 21q22.3 | Hs.352107 | L08044 |
| PIRI21 | 5q34 | Hs.258503 | L47738 |
| GSN | 9q33 | Hs.290070 | X04412 |
| UNK_U95626 | 3q21-q23 | Hs.105938 | U95626 |
| ALOX5AP | 13q12 | Hs.100194 | AI806222 |
| BPI | 20q11.23-q12 | Hs.89535 | J04739 |
| PRTN3 | 19p13.3 | Hs.928 | X55668 |
| PGLYRP | 19q13.2-q13.3 | Hs.137583 | AF076483 |
| CTSG | 14q11.2 | Hs.100764 | M16117 |
| AZU1 | 19p13.3 | Hs.72885 | M96326 |
| CEACAM6 | 19q13.2 | Hs.73848 | M18728 |
| TCN1 | 11q11-q12 | Hs.2012 | J05068 |
| DEFA1 | 8p23.2-p23.1, | Hs.274463 | AL036554 |
| | 8pter-p23.3 | | |
| NCF4 | 22q13.1 | Hs.196352 | X77094 |
| S100P | 4p16 | Hs.2962 | AA131149 |
| PPID | 4q31.3 | Hs.143482 | D63861 |
| HSPCB | 6p12 | Hs.74335 | M16660 |
| HSPCB | 6p12 | Hs.74335 | J04988 |
| UQCRC2 | 16p12 | Hs.173554 | J04973 |
| APEX | 14q11.2-q12 | Hs.73722 | M80261 |
| CCT8 | 21q22.11 | Hs.15071 | D13627 |
| NDUFS4 | 5q11.1 | Hs.10758 | AA203303 |
| FARSL | 19p13.2 | Hs.23111 | AD000092 |
| KARS | 16q23-q24 | Hs.3100 | D32053 |
| NDUFB5 | 3q27.1 | Hs.19236 | AF047181 |
| CCT3 | 1q23 | Hs.1708 | X74801 |
| CCT2 | 12q13.2 | Hs.6456 | AF026166 |
| ESD | 13q14.1-q14.2 | Hs.82193 | AF112219 |
| NPM1 | 5q35 | Hs.9614 | U89322 |
| HRMT1L2 | 19q13.3 | Hs.20521 | Y10805 |
| OA48-18 | 17, 17q21 | Hs.278670 | AF069250 |
| SET | 9q34 | Hs.145279 | M93651 |
| FNTA | 8p22-q11 | Hs.356463 | L10413 |
| EIF3S7 | 22q13.1 | Hs.55682 | U54558 |
| SNRPE | 1q32 | Hs.334612 | AA733050 |
| UNK_U47077 | 16p13.11, 8q11 | Hs.155637 | U47077 |
| ADE2H1 | 4pter-q21 | Hs.117950 | X53793 |
| LDHB | 12p12.2-p12.1 | Hs.234489 | X13794 |
| HADHB | 2p23 | Hs.146812 | D16481 |
| GA17 | X | Hs.69469 | AF064603 |
| RAN | | | M31469 |
| ACADM | 1p31 | Hs.79158 | M91432 |
| NAP1L1 | 12q14.1 | Hs.302649 | M86667 |
| ADA | 20q12-q13.11 | Hs.1217 | X02994 |
| ATP5A1 | 18q12-q21 | Hs.155101 | D14710 |
| EIF3S3 | 8q24.11 | Hs.58189 | U54559 |
| GPR35 | 2q37.3 | Hs.239891 | AF027957 |
| FTH1 | 11q13 | Hs.62954 | L20941 |
| TNFRSF1B | 1p36.3-p36.2 | Hs.256278 | M32315 |
| USP12 | 15q15-q21.1, | Hs.42400 | AF022789 |
| | 5q33-q34 | | |
| MGAT1 | 5q35 | Hs.151513 | M55621 |
| MMPL1 | 16p13.3 | Hs.198265, | AJ003147 |
| | | Hs.290222 | |
| UNK_AA725102 | 22q13.1 | Hs.51305 | AA725102 |
| KIAA0077 | 2p16.2 | Hs.112396 | D38521 |
| PHF1 | 6p21.3 | Hs.166204 | AL021366 |
| MADD | 11p11.2 | Hs.82548 | AB002356 |
| VDUP1 | 1q12 | Hs.179526 | S73591 |
| MYO1B | 17p13 | Hs.286226 | X98507 |
| UNK_AL096714 | 11q13.1 | Hs.108504 | AL096714 |
| SGSH | 17q25.3 | Hs.31074 | U30894 |
| SAT | Xp22.1 | Hs.28491 | AL050290 |
| SAT | | | U40369 |
| DKFZP586G0522 | 6q21 | Hs.7446 | AL050289 |
| IFIT1 | 10q25-q26 | Hs.20315 | M24594 |
| KRTHB6 | 12q13 | Hs.278658 | X99142 |
| H6PD | 1p36 | Hs.194728 | AJ012590 |
| CEACAM3 | 19q13.2 | Hs.11 | L00693 |
| MAPKAPK2 | 1q32 | Hs.75074 | U12779 |
| PPP1R10 | 6p21.3 | Hs.106019 | Y13247 |
| KIAA0230 | 2pter-p25.1 | Hs.118893 | D86983 |
| UP | 7 | Hs.77573 | X90858 |
| BNIP3L | 8p21 | Hs.132955 | AF079221 |
| RELA | 11q13 | Hs.75569 | L19067 |
| RELA | 11q13 | Hs.75569 | L19067 |
| DKFZP434C091 | 1q44 | Hs.51692 | AL080170 |
| KIAA1030 | 11q25 | Hs.204121 | AB028953 |
| STXBP1 | 9q34.1 | Hs.239356 | AF004563 |

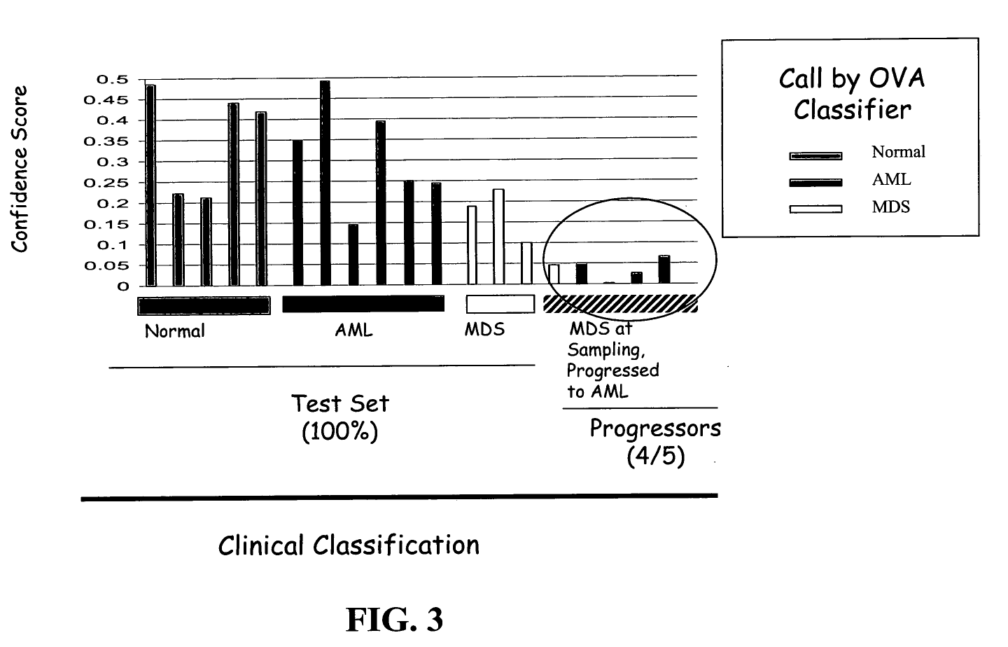

The 93-gene classifier was further evaluated by using the test set of samples. All samples in the test set were accurately predicted as disease-free, AML, or MDS, respectively (FIG. 3). For illustrative purposes, the samples are grouped and ordered along the x-axis according to their clinical and histopathological classification. The magnitudes of the prediction strengths for each sample using the 93-gene classifier model are plotted in the y-dimension (confidence score). These studies demonstrate the feasibility of predicting disease-free, AML or MDS status using expression patterns found in a limited number of gene transcripts in bone marrow mononuclear cells.

Under a weighted voting method the expression level of each gene in the classifier set contributes to an overall prediction strength which determines the classification of the sample. The prediction strength can be measured as a combined variable that indicates the number of “votes” for either one class or another, and can vary between 0 (narrow margin of victory) and 1 (wide margin of victory) in favor of the predicted class. To quantitate the accuracy of this prediction method, a value (such as 0.3) can be imposed as the prediction strength threshold above which calls could confidently be made. In many cases, a prediction strength of less than 0.3 may also have evidentiary value in the prediction of disease status or progression.

The 93-gene classifier on BMMC profiles from MDS patients who ultimately progressed to AML was evaluated. In this study there were five patients histopathologically classified by both pathologists as MDS at the time of bone marrow sampling. These five MDS patients later progressed to AML (median time to progression=137 days). Unsupervised analyses (see FIG. 1) suggested that the overall transcriptomes of these patients' BMMCs were molecularly more similar to BMMC profiles from patients with AML than to BMMC profiles from patients with MDS. The prediction by the 93-gene classifier indicated AML for four of the five MDS patient and MDS for the remaining patient. The confidence scores for these predictions, however, were below 0.05. This result suggests that a prediction of AML on MDS patients, or a prediction with a low confidence score (such as below 0.05), can be used as an indicator of AML progression from MDS prior to clinical diagnosis.

### Example 6

### Identification of AML and MDS Disease Genes

Expression profiles in bone marrow leukocytes from 31 AML patients, 13 MDS patients, and 18 disease-free volunteers were obtained and analyzed using procedures similar to those described in Examples 1-4. Bone marrow leukocytes can be purified from bone marrow aspirates using Ficoll-Hypaque gradients. 531 AML disease genes (Tables 8a and 8b) and 241 MDS disease genes (Tables 9a and 9b) were identified. The average expression level of each of these genes in AML or MDS bone marrow leukocytes is at least 2-fold higher or lower than that in disease-free bone marrow leukocytes. The p value of a Student's t-test (unequal variances) for the difference between the average expression levels of each of these disease genes in AML or MDS versus disease-free bone marrow cells is no more than 0.05. “COV” denotes coefficient of variance.

A similar approach was used to identify genes that are differentially expressed in AML bone marrow cells as compared to MDS bone marrow cells. The qualifiers thus identified are depicted in Table 10a, and the corresponding genes are illustrated in Table 10b. Like other AML or MDS disease genes identified in the present invention, the genes in Tables 10a and 10b can be used for the diagnosis of AML or MDS.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

TABLE 8a
Expression Profiles of AML Disease Genes

| | No. of Present Calls | No. of Present Calls | COV | COV | | P value (unequal) |
|---|---|---|---|---|---|---|
| Qualifier | (Disease-Free n = 18) | (AML n = 31) | (Disease-Free) | (AML) | AML/Disease-Free | (AML vs Disease-Free) |
| 1065_at | 11 | 31 | 25.44% | 97.87% | 8.82 | 2.06451E−05 |
| 41071_at | 16 | 30 | 35.68% | 87.89% | 7.88 | 5.23834E−06 |
| 37754_at | 0 | 18 | 0.00% | 142.43% | 7.55 | 0.001968509 |
| 37809_at | 0 | 25 | 0.00% | 99.15% | 7.45 | 3.4411E−05 |
| 31623_f_at | 2 | 15 | 0.00% | 122.84% | 6.65 | 0.000575118 |
| 34583_at | 0 | 31 | 36.01% | 110.90% | 5.94 | 0.000238341 |
| 38487_at | 0 | 28 | 44.88% | 67.10% | 5.79 | 1.25823E−07 |
| 39610_at | 0 | 21 | 0.00% | 88.20% | 5.39 | 1.56635E−05 |
| 40775_at | 18 | 30 | 34.67% | 96.99% | 5.05 | 7.26944E−05 |
| 32755_at | 0 | 26 | 29.10% | 72.62% | 4.94 | 1.02099E−06 |
| 41138_at | 18 | 31 | 22.60% | 66.21% | 4.57 | 2.85435E−07 |
| 32609_at | 18 | 31 | 30.89% | 45.93% | 4.56 | 1.24239E−10 |
| 41470_at | 15 | 28 | 12.12% | 93.45% | 4.50 | 6.57627E−05 |
| 39175_at | 1 | 30 | 29.10% | 54.10% | 4.49 | 5.75084E−09 |
| 40490_at | 14 | 31 | 37.70% | 65.77% | 4.34 | 3.35019E−07 |
| 39421_at | 5 | 29 | 36.38% | 66.21% | 4.19 | 4.6203E−07 |
| 39317_at | 16 | 31 | 26.24% | 73.27% | 4.10 | 2.90329E−06 |
| 41188_at | 18 | 27 | 24.25% | 133.28% | 4.06 | 0.003719729 |
| 1914_at | 5 | 27 | 32.87% | 183.35% | 4.04 | 0.029706945 |
| 41654_at | 13 | 31 | 36.94% | 61.26% | 4.00 | 1.4416E−07 |
| 36536_at | 1 | 25 | 29.10% | 85.22% | 3.98 | 3.26632E−05 |
| 671_at | 12 | 28 | 38.49% | 122.22% | 3.95 | 0.001970801 |
| 34397_at | 17 | 31 | 37.78% | 38.14% | 3.87 | 2.76445E−12 |
| 1475_s_at | 0 | 22 | 29.10% | 66.57% | 3.80 | 8.93688E−07 |
| 32905_s_at | 2 | 16 | 42.63% | 162.13% | 3.77 | 0.017446944 |
| 33777_at | 4 | 27 | 54.45% | 67.35% | 3.77 | 1.27386E−06 |
| 33352_at | 16 | 31 | 30.97% | 63.72% | 3.76 | 4.41832E−07 |
| 35127_at | 1 | 22 | 22.33% | 132.96% | 3.76 | 0.004517748 |
| 1997_s_at | 3 | 29 | 41.26% | 68.51% | 3.75 | 1.65438E−06 |
| 943_at | 2 | 30 | 35.00% | 64.35% | 3.67 | 6.29477E−07 |
| 39070_at | 17 | 30 | 58.27% | 72.52% | 3.67 | 5.40362E−06 |
| 32245_at | 0 | 28 | 34.30% | 39.36% | 3.66 | 1.18452E−11 |
| 35731_at | 10 | 31 | 44.56% | 68.86% | 3.65 | 2.16558E−06 |
| 32251_at | 15 | 29 | 36.07% | 72.22% | 3.56 | 5.32662E−06 |
| 37283_at | 0 | 17 | 22.33% | 127.81% | 3.51 | 0.004054936 |
| 40282_s_at | 18 | 31 | 31.91% | 80.74% | 3.46 | 3.2356E−05 |
| 37532_at | 14 | 31 | 45.83% | 40.29% | 3.46 | 3.66945E−11 |
| 40274_at | 1 | 7 | 91.39% | 37.78% | 3.46 | 8.80759E−10 |
| 35576_f_at | 11 | 31 | 38.57% | 51.20% | 3.41 | 1.26259E−08 |
| 39077_at | 13 | 31 | 58.58% | 73.96% | 3.38 | 1.26591E−05 |
| 39971_at | 15 | 30 | 39.64% | 39.10% | 3.38 | 2.063E−11 |
| 38717_at | 16 | 31 | 37.04% | 46.64% | 3.37 | 1.78133E−09 |
| 630_at | 14 | 31 | 24.25% | 31.54% | 3.35 | 7.53431E−14 |
| 1519_at | 15 | 31 | 24.67% | 63.50% | 3.35 | 9.22657E−07 |
| 31522_f_at | 6 | 31 | 36.38% | 52.53% | 3.29 | 3.0289E−08 |
| 41562_at | 17 | 31 | 17.70% | 89.22% | 3.25 | 0.000161176 |
| 2067_f_at | 0 | 25 | 42.63% | 38.65% | 3.19 | 3.11516E−11 |
| 36908_at | 1 | 21 | 35.00% | 110.36% | 3.19 | 0.001699281 |
| 36215_at | 10 | 31 | 12.12% | 73.80% | 3.17 | 1.48015E−05 |
| 39032_at | 12 | 31 | 17.12% | 86.04% | 3.16 | 0.000120285 |
| 33986_r_at | 18 | 31 | 34.85% | 39.74% | 3.14 | 7.41493E−11 |
| 32096_at | 0 | 22 | 42.43% | 68.90% | 3.11 | 6.98647E−06 |
| 37749_at | 7 | 25 | 31.14% | 162.56% | 3.10 | 0.027617609 |
| 34660_at | 18 | 29 | 32.17% | 105.61% | 3.09 | 0.001297738 |
| 34320_at | 0 | 7 | 39.28% | 139.88% | 3.09 | 0.01193055 |
| 36347_f_at | 18 | 31 | 25.22% | 53.14% | 3.09 | 6.82196E−08 |
| 38213_at | 16 | 30 | 31.09% | 49.07% | 3.08 | 1.37603E−08 |
| 2042_s_at | 18 | 31 | 34.63% | 41.43% | 3.04 | 3.13982E−10 |
| 39315_at | 6 | 28 | 34.13% | 93.86% | 3.04 | 0.000433247 |
| 33132_at | 3 | 21 | 48.79% | 83.17% | 3.04 | 0.000118008 |
| 286_at | 18 | 31 | 22.27% | 41.66% | 3.02 | 4.43294E−10 |
| 38826_at | 17 | 31 | 28.39% | 58.25% | 3.00 | 5.1589E−07 |
| 35766_at | 0 | 19 | 22.33% | 101.72% | 2.99 | 0.00102621 |
| 34862_at | 14 | 30 | 34.30% | 66.67% | 2.98 | 5.41787E−06 |
| 36785_at | 17 | 30 | 29.02% | 59.85% | 2.98 | 8.78388E−07 |
| 38671_at | 16 | 31 | 64.75% | 57.21% | 2.97 | 8.28543E−07 |
| 33131_at | 15 | 30 | 44.32% | 53.92% | 2.95 | 1.69328E−07 |
| 41027_at | 0 | 16 | 0.00% | 159.77% | 2.94 | 0.028725782 |
| 32819_at | 16 | 31 | 33.61% | 56.38% | 2.92 | 3.80833E−07 |
| 2025_s_at | 18 | 31 | 29.25% | 43.44% | 2.91 | 1.67689E−09 |
| 39710_at | 18 | 31 | 45.27% | 53.92% | 2.89 | 2.12428E−07 |
| 40184_at | 11 | 30 | 70.03% | 41.90% | 2.89 | 1.06582E−08 |
| 39693_at | 10 | 27 | 48.22% | 44.84% | 2.85 | 6.80331E−09 |
| 1470_at | 0 | 14 | 41.16% | 53.06% | 2.85 | 1.74788E−07 |
| 36937_s_at | 10 | 29 | 27.22% | 73.90% | 2.85 | 3.45003E−05 |
| 39691_at | 16 | 31 | 36.38% | 38.55% | 2.83 | 1.40903E−10 |
| 40610_at | 18 | 31 | 36.94% | 63.77% | 2.83 | 4.42003E−06 |
| 40365_at | 18 | 31 | 45.93% | 58.46% | 2.81 | 1.28239E−06 |
| 36617_at | 16 | 31 | 41.20% | 109.19% | 2.81 | 0.002846541 |
| 31523_f_at | 16 | 31 | 29.70% | 56.28% | 2.81 | 5.27722E−07 |
| 37724_at | 10 | 27 | 35.65% | 92.43% | 2.81 | 0.000587691 |
| 1693_s_at | 17 | 29 | 46.35% | 75.58% | 2.80 | 6.2935E−05 |
| 39054_at | 0 | 7 | 76.70% | 111.70% | 2.77 | 0.004489303 |
| 37456_at | 2 | 14 | 56.43% | 121.16% | 2.76 | 0.007306436 |
| 754_s_at | 0 | 10 | 44.96% | 90.12% | 2.73 | 0.000577744 |
| 38811_at | 16 | 31 | 23.75% | 39.21% | 2.72 | 3.56607E−10 |
| 38585_at | 18 | 30 | 117.58% | 102.22% | 2.72 | 0.004300029 |
| 33528_at | 4 | 27 | 20.92% | 120.20% | 2.69 | 0.006851923 |
| 40634_at | 17 | 31 | 35.00% | 41.09% | 2.69 | 1.38515E−09 |
| 1920_s_at | 12 | 31 | 15.32% | 52.33% | 2.69 | 2.08008E−07 |
| 33989_f_at | 18 | 31 | 42.07% | 51.57% | 2.69 | 2.09691E−07 |
| 37716_at | 2 | 21 | 32.87% | 103.64% | 2.68 | 0.002231443 |
| 37187_at | 17 | 28 | 17.15% | 98.75% | 2.66 | 0.001430932 |
| 38097_at | 17 | 31 | 32.67% | 42.70% | 2.66 | 3.82032E−09 |
| 907_at | 11 | 30 | 12.12% | 64.86% | 2.65 | 8.79165E−06 |
| 33836_at | 8 | 31 | 41.98% | 43.65% | 2.65 | 8.83079E−09 |
| 40485_at | 13 | 31 | 24.06% | 51.19% | 2.65 | 1.65559E−07 |
| 36780_at | 18 | 31 | 30.24% | 114.24% | 2.65 | 0.005184188 |
| 35523_at | 0 | 12 | 0.00% | 137.20% | 2.65 | 0.017128396 |
| 36943_r_at | 17 | 31 | 25.46% | 50.65% | 2.64 | 1.41417E−07 |
| 41213_at | 18 | 31 | 23.50% | 47.01% | 2.64 | 3.21956E−08 |
| 37033_s_at | 18 | 31 | 21.60% | 31.01% | 2.62 | 1.3737E−12 |
| 36881_at | 6 | 28 | 73.50% | 60.09% | 2.61 | 1.37317E−05 |
| 34651_at | 9 | 29 | 46.81% | 41.89% | 2.61 | 6.88754E−09 |
| 1751_g_at | 14 | 29 | 37.70% | 51.39% | 2.60 | 2.74451E−07 |
| 37376_at | 16 | 31 | 63.54% | 44.25% | 2.59 | 1.1301E−07 |
| 38747_at | 8 | 25 | 27.97% | 90.05% | 2.59 | 0.00072012 |
| 39091_at | 7 | 30 | 24.25% | 43.35% | 2.58 | 7.68646E−09 |
| 33412_at | 18 | 31 | 28.81% | 67.43% | 2.58 | 2.24344E−05 |
| 1456_s_at | 16 | 31 | 39.41% | 52.25% | 2.57 | 4.51772E−07 |
| 32668_at | 9 | 30 | 34.30% | 73.10% | 2.56 | 7.5538E−05 |
| 41812_s_at | 8 | 27 | 38.88% | 49.22% | 2.55 | 1.63303E−07 |
| 39698_at | 11 | 22 | 26.76% | 112.08% | 2.55 | 0.0053672 |
| 37705_at | 0 | 4 | 46.49% | 38.40% | 2.54 | 1.92327E−09 |
| 35255_at | 8 | 31 | 15.32% | 34.77% | 2.52 | 6.98179E−11 |
| 37179_at | 18 | 30 | 35.72% | 70.81% | 2.52 | 5.87242E−05 |
| 36749_at | 12 | 23 | 23.75% | 112.26% | 2.52 | 0.005727397 |
| 207_at | 5 | 28 | 62.81% | 38.01% | 2.51 | 2.78216E−08 |
| 1520_s_at | 2 | 19 | 55.00% | 104.04% | 2.51 | 0.003820876 |
| 38675_at | 1 | 27 | 48.22% | 45.83% | 2.51 | 9.04471E−08 |
| 1752_at | 0 | 13 | 29.10% | 112.73% | 2.50 | 0.006237946 |
| 1474_s_at | 16 | 30 | 18.19% | 43.19% | 2.49 | 1.17412E−08 |
| 34892_at | 11 | 30 | 32.87% | 67.61% | 2.49 | 3.4299E−05 |
| 203_at | 0 | 21 | 35.00% | 95.39% | 2.48 | 0.001715515 |
| 39023_at | 12 | 30 | 33.91% | 65.61% | 2.48 | 2.41203E−05 |
| 40422_at | 7 | 25 | 48.26% | 84.22% | 2.46 | 0.000623441 |
| 1161_at | 18 | 31 | 27.08% | 27.64% | 2.45 | 1.54239E−13 |
| 631_g_at | 18 | 31 | 21.82% | 24.39% | 2.45 | 2.78828E−15 |
| 538_at | 7 | 29 | 23.76% | 88.45% | 2.44 | 0.000869343 |
| 1750_at | 4 | 28 | 60.20% | 55.44% | 2.44 | 6.32211E−06 |
| 34378_at | 15 | 31 | 50.67% | 63.46% | 2.43 | 2.68706E−05 |
| 36618_g_at | 5 | 22 | 34.30% | 108.94% | 2.43 | 0.005705418 |
| 33173_g_at | 5 | 29 | 36.07% | 57.98% | 2.42 | 5.0629E−06 |
| 37348_s_at | 18 | 31 | 18.49% | 48.64% | 2.42 | 1.87753E−07 |
| 33425_at | 3 | 31 | 51.10% | 41.17% | 2.42 | 3.58159E−08 |
| 32543_at | 7 | 26 | 51.02% | 45.14% | 2.42 | 1.65001E−07 |
| 39775_at | 4 | 24 | 83.96% | 100.12% | 2.42 | 0.004967931 |
| 948_s_at | 13 | 31 | 36.69% | 32.51% | 2.42 | 4.87322E−11 |
| 40774_at | 18 | 30 | 23.57% | 39.01% | 2.41 | 2.03732E−09 |
| 41332_at | 7 | 23 | 46.02% | 47.34% | 2.40 | 2.94332E−07 |
| 39936_at | 0 | 13 | 0.00% | 101.86% | 2.39 | 0.003441962 |
| 40979_at | 17 | 31 | 35.36% | 30.93% | 2.39 | 1.74395E−11 |
| 39672_at | 8 | 29 | 46.82% | 39.99% | 2.38 | 1.88558E−08 |
| 41108_at | 4 | 26 | 67.36% | 27.86% | 2.38 | 4.39098E−08 |
| 40767_at | 13 | 31 | 17.12% | 63.83% | 2.37 | 2.13536E−05 |
| 1643_g_at | 16 | 31 | 20.25% | 37.39% | 2.37 | 9.90261E−10 |
| 34889_at | 18 | 30 | 24.50% | 42.61% | 2.37 | 1.90624E−08 |
| 38745_at | 14 | 30 | 41.16% | 53.59% | 2.37 | 2.32275E−06 |
| 38454_g_at | 15 | 30 | 39.46% | 52.56% | 2.36 | 1.65859E−06 |
| 37194_at | 10 | 29 | 23.15% | 71.03% | 2.36 | 0.000100912 |
| 39061_at | 18 | 31 | 25.75% | 42.83% | 2.35 | 2.43956E−08 |
| 40916_at | 18 | 31 | 32.94% | 58.42% | 2.34 | 8.10952E−06 |
| 39298_at | 7 | 25 | 29.10% | 61.11% | 2.34 | 1.49464E−05 |
| 262_at | 18 | 31 | 25.46% | 40.97% | 2.34 | 1.0422E−08 |
| 32241_at | 17 | 31 | 26.52% | 25.30% | 2.33 | 4.0304E−14 |
| 36138_at | 17 | 31 | 43.36% | 49.63% | 2.33 | 9.01249E−07 |
| 40827_at | 14 | 31 | 28.86% | 38.50% | 2.33 | 2.95527E−09 |
| 33809_at | 0 | 18 | 0.00% | 95.23% | 2.32 | 0.00231603 |
| 36597_at | 16 | 31 | 33.35% | 33.81% | 2.32 | 2.1342E−10 |
| 40718_at | 2 | 23 | 45.38% | 68.73% | 2.32 | 0.000108949 |
| 1472_g_at | 11 | 29 | 17.12% | 53.19% | 2.32 | 1.61503E−06 |
| 31528_f_at | 7 | 26 | 32.91% | 44.30% | 2.32 | 7.33689E−08 |
| 35771_at | 18 | 31 | 30.36% | 53.15% | 2.32 | 1.89747E−06 |
| 36711_at | 18 | 30 | 51.66% | 96.58% | 2.31 | 0.003491661 |
| 31622_f_at | 5 | 17 | 54.85% | 56.49% | 2.31 | 1.35434E−05 |
| 32542_at | 5 | 23 | 29.10% | 77.01% | 2.30 | 0.000341076 |
| 35371_at | 13 | 31 | 29.10% | 77.72% | 2.29 | 0.000391279 |
| 37242_at | 6 | 24 | 29.10% | 77.72% | 2.29 | 0.000391279 |
| 32165_at | 15 | 29 | 12.12% | 65.32% | 2.29 | 4.20184E−05 |
| 263_g_at | 18 | 31 | 39.32% | 37.76% | 2.28 | 7.27094E−09 |
| 32825_at | 18 | 31 | 24.92% | 33.41% | 2.27 | 1.40403E−10 |
| 31801_at | 13 | 30 | 35.00% | 34.15% | 2.27 | 5.76872E−10 |
| 40854_at | 18 | 31 | 31.91% | 24.37% | 2.26 | 1.95169E−13 |
| 35741_at | 16 | 31 | 34.30% | 41.56% | 2.26 | 3.73545E−08 |
| 39056_at | 18 | 31 | 35.66% | 34.72% | 2.25 | 1.1393E−09 |
| 37384_at | 11 | 27 | 32.87% | 84.39% | 2.25 | 0.001109499 |
| 478_g_at | 9 | 31 | 54.85% | 52.24% | 2.24 | 8.0969E−06 |
| 34023_at | 16 | 26 | 44.10% | 139.20% | 2.24 | 0.036980349 |
| 38704_at | 11 | 30 | 42.28% | 46.27% | 2.24 | 5.46027E−07 |
| 36684_at | 18 | 31 | 28.18% | 38.05% | 2.24 | 5.04384E−09 |
| 38123_at | 18 | 31 | 37.34% | 30.96% | 2.23 | 1.8305E−10 |
| 41322_s_at | 14 | 31 | 30.68% | 35.67% | 2.23 | 1.50484E−09 |
| 35182_f_at | 0 | 9 | 0.00% | 76.69% | 2.23 | 0.000383787 |
| 36955_at | 1 | 15 | 62.37% | 54.56% | 2.23 | 2.66863E−05 |
| 31670_s_at | 18 | 30 | 20.23% | 38.13% | 2.22 | 4.82586E−09 |
| 39638_at | 15 | 31 | 20.23% | 41.71% | 2.22 | 3.33484E−08 |
| 41357_at | 9 | 31 | 40.60% | 28.79% | 2.22 | 1.56359E−10 |
| 32087_at | 5 | 30 | 34.30% | 77.47% | 2.22 | 0.00055134 |
| 39968_at | 4 | 28 | 37.34% | 55.67% | 2.21 | 9.71648E−06 |
| 31524_f_at | 13 | 30 | 26.26% | 44.00% | 2.21 | 1.24178E−07 |
| 37018_at | 4 | 24 | 29.10% | 91.92% | 2.21 | 0.002675366 |
| 39471_at | 18 | 31 | 15.83% | 26.99% | 2.20 | 9.09719E−13 |
| 40877_s_at | 18 | 31 | 33.18% | 46.75% | 2.20 | 5.29412E−07 |
| 35292_at | 18 | 31 | 31.84% | 25.24% | 2.20 | 1.19365E−12 |
| 39799_at | 18 | 30 | 25.92% | 50.32% | 2.20 | 1.54524E−06 |
| 40698_at | 18 | 31 | 56.07% | 43.46% | 2.19 | 1.49634E−06 |
| 37726_at | 18 | 31 | 27.58% | 26.89% | 2.18 | 1.88236E−12 |
| 41379_at | 18 | 31 | 28.01% | 31.01% | 2.18 | 7.11505E−11 |
| 33415_at | 18 | 31 | 16.05% | 35.05% | 2.18 | 1.01977E−09 |
| 38416_at | 18 | 31 | 27.25% | 33.11% | 2.18 | 3.3549E−10 |
| 36958_at | 5 | 24 | 86.88% | 71.35% | 2.18 | 0.001376025 |
| 35801_at | 17 | 31 | 21.74% | 30.18% | 2.18 | 2.41818E−11 |
| 38780_at | 18 | 31 | 23.27% | 40.32% | 2.17 | 2.57396E−08 |
| 1196_at | 9 | 29 | 24.67% | 36.99% | 2.16 | 4.58197E−09 |
| 32548_at | 18 | 31 | 20.83% | 27.27% | 2.16 | 1.72277E−12 |
| 34961_at | 6 | 25 | 32.87% | 94.75% | 2.16 | 0.004141212 |
| 40962_s_at | 15 | 31 | 27.74% | 57.24% | 2.16 | 1.58695E−05 |
| 33219_at | 18 | 31 | 35.35% | 38.73% | 2.16 | 2.79321E−08 |
| 38473_at | 16 | 31 | 17.53% | 29.68% | 2.16 | 1.93377E−11 |
| 37399_at | 18 | 29 | 33.16% | 95.34% | 2.15 | 0.00437926 |
| 38052_at | 14 | 25 | 47.92% | 118.01% | 2.15 | 0.019859383 |
| 38376_at | 14 | 31 | 34.39% | 24.29% | 2.14 | 4.82448E−12 |
| 33925_at | 0 | 12 | 43.75% | 100.14% | 2.14 | 0.007232957 |
| 40842_at | 18 | 31 | 26.89% | 27.06% | 2.14 | 3.82083E−12 |
| 32803_at | 18 | 31 | 14.73% | 25.53% | 2.14 | 3.85864E−13 |
| 34251_at | 0 | 13 | 0.00% | 104.85% | 2.13 | 0.008510559 |
| 1449_at | 18 | 31 | 22.89% | 26.79% | 2.12 | 1.95619E−12 |
| 351_f_at | 18 | 31 | 31.77% | 27.00% | 2.12 | 1.58207E−11 |
| 38642_at | 14 | 31 | 17.12% | 63.57% | 2.12 | 7.27084E−05 |
| 39341_at | 14 | 24 | 39.58% | 68.50% | 2.12 | 0.000262557 |
| 37774_at | 2 | 23 | 20.79% | 43.75% | 2.12 | 2.07329E−07 |
| 39767_at | 18 | 31 | 50.06% | 25.65% | 2.11 | 1.04039E−08 |
| 37692_at | 18 | 31 | 23.27% | 53.96% | 2.11 | 8.05542E−06 |
| 32232_at | 18 | 31 | 28.03% | 26.05% | 2.11 | 3.00414E−12 |
| 38072_at | 15 | 31 | 35.35% | 35.33% | 2.11 | 8.53696E−09 |
| 38399_at | 18 | 31 | 25.75% | 20.00% | 2.11 | 4.14236E−15 |
| 41202_s_at | 17 | 31 | 57.84% | 39.50% | 2.10 | 2.01222E−06 |
| 1942_s_at | 10 | 29 | 43.29% | 43.18% | 2.10 | 7.85909E−07 |
| 32844_at | 15 | 25 | 34.30% | 50.93% | 2.10 | 5.19131E−06 |
| 35342_at | 18 | 31 | 34.30% | 45.74% | 2.10 | 9.27432E−07 |
| 41123_s_at | 0 | 13 | 0.00% | 122.74% | 2.10 | 0.024265228 |
| 38233_at | 8 | 28 | 42.66% | 90.40% | 2.09 | 0.003973476 |
| 2012_s_at | 17 | 31 | 25.46% | 55.53% | 2.09 | 1.53768E−05 |
| 35848_at | 17 | 31 | 20.79% | 45.83% | 2.09 | 6.1542E−07 |
| 41163_at | 3 | 27 | 33.94% | 56.86% | 2.08 | 2.88028E−05 |
| 41375_at | 18 | 31 | 24.12% | 32.35% | 2.07 | 5.32944E−10 |
| 38443_at | 18 | 31 | 22.69% | 35.67% | 2.07 | 4.76639E−09 |
| 39792_at | 18 | 31 | 22.74% | 30.55% | 2.07 | 1.23507E−10 |
| 37392_at | 17 | 31 | 23.74% | 35.22% | 2.07 | 3.89926E−09 |
| 38011_at | 18 | 31 | 30.84% | 27.32% | 2.07 | 3.84248E−11 |
| 39507_at | 18 | 31 | 26.95% | 37.24% | 2.07 | 1.61059E−08 |
| 1476_s_at | 16 | 30 | 24.77% | 46.64% | 2.07 | 1.11208E−06 |
| 34325_at | 16 | 31 | 29.10% | 46.40% | 2.06 | 1.24168E−06 |
| 36185_at | 13 | 27 | 39.52% | 63.22% | 2.06 | 0.000146283 |
| 35818_at | 18 | 31 | 26.53% | 28.19% | 2.06 | 3.23044E−11 |
| 38808_at | 1 | 14 | 47.62% | 42.49% | 2.06 | 1.67774E−06 |
| 40133_s_at | 18 | 31 | 30.29% | 54.09% | 2.05 | 1.52523E−05 |
| 1287_at | 18 | 31 | 22.19% | 42.49% | 2.05 | 2.13822E−07 |
| 41725_at | 14 | 24 | 76.31% | 41.72% | 2.05 | 7.07035E−05 |
| 1499_at | 18 | 31 | 32.78% | 28.74% | 2.05 | 2.526E−10 |
| 41535_at | 18 | 31 | 21.24% | 32.92% | 2.05 | 9.53138E−10 |
| 36892_at | 0 | 7 | 32.87% | 100.12% | 2.05 | 0.008905977 |
| 38695_at | 18 | 31 | 20.26% | 28.08% | 2.04 | 1.71789E−11 |
| 39139_at | 17 | 31 | 39.85% | 40.13% | 2.04 | 3.24841E−07 |
| 1660_at | 17 | 30 | 38.85% | 29.76% | 2.04 | 2.95158E−09 |
| 1151_at | 18 | 31 | 19.27% | 21.80% | 2.04 | 1.123E−14 |
| 32184_at | 18 | 31 | 27.70% | 27.66% | 2.04 | 3.48204E−11 |
| 35184_at | 15 | 31 | 27.62% | 41.06% | 2.04 | 1.60686E−07 |
| 41243_at | 18 | 31 | 33.18% | 31.17% | 2.04 | 1.5211E−09 |
| 153_f_at | 0 | 21 | 35.00% | 96.89% | 2.03 | 0.007598479 |
| 1826_at | 0 | 6 | 0.00% | 115.39% | 2.03 | 0.020294144 |
| 1521_at | 16 | 31 | 28.75% | 42.41% | 2.03 | 3.42344E−07 |
| 36624_at | 18 | 31 | 24.58% | 48.74% | 2.03 | 3.25606E−06 |
| 32696_at | 16 | 29 | 17.70% | 76.89% | 2.02 | 0.001012887 |
| 40467_at | 16 | 31 | 37.04% | 41.49% | 2.02 | 5.00766E−07 |
| 40638_at | 15 | 31 | 27.10% | 37.35% | 2.02 | 3.02465E−08 |
| 32051_at | 18 | 30 | 25.09% | 33.76% | 2.01 | 3.30416E−09 |
| 32853_at | 18 | 30 | 30.24% | 32.40% | 2.01 | 2.72994E−09 |
| 34099_f_at | 18 | 31 | 17.84% | 32.82% | 2.01 | 1.29593E−09 |
| 1009_at | 18 | 31 | 24.49% | 28.43% | 2.01 | 6.16363E−11 |
| 40441_g_at | 18 | 31 | 32.55% | 35.93% | 2.01 | 3.02838E−08 |
| 37281_at | 16 | 31 | 32.87% | 40.73% | 2.00 | 3.00008E−07 |
| 34893_at | 17 | 31 | 29.81% | 29.77% | 2.00 | 5.07606E−10 |
| 36465_at | 8 | 30 | 30.36% | 55.20% | 2.00 | 3.00779E−05 |
| 33891_at | 18 | 30 | 20.58% | 55.38% | 2.00 | 2.51492E−05 |
| 39639_s_at | 11 | 8 | 35.24% | 41.28% | 0.50 | 1.20269E−05 |
| 33439_at | 18 | 30 | 30.22% | 79.25% | 0.50 | 1.10812E−05 |
| 35012_at | 18 | 30 | 39.67% | 78.52% | 0.50 | 0.000139013 |
| 36375_at | 1 | 2 | 29.16% | 33.99% | 0.50 | 7.144E−07 |
| 39059_at | 18 | 28 | 20.29% | 23.95% | 0.50 | 1.17807E−09 |
| 37924_g_at | 12 | 18 | 39.50% | 54.40% | 0.50 | 5.75709E−05 |
| 1237_at | 18 | 22 | 63.20% | 103.41% | 0.50 | 0.007336265 |
| 36277_at | 18 | 24 | 32.47% | 46.74% | 0.50 | 3.58675E−06 |
| 38113_at | 15 | 11 | 58.53% | 33.07% | 0.50 | 0.002065354 |
| 35590_s_at | 11 | 20 | 22.07% | 44.49% | 0.49 | 3.90629E−09 |
| 34912_at | 15 | 22 | 22.71% | 35.09% | 0.49 | 5.8373E−09 |
| 39822_s_at | 18 | 29 | 57.23% | 65.20% | 0.49 | 0.00216477 |
| 34161_at | 11 | 19 | 31.35% | 31.82% | 0.49 | 1.92722E−06 |
| 35091_at | 15 | 22 | 41.01% | 41.77% | 0.49 | 6.8334E−05 |
| 37536_at | 18 | 26 | 43.14% | 72.89% | 0.49 | 0.000210603 |
| 214_at | 16 | 25 | 30.31% | 40.38% | 0.49 | 1.07055E−06 |
| 721_g_at | 14 | 18 | 35.95% | 38.14% | 0.49 | 1.24453E−05 |
| 179_at | 9 | 15 | 28.18% | 43.38% | 0.49 | 2.86551E−07 |
| 100_g_at | 2 | 12 | 34.05% | 26.76% | 0.49 | 5.63668E−06 |
| 33080_s_at | 14 | 21 | 24.10% | 60.24% | 0.49 | 5.53746E−08 |
| 38229_at | 10 | 10 | 32.75% | 53.76% | 0.49 | 3.6205E−06 |
| 35485_at | 14 | 23 | 23.91% | 25.33% | 0.48 | 1.7272E−08 |
| 32228_at | 6 | 7 | 39.45% | 54.51% | 0.48 | 3.73031E−05 |
| 37425_g_at | 9 | 19 | 22.71% | 43.98% | 0.48 | 3.742E−09 |
| 34060_g_at | 7 | 20 | 34.55% | 51.52% | 0.48 | 6.15346E−06 |
| 35367_at | 18 | 28 | 37.12% | 88.80% | 0.48 | 6.47235E−05 |
| 36378_at | 12 | 12 | 43.93% | 45.46% | 0.48 | 0.000102217 |
| 32193_at | 18 | 27 | 38.51% | 62.59% | 0.48 | 2.7918E−05 |
| 32747_at | 18 | 29 | 22.80% | 71.36% | 0.48 | 6.29854E−08 |
| 36916_at | 0 | 5 | 41.91% | 45.48% | 0.47 | 5.59657E−05 |
| 32469_at | 2 | 1 | 31.29% | 39.69% | 0.47 | 9.79315E−07 |
| 595_at | 18 | 31 | 48.91% | 71.91% | 0.47 | 0.000399778 |
| 32227_at | 18 | 31 | 30.96% | 29.45% | 0.47 | 8.87468E−07 |
| 33752_at | 18 | 29 | 33.78% | 37.58% | 0.47 | 2.93567E−06 |
| 38138_at | 18 | 24 | 56.43% | 107.37% | 0.47 | 0.00241521 |
| 888_s_at | 10 | 12 | 37.77% | 33.59% | 0.47 | 1.33527E−05 |
| 1106_s_at | 18 | 25 | 31.07% | 77.81% | 0.47 | 3.078E−06 |
| 39815_at | 1 | 8 | 43.31% | 84.78% | 0.47 | 0.000149368 |
| 35785_at | 18 | 31 | 30.22% | 52.50% | 0.47 | 5.10061E−07 |
| 33333_at | 15 | 25 | 34.67% | 42.05% | 0.47 | 3.8012E−06 |
| 1894_f_at | 18 | 31 | 58.56% | 68.08% | 0.47 | 0.001596272 |
| 38162_at | 1 | 3 | 48.50% | 75.13% | 0.46 | 0.000311012 |
| 38735_at | 10 | 18 | 25.38% | 30.90% | 0.46 | 2.13138E−08 |
| 38406_f_at | 15 | 22 | 28.67% | 41.46% | 0.46 | 1.53789E−07 |
| 37898_r_at | 14 | 14 | 25.31% | 36.09% | 0.46 | 1.47528E−08 |
| 39527_at | 1 | 4 | 33.05% | 38.70% | 0.45 | 1.361E−06 |
| 31815_r_at | 0 | 1 | 18.56% | 35.73% | 0.45 | 9.16328E−12 |
| 38976_at | 18 | 30 | 25.29% | 54.55% | 0.45 | 1.12942E−08 |
| 41038_at | 18 | 28 | 49.59% | 76.64% | 0.45 | 0.000289337 |
| 36207_at | 15 | 23 | 40.15% | 74.95% | 0.45 | 2.78811E−05 |
| 31687_f_at | 18 | 31 | 26.92% | 55.40% | 0.45 | 3.56332E−08 |
| 32675_at | 18 | 30 | 28.44% | 63.76% | 0.45 | 1.24025E−07 |
| 649_s_at | 18 | 31 | 36.16% | 57.65% | 0.45 | 4.38053E−06 |
| 2077_at | 3 | 1 | 78.50% | 35.86% | 0.45 | 0.008429968 |
| 404_at | 18 | 22 | 30.94% | 52.64% | 0.45 | 3.51928E−07 |
| 36114_r_at | 1 | 2 | 82.12% | 78.02% | 0.44 | 0.012156331 |
| 1105_s_at | 18 | 30 | 32.94% | 80.33% | 0.44 | 2.17031E−06 |
| 39399_at | 2 | 4 | 53.94% | 60.28% | 0.44 | 0.000448782 |
| 32673_at | 16 | 28 | 42.90% | 46.16% | 0.44 | 3.2091E−05 |
| 33758_f_at | 4 | 6 | 28.13% | 34.52% | 0.44 | 6.24105E−08 |
| 31692_at | 16 | 25 | 51.38% | 73.22% | 0.44 | 0.000297492 |
| 34112_r_at | 6 | 4 | 44.56% | 37.51% | 0.44 | 4.80597E−05 |
| 41840_r_at | 7 | 9 | 88.05% | 142.81% | 0.44 | 0.023819357 |
| 37556_at | 17 | 27 | 73.47% | 44.47% | 0.43 | 0.004688749 |
| 32916_at | 18 | 29 | 36.74% | 75.21% | 0.43 | 5.35217E−06 |
| 34655_at | 7 | 5 | 30.53% | 39.75% | 0.43 | 1.97111E−07 |
| 40699_at | 16 | 27 | 32.65% | 100.14% | 0.43 | 4.48235E−06 |
| 38895_i_at | 17 | 25 | 50.21% | 44.81% | 0.43 | 0.000159408 |
| 31562_at | 3 | 1 | 92.40% | 117.84% | 0.43 | 0.023030134 |
| 1150_at | 18 | 31 | 27.42% | 65.95% | 0.43 | 2.96313E−08 |
| 1104_s_at | 16 | 23 | 55.78% | 74.20% | 0.42 | 0.000522882 |
| 36640_at | 2 | 1 | 25.55% | 32.72% | 0.42 | 7.57758E−09 |
| 31357_at | 2 | 0 | 103.57% | 166.50% | 0.42 | 0.045622448 |
| 40215_at | 13 | 12 | 64.75% | 57.77% | 0.42 | 0.001670849 |
| 32897_at | 2 | 1 | 31.42% | 70.88% | 0.42 | 3.34375E−07 |
| 40876_at | 18 | 31 | 34.97% | 33.36% | 0.42 | 1.49693E−06 |
| 36488_at | 17 | 27 | 43.14% | 90.38% | 0.42 | 4.50106E−05 |
| 40278_at | 17 | 30 | 92.89% | 110.55% | 0.42 | 0.02150859 |
| 40089_at | 7 | 25 | 45.21% | 83.65% | 0.42 | 6.06976E−05 |
| 40739_at | 18 | 21 | 36.30% | 33.40% | 0.42 | 2.40987E−06 |
| 32525_r_at | 0 | 2 | 34.29% | 43.71% | 0.42 | 9.08927E−07 |
| 31621_s_at | 2 | 0 | 26.29% | 42.89% | 0.42 | 7.12527E−09 |
| 110_at | 2 | 0 | 25.65% | 53.16% | 0.42 | 3.52686E−09 |
| 32904_at | 8 | 3 | 96.62% | 175.28% | 0.41 | 0.033900503 |
| 32407_f_at | 16 | 11 | 47.01% | 70.87% | 0.41 | 6.60062E−05 |
| 416_s_at | 0 | 1 | 56.57% | 63.69% | 0.41 | 0.000440551 |
| AFFX- | 3 | 1 | 56.98% | 107.95% | 0.41 | 0.000763643 |
| M27830_3_at | | | | | | |
| 32815_at | 17 | 30 | 45.96% | 70.49% | 0.41 | 4.94004E−05 |
| 1339_s_at | 8 | 20 | 73.21% | 81.70% | 0.41 | 0.004055387 |
| 38417_at | 18 | 30 | 29.55% | 32.39% | 0.41 | 8.04458E−08 |
| 38894_g_at | 18 | 31 | 35.39% | 37.48% | 0.41 | 1.22887E−06 |
| 36459_at | 18 | 15 | 32.38% | 53.38% | 0.41 | 2.3984E−07 |
| 32901_s_at | 18 | 30 | 45.34% | 46.06% | 0.41 | 3.29319E−05 |
| 34965_at | 18 | 31 | 33.69% | 65.85% | 0.41 | 4.93896E−07 |
| 34415_at | 7 | 10 | 86.76% | 90.29% | 0.41 | 0.01165965 |
| 35714_at | 16 | 14 | 46.26% | 57.74% | 0.40 | 4.09456E−05 |
| 37351_at | 18 | 18 | 71.03% | 90.27% | 0.40 | 0.002952828 |
| 35911_r_at | 18 | 30 | 30.18% | 31.40% | 0.40 | 9.00882E−08 |
| 1780_at | 18 | 28 | 20.81% | 70.76% | 0.40 | 7.32967E−11 |
| 39598_at | 4 | 1 | 45.71% | 52.87% | 0.40 | 2.98363E−05 |
| 31525_s_at | 18 | 31 | 27.55% | 60.28% | 0.39 | 7.30148E−09 |
| 40419_at | 18 | 31 | 19.04% | 41.95% | 0.39 | 1.16003E−12 |
| 34627_at | 1 | 0 | 84.76% | 92.48% | 0.39 | 0.008597692 |
| 34095_f_at | 13 | 8 | 56.43% | 102.64% | 0.39 | 0.000398498 |
| 35530_f_at | 18 | 13 | 52.47% | 62.28% | 0.39 | 0.000130676 |
| 725_i_at | 10 | 8 | 65.69% | 29.50% | 0.39 | 0.001061199 |
| 33963_at | 18 | 29 | 29.99% | 85.08% | 0.39 | 7.2092E−08 |
| 330_s_at | 18 | 28 | 30.55% | 54.80% | 0.39 | 4.66778E−08 |
| 40227_at | 0 | 1 | 35.42% | 66.24% | 0.39 | 6.37202E−07 |
| 1096_g_at | 17 | 20 | 26.79% | 34.34% | 0.38 | 6.35174E−09 |
| 35955_at | 17 | 28 | 33.31% | 64.81% | 0.38 | 1.99716E−07 |
| 41641_at | 4 | 0 | 45.11% | 29.70% | 0.38 | 1.97404E−05 |
| 33021_at | 6 | 2 | 97.55% | 203.49% | 0.38 | 0.028714402 |
| 39609_at | 9 | 7 | 33.08% | 37.52% | 0.38 | 2.23066E−07 |
| 31586_f_at | 13 | 20 | 83.19% | 82.15% | 0.38 | 0.006527146 |
| 1937_at | 18 | 31 | 41.34% | 84.63% | 0.38 | 6.74592E−06 |
| 35379_at | 2 | 1 | 53.71% | 73.91% | 0.38 | 0.000142794 |
| 38513_at | 1 | 0 | 110.74% | 54.90% | 0.37 | 0.028933516 |
| 38968_at | 18 | 26 | 17.84% | 105.02% | 0.37 | 1.07532E−09 |
| 33979_at | 18 | 31 | 24.29% | 85.11% | 0.37 | 9.02365E−10 |
| 37623_at | 18 | 24 | 50.87% | 68.12% | 0.37 | 6.61975E−05 |
| 31578_at | 2 | 4 | 97.66% | 82.08% | 0.37 | 0.01532256 |
| 35566_f_at | 18 | 28 | 44.23% | 84.74% | 0.37 | 1.2313E−05 |
| 37579_at | 18 | 23 | 29.33% | 44.08% | 0.37 | 1.6566E−08 |
| 38508_s_at | 0 | 3 | 43.71% | 46.69% | 0.37 | 9.32683E−06 |
| 32254_at | 18 | 31 | 25.46% | 29.68% | 0.37 | 2.0912E−09 |
| 37701_at | 18 | 30 | 43.47% | 67.61% | 0.37 | 8.02813E−06 |
| 35674_at | 18 | 15 | 45.56% | 77.57% | 0.36 | 1.54895E−05 |
| 36237_at | 9 | 7 | 108.27% | 174.61% | 0.36 | 0.031818634 |
| 1389_at | 11 | 8 | 33.02% | 37.96% | 0.36 | 1.39557E−07 |
| 1797_at | 18 | 31 | 25.69% | 41.12% | 0.36 | 1.07928E−09 |
| 34702_f_at | 8 | 4 | 59.48% | 99.01% | 0.36 | 0.000347016 |
| 34832_s_at | 17 | 22 | 26.03% | 35.90% | 0.36 | 1.87104E−09 |
| 39640_at | 1 | 1 | 52.28% | 48.33% | 0.36 | 6.80077E−05 |
| 33499_s_at | 18 | 29 | 58.87% | 117.19% | 0.36 | 0.000358165 |
| 33757_f_at | 9 | 17 | 37.23% | 39.96% | 0.36 | 8.06946E−07 |
| 33143_s_at | 18 | 25 | 57.22% | 73.52% | 0.35 | 0.000181153 |
| 39706_at | 18 | 28 | 39.70% | 39.13% | 0.35 | 1.96229E−06 |
| 37434_at | 1 | 16 | 110.01% | 43.61% | 0.35 | 0.023546211 |
| 36979_at | 18 | 31 | 33.62% | 61.20% | 0.35 | 9.62052E−08 |
| 37061_at | 18 | 16 | 25.93% | 38.15% | 0.35 | 1.22689E−09 |
| 32162_r_at | 10 | 6 | 44.34% | 80.45% | 0.35 | 7.7218E−06 |
| 2002_s_at | 18 | 31 | 49.72% | 62.78% | 0.35 | 3.26277E−05 |
| 1117_at | 18 | 25 | 24.85% | 52.67% | 0.35 | 2.00178E−10 |
| 32579_at | 18 | 31 | 115.35% | 162.94% | 0.35 | 0.034637693 |
| 38868_at | 16 | 7 | 53.51% | 44.36% | 0.35 | 7.21529E−05 |
| 37078_at | 18 | 20 | 47.49% | 142.14% | 0.34 | 4.64157E−05 |
| 37420_i_at | 18 | 28 | 26.57% | 84.95% | 0.34 | 8.60628E−10 |
| 33501_r_at | 18 | 23 | 57.68% | 116.03% | 0.34 | 0.000203796 |
| 34350_at | 11 | 24 | 96.89% | 28.06% | 0.34 | 0.010066774 |
| 33500_i_at | 18 | 28 | 59.35% | 115.29% | 0.34 | 0.000257475 |
| 32793_at | 18 | 20 | 36.25% | 96.35% | 0.34 | 3.07691E−07 |
| 39245_at | 15 | 28 | 49.62% | 55.50% | 0.34 | 2.52058E−05 |
| 33244_at | 3 | 16 | 120.82% | 146.92% | 0.34 | 0.037567435 |
| 36548_at | 1 | 1 | 110.11% | 124.61% | 0.34 | 0.023266455 |
| 32794_g_at | 18 | 18 | 45.86% | 140.68% | 0.33 | 2.31238E−05 |
| 40159_r_at | 8 | 6 | 44.43% | 140.70% | 0.33 | 1.54102E−05 |
| 34703_f_at | 16 | 16 | 59.79% | 94.56% | 0.33 | 0.000211214 |
| 32620_at | 1 | 0 | 122.71% | 178.73% | 0.33 | 0.040885704 |
| 1353_g_at | 17 | 4 | 51.15% | 54.75% | 0.33 | 3.06925E−05 |
| 35449_at | 17 | 16 | 42.63% | 73.99% | 0.32 | 2.42596E−06 |
| 38194_s_at | 18 | 29 | 56.79% | 124.53% | 0.32 | 0.000127709 |
| 33914_r_at | 13 | 24 | 121.82% | 225.82% | 0.32 | 0.040461494 |
| 34105_f_at | 15 | 12 | 57.70% | 160.83% | 0.32 | 0.000220334 |
| 916_at | 3 | 0 | 60.52% | 138.44% | 0.32 | 0.000259644 |
| 37137_at | 17 | 14 | 59.57% | 133.38% | 0.32 | 0.000208632 |
| 40729_s_at | 18 | 25 | 31.54% | 66.91% | 0.32 | 1.13648E−08 |
| 39765_at | 16 | 26 | 102.09% | 189.61% | 0.32 | 0.01593935 |
| 37975_at | 18 | 25 | 70.09% | 126.12% | 0.31 | 0.00086801 |
| 41694_at | 18 | 31 | 21.22% | 41.65% | 0.31 | 3.83445E−12 |
| 40171_at | 13 | 10 | 37.94% | 57.29% | 0.31 | 3.12059E−07 |
| 33304_at | 16 | 13 | 31.93% | 84.12% | 0.31 | 9.72956E−09 |
| 33371_s_at | 18 | 27 | 37.50% | 63.49% | 0.31 | 2.36086E−07 |
| 35966_at | 18 | 23 | 38.19% | 87.60% | 0.31 | 2.76018E−07 |
| 36591_at | 18 | 23 | 24.32% | 66.57% | 0.31 | 2.13381E−11 |
| 34509_at | 16 | 11 | 61.31% | 40.89% | 0.31 | 0.000163751 |
| 189_s_at | 18 | 26 | 41.09% | 86.03% | 0.30 | 8.00735E−07 |
| 31499_s_at | 16 | 18 | 103.12% | 86.51% | 0.30 | 0.010971632 |
| 732_f_at | 18 | 17 | 103.04% | 220.85% | 0.30 | 0.015592426 |
| 41164_at | 18 | 27 | 39.97% | 96.50% | 0.30 | 4.85421E−07 |
| 36983_f_at | 11 | 1 | 67.51% | 50.33% | 0.29 | 0.000366949 |
| 37864_s_at | 18 | 26 | 70.35% | 140.64% | 0.29 | 0.000703361 |
| 41165_g_at | 18 | 22 | 33.85% | 95.48% | 0.29 | 2.16819E−08 |
| 41096_at | 18 | 31 | 21.04% | 48.42% | 0.29 | 1.14877E−12 |
| 32606_at | 18 | 20 | 40.24% | 46.42% | 0.29 | 6.75936E−07 |
| 31315_at | 15 | 13 | 53.63% | 120.99% | 0.29 | 3.38912E−05 |
| 31666_f_at | 7 | 3 | 128.18% | 263.89% | 0.29 | 0.041791352 |
| 41166_at | 15 | 16 | 46.51% | 92.51% | 0.29 | 3.87871E−06 |
| 33849_at | 18 | 30 | 43.27% | 70.56% | 0.28 | 1.36491E−06 |
| 35013_at | 9 | 7 | 28.13% | 37.28% | 0.28 | 1.79518E−09 |
| 39128_r_at | 4 | 20 | 42.36% | 81.48% | 0.28 | 9.06728E−07 |
| 307_at | 18 | 23 | 27.53% | 71.70% | 0.28 | 2.24756E−10 |
| 36071_at | 9 | 25 | 123.49% | 202.02% | 0.28 | 0.029761124 |
| 37099_at | 18 | 31 | 24.97% | 72.67% | 0.28 | 1.7646E−11 |
| 31574_i_at | 3 | 1 | 134.33% | 281.06% | 0.28 | 0.047954818 |
| 38017_at | 12 | 6 | 54.75% | 40.64% | 0.28 | 3.0607E−05 |
| 36674_at | 18 | 8 | 61.09% | 32.20% | 0.28 | 0.000101249 |
| 34498_at | 18 | 25 | 46.60% | 88.80% | 0.27 | 3.09027E−06 |
| 36338_at | 2 | 17 | 37.93% | 67.62% | 0.27 | 1.34275E−07 |
| 37054_at | 18 | 25 | 31.80% | 102.65% | 0.27 | 3.06662E−09 |
| 37105_at | 18 | 31 | 24.16% | 74.05% | 0.27 | 5.39076E−12 |
| 32607_at | 18 | 31 | 27.24% | 72.53% | 0.27 | 1.27882E−10 |
| 39872_at | 18 | 30 | 34.90% | 53.19% | 0.27 | 3.93116E−08 |
| 41827_f_at | 18 | 29 | 48.32% | 88.39% | 0.26 | 4.46644E−06 |
| 35094_f_at | 18 | 22 | 39.55% | 61.31% | 0.26 | 2.5256E−07 |
| 2090_i_at | 7 | 14 | 21.64% | 78.89% | 0.26 | 1.54043E−13 |
| 37066_at | 18 | 24 | 43.80% | 124.24% | 0.26 | 9.59951E−07 |
| 37121_at | 18 | 26 | 29.82% | 108.21% | 0.26 | 5.36898E−10 |
| 37200_at | 16 | 25 | 75.39% | 115.77% | 0.26 | 0.000708923 |
| 35536_at | 4 | 5 | 57.63% | 52.92% | 0.26 | 4.00463E−05 |
| 1350_at | 15 | 12 | 100.74% | 88.67% | 0.25 | 0.00625924 |
| 37467_at | 15 | 12 | 88.75% | 44.64% | 0.25 | 0.002372052 |
| 41471_at | 18 | 31 | 26.49% | 59.53% | 0.25 | 9.63299E−11 |
| 32529_at | 18 | 25 | 29.72% | 80.53% | 0.25 | 5.48988E−10 |
| 35315_at | 18 | 19 | 40.89% | 48.04% | 0.24 | 3.32284E−07 |
| 32451_at | 18 | 28 | 25.90% | 88.61% | 0.24 | 1.10084E−11 |
| 32275_at | 18 | 21 | 24.64% | 82.16% | 0.24 | 3.63726E−12 |
| 33273_f_at | 18 | 31 | 56.97% | 99.30% | 0.23 | 2.22586E−05 |
| 679_at | 18 | 31 | 28.78% | 73.56% | 0.23 | 2.51029E−10 |
| 36197_at | 18 | 15 | 44.65% | 118.14% | 0.23 | 6.40232E−07 |
| 36372_at | 18 | 18 | 40.34% | 162.15% | 0.23 | 1.38073E−07 |
| 33274_f_at | 18 | 31 | 56.21% | 99.48% | 0.23 | 1.78847E−05 |
| 37145_at | 18 | 18 | 59.72% | 150.28% | 0.23 | 3.9433E−05 |
| 37096_at | 18 | 31 | 24.93% | 87.25% | 0.22 | 3.14072E−12 |
| 31506_s_at | 18 | 30 | 23.98% | 65.61% | 0.22 | 4.17003E−12 |
| 36447_at | 18 | 29 | 32.11% | 138.32% | 0.22 | 8.75583E−10 |
| 36479_at | 0 | 2 | 22.04% | 67.27% | 0.21 | 3.55976E−13 |
| 988_at | 18 | 19 | 46.11% | 88.68% | 0.21 | 8.7939E−07 |
| 33093_at | 16 | 3 | 69.35% | 17.40% | 0.20 | 0.000144223 |
| 38533_s_at | 17 | 17 | 38.42% | 89.09% | 0.20 | 4.03602E−08 |
| 34319_at | 18 | 29 | 30.01% | 91.40% | 0.20 | 2.4963E−10 |
| 2041_i_at | 16 | 28 | 135.87% | 323.92% | 0.20 | 0.028899931 |
| 681_at | 18 | 25 | 35.13% | 129.48% | 0.20 | 3.54977E−09 |
| 37897_s_at | 10 | 4 | 43.31% | 91.93% | 0.19 | 2.5815E−07 |
| 37233_at | 18 | 17 | 36.83% | 61.33% | 0.19 | 2.51099E−08 |
| 35919_at | 18 | 20 | 46.18% | 103.51% | 0.19 | 5.78683E−07 |
| 266_s_at | 18 | 29 | 33.08% | 90.85% | 0.19 | 1.75885E−09 |
| 1962_at | 18 | 23 | 38.04% | 90.59% | 0.18 | 2.29312E−08 |
| 31495_at | 18 | 6 | 32.65% | 40.26% | 0.17 | 3.81369E−09 |
| 34546_at | 18 | 27 | 23.10% | 127.06% | 0.17 | 3.81343E−14 |
| 31792_at | 18 | 24 | 37.96% | 99.81% | 0.16 | 1.68411E−08 |
| 36984_f_at | 18 | 27 | 43.61% | 106.10% | 0.16 | 1.59164E−07 |
| 36105_at | 18 | 22 | 27.69% | 104.42% | 0.16 | 1.78868E−11 |
| 31477_at | 18 | 8 | 39.08% | 59.86% | 0.15 | 3.76424E−08 |
| 31793_at | 18 | 29 | 26.05% | 93.55% | 0.15 | 6.72536E−12 |
| 38326_at | 18 | 24 | 63.89% | 138.92% | 0.14 | 2.2739E−05 |
| 33530_at | 18 | 28 | 24.85% | 100.49% | 0.13 | 1.73645E−12 |
| 39318_at | 16 | 6 | 58.49% | 36.21% | 0.12 | 7.10897E−06 |
| 31381_at | 18 | 8 | 44.53% | 143.83% | 0.12 | 1.05526E−07 |
| 38615_at | 18 | 12 | 34.99% | 105.92% | 0.10 | 2.15528E−09 |
| 37149_s_at | 18 | 27 | 21.71% | 146.84% | 0.10 | 1.1204E−14 |
| 31859_at | 18 | 23 | 46.44% | 144.63% | 0.08 | 1.40049E−07 |
| 36464_at | 18 | 16 | 43.19% | 163.30% | 0.08 | 4.156E−08 |
| 38879_at | 18 | 25 | 34.29% | 131.77% | 0.08 | 1.04878E−09 |
| 36710_at | 18 | 26 | 26.79% | 159.66% | 0.06 | 7.44852E−12 |
| 32821_at | 18 | 25 | 27.51% | 133.26% | 0.05 | 2.23925E−11 |

TABLE 8b
Examples of AML Disease Genes

| Qualifier | Gene Name | Gene Title | Entrez No. | Cyto Band | Unigene No. | Description |
|---|---|---|---|---|---|---|
| 1065_at | FLT3 | fms-related tyrosine kinase 3 | U02687 | 13q12 | Hs.385 | |
| 41071_at | SPINK2 | serine protease inhibitor, Kazal | X57655 | 4q11 | Hs.98243 | |
| | | type, 2 (acrosin-trypsin inhibitor) | | | | |
| 37754_at | LGALS3BP | lectin, galactoside-binding, soluble, | L13210 | 17q25 | Hs.79339 | |
| | | 3 binding protein (galectin 6 | | | | |
| | | binding protein) | | | | |
| 37809_at | HOXA9 | homeo box A9 | U41813 | 7p15-p14 | Hs.127428 | |
| 31623_f_at | MT1A | metallothionein 1A (functional) | K01383 | | | |
| 34583_at | FLT3 | fms-related tyrosine kinase 3 | U02687 | 13q12 | Hs.385 | |
| 38487_at | KIAA0246 | KIAA0246 protein | D87433 | 3p21.31 | Hs.301989 | |
| 39610_at | HOXB2 | homeo box B2 | X16665 | 17q21-q22 | Hs.2733 | |
| 40775_at | ITM2A | integral membrane protein 2A | AL021786 | | | |
| 32755_at | ACTA2 | actin, alpha 2, smooth muscle, | X13839 | 10q23.3 | Hs.195851 | |
| | | aorta | | | | |
| 41138_at | MIC2 | antigen identified by monoclonal | M16279 | Xp22.32, | Hs.177543 | |
| | | antibodies 12E7, F21 and O13 | | Yp11.3 | | |
| 32609_at | H2AFO | H2A histone family, member O | AI885852 | 1q21.3 | Hs.795 | |
| 41470_at | PROML1 | prominin (mouse)-like 1 | AF027208 | 4p15.33 | Hs.112360 | |
| 39175_at | PFKP | phosphofructokinase, platelet | D25328 | 10p15.3-p15.2 | Hs.99910 | |
| 40490_at | DDX21 | DEAD/H (Asp-Glu-Ala-Asp/His) | U41387 | 10q21 | Hs.169531 | |
| | | box polypeptide 21 | | | | |
| 39421_at | RUNX1 | runt-related transcription factor 1 | D43969 | 21q22.3 | Hs.129914 | |
| | | (acute myeloid leukemia 1; aml1 | | | | |
| | | oncogene) | | | | |
| 39317_at | CMAH | cytidine monophosphate-N- | D86324 | 6p22-p23 | Hs.24697 | |
| | | acetylneuraminic acid hydroxylase | | | | |
| | | (CMP-N-acetylneuraminate | | | | |
| | | monooxygenase) | | | | |
| 41188_at | UNK_W28186 | ESTs, Weakly similar | W28186 | 8q22.1 | Hs.296398 | Also know as LAPTM4B |
| | | to GOLGI 4-TRANSMEMBRANE | | | | (lysosomal associated |
| | | SPANNING TRANSPORTER | | | | protein transmembrane 4 |
| | | MTP [H. sapiens] | | | | beta) |
| 1914_at | CCNA1 | cyclin A1 | U66838 | 13q12.3-q13 | Hs.79378 | |
| 41654_at | ADA | adenosine deaminase | X02994 | 20q12-q13.11 | Hs.1217 | |
| 36536_at | SCHIP-1 | schwannomin interacting protein 1 | AF070614 | 3q25.32 | Hs.61490 | |
| 671_at | SPARC | secreted protein, acidic, cysteine- | J03040 | 5q31.3-q32 | Hs.111779 | |
| | | rich (osteonectin) | | | | |
| 34397_at | OA48-18 | acid-inducible phosphoprotein | AF069250 | 17, 17q21 | Hs.278670 | |
| 1475_s_at | MYB | v-myb avian myeloblastosis viral | U22376 | 6q22-q23 | Hs.1334 | |
| | | oncogene homolog | | | | |
| 32905_s_at | TPS1 | tryptase, alpha | M30038 | 16p13.3 | Hs.334455 | |
| 33777_at | TBXAS1 | thromboxane A synthase 1 | D34625 | 7q34-q35 | Hs.2001 | |
| | | (platelet, cytochrome P450, | | | | |
| | | subfamily V) | | | | |
| 33352_at | H2BFQ | H2B histone family, member Q | X57985 | 1q21-q23 | Hs.2178 | |
| 35127_at | H2AFA | H2A histone family, member A | AI039144 | 6p22.2-p21.1 | Hs.121017 | |
| 1997_s_at | BAX | BCL2-associated X protein | U19599 | 19q13.3-q13.4 | Hs.159428 | |
| 943_at | RUNX1 | runt-related transcription factor 1 | D43968 | 21q22.3 | Hs.129914 | |
| | | (acute myeloid leukemia 1; aml1 | | | | |
| | | oncogene) | | | | |
| 39070_at | SNL | singed (Drosophila)-like (sea | U03057 | 7p22 | Hs.118400 | |
| | | urchin fascin homolog like) | | | | |
| 32245_at | UNK_AF014837 | Homo sapiens m6A | AF014837 | 14q11.1 | Hs.268149 | also known as METTL3 |
| | | methyltransferase (MT-A70) gene, | | | | (methyltransferase |
| | | complete cds | | | | like 3), |
| | | | | | | Unigene No. Hs.168799 |
| 35731_at | ITGA4 | integrin, alpha 4 (antigen CD49D, | X16983 | 2q31-q32 | Hs.40034 | |
| | | alpha 4 subunit of VLA-4 receptor) | | | | |
| 32251_at | UNK_AA149307 | Cluster Incl AA149307: zl25h05.s1 | AA149307 | Xq22.1, | Hs.194329 | also known as FLJ21174 |
| | | Soares_pregnant_uterus_NbHPU | | Xq22.1-q22.3 | | (hypothetical protein |
| | | Homo sapiens cDNA clone | | | | FLJ21174) |
| | | IMAGE: 503001 3′, mRNA | | | | |
| | | sequence. | | | | |
| 37283_at | MN1 | meningioma (disrupted in balanced | X82209 | 22q12.1 | Hs.268515 | |
| | | translocation) 1 | | | | |
| 40282_s_at | DF | D component of complement | M84526 | 19p13.3 | Hs.155597 | |
| | | (adipsin) | | | | |
| 37532_at | ACADM | acyl-Coenzyme A dehydrogenase, | M91432 | 1p31 | Hs.79158 | |
| | | C-4 to C-12 straight chain | | | | |
| 40274_at | DBP | D site of albumin promoter | U48213 | 19q13.3 | Hs.155402 | |
| | | (albumin D-box) binding protein | | | | |
| 35576_f_at | H2BFC | H2B histone family, member C | AL009179 | 6p21.3, | Hs.137594, | |
| | | | | 6p22-p21.3 | Hs.151506, | |
| | | | | | Hs.154576, | |
| | | | | | Hs.180779, | |
| | | | | | Hs.182138, | |
| | | | | | Hs.182140, | |
| | | | | | Hs.352109, | |
| | | | | | Hs.356901 | |
| 39077_at | DRAP1 | DR1-associated protein 1 (negative | U41843 | 11q13.3 | Hs.356742 | |
| | | cofactor 2 alpha) | | | | |
| 39971_at | LYL1 | lymphoblastic leukemia derived | M22637 | 19p13.2 | Hs.46446 | |
| | | sequence 1 | | | | |
| 38717_at | DKFZP586A0522 | DKFZP586A0522 protein | AL050159 | 12q11 | Hs.288771 | |
| 630_at | DCTD | dCMP deaminase | L39874 | 4q35.1 | Hs.76894 | |
| 1519_at | ETS2 | v-ets avian erythroblastosis virus | J04102 | 21q22.2 | Hs.85146 | |
| | | E26 oncogene homolog 2 | | | | |
| 31522_f_at | H2BFG | H2B histone family, member G | Z80779 | 6p21.3 | Hs.182137 | |
| 41562_at | BMI1 | murine leukemia viral (bmi-1) | L13689 | 10p13 | Hs.431 | |
| | | oncogene homolog | | | | |
| 2067_f_at | BAX | BCL2-associated X protein | L22475 | 19q13.3-q13.4 | Hs.159428 | |
| 36908_at | MRC1 | mannose receptor, C type 1 | M93221 | 10p13 | Hs.75182 | |
| 36215_at | PRKACB | protein kinase, cAMP-dependent, | M34181 | 1p36.1 | Hs.87773 | |
| | | catalytic, beta | | | | |
| 39032_at | TSC22 | transforming growth factor beta- | AJ222700 | 13q14 | Hs.114360 | |
| | | stimulated protein TSC-22 | | | | |
| 33986_r_at | HSPCB | heat shock 90 kD protein 1, beta | W28616 | 6p12 | Hs.74335 | |
| 32096_at | LYL1 | lymphoblastic leukemia derived | AC005546 | 19p13.13 | Hs.158947 | |
| | | sequence 1 | | | | |
| 37749_at | MEST | mesoderm specific transcript | D78611 | 7q32 | Hs.79284 | |
| | | (mouse) homolog | | | | |
| 34660_at | RNASE6 | ribonuclease, RNase A family, k6 | AI142565 | 14q11.1 | Hs.23262 | |
| 34320_at | UNK_AL050224 | Homo sapiens mRNA; cDNA | AL050224 | 17q21.2 | Hs.29759 | also known as PTRF |
| | | DKFZp586L2123 (from clone | | | | (polymerase I and |
| | | DKFZp586L2123) | | | | transcript release factor), |
| | | | | | | Unigene No. Hs.437191 |
| 36347_f_at | H2BFD | H2B histone family, member D | AA873858 | 6p21.3, | Hs.154576 | |
| | | | | 6p22-p21.3 | | |
| 38213_at | UNK_U78027 | Human BTK region clone ftp-3 | U78027 | Xq21.33-q22 | Hs.159494 | also known as BTK |
| | | mRNA | | | | (Bruton |
| | | | | | | agammaglobulinemia |
| | | | | | | tyrosine kinase) |
| 2042_s_at | MYB | v-myb avian myeloblastosis viral | M15024 | 6q22-q23 | Hs.1334 | |
| | | oncogene homolog | | | | |
| 39315_at | ANGPT1 | angiopoietin 1 | D13628 | 8q22.3-q23 | Hs.2463 | |
| 33132_at | HSU37012 | cleavage and polyadenylation | U37012 | 8q24.23 | Hs.83727 | |
| | | specificity factor | | | | |
| 286_at | H2AFO | H2A histone family, member O | L19779 | 1q21.3 | Hs.795 | |
| 38826_at | KIAA0128 | KIAA0128 protein; septin 2 | D50918 | Xq24 | Hs.90998 | |
| 35766_at | KRT18 | keratin 18 | M26326 | 12q13 | Hs.65114 | |
| 34862_at | UNK_AA005018 | ESTs, Highly similar to CGI-49 | AA005018 | 1q44 | Hs.238126 | also known as CGI-49 |
| | | protein [H. sapiens] | | | | (CGI-49 protein), |
| 36785_at | HSPB1 | heat shock 27 kD protein 1 | Z23090 | 7p12.3 | Hs.76067 | |
| 38671_at | KIAA0620 | KIAA0620 protein | AB014520 | 3q22.1 | Hs.301685 | |
| 33131_at | SOX4 | SRY (sex determining region Y)- | X70683 | 17p11.2, | Hs.83484 | |
| | | box 4 | | 6p22.3 | | |
| 41027_at | FOXC1 | forkhead box C1 | AF078096 | | | |
| 32819_at | UNK_AJ223352 | Homo sapiens mRNA for for | AJ223352 | 6p21.33 | Hs.247817 | also known as |
| | | histone H2B, clone pjG4-5-14 | | | | HIST1H2BK (histone 1, |
| | | | | | | H2bk) |
| 2025_s_at | APEX | APEX nuclease (multifunctional | M80261 | 14q11.2-q12 | Hs.73722 | |
| | | DNA repair enzyme) | | | | |
| 39710_at | P311 | P311 protein | U30521 | 5q21.3 | Hs.142827 | |
| 40184_at | CSNK1A1 | casein kinase 1, alpha 1 | L37042 | 13q13, 5 | Hs.283738 | |
| 39693_at | UNK_N53547 | Homo sapiens clone 25036 mRNA | N53547 | 11q13.1 | Hs.13662 | also known as MGC5508 |
| | | sequence | | | | (hypothetical protein |
| | | | | | | MGC5508) |
| 1470_at | POLD2 | polymerase (DNA directed), delta | U21090 | 7p15.1 | Hs.74598 | |
| | | 2, regulatory subunit (50 kD) | | | | |
| 36937_s_at | CLIM1 | carboxy terminal LIM domain | U90878 | 10q22-q26.3 | Hs.75807 | |
| | | protein 1 | | | | |
| 39691_at | UNK_AB007960 | Chromosome 1 specific transcript | AB007960 | 1p22 | Hs.136309 | also known as SH3GLB1 |
| | | KIAA0491 | | | | (SH3-domain GRB2-like |
| | | | | | | endophilin B1) |
| 40610_at | UNK_AI743507 | ESTs, Highly similar to M-phase | AI743507 | 5p13.2 | Hs.173518 | also known as ZFR (zinc |
| | | phosphoprotein homolog | | | | finger RNA binding |
| | | [H. sapiens] | | | | protein) |
| 40365_at | GNA15 | guanine nucleotide binding protein | M63904 | 19p13.3 | Hs.73797 | |
| | | (G protein), alpha 15 (Gq class) | | | | |
| 36617_at | ID1 | inhibitor of DNA binding 1, | X77956 | 20q11 | Hs.75424 | |
| | | dominant negative helix-loop-helix | | | | |
| | | protein | | | | |
| 31523_f_at | H2BFH | H2B histone family, member H | Z80780 | 21q22.3, | Hs.137594, | |
| | | | | 6p21.3, | Hs.151506, | |
| | | | | 6p21.31, | Hs.154576, | |
| | | | | 6p21.33, | Hs.180779, | |
| | | | | 6p22-p21.3 | Hs.182137, | |
| | | | | | Hs.182138, | |
| | | | | | Hs.247817, | |
| | | | | | Hs.285735, | |
| | | | | | Hs.352109, | |
| | | | | | Hs.356901, | |
| | | | | | Hs.367748 | |
| 37724_at | MYC | v-myc avian myelocytomatosis | V00568 | 8q24.12-q24.13 | Hs.79070 | |
| | | viral oncogene homolog | | | | |
| 1693_s_at | TIMP1 | tissue inhibitor of | D11139 | Xp11.3-p11.23 | Hs.5831 | |
| | | metalloproteinase 1 (erythroid | | | | |
| | | potentiating activity, collagenase | | | | |
| | | inhibitor) | | | | |
| 39054_at | GSTM4 | glutathione S-transferase M4 | X08020 | 1p13.3 | Hs.301961 | |
| 37456_at | LGALS2 | lectin, galactoside-binding, soluble, | AL022315 | 22q13.1 | Hs.113987 | |
| | | 2 (galectin 2) | | | | |
| 754_s_at | UNK_D87002 | Cluster Incl D87002: Homo | D87002 | 22q11.23 | Hs.234799 | Aligns to chr22: |
| | | sapiens immunoglobulin lambda | | | | 21303174-21303591 (+) |
| | | gene locus DNA, clone: 31F3. | | | | |
| 38811_at | ATIC | 5-aminoimidazole-4-carboxamide | D82348 | 2q35 | Hs.90280 | |
| | | ribonucleotide | | | | |
| | | formyltransferase/IMP | | | | |
| | | cyclohydrolase | | | | |
| 38585_at | HBG2 | hemoglobin, gamma G | M91036 | 11p15.5 | Hs.266959, | |
| | | | | | Hs.283108 | |
| 33528_at | KIAA0125 | KIAA0125 gene product | D50915 | 14q32.33 | Hs.38365 | |
| 40634_at | NAP1L1 | nucleosome assembly protein 1- | M86667 | 12q14.1 | Hs.302649 | |
| | | like 1 | | | | |
| 1920_s_at | CCNG1 | cyclin G1 | X77794 | 5q32-q34 | Hs.79101 | |
| 33989_f_at | TEGT | testis enhanced gene transcript | W28869 | 12q12-q13 | Hs.74637 | |
| 37716_at | MOX2 | antigen identified by monoclonal | X05323 | 3q12-q13 | Hs.79015 | |
| | | antibody MRC OX-2 | | | | |
| 37187_at | GRO2 | GRO2 oncogene | M36820 | 4q21 | Hs.75765 | |
| 38097_at | UNK_AF010313 | Homo sapiens Pig8 (PIG8) mRNA, | AF010313 | 11q24 | Hs.343911 | also known as EI24 |
| | | complete cds | | | | (etoposide induced 2.4 |
| | | | | | | mRNA) |
| 907_at | ADA | adenosine deaminase | M13792 | 20q12-q13.11 | Hs.1217 | |
| 33836_at | NPIP | nuclear pore complex interacting | AC002045 | | | |
| | | protein | | | | |
| 40485_at | UNK_AA176780 | Cluster Incl AA176780: | AA176780 | 11p11.2 | Hs.14512 | also known as TRIM44 |
| | | zp32a10.s1 Stratagene | | | | (tripartite |
| | | neuroepithelium (#937231) Homo | | | | motif-containing 44) |
| | | sapiens cDNA clone | | | | |
| | | IMAGE: 611130 3′ similar to | | | | |
| | | contains Alu repetitive element;, | | | | |
| | | mRNA sequence. | | | | |
| 36780_at | CLU | clusterin (complement lysis | M25915 | 8p21-p12 | Hs.75106 | |
| | | inhibitor, SP-40, 40, sulfated | | | | |
| | | glycoprotein 2, testosterone- | | | | |
| | | repressed prostate message 2, | | | | |
| | | apolipoprotein J) | | | | |
| 35523_at | PGDS | prostaglandin D2 synthase, | AF150241 | 4q22.1 | Hs.128433 | |
| | | hematopoietic | | | | |
| 36943_r_at | PLAGL1 | pleomorphic adenoma gene-like 1 | U81992 | 6q24-q25 | Hs.75825 | |
| 41213_at | PAGA | proliferation-associated gene A | X67951 | 1p34.1 | Hs.180909 | |
| | | (natural killer-enhancing factor A) | | | | |
| 37033_s_at | GPX1 | glutathione peroxidase 1 | X13710 | 3p21.3 | Hs.76686 | |
| 36881_at | ETFB | electron-transfer-flavoprotein, beta | X71129 | 19q13.3 | Hs.74047 | |
| | | polypeptide | | | | |
| 34651_at | COMT | catechol-O-methyltransferase | M58525 | 22q11.21 | Hs.240013 | |
| 1751_g_at | FARSL | phenylalanine-tRNA synthetase- | AD000092 | 19p13.2 | Hs.23111 | |
| | | like | | | | |
| 37376_at | LOC51035 | ORF | M68864 | 11q13.1 | Hs.77868 | |
| 38747_at | CD34 | CD34 antigen | M81945 | 1q32 | Hs.367690 | |
| 39091_at | JWA | vitamin A responsive; cytoskeleton | AF070523 | 3p14 | Hs.92384 | |
| | | related | | | | |
| 33412_at | LGALS1 | lectin, galactoside-binding, soluble, | AI535946 | 22q13.1 | Hs.227751 | |
| | | 1 (galectin 1) | | | | |
| 1456_s_at | IFI16 | interferon, gamma-inducible | M63838 | 1q22 | Hs.155530 | |
| | | protein 16 | | | | |
| 32668_at | SSBP2 | single-stranded-DNA-binding | AL080076 | 5q14.1 | Hs.169833 | |
| | | protein | | | | |
| 41812_s_at | KIAA0906 | KIAA0906 protein | AB020713 | 3p25.1 | Hs.56966 | |
| 39698_at | UNK_U51712 | Cluster Incl U51712: HSU51712 | U51712 | 4q11-q12 | Hs.13775 | also known as HOP |
| | | Human normal gingiva Homo | | | | (homeodomain-only |
| | | sapiens cDNA, mRNA sequence. | | | | protein) |
| 37705_at | PPP1R8 | protein phosphatase 1, regulatory | U14575 | 1p35 | Hs.356590 | |
| | | (inhibitor) subunit 8 | | | | |
| 35255_at | RANBP7 | RAN binding protein 7 | AF098799 | 11p15.3 | Hs.5151 | |
| 37179_at | NFE2 | nuclear factor (erythroid-derived | S77763 | 12q13 | Hs.75643 | |
| | | 2), 45 kD | | | | |
| 36749_at | CPA3 | carboxypeptidase A3 (mast cell) | M73720 | 3q21-q25 | Hs.646 | |
| 207_at | STIP1 | stress-induced-phosphoprotein 1 | M86752 | 11q13 | Hs.75612 | |
| | | (Hsp70/Hsp90-organizing protein) | | | | |
| 1520_s_at | EDN1 | endothelin 1 | J05008 | 2q14 | Hs.126256 | |
| 38675_at | SNRPC | small nuclear ribonucleoprotein | AI087268 | 6p21.2 | Hs.1063 | |
| | | polypeptide C | | | | |
| 1752_at | CALR | calreticulin | AD000092 | 19p13.3-p13.2 | Hs.16488 | |
| 1474_s_at | MYB | v-myb avian myeloblastosis viral | U22376 | 6q22-q23 | Hs.1334 | |
| | | oncogene homolog | | | | |
| 34892_at | TNFRSF10B | tumor necrosis factor receptor | AF016266 | 8p22-p21 | Hs.51233 | |
| | | superfamily, member 10b | | | | |
| 203_at | GATA2 | GATA-binding protein 2 | M68891 | 3q21, | Hs.367725 | |
| | | | | 3q22.1 | | |
| 39023_at | IDH1 | isocitrate dehydrogenase 1 | AF020038 | 2q33.3 | Hs.11223 | |
| | | (NADP+), soluble | | | | |
| 40422_at | IGFBP2 | insulin-like growth factor binding | X16302 | 2q33-q34 | Hs.162 | |
| | | protein 2 (36 kD) | | | | |
| 1161_at | HSPCB | heat shock 90 kD protein 1, beta | J04988 | 6p12 | Hs.74335 | |
| 631_g_at | DCTD | dCMP deaminase | L39874 | 4q35.1 | Hs.76894 | |
| 538_at | CD34 | CD34 antigen | S53911 | 15, 1q32 | Hs.367690 | |
| 1750_at | FARSL | phenylalanine-tRNA synthetase- | AD000092 | 19p13.2 | Hs.23111 | |
| | | like | | | | |
| 34378_at | ADFP | adipose differentiation-related | X97324 | 9p21.2 | Hs.3416 | |
| | | protein; adipophilin | | | | |
| 36618_g_at | ID1 | inhibitor of DNA binding 1, | X77956 | 20q11 | Hs.75424 | |
| | | dominant negative helix-loop-helix | | | | |
| | | protein | | | | |
| 33173_g_at | UNK_T75292 | Cluster Incl T75292: yc89b05.r1 | T75292 | 4q13.3 | Hs.8768 | also known as FLJ10849 |
| | | Soares infant brain 1NIB Homo | | | | (hypothetical protein |
| | | sapiens cDNA clone | | | | FLJ10849), Unigene No. |
| | | IMAGE: 23231 5′, mRNA | | | | Hs.386784 |
| | | sequence. | | | | |
| 37348_s_at | TRIP7 | thyroid hormone receptor | AA845349 | 6q15 | Hs.77558 | |
| | | interactor 7 | | | | |
| 33425_at | TIF1B | KRAB-associated protein 1 | X97548 | 5 | Hs.228059 | |
| 32543_at | CALR | calreticulin | M84739 | 13q14.3, | Hs.16488 | |
| | | | | 19p13.3-p13.2 | | |
| 39775_at | C1NH | complement component 1 inhibitor | X54486 | 11q12-q13.1 | Hs.151242 | |
| | | (angioedema, hereditary) | | | | |
| 948_s_at | PPID | peptidylprolyl isomerase D | D63861 | 4q31.3 | Hs.143482 | |
| | | (cyclophilin D) | | | | |
| 40774_at | CCT3 | chaperonin containing TCP1, | X74801 | 1q23 | Hs.1708 | |
| | | subunit 3 (gamma) | | | | |
| 41332_at | POLR2E | polymerase (RNA) II (DNA | D38251 | 19p13.3 | Hs.24301 | |
| | | directed) | | | | |
| | | polypeptide E (25 kD) | | | | |
| 39936_at | CCR2 | chemokine (C—C motif) | U95626 | 3p21 | Hs.395 | |
| | | receptor 2 | | | | |
| 40979_at | C14ORF3 | chromosome 14 open reading | AJ243310 | 14q23.3-31 | Hs.204041 | |
| | | frame 3 | | | | |
| 39672_at | PTPN7 | protein tyrosine phosphatase, non- | M64322 | 1q32.1 | Hs.35 | |
| | | receptor type 7 | | | | |
| 41108_at | UNK_Y14391 | Homo sapiens mRNA for putative | Y14391 | Xp22.33 | Hs.372587 | also known as PGPL |
| | | GTP-binding protein | | | | (pseudoautosomal GTP- |
| | | | | | | binding protein-like), |
| | | | | | | Unigene No. Hs.522840 |
| 40767_at | TFPI | tissue factor pathway inhibitor | M59499 | | | |
| | | (lipoprotein-associated coagulation | | | | |
| | | inhibitor) | | | | |
| 1643_g_at | MTA1 | metastasis associated 1 | U35113 | 14q32.3 | Hs.101448 | |
| 34889_at | UNK_AA056747 | ESTs, Moderately similar to | AA056747 | 3q13.31 | Hs.281866 | also known |
| | | alternatively spliced product using | | | | as ATP6V1A (ATPase, |
| | | exon 13A [H. sapiens] | | | | H+ transporting, |
| | | | | | | lysosomal 70 kDa, V1 |
| | | | | | | subunit A), Unigene No. |
| | | | | | | Hs.409131 |
| 38745_at | LIPA | lipase A, lysosomal acid, | X76488 | 10q23.2-q23.3 | Hs.85226 | |
| | | cholesterol esterase (Wolman | | | | |
| | | disease) | | | | |
| 38454_g_at | ICAM2 | intercellular adhesion molecule 2 | X15606 | 17q23-q25 | Hs.347326 | |
| 37194_at | GATA2 | GATA-binding protein 2 | M68891 | 3q21, | Hs.367725 | |
| | | | | 3q22.1 | | |
| 39061_at | BST2 | bone marrow stromal cell antigen 2 | D28137 | 19p13.2 | Hs.118110 | |
| 40916_at | UNK_AL035494 | Human DNA sequence from clone | AL035494 | | | also known as FLJ10097 |
| | | 635G19 on chromosome | | | | (hypothetical protein |
| | | Xq22.1-22.3 | | | | FLJ10097), Unigene No. |
| | | Contains a LAMR1 | | | | Hs.184736 |
| | | (Laminin Receptor | | | | |
| | | 1 (67 kD) (RPSA, 40S | | | | |
| | | Ribosomal Protein SA, P40)) | | | | |
| | | pseudogene and part of a novel | | | | |
| | | protein. Contains ESTs and GSSs | | | | |
| 39298_at | ST3GALVI | alpha2,3-sialyltransferase | AB022918 | 3q12.2 | Hs.34578 | |
| 262_at | AMD1 | S-adenosylmethionine | M21154 | 6q21-q22 | Hs.262476 | |
| | | decarboxylase 1 | | | | |
| 32241_at | TARDBP | TAR DNA binding protein | AL050265 | 1p36.22 | Hs.193989 | |
| 36138_at | CAPN4 | calpain, small polypeptide | X04106 | 19q13.13 | Hs.74451 | |
| 40827_at | IARS | isoleucine-tRNA synthetase | U04953 | 9q21 | Hs.172801 | |
| 33809_at | GNAI1 | guanine nucleotide binding protein | AL049933 | 7q21 | Hs.203862 | |
| | | (G protein), alpha inhibiting | | | | |
| | | activity polypeptide 1 | | | | |
| 36597_at | P130 | nucleolar phosphoprotein p130 | D21262 | 10q24.32 | Hs.75337 | |
| 40718_at | CTSW | cathepsin W (lymphopain) | AF013611 | 11q13.1 | Hs.87450 | |
| 1472_g_at | MYB | v-myb avian myeloblastosis viral | U22376 | 6q22-q23 | Hs.1334 | |
| | | oncogene homolog | | | | |
| 31528_f_at | H2BFE | H2B histone family, member E | Z83738 | 6p22-p21.3 | Hs.182432 | |
| 35771_at | SPN | suppressin (nuclear deformed | AF049460 | 11p15.5 | Hs.6574 | |
| | | epidermal autoregulatory factor-1 | | | | |
| | | (DEAF-1)-related) | | | | |
| 36711_at | MAFF | v-maf musculoaponeurotic | AL021977 | 22q13.1 | Hs.51305 | |
| | | fibrosarcoma (avian)oncogene | | | | |
| | | family, protein F | | | | |
| 31622_f_at | MT1F | metallothionein 1F (functional) | M10943 | | | |
| 32542_at | UNK_AF063002 | Cluster Incl AF063002: Homo | AF063002 | Xq26 | Hs.239069 | also known |
| | | sapiens LIM protein SLIMMER | | | | as FHL1 (four and |
| | | mRNA, complete cds. | | | | a half LIM domains 1), |
| | | | | | | Unigene No. |
| | | | | | | Hs.421383 |
| 35371_at | CDC4L | cell division cycle 4-like | M83822 | 4q31.22-q31.23 | Hs.62354 | |
| 37242_at | UNK_U79260 | Human clone 23745 mRNA, | U79260 | 16q12.2 | Hs.284741 | also known as FTO |
| | | complete cds | | | | (fatso), Unigene No. |
| | | | | | | Hs.284741 |
| 32165_at | SFRS7 | splicing factor, arginine/serine-rich | L41887 | 2p22-p21 | Hs.184167 | |
| | | 7 (35 kD) | | | | |
| 263_g_at | AMD1 | S-adenosylmethionine | M21154 | 6q21-q22 | Hs.262476 | |
| | | decarboxylase 1 | | | | |
| 32825_at | HRMT1L2 | HMT1 (hnRNP methyltransferase, | Y10805 | 19q13.3 | Hs.20521 | |
| | | S. cerevisiae)-like 2 | | | | |
| 31801_at | UNK_AI808712 | Homo sapiens mRNA; cDNA | AI808712 | | | Unigene No. Hs.400872 |
| | | DKFZp586L141 (from clone | | | | |
| | | DKFZp586L141) | | | | |
| 40854_at | UQCRC2 | ubiquinol-cytochrome c reductase | J04973 | 16p12 | Hs.173554 | |
| | | core protein II | | | | |
| 35741_at | PIP5K2B | phosphatidylinositol-4-phosphate | U85245 | 17q12 | Hs.6335 | |
| | | 5-kinase, type II, beta | | | | |
| 39056_at | ADE2H1 | multifunctional polypeptide similar | X53793 | 4pter-q21 | Hs.117950 | |
| | | to SAICAR synthetase and AIR | | | | |
| | | carboxylase | | | | |
| 37384_at | KIAA0015 | KIAA0015 gene product | D13640 | 22q11.22 | Hs.278441 | |
| 478_g_at | IRF5 | interferon regulatory factor 5 | U51127 | 7q32 | Hs.334450 | |
| 34023_at | FCER1A | Fc fragment of IgE, high affinity I, | X06948 | 1q23 | Hs.897 | |
| | | receptor for; alpha polypeptide | | | | |
| 38704_at | ACF7 | actin binding protein; macrophin | AB007934 | 1p32-p31 | Hs.108258 | |
| | | (microfilament and actin filament | | | | |
| | | cross-linker protein) | | | | |
| 36684_at | AMD1 | S-adenosylmethionine | M21154 | 6q21-q22 | Hs.262476 | |
| | | decarboxylase 1 | | | | |
| 38123_at | D123 | D123 gene product | D14878 | 10p13 | Hs.82043 | |
| 41322_s_at | UNK_AI816034 | ESTs, Highly similar to 40% | AI816034 | 5q35.3 | Hs.23990 | also known as NOLA2 |
| | | similar to yeast high mobility | | | | (nucleolar protein family |
| | | group-like nuclear protein, P32495 | | | | A, member 2 (H/ACA |
| | | [H. sapiens] | | | | small nucleolar RNPs)), |
| | | | | | | Unigene No. Hs.386392 |
| 35182_f_at | UNK_W25874 | Homo sapiens mRNA; cDNA | W25874 | 4q13.3 | Hs.8768 | NM_018243, |
| | | DKFZp566C224 (from clone | | | | hypothetical |
| | | DKFZp566C224) | | | | protein FLJ10849, chr4: |
| | | | | | | 78329289-78418162 (+) |
| 36955_at | GP36B | endoplasmic reticulum | U10362 | 5q35.3 | Hs.75864 | |
| | | glycoprotein | | | | |
| 31670_s_at | CAMK2G | calcium/calmodulin-dependent | U81554 | 10q22, 1q32 | Hs.250857 | |
| | | protein kinase (CaM kinase) II | | | | |
| | | gamma | | | | |
| 39638_at | TFAP4 | transcription factor AP-4 | S73885 | 16p13 | Hs.3005 | |
| | | (activating enhancer-binding | | | | |
| | | protein 4) | | | | |
| 41357_at | ATP5B | ATP synthase, H+ transporting, | W27997 | 12p13-qter | Hs.25 | |
| | | mitochondrial F1 complex, beta | | | | |
| | | polypeptide | | | | |
| 32087_at | HSF2 | heat shock transcription factor 2 | M65217 | 6q22.33 | Hs.158195 | |
| 39968_at | LTC4S | leukotriene C4 synthase | U50136 | 5q35 | Hs.456 | |
| 31524_f_at | H2BFK | H2B histone family, member K | Z80782 | 6p21.3 | Hs.182140 | |
| 37018_at | H1F2 | H1 histone family, member 2 | AI189287 | 6p21.3 | Hs.7644 | |
| 39471_at | M11S1 | membrane component, | Z48042 | 11p13 | Hs.278672 | |
| | | chromosome 11, surface marker 1 | | | | |
| 40877_s_at | UNK_AF041080 | Homo sapiens D15F37 | AF041080 | 15q11-q13 | Hs.286132 | also known as MN7 |
| | | pseudogene, S3 allele, mRNA | | | | (D15F37 (pseudogene)), |
| | | sequence | | | | Unigene No. Hs.458334 |
| 35292_at | D6S81E | HLA-B associated transcript-1 | Z37166 | 6p21.3, | Hs.55296 | |
| | | | | 9p13 | | |
| 39799_at | FABP5 | fatty acid binding protein 5 | M94856 | 8q21.13 | Hs.153179 | |
| | | (psoriasis-associated) | | | | |
| 40698_at | CLECSF2 | C-type (calcium dependent, | X96719 | 12p13-p12 | Hs.85201 | |
| | | carbohydrate-recognition domain) | | | | |
| | | lectin, superfamily member 2 | | | | |
| | | (activation-induced) | | | | |
| 37726_at | RPML3 | ribosomal protein, mitochondrial, | X06323 | 3q21-q23 | Hs.79086 | |
| | | L3 | | | | |
| 41379_at | KIAA0594 | KIAA0594 protein | AB011166 | 9q21.12 | Hs.103283 | |
| 33415_at | NME2 | non-metastatic cells 2, protein | X58965 | 17q21.3 | Hs.275163 | |
| | | (NM23B) expressed in | | | | |
| 38416_at | CCT6A | chaperonin containing TCP1, | L27706 | 7p14.1 | Hs.82916 | |
| | | subunit 6A (zeta 1) | | | | |
| 36958_at | ZYX | zyxin | X95735 | 13q12, 7q32 | Hs.75873 | |
| 35801_at | UNK_AF026816 | Homo sapiens putative oncogene | AF026816 | 20p | Hs.6817 | also known as ITPA |
| | | protein mRNA, partial cds | | | | (inosine triphosphatase |
| | | | | | | (nucleoside triphosphate |
| | | | | | | pyrophosphatase)) |
| 38780_at | AKR1A1 | aldo-keto reductase family 1, | J04794 | 1p33-p32 | Hs.89529 | |
| | | member A1 (aldehyde reductase) | | | | |
| 1196_at | CHC1 | chromosome condensation 1 | D00591 | 1p36.1 | Hs.84746 | |
| 32548_at | P23 | unactive | L24804 | 12q12 | Hs.278270 | |
| | | progesterone receptor, 23 kD | | | | |
| 34961_at | TACTILE | T cell activation, increased late | M88282 | 3q13.2 | Hs.142023 | |
| | | expression | | | | |
| 40962_s_at | SMARCA2 | SWI/SNF related, matrix | D26155 | 9p22.3 | Hs.198296 | |
| | | associated, actin dependent | | | | |
| | | regulator of chromatin, subfamily | | | | |
| | | a, member 2 | | | | |
| 33219_at | KIAA1097 | KIAA1097 protein | AB029020 | 1p31.1 | Hs.173694 | |
| 38473_at | TARS | threonyl-tRNA synthetase | M63180 | 5p13-cen | Hs.84131 | |
| 37399_at | AKR1C3 | aldo-keto reductase family 1, | D17793 | 10p15-p14 | Hs.78183 | |
| | | member C3 (3-alpha | | | | |
| | | hydroxysteroid dehydrogenase, | | | | |
| | | type II) | | | | |
| 38052_at | F13A1 | coagulation factor XIII, A1 | M14539 | 6p25.3-p24.3 | Hs.80424 | |
| | | polypeptide | | | | |
| 38376_at | ACADVL | acyl-Coenzyme A dehydrogenase, | L46590 | 17p13-p11 | Hs.82208 | |
| | | very long chain | | | | |
| 33925_at | NRGN | neurogranin (protein kinase C | X99076 | 11q24 | Hs.26944 | |
| | | substrate, RC3) | | | | |
| 40842_at | SNRPA | small nuclear ribonucleoprotein | M60784 | 19q13.1 | Hs.173255 | |
| | | polypeptide A | | | | |
| 32803_at | CNIL | cornichon-like | AF104398 | 14q22.1 | Hs.201673 | |
| 34251_at | HOXB5 | homeo box B5 | M92299 | 17q21-q22 | Hs.22554 | |
| 1449_at | PSMA4 | proteasome (prosome, macropain) | D00763 | 15q24.2 | Hs.251531 | |
| | | subunit, alpha type, 4 | | | | |
| 351_f_at | UNK_D28423 | Cluster Incl D28423: Human | D28423 | | | also known as SFRS3 |
| | | mRNA for pre-mRNA splicing | | | | (splicing factor, |
| | | factor SRp20, 5′UTR (sequence | | | | arginine/serine-rich 3), |
| | | from the 5′cap to the start codon). | | | | Unigene No. Hs.405144 |
| 38642_at | ALCAM | activated leucocyte cell adhesion | Y10183 | 3q13.1 | Hs.10247 | |
| | | molecule | | | | |
| 39341_at | TRIP6 | thyroid hormone | AJ001902 | 17p13.3, | Hs.119498 | |
| | | receptor interactor 6 | | 7q22 | | |
| 37774_at | UNK_AI819942 | Cluster Incl AI819942: wj88e02.x1 | AI819942 | Xq24 | Hs.90998 | also known as 6-Sep |
| | | NCI_CGAP_Lym12 | | | | (septin 6) |
| | | Homo sapiens cDNA | | | | |
| | | clone IMAGE: 2409914 3′ | | | | |
| | | similar to SW: GBB5_HUMAN | | | | |
| | | O14775 GUANINE | | | | |
| | | NUCLEOTIDE-BINDING | | | | |
| | | PROTEIN BETA SUBUNIT 5;, | | | | |
| | | mRNA sequence. | | | | |
| 39767_at | CCT8 | chaperonin containing TCP1, | D13627 | 21q22.11 | Hs.15071 | |
| | | subunit 8 (theta) | | | | |
| 37692_at | DBI | diazepam binding inhibitor (GABA | AI557240 | 2q12-q21 | Hs.78888 | |
| | | receptor modulator, acyl- | | | | |
| | | Coenzyme A binding protein) | | | | |
| 32232_at | NDUFB5 | NADH dehydrogenase | AF047181 | 3q27.1 | Hs.19236 | |
| | | (ubiquinone) 1 beta subcomplex, 5 | | | | |
| | | (16 kD, SGDH) | | | | |
| 38072_at | UNK_AL031432 | Human DNA sequence from clone | AL031432 | 1p36.13-p35.1 | Hs.8084 | also known as |
| | | 465N24 on chromosome | | | | DJ465N24.2.1 |
| | | 1p35.1-36.13. | | | | (pothetical protein |
| | | Contains two novel genes, | | | | dJ465N24.2.1), Unigene |
| | | ESTs, GSSs and CpG islands | | | | No. Hs.259412 |
| 38399_at | SNRPB2 | small nuclear ribonucleoprotein | AL034428 | 20p12.2-p11.22 | Hs.82575 | |
| | | polypeptide B″ | | | | |
| 41202_s_at | OS4 | conserved gene amplified in | AF000152 | 12q13-q15 | Hs.180669 | |
| | | osteosarcoma | | | | |
| 1942_s_at | CDK4 | cyclin-dependent kinase 4 | U37022 | 12q14 | Hs.95577 | |
| 32844_at | EIF4G1 | eukaryotic translation initiation | AF104913 | 3q27-qter | Hs.211568 | |
| | | factor 4 gamma, 1 | | | | |
| 35342_at | UNK_AF052159 | Homo sapiens clone 24416 mRNA | AF052159 | | Hs.5957 | also known as PTPLB |
| | | sequence | | | | (protein tyrosine |
| | | | | | | phosphatase-like (proline |
| | | | | | | instead of catalytic |
| | | | | | | arginine), member b) |
| 41123_s_at | PDNP2 | phosphodiesterase I/nucleotide | L35594 | 8q24.1 | Hs.174185 | |
| | | pyrophosphatase 2 (autotaxin) | | | | |
| 38233_at | HOMER-3 | Homer, neuronal immediate early | AF093265 | 19p13.11 | Hs.166146 | |
| | | gene, 3 | | | | |
| 2012_s_at | UNK_U34994 | Human DNA-dependent protein | U34994 | 8q11 | Hs.155637 | also known as PRKDC |
| | | kinase catalytic subunit (DNA- | | | | (protein kinase, DNA- |
| | | PKcs) mRNA, complete cds | | | | activated, catalytic |
| | | | | | | polypeptide), |
| | | | | | | Unigene No. |
| | | | | | | Hs.415749 |
| 35848_at | UNK_AL049432 | Homo sapiens mRNA; cDNA | AL049432 | 10q23.2 | Hs.7252 | also known as RAI17 |
| | | DKFZp586J231 (from clone | | | | (retinoic acid induced |
| | | DKFZp586J231) | | | | 17), Unigene No. |
| | | | | | | Hs.438767 |
| 41163_at | P24B | integral type I protein | AL109672 | 15q24-q25 | Hs.179516 | |
| 41375_at | UNK_AJ245416 | Homo sapiens mRNA for G7b | AJ245416 | 6p21.3 | Hs.103106 | also known as LSM2 |
| | | protein (G7b gene, located in the | | | | (LSM2 homolog, |
| | | class III region of the major | | | | U6 small nuclear RNA |
| | | histocompatibility complex | | | | associated |
| | | | | | | (S. cerevisiae)) |
| 38443_at | UNK_U79291 | Human clone 23721 mRNA | U79291 | 12q24.11 | Hs.83572 | also known as PTPN11 |
| | | sequence | | | | (protein tyrosine |
| | | | | | | phosphatase, |
| | | | | | | non-receptor |
| | | | | | | type 11 (Noonan |
| | | | | | | syndrome 1)) |
| 39792_at | HNRPR | heterogeneous nuclear | AF000364 | 1p36.11 | Hs.15265 | |
| | | ribonucleoprotein R | | | | |
| 37392_at | PHKB | phosphorylase kinase, beta | X84908 | 16q12-q13 | Hs.78060 | |
| 38011_at | RMP | RPB5-mediating protein | AB006572 | 19q12 | Hs.7943 | |
| 39507_at | OGT | O-linked N-acetylglucosamine | AL050366 | Xq13 | Hs.100293 | |
| | | (GlcNAc) transferase (UDP-N- | | | | |
| | | acetylglucosamine: polypeptide-N- | | | | |
| | | acetylglucosaminyl transferase) | | | | |
| 1476_s_at | MYB | v-myb avian myeloblastosis viral | U22376 | 6q22-q23 | Hs.1334 | |
| | | oncogene homolog | | | | |
| 34325_at | PQBP1 | polyglutamine binding protein 1 | AJ005893 | Xp11.23 | Hs.30570 | |
| 36185_at | AARS | alanyl-tRNA synthetase | D32050 | 16q22 | Hs.75102 | |
| 35818_at | CYC1 | cytochrome c-1 | D00265 | 7p21.2, | Hs.169248 | |
| | | | | Xq22.1 | | |
| 38808_at | GP110 | cell membrane glycoprotein, | D64154 | 20q13.33 | Hs.90107 | |
| | | 110000M(r) (surface antigen) | | | | |
| 40133_s_at | UNK_W28944 | ESTs, Weakly similar to 3- | W28944 | 9q12 | Hs.155742 | also known as GRHPR |
| | | phosphoglycerate dehydrogenase | | | | (glyoxylate |
| | | [H. sapiens] | | | | reductase/ |
| | | | | | | hydroxypyruvate |
| | | | | | | reductase) |
| 1287_at | ADPRT | ADP-ribosyltransferase (NAD+; | J03473 | 1q41-q42 | Hs.177766 | |
| | | poly (ADP-ribose) polymerase) | | | | |
| 41725_at | CSNK1G2 | casein kinase 1, gamma 2 | U89896 | 19p13.3 | Hs.181390 | |
| 1499_at | FNTA | farnesyltransferase, CAAX box, | L10413 | 8p22-q11 | Hs.356463 | |
| | | alpha | | | | |
| 41535_at | DOC1 | deleted in oral cancer (mouse, | AF006484 | 12q24.31 | Hs.3436 | |
| | | homolog) 1 | | | | |
| 36892_at | ITGA7 | integrin, alpha 7 | AF032108 | 12q13 | Hs.74369 | |
| 38695_at | NDUFS4 | NADH dehydrogenase | AA203303 | 5q11.1 | Hs.10758 | |
| | | (ubiquinone) Fe—S | | | | |
| | | protein 4 (18 kD) | | | | |
| | | (NADH-coenzyme Q reductase) | | | | |
| 39139_at | SPC18 | signal peptidase complex (18 kD) | AI357653 | 15q24.3 | Hs.9534 | |
| 1660_at | UBE2N | ubiquitin-conjugating enzyme E2N | D83004 | 12q21.33 | Hs.75355 | |
| | | (homologous to yeast UBC13) | | | | |
| 1151_at | RPL22 | ribosomal protein L22 | X59357 | | | |
| 32184_at | LMO2 | LIM domain only 2 (rhombotin- | X61118 | 11p13 | Hs.184585 | |
| | | like 1) | | | | |
| 35184_at | KIAA0546 | KIAA0546 protein | AB011118 | 12q13.3 | Hs.26764 | |
| 41243_at | UNK_AB007916 | Homo sapiens mRNA; cDNA | AB007916 | 1p36.33 | Hs.214646 | also known as SLC35E2 |
| | | DKFZp564C093 (from clone | | | | (solute carrier family 35, |
| | | DKFZp564C093) | | | | member E2), |
| | | | | | | Unigene No. Hs.458492 |
| 153_f_at | H2BFR | H2B histone family, | X00088 | 6p21.31 | Hs.285735 | |
| | | member R | | | | |
| 1826_at | ARHB | ras homolog | M12174 | 2pter-p12 | Hs.204354 | |
| | | gene family, member B | | | | |
| 1521_at | NME1 | non-metastatic cells 1, protein | X17620 | 17q21.3 | Hs.118638 | |
| | | (NM23A) expressed in | | | | |
| 36624_at | IMPDH2 | IMP (inosine monophosphate) | L33842 | 3p21.2 | Hs.75432 | |
| | | dehydrogenase 2 | | | | |
| 32696_at | PBX3 | pre-B-cell leukemia transcription | X59841 | 9q33-q34 | Hs.294101 | |
| | | factor 3 | | | | |
| 40467_at | SDHD | succinate dehydrogenase complex, | AB006202 | 11q23 | Hs.168289 | |
| | | subunit D, integral membrane | | | | |
| | | protein | | | | |
| 40638_at | SFPQ | splicing factor proline/glutamine | X70944 | 1p34.2 | Hs.180610 | |
| | | rich (polypyrimidine tract-binding | | | | |
| | | protein-associated) | | | | |
| 32051_at | UNK_AJ224875 | Homo sapiens mRNA for putative | AJ224875 | 11pter-p15.5 | Hs.155356 | also known as ALG8 |
| | | glucosyltransferase, partial cds | | | | (asparagine-linked |
| | | | | | | glycosylation 8 homolog |
| | | | | | | (yeast, alpha-1,3- |
| | | | | | | glucosyltransferase)), |
| | | | | | | Unigene No. Hs.440117 |
| 32853_at | TOMM70A | translocase of outer mitochondrial | AB018262 | 3q12.3 | Hs.21198 | |
| | | membrane 70 (yeast) homolog A | | | | |
| 34099_f_at | UNK_W26056 | ESTs, Moderately similar to | W26056 | 12q14.1 | Hs.302649 | also known as NAP1L1 |
| | | NUCLEOSOME ASSEMBLY | | | | (nucleosome assembly |
| | | PROTEIN 1-LIKE 1 [H. sapiens] | | | | protein 1-like |
| | | | | | | 1), Unigene |
| | | | | | | No. Hs.419776 |
| 1009_at | HINT | histidine triad nucleotide-binding | U51004 | 5q31.2 | Hs.256697 | |
| | | protein | | | | |
| 40441_g_at | DKFZP564M2423 | DKFZP564M2423 protein | AL080119 | 1p31-p22 | Hs.165998 | |
| 37281_at | KIAA0233 | KIAA0233 gene product | D87071 | 16q24.3 | Hs.79077 | |
| 34893_at | NDUFV2 | NADH dehydrogenase | AI557064 | 18p11.31-p11.2 | Hs.51299 | |
| | | (ubiquinone) | | | | |
| | | flavoprotein 2 (24 kD) | | | | |
| 36465_at | IRF5 | interferon regulatory factor 5 | U51127 | 7q32 | Hs.334450 | |
| 33891_at | CLIC4L | chloride intracellular channel 4 like | AL080061 | 1p36.11 | Hs.25035 | |
| 39639_s_at | TNP1 | transition protein 1 (during histone | X07948 | 2q35-q36 | Hs.3017 | |
| | | to protamine replacement) | | | | |
| 33439_at | TCF8 | transcription factor 8 (represses | D15050 | 10p11.2 | Hs.232068 | |
| | | interleukin 2 expression) | | | | |
| 35012_at | MNDA | myeloid cell nuclear differentiation | M81750 | 1q22 | Hs.153837 | |
| | | antigen | | | | |
| 36375_at | ODF1 | outer dense fibre of sperm tails 1 | X74614 | 8q22 | Hs.159274 | |
| 39059_at | DHCR7 | 7-dehydrocholesterol reductase | AF034544 | 11q13.2-q13.5 | Hs.11806 | |
| 37924_g_at | UNK_AA846749 | Homo sapiens mRNA for G3a | AA846749 | 6p21.31 | Hs.247129 | also known as APOM |
| | | protein (G3a gene, located in the | | | | (apolipoprotein M), |
| | | class III region of the major | | | | Unigene No. Hs.247323 |
| | | histocompatibility complex) | | | | |
| 1237_at | IER3 | immediate early response 3 | S81914 | 6p21.3 | Hs.76095 | |
| 36277_at | CD3E | CD3E antigen, epsilon polypeptide | M23323 | 11q23 | Hs.3003 | |
| | | (TiT3 complex) | | | | |
| 38113_at | KIAA0796 | KIAA0796 protein | AB018339 | 6q25.1 | Hs.8182 | |
| 35590_s_at | GIPR | gastric inhibitory polypeptide | X81832 | 19q13.3 | Hs.251412 | |
| | | receptor | | | | |
| 34912_at | DAPK2 | death-associated protein kinase 2 | AF052941 | 15q22.1 | Hs.129208 | |
| 39822_s_at | GADD45B | growth arrest and DNA-damage- | AF078077 | 19p13.3 | Hs.110571 | |
| | | inducible, beta | | | | |
| 34161_at | LPO | lactoperoxidase | U39573 | 17q23.1 | Hs.234742 | |
| 35091_at | NRG2 | neuregulin 2 | AA706226 | 5q23-q33 | Hs.113264 | |
| 37536_at | CD83 | CD83 antigen (activated B | Z11697 | 6p23 | Hs.79197 | |
| | | lymphocytes, immunoglobulin | | | | |
| | | superfamily) | | | | |
| 214_at | MSX1 | msh (Drosophila) homeo box | M97676 | 4p16.3-p16.1 | Hs.1494 | |
| | | homolog 1 (formerly homeo box 7) | | | | |
| 721_g_at | HSF4 | heat shock transcription factor 4 | D87673 | 16q21 | Hs.75486 | |
| 179_at | PMS2L11 | postmeiotic segregation increased | U38980 | 7q | Hs.306174 | |
| | | 2-like 11 | | | | |
| 100_g_at | RABGGTA | Rab geranylgeranyltransferase, | Y08200 | 14q11.2 | Hs.78920 | |
| | | alpha subunit | | | | |
| 33080_s_at | KIAA0474 | KIAA0474 gene product | AB007943 | 1p36.1-p35 | Hs.75151 | |
| 38229_at | UNK_X90579 | H. sapiens DNA for cyp related | X90579 | | Hs.166079 | also known as CYP3A5 |
| | | pseudogene | | | | (cytochrome P450, |
| | | | | | | family |
| | | | | | | 3, subfamily A, |
| | | | | | | polypeptide 5), Unigene |
| | | | | | | No. Hs.150276 |
| 35485_at | GRM4 | glutamate receptor, metabotropic 4 | X80818 | 6p21.3 | Hs.178078 | |
| 32228_at | ADTAB | adaptor-related protein complex 2, | AB020706 | 11 | Hs.19121 | |
| | | alpha 2 subunit | | | | |
| 37425_g_at | UNK_AB029343 | Homo sapiens HCR (a-helix | AB029343 | 6p21.3 | Hs.110746 | also known as C6orf18 |
| | | coiled-coil rod homologue) | | | | (chromosome 6 open |
| | | mRNA, complete cds | | | | reading frame 18) |
| 34060_g_at | UNK_AA586695 | Cluster Incl AA586695: | AA586695 | 8q24 | Hs.8854 | Unigene No. Hs.459222; |
| | | nn42h06.s1 NCI_CGAP_GC5 | | | | Homo sapiens cDNA |
| | | Homo sapiens cDNA clone | | | | FLJ26234 fis, clone |
| | | IMAGE: 10865873′, mRNA | | | | ADG09627 |
| | | sequence. | | | | |
| 35367_at | LGALS3 | lectin, galactoside-binding, soluble, | AB006780 | 14q21-q22 | Hs.621 | |
| | | 3 (galectin 3) | | | | |
| 36378_at | UPK1A | uroplakin 1A | AF085807 | 19q13.13 | Hs.159309 | |
| 32193_at | PLXNC1 | plexin C1 | AF030339 | 12q23.3 | Hs.286229 | |
| 32747_at | ALDH2 | aldehyde dehydrogenase 2, | X05409 | 12q24.2 | Hs.195432 | |
| | | mitochondrial | | | | |
| 36916_at | SIAT4C | sialyltransferase 4C (beta- | X74570 | 11q23-q24 | Hs.75268 | |
| | | galactosidase alpha-2,3- | | | | |
| | | sialytransferase) | | | | |
| 32469_at | CEACAM3 | carcinoembryonic antigen-related | L00693 | 19q13.2 | Hs.11 | |
| | | cell adhesion molecule 3 | | | | |
| 595_at | TNFAIP3 | tumor necrosis factor, alpha- | M59465 | 6q23.1-q25.3 | Hs.211600 | |
| | | induced protein 3 | | | | |
| 32227_at | PRG1 | proteoglycan 1, secretory granule | X17042 | 10q22.1 | Hs.1908 | |
| 33752_at | NS1-BP | NS1-binding protein | AB020657 | 1q25.1-q31.1 | Hs.197298 | |
| 38138_at | S100A11 | S100 calcium-binding protein A11 | D38583 | 1q21, 7q22-q31.1 | Hs.256290 | |
| | | (calgizzarin) | | | | |
| 888_s_at | GDF1 | growth differentiation factor 1 | M62302 | 19p12 | Hs.339810, | |
| | | | | | Hs.348258 | |
| 1106_s_at | TRA@ | T cell receptor alpha locus | M12959 | 14q11.2 | Hs.74647 | |
| 39815_at | UNK_AA883101 | ESTs, Weakly similar to putative | AA883101 | 1q32.2 | Hs.109494 | also known as SPUF |
| | | progesterone binding protein | | | | (secreted protein of |
| | | [H. sapiens] | | | | unknown function) |
| 35785_at | UNK_W28281 | ESTs, Moderately similar to | W28281 | 12p13.1 | Hs.336429 | also known as |
| | | MM46 [H. sapiens] | | | | GABARAPL1 |
| | | | | | | (GABA(A) receptor- |
| | | | | | | associated protein |
| | | | | | | like 1) |
| 33333_at | KIAA0403 | KIAA0403 protein | AB007863 | 6q25.2 | Hs.185140 | |
| 1894_f_at | UNK_L27065 | Neurofibromatosis 2 Tumor | L27065 | | | Accession No. |
| | | Suppressor | | | | HG3236-HT3413 |
| 38162_at | KIAA0751 | KIAA0751 gene product | AF007156 | 8q22.3 | Hs.153610 | |
| 38735_at | KIAA0513 | KIAA0513 gene product | AB011085 | 16q23.3 | Hs.301658 | |
| 38406_f_at | PTGDS | prostaglandin D2 synthase (21 kD, | AI207842 | 9q34.2-q34.3 | Hs.8272 | |
| | | brain) | | | | |
| 37898_r_at | TFF3 | trefoil factor 3 (intestinal) | AI985964 | 21q22.3 | Hs.82961 | |
| 39527_at | UNK_AF090102 | Homo sapiens mRNA; cDNA | AF090102 | 2p12 | Hs.102657 | Unigene No. Hs.416735 |
| | | DKFZp564L2223 (from clone | | | | Homo sapiens clone |
| | | DKFZp564L2223) | | | | IMAGE 21785 |
| 31815_r_at | LRP3 | low density lipoprotein receptor- | AB009462 | 19q13.12 | Hs.143641 | |
| | | related protein 3 | | | | |
| 38976_at | CORO1A | coronin, actin-binding protein, 1A | D44497 | 16q13 | Hs.109606 | |
| 41038_at | NCF2 | neutrophil cytosolic factor 2 | M32011 | 1q25 | Hs.949 | |
| | | (65 kD, chronic granulomatous | | | | |
| | | disease, autosomal 2) | | | | |
| 36207_at | SEC14L1 | SEC14 (S. cerevisiae)-like 1 | D67029 | 17q25.1-17q25.2 | Hs.75232 | |
| 31687_f_at | HBB | hemoglobin, beta | M25079 | 11p15.5 | Hs.155376 | |
| 32675_at | BST1 | bone marrow stromal cell antigen 1 | D21878 | 4p15 | Hs.169998 | |
| 649_s_at | CXCR4 | chemokine (C—X—C | L06797 | 2q21 | Hs.89414 | |
| | | motif), receptor | | | | |
| | | 4 (fusin) | | | | |
| 2077_at | UNK_L40385 | Homo sapiens integrin alpha 6 | L40385 | 2q31.1 | Hs.227730 | NM_000210; integrin |
| | | (ITGA6) subunit gene, exons. | | | | alpha chain, alpha 6; |
| | | | | | | chr2: |
| | | | | | | 173495078-173571644 |
| | | | | | | (+) |
| | | | | | | L40385exons#1-3 |
| 404_at | IL4R | interleukin 4 receptor | X52425 | 16p11.2-12.1 | Hs.75545 | |
| 36114_r_at | TNNT1 | troponin T1, skeletal, slow | M19309 | 19q13.4 | Hs.73980 | |
| 1105_s_at | TRB@ | T cell receptor beta locus | M12886 | 7q34 | Hs.303157 | |
| 39399_at | TBCD | tubulin-specific chaperone d | AJ006417 | 17q25.3 | Hs.12570 | |
| 32673_at | BTN2A1 | butyrophilin, subfamily 2, member | U90543 | 6p22.1 | Hs.169963 | |
| | | A1 | | | | |
| 33758_f_at | PSG11 | pregnancy specific beta-1- | U25988 | 19q13.2 | Hs.334408 | |
| | | glycoprotein 11 | | | | |
| 31692_at | HSPA1B | heat shock 70 kD protein 1 | M59830 | 6p21.3 | Hs.274402, | |
| | | | | | Hs.8997 | |
| 34112_r_at | UNK_AL050065 | Homo sapiens mRNA; cDNA | AL050065 | | Hs.212587 | also known as |
| | | DKFZp566M043 (from clone | | | | HRMT1L1 (HMT1 |
| | | DKFZp566M043) | | | | hnRNP |
| | | | | | | methyltransferase- |
| | | | | | | like 1 |
| | | | | | | (S. cerevisiae)), Unigene |
| | | | | | | No. Hs.154163 |
| 41840_r_at | UNK_H08175 | Homo sapiens clone IMAGE | H08175 | | Hs.6524 | |
| | | 25997 | | | | |
| 37556_at | GCL | grancalcin | M81637 | 2q24.3 | Hs.79381 | |
| 32916_at | PTPRE | protein tyrosine phosphatase, | X54134 | 10q26 | Hs.31137 | |
| | | receptor type, epsilon polypeptide | | | | |
| 34655_at | MPP2 | membrane protein, palmitoylated 2 | AI951832 | 17q12-q21 | Hs.23205 | |
| | | (MAGUK p55 subfamily member | | | | |
| | | 2) | | | | |
| 40699_at | CD8A | CD8 antigen, alpha polypeptide | M12824 | 2p12 | Hs.85258 | |
| | | (p32) | | | | |
| 38895_i_at | NCF4 | neutrophil cytosolic factor 4 | X77094 | 22q13.1 | Hs.196352 | |
| | | (40 kD) | | | | |
| 31562_at | RHOK | rhodopsin kinase | U63973 | 13q34 | Hs.103501 | |
| 1150_at | PTPRE | protein tyrosine phosphatase, | X54134 | | | |
| | | receptor type, epsilon polypeptide | | | | |
| 1104_s_at | HSPA1A | heat shock 70 kD protein 1 | M11717 | 6p21.3 | Hs.274402, | |
| | | | | | Hs.8997 | |
| 36640_at | MYL2 | myosin, light polypeptide 2, | X66141 | 12q23-q24.3 | Hs.75535 | |
| | | regulatory, cardiac, slow | | | | |
| 31357_at | UNK_W26214 | Cluster Incl W26214: 22d11 | W26214 | | | Accession No. W26214 |
| | | Human retina cDNA randomly | | | | |
| | | primed sublibrary Homo sapiens | | | | |
| | | cDNA, mRNA sequence. | | | | |
| 40215_at | UGCG | UDP-glucose ceramide | D50840 | 9q31 | Hs.152601 | |
| | | glucosyltransferase | | | | |
| 32897_at | MTHFR | 5,10-methylenetetrahydrofolate | AJ237672 | 1p36.3 | Hs.214142 | |
| | | reductase (NADPH) | | | | |
| 40876_at | GYG | glycogenin | U31525 | 3q24-q25.1 | Hs.174071 | |
| 36488_at | EGFL5 | EGF-like-domain, multiple 5 | AB011542 | 9q32-q33.3 | Hs.5599 | |
| 40278_at | KIAA1080 | KIAA1080 protein | AB029003 | 16p12 | Hs.155546 | |
| 40089_at | UNK_AJ224442 | Homo sapiens mRNA for putative | AJ224442 | | Hs.155020 | also |
| | | methyltransferase | | | | known as WBSCR22 |
| | | | | | | (Williams Beuren |
| | | | | | | syndrome chromosome |
| | | | | | | region 22), Unigene No. |
| | | | | | | Hs.413036 |
| 40739_at | CA4 | carbonic anhydrase IV | M83670 | 17q23 | Hs.89485 | |
| 32525_r_at | UNK_W29012 | Cluster Incl W29012: 55a6 Human | W29012 | 11q25 | Hs.334703 | also known as JAM3 |
| | | retina cDNA randomly primed | | | | (junctional adhesion |
| | | sublibrary Homo sapiens cDNA, | | | | molecule 3), |
| | | mRNA sequence. | | | | Unigene No. Hs.419149 |
| 31621_s_at | ELN | elastin (supravalvular aortic | M36860 | 7q11.23 | Hs.9295 | |
| | | stenosis, Williams-Beuren | | | | |
| | | syndrome) | | | | |
| 110_at | CSPG4 | chondroitin sulfate proteoglycan 4 | X96753 | 15 | Hs.9004 | |
| | | (melanoma-associated) | | | | |
| 32904_at | PRF1 | perforin 1 (preforming protein) | M28393 | 10q22 | Hs.2200 | |
| 32407_f_at | UNK_U92818 | Homo sapiens c33.28 unnamed | U92818 | | | Accession No. U92818 |
| | | HERV-H protein mRNA, partial | | | | |
| | | cds | | | | |
| 416_s_at | UNK_X61755 | Human partial mRNA for | X61755 | 12q12-q13 | Hs.111473 | Accession No. X61755 |
| | | homeodomain protein | | | | |
| AFFX- | 28SRNA3_Hs_AFFX | 28SRNA3 control sequence | M27830 | | | |
| | | (H. sapiens) | | | | |
| M27830_3_at | | [AFFX] | | | | |
| 32815_at | UNK_AI687419 | Cluster Incl AI687419: tp95h03.x1 | AI687419 | | | Accession No. AI687419 |
| | | NCI_CGAP_Ut3 Homo sapiens | | | | |
| | | cDNA clone IMAGE: 2207093 3′ | | | | |
| | | similar to contains L1.b3 L1 | | | | |
| | | repetitive element;, mRNA | | | | |
| | | sequence. | | | | |
| 1339_s_at | UNK_X14675 | Cluster Incl X14675: Human bcr- | X14675 | | | Unigene No. Hs.526684 |
| | | abl mRNA 5′ fragment (clone 3c). | | | | Human bcr-abl mRNA 5′ |
| | | | | | | fragment (clone 3c) |
| 38417_at | AMPD2 | adenosine monophosphate | M91029 | | | |
| | | deaminase 2 (isoform L) | | | | |
| 38894_g_at | NCF4 | neutrophil cytosolic factor 4 | AL008637 | 22q13.1 | Hs.196352 | |
| | | (40 kD) | | | | |
| 36459_at | KIAA0879 | KIAA0879 protein | AB020686 | 6p12.3 | Hs.54037 | |
| 32901_s_at | NPM1P14 | nucleophosmin 1 (nucleolar | AC005192 | 7q22-q31 | Hs.7879 | |
| | | phosphoprotein B23, numatrin) | | | | |
| | | pseudogene 14 | | | | |
| 34965_at | CST7 | cystatin F (leukocystatin) | AF031824 | 20p11.21 | Hs.143212 | |
| 34415_at | ACVR1B | activin A receptor, type IB | Z22536 | 12q13 | Hs.99954 | |
| 35714_at | PDXK | pyridoxal (pyridoxine, vitamin B6) | U89606 | 21q22.3 | Hs.38041 | |
| | | kinase | | | | |
| 37351_at | UP | uridine phosphorylase | X90858 | 7 | Hs.77573 | |
| 35911_r_at | MMPL1 | matrix metalloproteinase-like 1 | AJ003147 | 16p13.3 | Hs.198265, | |
| | | | | | Hs.290222 | |
| 1780_at | FGR | Gardner-Rasheed feline sarcoma | M19722 | 1p36.2-p36.1 | Hs.1422 | |
| | | viral (v-fgr) oncogene homolog | | | | |
| 39598_at | GJB1 | gap junction protein, beta 1, 32 kD | X04325 | Xq13.1 | Hs.333303 | |
| | | (connexin 32, Charcot-Marie- | | | | |
| | | Tooth neuropathy, X-linked) | | | | |
| 31525_s_at | HBA2 | hemoglobin, alpha 2 | J00153 | 16p13.3 | Hs.272572, | |
| | | | | | Hs.347939 | |
| 40419_at | EPB72 | erythrocyte membrane protein band | X85116 | 9q34.1 | Hs.160483 | |
| | | 7.2 (stomatin) | | | | |
| 34627_at | KRTHA5 | keratin, hair, acidic, 5 | X90763 | 17q12-q21 | Hs.73082 | |
| 34095_f_at | UNK_U80114 | Human immunoglobulin heavy | U80114 | | | Accession No. U80114 |
| | | chain variable | | | | |
| | | region (V4-31) gene, | | | | |
| | | partial cds | | | | |
| 35530_f_at | IGL@ | immunoglobulin lambda locus | X92997 | 22q11.1-q11.2 | Hs.181125 | |
| 725_i_at | CSH1 | chorionic somatomammotropin | K02401 | | | |
| | | hormone 1 (placental lactogen) | | | | |
| 33963_at | AZU1 | azurocidin 1 (cationic antimicrobial | M96326 | 19p13.3 | Hs.72885 | |
| | | protein 37) | | | | |
| 330_s_at | TUBA1 | tubulin, alpha 1 (testis specific) | X06956 | | | |
| 40227_at | UNK_D29810 | Human mRNA for unknown | D29810 | 3q12.2-q12.3 | Hs.173374 | also known as ESDN |
| | | product, partial cds | | | | (endothelial and smooth |
| | | | | | | muscle cell-derived |
| | | | | | | neuropilin-like protein) |
| 1096_g_at | CD19 | CD19 antigen | M28170 | 16p11.2 | Hs.96023 | |
| 35955_at | CYCL | cytochrome c-like antigen | S80864 | | | |
| 41641_at | C4.4A | GPI-anchored metastasis- | AJ223603 | 19q13.32 | Hs.11950 | |
| | | associated protein homolog | | | | |
| 33021_at | UNK_AF035314 | Homo sapiens clone 23651 mRNA | AF035314 | | Hs.134526 | Unigene No. Hs.134526 |
| | | sequence | | | | Homo sapiens |
| | | | | | | clone 23651 |
| | | | | | | mRNA sequence |
| 39609_at | SIM2 | single-minded (Drosophila) | U80457 | 21q22.13 | Hs.27311 | |
| | | homolog 2 | | | | |
| 31586_f_at | UNK_X72475 | H. sapiens mRNA | X72475 | | Hs.367983 | Unigene No. Hs.512131 |
| | | for rearranged | | | | Homo sapiens clone H10 |
| | | Ig kappa light chain | | | | anti-HLA-A2/A28 |
| | | variable region (I.114) | | | | immunoglobulin light |
| | | | | | | chain variable region |
| | | | | | | mRNA, partial cds |
| 1937_at | RB1 | retinoblastoma 1 (including | M33647 | | | |
| | | osteosarcoma) | | | | |
| 35379_at | COL9A1 | collagen, type IX, alpha 1 | X54412 | 6q12-q14 | Hs.154850 | |
| 38513_at | KIAA0061 | KIAA0061 protein | D31765 | 8q22.1 | Hs.170114 | |
| 38968_at | SH3BP5 | SH3-domain binding protein 5 | AB005047 | 3p24.3 | Hs.109150 | |
| | | (BTK-associated) | | | | |
| 33979_at | RNASE3 | ribonuclease, RNase A family, 3 | X55990 | 14q24-q31 | Hs.73839 | |
| | | (eosinophil cationic protein) | | | | |
| 37623_at | NR4A2 | nuclear receptor | X75918 | 2q22-q23 | Hs.82120 | |
| | | subfamily 4, group | | | | |
| | | A, member 2 | | | | |
| 31578_at | UNK_M96936 | Cluster Incl M96936: Homo | M96936 | | | Accession No. M96936 |
| | | sapiens cystic fibrosis | | | | |
| | | transmembrane conductance | | | | |
| | | regulator (CFTR) gene, exons 23, | | | | |
| | | 24a, and 24. | | | | |
| 35566_f_at | UNK_AF015128 | Human rearranged | AF015128 | | | Unigene No. Hs.448957 |
| | | immunoglobulin heavy chain | | | | Homo sapiens partial |
| | | mRNA, partial cds | | | | mRNA for IgM |
| | | | | | | immunoglobulin heavy |
| | | | | | | chain variable region |
| | | | | | | (IGHV gene), clone |
| | | | | | | LIBPM376 |
| 37579_at | PIR121 | p53 inducible protein | L47738 | 5q34 | Hs.258503 | |
| 38508_s_at | TNXA | tenascin XA | U89337 | 6p21.3 | Hs.169886 | |
| 32254_at | UNK_AL050223 | Homo sapiens mRNA; cDNA | AL050223 | 17p13.1 | Hs.194534 | also known as VAMP2 |
| | | DKFZp586L1323 (from clone | | | | (vesicle-associated |
| | | DKFZp586L1323) | | | | membrane protein 2 |
| | | | | | | (synaptobrevin 2)), |
| | | | | | | Unigene No. Hs.25348 |
| 37701_at | RGS2 | regulator of G-protein signalling 2, | L13463 | 1q31 | Hs.78944 | |
| | | 24 kD | | | | |
| 35674_at | PDI2 | peptidyl arginine deiminase, type II | AB023211 | 1p35.2-p35.1 | Hs.33455 | |
| 36237_at | SLC22A6 | solute carrier family 22 (organic | AB009698 | 11q13.1-q13.2 | Hs.23965 | |
| | | anion transporter), member 6 | | | | |
| 1389_at | MME | membrane metallo-endopeptidase | J03779 | 3q25.1-q25.2 | Hs.1298 | |
| | | (neutral endopeptidase, | | | | |
| | | enkephalinase, CALLA, CD10) | | | | |
| 1797_at | CDKN2D | cyclin-dependent kinase inhibitor | U40343 | 19p13 | Hs.29656 | |
| | | 2D (p19, inhibits CDK4) | | | | |
| 34702_f_at | HUMRTVLH3 | endogenous retroviral protease | M27826 | 8q24 | Hs.373503 | |
| 34832_s_at | KIAA0763 | KIAA0763 gene product | AB018306 | 3p25.1 | Hs.4764 | |
| 39640_at | GFPT2 | glutamine-fructose-6-phosphate | AB016789 | 5q34-q35 | Hs.30332 | |
| | | transaminase 2 | | | | |
| 33499_s_at | IGHA1 | immunoglobulin heavy constant | AF067420 | | Hs.293441 | |
| | | alpha 1 | | | | |
| 33757_f_at | PSG11 | pregnancy specific beta-1- | M69245 | 19q13.2 | Hs.334408 | |
| | | glycoprotein 11 | | | | |
| 33143_s_at | SLC16A3 | solute carrier family 16 | U81800 | 22q12.3-q13.2 | Hs.85838 | |
| | | (monocarboxylic acid transporters), | | | | |
| | | member 3 | | | | |
| 39706_at | CPNE3 | copine III | AB014536 | 8q21.2 | Hs.14158 | |
| 37434_at | UNK_W28907 | Cluster Incl W28907: 53e12 | W28907 | 16p11.2 | Hs.111429 | also known as MGC3248 |
| | | Human retina cDNA randomly | | | | (dynactin 4), |
| | | primed sublibrary Homo sapiens | | | | Unigene No. Hs.435941 |
| | | cDNA, mRNA sequence. | | | | |
| 36979_at | SLC2A3 | solute carrier family 2 (facilitated | M20681 | 12p13.3 | Hs.7594 | |
| | | glucose transporter), member 3 | | | | |
| 37061_at | CHIT1 | chitinase 1 (chitotriosidase) | U29615 | 1q31-q32 | Hs.91093 | |
| 32162_r_at | UNK_AI817548 | Cluster Incl AI817548: | AI817548 | | | Unigene No. Hs.483452 |
| | | wk24e08.x1 NCI_CGAP_Lym12 | | | | Homo sapiens |
| | | Homo sapiens cDNA clone | | | | transcribed sequences |
| | | IMAGE: 2413286 3′ similar to | | | | |
| | | TR: Q83371 Q83371 REVERSE | | | | |
| | | TRANSCRIPTASE;, mRNA | | | | |
| | | sequence. | | | | |
| 2002_s_at | BCL2A1 | BCL2-related protein A1 | U27467 | 15q24.3 | Hs.227817 | |
| 1117_at | CDA | cytidine deaminase | L27943 | 1p36.2-p35 | Hs.72924 | |
| 32579_at | SMARCA4 | SWI/SNF related, matrix | U29175 | 19p13.2 | Hs.78202 | |
| | | associated, actin dependent | | | | |
| | | regulator of chromatin, subfamily | | | | |
| | | a, member 4 | | | | |
| 38868_at | FCAR | Fc fragment of IgA, receptor for | U43774 | 19q13.2-q13.4 | Hs.193122 | |
| 37078_at | CD3Z | CD3Z antigen, zeta polypeptide | J04132 | 1q22-q23 | Hs.97087 | |
| | | (TiT3 complex) | | | | |
| 37420_i_at | UNK_AL022723 | Human DNA sequence from clone | AL022723 | 6p21.3 | Hs.110309 | also known as HLA-F |
| | | 377H14 on chromosome | | | | (major histocompatibility |
| | | 6p21.32-22.1. | | | | complex, class I, F), |
| | | Contains the HLA-G | | | | Unigene No. Hs.411958 |
| | | gene for major | | | | |
| | | histocompatibility complex, | | | | |
| | | class I, G (HLA 6.0) two MHC | | | | |
| | | class I pseudogenes, an RPL7A | | | | |
| | | (60S Ribosomal Protein L7A) | | | | |
| | | pseudogene, a gene for a novel | | | | |
| | | MHC class 1 | | | | |
| | | protein, an interferon- | | | | |
| | | inducible protein 1-8U | | | | |
| | | pseudogene, an RPL23A (60S | | | | |
| | | Ribosomal Protein L23A) | | | | |
| | | pseudogene, an HCGIX | | | | |
| | | pseudogene, an MICB or . . . | | | | |
| 33501_r_at | IGHA1 | immunoglobulin heavy constant | S71043 | | | |
| | | alpha 1 | | | | |
| 34350_at | RSN | restin (Reed-Steinberg cell- | X64838 | 12q24.3 | Hs.31638 | |
| | | expressed intermediate filament- | | | | |
| | | associated protein) | | | | |
| 33500_i_at | IGHA1 | immunoglobulin heavy constant | S71043 | | | |
| | | alpha 1 | | | | |
| 32793_at | TRB@ | T cell receptor beta locus | X00437 | 7q34 | Hs.303157 | |
| 39245_at | UNK_U72507 | Human 40871 mRNA partial | U72507 | | Hs.234216 | also known as C3F |
| | | sequence | | | | (putative protein |
| | | | | | | similar to |
| | | | | | | nessy (Drosophila)), |
| | | | | | | Unigene No. Hs.300423 |
| 33244_at | CHN2 | chimerin (chimaerin) 2 | U07223 | 7p15.3 | Hs.286055 | |
| 36548_at | KIAA0895 | KIAA0895 protein | AB020702 | 7p15.3 | Hs.6224 | |
| 32794_g_at | TRB@ | T cell receptor beta locus | X00437 | 7q34 | Hs.303157 | |
| 40159_r_at | NCF1 | neutrophil cytosolic factor 1 | M55067 | 7q11.23 | Hs.1583 | |
| | | (47 kD, chronic granulomatous | | | | |
| | | disease, autosomal 1) | | | | |
| 34703_f_at | UNK_AA151971 | Cluster Incl AA151971: | AA151971 | 8q24 | Hs.373503 | Accession |
| | | zo30b03.r1 Stratagene colon | | | | No. AA151971 |
| | | (#937204) Homo sapiens cDNA | | | | |
| | | clone IMAGE: 588365 5′ similar to | | | | |
| | | contains LTR7.b3 LTR7 repetitive | | | | |
| | | element;, mRNA sequence. | | | | |
| 32620_at | FETUB | fetuin B | AB017551 | 3q27 | Hs.81073 | |
| 1353_g_at | IL8RA | interleukin 8 receptor, alpha | U11870 | 2q35 | Hs.194778 | |
| 35449_at | KLRB1 | killer cell lectin-like receptor | U11276 | 12p13 | Hs.169824 | |
| | | subfamily B, member 1 | | | | |
| 38194_s_at | IGKV1D-8 | immunoglobulin kappa variable | M63438 | 2p12 | Hs.156110 | |
| | | 1D-8 | | | | |
| 33914_r_at | FECH | ferrochelatase (protoporphyria) | D00726 | 18q21.3 | Hs.26 | |
| 34105_f_at | UNK_AI147237 | Homo sapiens isolate RP | AI147237 | 14q32.33 | Hs.300697 | Unigene No. Hs.64568 |
| | | immunoglobulin heavy chain FW2- | | | | Homo sapiens sequence |
| | | JH region gene, partial cds | | | | ra44b-8G9 |
| | | | | | | immunoglobulin heavy |
| | | | | | | chain variable region |
| | | | | | | mRNA, partial cds. |
| 916_at | PTPRN | protein tyrosine phosphatase, | L18983 | 2q35-q36.1 | Hs.89655 | |
| | | receptor type, N | | | | |
| 37137_at | GZMB | granzyme B (granzyme 2, | M17016 | 14q11.2 | Hs.1051 | |
| | | cytotoxic T-lymphocyte-associated | | | | |
| | | serine esterase 1) | | | | |
| 40729_s_at | UNK_Y14768 | Homo sapiens | Y14768 | 6p21.3 | Hs.890 | also known as LTB |
| | | DNA, cosmid | | | | (lymphotoxin beta (TNF |
| | | clones TN62 and TN82 | | | | superfamily, member 3)), |
| | | | | | | Unigene No. Hs.376208 |
| 39765_at | KIAA0320 | KIAA0320 protein | AB002318 | 15q15-q21 | Hs.150443 | |
| 37975_at | CYBB | cytochrome b-245, beta | X04011 | Xp21.1 | Hs.88974 | |
| | | polypeptide (chronic | | | | |
| | | granulomatous disease) | | | | |
| 41694_at | BN51T | BN51 (BHK21) temperature | M17754 | 8q21 | Hs.1276 | |
| | | sensitivity complementing | | | | |
| 40171_at | FRAT2 | GSK-3 binding protein FRAT2 | AF062739 | 10q23-q24.1 | Hs.140720 | |
| 33304_at | ISG20 | interferon stimulated gene (20 kD) | U88964 | 15q26 | Hs.183487 | |
| 33371_s_at | RAB31 | RAB31, member RAS oncogene | U59877 | 18p11.3 | Hs.223025 | |
| | | family | | | | |
| 35966_at | QPCT | glutaminyl-peptide | X71125 | 2p22.3 | Hs.79033 | |
| | | cyclotransferase (glutaminyl | | | | |
| | | cyclase) | | | | |
| 36591_at | TUBA1 | tubulin, alpha 1 (testis specific) | X06956 | 2q36.2 | Hs.75318 | |
| 34509_at | MGAM | maltase-glucoamylase (alpha- | AF016833 | 7q32.3 | Hs.122785 | |
| | | glucosidase) | | | | |
| 189_s_at | PLAUR | plasminogen activator, urokinase | U09937 | 19q13 | Hs.179657 | |
| | | receptor | | | | |
| 31499_s_at | FCGR3B | Fc fragment of IgG, low affinity | X16863 | 1q23 | Hs.372679 | |
| | | IIIb, receptor for (CD16) | | | | |
| 732_f_at | MUC3 | mucin 3, intestinal | M55406 | | | |
| 41164_at | IGHM | immunoglobulin heavy constant | X67301 | 14q32.33 | Hs.153261 | |
| | | mu | | | | |
| 36983_f_at | HP | haptoglobin | X00442 | 16q22.1 | Hs.75990 | |
| 37864_s_at | IGHG3 | immunoglobulin heavy constant | Y14737 | 14q32.33 | Hs.300697 | |
| | | gamma 3 (G3m marker) | | | | |
| 41165_g_at | IGHM | immunoglobulin heavy constant | X67301 | 14q32.33 | Hs.153261 | |
| | | mu | | | | |
| 41096_at | S100A8 | S100 calcium-binding protein A8 | AI126134 | 1q21 | Hs.100000 | |
| | | (calgranulin A) | | | | |
| 32606_at | BASP1 | brain acid-soluble protein 1 | AA135683 | 5p15.1-p14 | Hs.79516 | |
| 31315_at | UNK_D84143 | Human immunoglobulin (mAb59) | D84143 | 22q11.1-q11.2 | Hs.181125 | also known as IGLJ3 |
| | | light chain V region mRNA, partial | | | | (immunoglobulin lambda |
| | | sequence | | | | joining 3), Unigene No. |
| | | | | | | Hs.449592 |
| 31666_f_at | KIAA0168 | KIAA0168 gene product | W28731 | 20pter-p12.1 | Hs.80905 | |
| 41166_at | IGHM | immunoglobulin heavy constant | X58529 | 14q32.33 | Hs.153261 | |
| | | mu | | | | |
| 33849_at | PBEF | pre-B-cell colony-enhancing factor | U02020 | 7q11.23 | Hs.239138 | |
| 35013_at | UNK_AF013512 | Cluster Incl AF013512: Homo | AF013512 | 20q11.23-q12 | Hs.154078 | also known as LBP |
| | | sapiens lipopolysaccharide binding | | | | (lipopolysaccharide |
| | | protein (LBP) exon 15, complete | | | | binding protein), |
| | | sequence and complete cds. | | | | Unigene No. Hs.154078 |
| 39128_r_at | PPP2R4 | protein phosphatase 2A, regulatory | X73478 | 9q34 | Hs.236963 | |
| | | subunit B′ (PR 53) | | | | |
| 307_at | ALOX5 | arachidonate 5-lipoxygenase | J03600 | 10q11.2 | Hs.89499 | |
| 36071_at | UNK_AF070633 | Homo sapiens clone 24672 mRNA | AF070633 | | Hs.5010 | also known as IPO9 |
| | | sequence | | | | (importin 9), Unigene |
| | | | | | | No. Hs.445587 |
| 37099_at | ALOX5AP | arachidonate 5-lipoxygenase- | AI806222 | 13q12 | Hs.100194 | |
| | | activating protein | | | | |
| 31574_i_at | UNK_M14087 | Human HL14 gene encoding beta- | M14087 | | | Accession No. M14087 |
| | | galactoside-binding lectin, 3′ end, | | | | |
| | | clone 2 | | | | |
| 38017_at | CD79A | CD79A antigen (immunoglobulin- | U05259 | 19q13.2 | Hs.79630 | |
| | | associated alpha) | | | | |
| 36674_at | SCYA4 | small inducible cytokine A4 | J04130 | 17q12 | Hs.75703 | |
| | | (homologous to mouse Mip-1b) | | | | |
| 34498_at | VNN2 | Vanin 2 | D89974 | 6q23-q24 | Hs.121102 | |
| 36338_at | UNK_W28504 | Cluster Incl W28504: 48e7 Human | W28504 | | Hs.348515 | also known |
| | | retina cDNA randomly primed | | | | as KIAA0601 |
| | | sublibrary Homo sapiens cDNA, | | | | (KIAA0601 protein) |
| | | mRNA sequence. | | | | |
| 37054_at | BPI | bactericidal/permeability- | J04739 | 20q11.23-q12 | Hs.89535 | |
| | | increasing protein | | | | |
| 37105_at | CTSG | cathepsin G | M16117 | 14q11.2 | Hs.100764 | |
| 32607_at | BASP1 | brain acid-soluble protein 1 | AF039656 | 5p15.1-p14 | Hs.79516 | |
| 39872_at | UNK_AL031588 | Human DNA sequence from clone | AL031588 | 22q13.2-q13.3 | Hs.122552 | also known |
| | | 1163J1 on | | | | as GTSE1 (G-2 and |
| | | chromosome 22q13.2-13.33. | | | | S-phase expressed 1) |
| | | Contains the | | | | |
| | | 3′ part of a | | | | |
| | | gene for a novel KIAA0279 LIKE | | | | |
| | | EGF-like domain containing | | | | |
| | | protein (similar to mouse Celsr1, | | | | |
| | | rat MEGF2), a novel gene for a | | | | |
| | | protein similar to C. elegans | | | | |
| | | B0035.16 and bacterial tRNA (5- | | | | |
| | | Methylaminomethyl-2- | | | | |
| | | thiouridylate)-Methyltransferases, | | | | |
| | | and the 3′ part of | | | | |
| | | a novel gene for a | | | | |
| | | protein similar to mouse B99 . . . | | | | |
| 41827_f_at | UNK_AI932613 | Human rearranged | AI932613 | | Hs.350074 | Unigene No. Hs.272302 |
| | | immunoglobulin lambda light | | | | Homo sapiens, clone |
| | | chain mRNA | | | | IMAGE: |
| | | | | | | 5728597, mRNA |
| 35094_f_at | LILRA3 | leukocyte immunoglobulin-like | AF025527 | 19q13.4 | Hs.113277 | |
| | | receptor, subfamily A (without TM | | | | |
| | | domain), member 3 | | | | |
| 2090_i_at | UNK_H12458 | yj12d03.s1 Soares placenta Nb2HP | H12458 | | | Accession No. H12458 |
| | | Homo sapiens cDNA clone | | | | |
| | | IMAGE: 148517 3′ similar to | | | | |
| | | SP: WNT6_MOUSE P22727 | | | | |
| | | WNT-6 PROTEIN;, mRNA | | | | |
| | | sequence. | | | | |
| 37066_at | PRTN3 | proteinase 3 (serine proteinase, | X55668 | 19p13.3 | Hs.928 | |
| | | neutrophil, Wegener | | | | |
| | | granulomatosis autoantigen) | | | | |
| 37121_at | NKG7 | natural killer cell group 7 sequence | S69115 | 19q13.41 | Hs.10306 | |
| 37200_at | FCGR3A | Fc fragment of IgG, low affinity | J04162 | 1q23 | Hs.176663 | |
| | | IIIa, receptor for (CD16) | | | | |
| 35536_at | KIAA0604 | KIAA0604 gene product | AB011176 | | Hs.129801 | |
| 1350_at | CYP4F2 | cytochrome P450, subfamily IVF, | U02388 | 19pter-p13.11 | Hs.101 | |
| | | polypeptide 2 | | | | |
| 37467_at | IGHD | immunoglobulin heavy constant | K02882 | | | |
| | | delta | | | | |
| 41471_at | S100A9 | S100 calcium-binding protein A9 | W72424 | 1q21 | Hs.112405 | |
| | | (calgranulin B) | | | | |
| 32529_at | P63 | transmembrane protein (63 kD), | X69910 | 12q23.3 | Hs.74368 | |
| | | endoplasmic reticulum/Golgi | | | | |
| | | intermediate compartment | | | | |
| 35315_at | ORM1 | orosomucoid 1 | X02544 | 9q31-q32, | Hs.572 | |
| | | | | 9q32 | | |
| 32451_at | MS4A3 | membrane-spanning 4-domains, | L35848 | 11q12-q13.1 | Hs.99960 | |
| | | subfamily A, member 3 | | | | |
| | | (hematopoietic cell-specific) | | | | |
| 32275_at | SLPI | secretory leukocyte protease | X04470 | 20q12 | Hs.251754 | |
| | | inhibitor (antileukoproteinase) | | | | |
| 33273_f_at | IGL@ | immunoglobulin lambda locus | X57809 | 22q11.1-q11.2, | Hs.8997 | |
| | | | | 6p21.3 | | |
| 679_at | CTSG | cathepsin G | J04990 | 14q11.2 | Hs.100764 | |
| 36197_at | CHI3L1 | chitinase 3-like 1 (cartilage | Y08374 | 1q31.1 | Hs.75184 | |
| | | glycoprotein-39) | | | | |
| 36372_at | HK3 | hexokinase 3 (white cell) | U51333 | 5q35.2 | Hs.159237 | |
| 33274_f_at | IGL@ | immunoglobulin lambda locus | M18645 | 22q11.1-q11.2 | Hs.181125 | |
| 37145_at | GNLY | granulysin | M85276 | 2p12-q11 | Hs.105806 | |
| 37096_at | ELA2 | elastase 2, neutrophil | M34379 | 19p13.3 | Hs.99863 | |
| 31506_s_at | DEFA3 | defensin, alpha 3, neutrophil- | L12691 | 8p23.2-p23.1, | Hs.274463, | |
| | | specific | | 8pter-p23.3 | Hs.294176 | |
| 36447_at | FCN1 | ficolin (collagen/fibrinogen | S80990 | 9q34 | Hs.252136 | |
| | | domain-containing) 1 | | | | |
| 36479_at | GAS11 | growth arrest specific 11 | AF050078 | 16q24.3 | Hs.54877 | |
| 988_at | CEACAM1 | carcinoembryonic antigen-related | X16354 | 19q13.2 | Hs.50964 | |
| | | cell adhesion molecule 1 (biliary | | | | |
| | | glycoprotein) | | | | |
| 33093_at | IL18RAP | interleukin 18 receptor accessory | AF077346 | 2p24.3-p24.1 | Hs.158315 | |
| | | protein | | | | |
| 38533_s_at | ITGAM | integrin, alpha M (complement | J03925 | 16p11.2 | Hs.172631 | |
| | | component receptor 3, alpha; also | | | | |
| | | known as CD11b (p170), | | | | |
| | | macrophage antigen alpha | | | | |
| | | polypeptide) | | | | |
| 34319_at | S100P | S100 calcium-binding protein P | AA131149 | 4p16 | Hs.2962 | |
| 2041_i_at | ABL1 | v-abl Abelson murine leukemia | M14752 | 9q34.1 | Hs.146355 | |
| | | viral oncogene homolog 1 | | | | |
| 681_at | MMP8 | matrix metalloproteinase 8 | J05556 | 11q22.3 | Hs.73862 | |
| | | (neutrophil collagenase) | | | | |
| 37897_s_at | TFF3 | trefoil factor 3 (intestinal) | AI985964 | 21q22.3 | Hs.82961 | |
| 37233_at | OLR1 | oxidised low density lipoprotein | AF079167 | 12p13.2-p12.3 | Hs.77729 | |
| | | (lectin-like) receptor 1 | | | | |
| 35919_at | TCN1 | transcobalamin I (vitamin B12 | J05068 | 11q11-q12 | Hs.2012 | |
| | | binding protein, R binder family) | | | | |
| 266_s_at | CD24 | CD24 antigen (small cell lung | L33930 | 6q21 | Hs.286124 | |
| | | carcinoma cluster 4 antigen) | | | | |
| 1962_at | ARG1 | arginase, liver | M14502 | 6q23 | Hs.332405 | |
| 31495_at | SCYC2 | small inducible cytokine subfamily | D63789 | 1q23, 1q23-q25 | Hs.174228, | |
| | | C, member 2 | | | Hs.3195 | |
| 34546_at | DEFA4 | defensin, alpha 4, corticostatin | AI250799 | 8p23 | Hs.2582 | |
| 31792_at | ANXA3 | annexin A3 | M20560 | 4q13-q22 | Hs.1378 | |
| 36984_f_at | HPR | haptoglobin-related protein | X89214 | 16q22.1 | Hs.328822 | |
| 36105_at | CEACAM6 | carcinoembryonic antigen-related | M18728 | 19q13.2 | Hs.73848 | |
| | | cell adhesion molecule 6 (non- | | | | |
| | | specific cross reacting antigen) | | | | |
| 31477_at | TFF3 | trefoil factor 3 (intestinal) | L08044 | 21q22.3 | Hs.352107 | |
| 31793_at | DEFA1 | defensin, alpha 1, myeloid-related | AL036554 | 8p23.2-p23.1, | Hs.274463 | |
| | | sequence | | 8pter-p23.3 | | |
| 38326_at | G0S2 | putative lymphocyte G0/G1 switch | M69199 | 1q32.2-q41 | Hs.95910 | |
| | | gene | | | | |
| 33530_at | CEACAM8 | carcinoembryonic antigen-related | M33326 | 19q13.2 | Hs.41 | |
| | | cell adhesion molecule 8 | | | | |
| 39318_at | TCL1A | T-cell leukemia/lymphoma 1A | X82240 | 14q32.1 | Hs.2484 | |
| 31381_at | PGLYRP | peptidoglycan recognition protein | AF076483 | 19q13.2-q13.3 | Hs.137583 | |
| 38615_at | GW112 | differentially expressed in | AF097021 | 13q14.2 | Hs.273321 | |
| | | hematopoietic lineages | | | | |
| 37149_s_at | UNK_U95626 | Cluster Incl U95626: Homo | U95626 | 3q21-q23 | Hs.105938 | also known as LTF |
| | | sapiens ccr2b (ccr2), ccr2a (ccr2), | | | | (lactotransferrin), |
| | | ccr5 (ccr5) and ccr6 (ccr6) genes, | | | | Unigene No. Hs.437457 |
| | | complete cds, and lactoferrin | | | | |
| | | (lactoferrin) gene, partial cds, | | | | |
| | | complete sequence. | | | | |
| 31859_at | MMP9 | matrix metalloproteinase 9 | J05070 | 20q11.2-q13.1 | Hs.151738 | |
| | | (gelatinase B, 92 kD gelatinase, | | | | |
| | | 92 kD type IV collagenase) | | | | |
| 36464_at | SGP28 | specific granule protein (28 kDa); | X94323 | 6p12.2 | Hs.54431 | |
| | | cysteine-rich secretory protein-3 | | | | |
| 38879_at | S100A12 | S100 calcium-binding protein A12 | D83664 | 1q21 | Hs.19413 | |
| | | (calgranulin C) | | | | |
| 36710_at | CAMP | cathelicidin antimicrobial peptide | Z38026 | 3p21.3 | Hs.51120 | |
| 32821_at | LCN2 | lipocalin 2 (oncogene 24p3) | AI762213 | 9q34 | Hs.204238 | |

TABLE 9a
Expression Profiles of MDS Disease Genes

| | No. of Present Calls | No. of Present Calls | COV | COV | | P value (unequal) |
|---|---|---|---|---|---|---|
| Qualifier | (Disease-Free n = 18) | (MDS n = 13) | (Disease-Free) | (MDS) | MDS/Disease-Free | (MDS vs Disease-Free) |
| 35920_at | 0 | 9 | 29.10% | 104.95% | 7.06 | 0.012178646 |
| 38585_at | 18 | 11 | 117.58% | 89.71% | 6.81 | 0.005067647 |
| 40490_at | 14 | 13 | 37.70% | 40.74% | 6.48 | 6.71631E−06 |
| 36617_at | 16 | 12 | 41.20% | 91.85% | 5.21 | 0.008067841 |
| 39610_at | 0 | 8 | 0.00% | 76.06% | 5.08 | 0.002498657 |
| 38012_at | 0 | 1 | 48.57% | 89.31% | 4.77 | 0.007813853 |
| 41188_at | 18 | 12 | 24.25% | 129.16% | 4.77 | 0.047654425 |
| 1115_at | 15 | 10 | 89.31% | 110.52% | 4.69 | 0.025236403 |
| 37809_at | 0 | 11 | 0.00% | 83.17% | 4.46 | 0.005638014 |
| 1520_s_at | 2 | 7 | 55.00% | 119.29% | 4.30 | 0.039120223 |
| 32609_at | 18 | 12 | 30.89% | 124.64% | 4.19 | 0.048020493 |
| 41071_at | 16 | 11 | 35.68% | 93.03% | 4.17 | 0.012284741 |
| 36618_g_at | 5 | 11 | 34.30% | 108.21% | 3.95 | 0.02866346 |
| 38487_at | 0 | 9 | 44.88% | 91.04% | 3.88 | 0.012527495 |
| 34397_at | 17 | 13 | 37.78% | 47.43% | 3.47 | 0.000144085 |
| 37508_f_at | 5 | 12 | 30.86% | 54.29% | 3.46 | 0.000483305 |
| AFFX- | 7 | 11 | 56.52% | 61.99% | 3.44 | 0.001392687 |
| HUMRGE/M10098_5_at | | | | | | |
| 41562_at | 17 | 13 | 17.70% | 58.91% | 3.41 | 0.00098372 |
| 1519_at | 15 | 13 | 24.67% | 80.44% | 3.39 | 0.008238929 |
| 39209_r_at | 13 | 10 | 82.39% | 91.15% | 3.38 | 0.017375516 |
| 36749_at | 12 | 10 | 23.75% | 111.65% | 3.37 | 0.042420015 |
| 34892_at | 11 | 12 | 32.87% | 71.17% | 3.36 | 0.003946798 |
| 39736_at | 3 | 7 | 72.76% | 97.10% | 3.35 | 0.023999629 |
| 32663_at | 15 | 7 | 66.15% | 108.53% | 3.34 | 0.039365381 |
| 37018_at | 4 | 11 | 29.10% | 102.39% | 3.28 | 0.030948384 |
| 39208_i_at | 17 | 12 | 83.21% | 109.89% | 3.25 | 0.044173391 |
| 33986_r_at | 18 | 13 | 34.85% | 59.31% | 3.14 | 0.001345491 |
| 31522_f_at | 6 | 11 | 36.38% | 77.73% | 3.00 | 0.009451339 |
| 35576_f_at | 11 | 12 | 38.57% | 66.28% | 2.98 | 0.003589233 |
| 40877_s_at | 18 | 12 | 33.18% | 63.66% | 2.95 | 0.002802887 |
| 33352_at | 16 | 12 | 30.97% | 52.90% | 2.89 | 0.000762596 |
| 31523_f_at | 16 | 12 | 29.70% | 54.33% | 2.85 | 0.001009003 |
| 39032_at | 12 | 12 | 17.12% | 87.83% | 2.81 | 0.021478975 |
| 39070_at | 17 | 12 | 58.27% | 89.56% | 2.79 | 0.025064037 |
| 1065_at | 11 | 9 | 25.44% | 90.81% | 2.77 | 0.026302125 |
| 36501_at | 11 | 12 | 35.57% | 54.34% | 2.77 | 0.001133254 |
| 38097_at | 17 | 13 | 32.67% | 58.94% | 2.74 | 0.002159961 |
| 33989_f_at | 18 | 13 | 42.07% | 41.41% | 2.74 | 0.000106479 |
| 39971_at | 15 | 11 | 39.64% | 62.43% | 2.72 | 0.003367512 |
| 32260_at | 1 | 4 | 72.11% | 101.67% | 2.72 | 0.047312823 |
| 33131_at | 15 | 11 | 44.32% | 82.36% | 2.69 | 0.018092408 |
| 35224_at | 12 | 11 | 35.85% | 98.29% | 2.66 | 0.041417677 |
| 286_at | 18 | 13 | 22.27% | 92.30% | 2.66 | 0.031521859 |
| 37194_at | 10 | 12 | 23.15% | 53.49% | 2.65 | 0.001227121 |
| 37179_at | 18 | 12 | 35.72% | 81.84% | 2.64 | 0.018365806 |
| 1257_s_at | 11 | 11 | 77.92% | 60.11% | 2.63 | 0.003372958 |
| 32819_at | 16 | 13 | 33.61% | 55.25% | 2.63 | 0.001616828 |
| 31528_f_at | 7 | 8 | 32.91% | 54.07% | 2.63 | 0.001387821 |
| 35672_at | 7 | 10 | 41.26% | 57.69% | 2.61 | 0.002309381 |
| 37185_at | 9 | 9 | 35.92% | 97.06% | 2.59 | 0.042455774 |
| 34378_at | 15 | 12 | 50.67% | 64.07% | 2.58 | 0.005047571 |
| 37532_at | 14 | 12 | 45.83% | 55.19% | 2.58 | 0.001776789 |
| 40878_f_at | 0 | 11 | 31.14% | 68.69% | 2.58 | 0.007568928 |
| 41470_at | 15 | 10 | 12.12% | 89.81% | 2.57 | 0.030475277 |
| 40775_at | 18 | 12 | 34.67% | 72.53% | 2.57 | 0.01053539 |
| 36347_f_at | 18 | 13 | 25.22% | 68.66% | 2.53 | 0.008040791 |
| 36780_at | 18 | 13 | 30.24% | 84.59% | 2.51 | 0.025278633 |
| 36713_at | 17 | 12 | 28.33% | 64.31% | 2.47 | 0.005980418 |
| 40698_at | 18 | 13 | 56.07% | 77.27% | 2.47 | 0.017994055 |
| 39698_at | 11 | 10 | 26.76% | 94.13% | 2.46 | 0.042796332 |
| AFFX- | 8 | 12 | 111.54% | 82.11% | 2.44 | 0.031140405 |
| HUMRGE/M10098_M_at | | | | | | |
| 36711_at | 18 | 13 | 51.66% | 82.24% | 2.34 | 0.029042807 |
| 41138_at | 18 | 13 | 22.60% | 53.99% | 2.32 | 0.002511871 |
| 31665_s_at | 10 | 12 | 32.87% | 75.36% | 2.32 | 0.018889285 |
| 40088_at | 17 | 13 | 23.01% | 73.80% | 2.28 | 0.017938692 |
| 39341_at | 14 | 8 | 39.58% | 92.02% | 2.27 | 0.049918619 |
| 857_at | 18 | 13 | 30.25% | 46.21% | 2.23 | 0.001003535 |
| 1842_at | 7 | 11 | 93.09% | 67.35% | 2.23 | 0.017339926 |
| 34320_at | 0 | 2 | 39.28% | 66.05% | 2.23 | 0.011404307 |
| 41357_at | 9 | 12 | 40.60% | 31.96% | 2.23 | 2.84508E−05 |
| 40076_at | 18 | 13 | 43.55% | 72.10% | 2.21 | 0.01911007 |
| 39420_at | 8 | 13 | 87.07% | 72.96% | 2.20 | 0.025390684 |
| 34850_at | 18 | 13 | 32.54% | 25.22% | 2.19 | 1.72013E−06 |
| 430_at | 18 | 13 | 36.75% | 72.34% | 2.19 | 0.019719786 |
| 37218_at | 16 | 13 | 27.62% | 48.96% | 2.18 | 0.001831072 |
| 33885_at | 8 | 12 | 24.06% | 72.65% | 2.16 | 0.02080869 |
| 36709_at | 14 | 13 | 28.50% | 70.92% | 2.16 | 0.018799858 |
| 31888_s_at | 8 | 9 | 38.36% | 85.62% | 2.16 | 0.044812427 |
| 32508_at | 18 | 13 | 25.92% | 42.25% | 2.15 | 0.000618951 |
| 35842_at | 13 | 13 | 27.61% | 62.07% | 2.15 | 0.009310168 |
| 34308_at | 17 | 13 | 28.76% | 51.91% | 2.13 | 0.003136259 |
| 37033_s_at | 18 | 13 | 21.60% | 47.64% | 2.13 | 0.001728844 |
| 40617_at | 14 | 13 | 39.96% | 47.11% | 2.12 | 0.001680557 |
| 32857_at | 12 | 13 | 56.52% | 74.24% | 2.11 | 0.028253025 |
| 1940_at | 18 | 13 | 24.84% | 47.69% | 2.08 | 0.002044048 |
| 38653_at | 8 | 4 | 59.23% | 33.33% | 2.08 | 0.000142237 |
| 39315_at | 6 | 10 | 34.13% | 72.09% | 2.08 | 0.024491819 |
| 262_at | 18 | 13 | 25.46% | 49.01% | 2.08 | 0.002487679 |
| 40365_at | 18 | 13 | 45.93% | 68.85% | 2.07 | 0.020597588 |
| 31895_at | 18 | 13 | 36.35% | 59.42% | 2.07 | 0.009155862 |
| 40827_at | 14 | 12 | 28.86% | 82.63% | 2.07 | 0.044838232 |
| 40979_at | 17 | 13 | 35.36% | 49.14% | 2.06 | 0.002772887 |
| 36785_at | 17 | 9 | 29.02% | 80.26% | 2.05 | 0.041247239 |
| 34610_at | 14 | 13 | 65.51% | 58.59% | 2.04 | 0.011020987 |
| 40815_g_at | 14 | 12 | 40.97% | 58.51% | 2.03 | 0.009509488 |
| 34857_at | 14 | 11 | 33.01% | 57.01% | 2.03 | 0.007970829 |
| 39969_at | 9 | 10 | 44.68% | 65.92% | 2.03 | 0.018532802 |
| 39389_at | 15 | 13 | 20.25% | 72.79% | 2.02 | 0.028207497 |
| 32241_at | 17 | 13 | 26.52% | 40.15% | 2.02 | 0.000657638 |
| 40916_at | 18 | 13 | 32.94% | 41.70% | 2.02 | 0.00092087 |
| 32808_at | 18 | 13 | 19.59% | 40.76% | 2.01 | 0.000776608 |
| 33219_at | 18 | 12 | 35.35% | 38.73% | 2.01 | 0.000508666 |
| 33758_f_at | 4 | 3 | 28.13% | 37.95% | 0.50 | 2.08686E−06 |
| 38363_at | 18 | 13 | 18.88% | 49.49% | 0.50 | 4.05913E−06 |
| 2077_at | 3 | 1 | 78.50% | 32.20% | 0.50 | 0.016451872 |
| 33501_r_at | 18 | 12 | 57.68% | 95.55% | 0.50 | 0.013028678 |
| 38201_at | 6 | 8 | 79.68% | 24.79% | 0.50 | 0.016864223 |
| AFFX- | 18 | 13 | 21.07% | 36.70% | 0.50 | 1.05322E−07 |
| HSAC07/X00351_5_at | | | | | | |
| 1353_g_at | 17 | 5 | 51.15% | 62.27% | 0.50 | 0.002046828 |
| 33143_s_at | 18 | 11 | 57.22% | 85.75% | 0.49 | 0.008476437 |
| 41694_at | 18 | 13 | 21.22% | 53.40% | 0.49 | 9.2816E−06 |
| 35012_at | 18 | 13 | 39.67% | 95.66% | 0.49 | 0.004044053 |
| 38391_at | 18 | 13 | 37.68% | 62.06% | 0.49 | 0.000235983 |
| 38112_g_at | 18 | 10 | 58.84% | 113.41% | 0.49 | 0.019464229 |
| 32673_at | 16 | 11 | 42.90% | 48.40% | 0.48 | 0.000187934 |
| 39706_at | 18 | 13 | 39.70% | 52.38% | 0.48 | 0.000113503 |
| 33500_i_at | 18 | 12 | 59.35% | 83.98% | 0.48 | 0.006709175 |
| 35536_at | 4 | 2 | 57.63% | 79.63% | 0.47 | 0.004736153 |
| 41198_at | 18 | 8 | 37.18% | 112.84% | 0.47 | 0.006254707 |
| 1832_at | 4 | 2 | 101.40% | 32.55% | 0.47 | 0.043166999 |
| 35807_at | 18 | 13 | 10.42% | 56.16% | 0.47 | 5.68149E−06 |
| 37623_at | 18 | 13 | 50.87% | 65.62% | 0.47 | 0.001079248 |
| 916_at | 3 | 0 | 60.52% | 42.96% | 0.46 | 0.002013152 |
| 35013_at | 9 | 5 | 28.13% | 36.26% | 0.46 | 2.9106E−07 |
| 39245_at | 15 | 10 | 49.62% | 40.19% | 0.45 | 0.000266256 |
| 36879_at | 14 | 5 | 87.83% | 98.14% | 0.45 | 0.030055229 |
| 37054_at | 18 | 12 | 31.80% | 86.91% | 0.45 | 0.000361415 |
| 31792_at | 18 | 13 | 37.96% | 76.88% | 0.45 | 0.000220236 |
| 1252_at | 18 | 11 | 20.97% | 40.27% | 0.44 | 1.15824E−08 |
| 37145_at | 18 | 9 | 59.72% | 103.49% | 0.44 | 0.005643396 |
| 36447_at | 18 | 13 | 32.11% | 79.54% | 0.43 | 9.39021E−05 |
| 31562_at | 3 | 0 | 92.40% | 28.69% | 0.43 | 0.018896631 |
| 37967_at | 18 | 12 | 33.68% | 76.49% | 0.43 | 6.68627E−05 |
| 31586_f_at | 13 | 9 | 83.19% | 51.16% | 0.43 | 0.011446694 |
| 37215_at | 18 | 13 | 39.48% | 69.02% | 0.43 | 7.36333E−05 |
| 39872_at | 18 | 13 | 34.90% | 55.96% | 0.43 | 7.26993E−06 |
| 988_at | 18 | 11 | 46.11% | 66.84% | 0.42 | 0.00018702 |
| 35094_f_at | 18 | 6 | 39.55% | 138.56% | 0.42 | 0.006111092 |
| 39591_s_at | 14 | 3 | 68.64% | 82.29% | 0.42 | 0.004790492 |
| 38194_s_at | 18 | 12 | 56.79% | 76.50% | 0.42 | 0.001211103 |
| 41627_at | 17 | 13 | 50.75% | 31.58% | 0.42 | 0.00015752 |
| 266_s_at | 18 | 12 | 33.08% | 61.15% | 0.42 | 5.58067E−06 |
| 34105_f_at | 15 | 6 | 57.70% | 88.99% | 0.42 | 0.001827057 |
| 1339_s_at | 8 | 7 | 73.21% | 30.05% | 0.41 | 0.003594742 |
| 39128_r_at | 4 | 5 | 42.36% | 82.58% | 0.41 | 0.000188125 |
| 33274_f_at | 18 | 13 | 56.21% | 112.60% | 0.41 | 0.003452736 |
| 1825_at | 17 | 12 | 46.55% | 55.77% | 0.41 | 8.25931E−05 |
| 37233_at | 18 | 10 | 36.83% | 75.84% | 0.40 | 3.05822E−05 |
| 36105_at | 18 | 11 | 27.69% | 98.61% | 0.40 | 0.000127581 |
| 36338_at | 2 | 9 | 37.93% | 63.80% | 0.40 | 1.15796E−05 |
| 33273_f_at | 18 | 13 | 56.97% | 114.28% | 0.40 | 0.002756147 |
| 33150_at | 16 | 13 | 109.08% | 39.43% | 0.39 | 0.031643532 |
| 35714_at | 16 | 4 | 46.26% | 57.99% | 0.39 | 4.83374E−05 |
| 41471_at | 18 | 13 | 26.49% | 58.46% | 0.39 | 1.54729E−07 |
| 1117_at | 18 | 11 | 24.85% | 34.37% | 0.38 | 1.52695E−09 |
| 33284_at | 18 | 13 | 21.17% | 79.81% | 0.38 | 4.08888E−06 |
| 38894_g_at | 18 | 13 | 35.39% | 47.36% | 0.38 | 7.99901E−07 |
| 31506_s_at | 18 | 13 | 23.98% | 72.29% | 0.37 | 7.02431E−07 |
| 34350_at | 11 | 6 | 96.89% | 38.11% | 0.37 | 0.014687626 |
| 38895_i_at | 17 | 7 | 50.21% | 40.18% | 0.37 | 6.12649E−05 |
| 39593_at | 17 | 5 | 58.61% | 133.17% | 0.37 | 0.003313989 |
| 33963_at | 18 | 12 | 29.99% | 111.89% | 0.37 | 0.000149708 |
| 35591_at | 2 | 0 | 83.16% | 32.55% | 0.37 | 0.005375037 |
| 37864_s_at | 18 | 12 | 70.35% | 92.32% | 0.37 | 0.002733896 |
| 36674_at | 18 | 5 | 61.09% | 57.01% | 0.36 | 0.000471728 |
| 37121_at | 18 | 12 | 29.82% | 104.83% | 0.36 | 4.54685E−05 |
| 32612_at | 18 | 13 | 31.55% | 59.48% | 0.36 | 2.28684E−07 |
| 39413_at | 3 | 0 | 99.98% | 0.00% | 0.36 | 0.014678409 |
| 41827_f_at | 18 | 11 | 48.32% | 94.43% | 0.36 | 0.000162925 |
| 41440_at | 9 | 5 | 81.53% | 47.54% | 0.35 | 0.00389068 |
| 33979_at | 18 | 13 | 24.29% | 135.31% | 0.35 | 0.000314127 |
| 38533_s_at | 17 | 9 | 38.42% | 68.35% | 0.35 | 2.86745E−06 |
| 32275_at | 18 | 11 | 24.64% | 54.99% | 0.35 | 4.21188E−09 |
| 35315_at | 18 | 11 | 40.89% | 82.29% | 0.35 | 1.35922E−05 |
| 33757_f_at | 9 | 5 | 37.23% | 33.47% | 0.34 | 5.7396E−07 |
| 40278_at | 17 | 12 | 92.89% | 47.40% | 0.34 | 0.008439255 |
| 36983_f_at | 11 | 1 | 67.51% | 36.57% | 0.33 | 0.000631776 |
| 37105_at | 18 | 13 | 24.16% | 73.06% | 0.33 | 3.39649E−08 |
| 34415_at | 7 | 2 | 86.76% | 14.42% | 0.33 | 0.004318332 |
| 41840_r_at | 7 | 0 | 88.05% | 21.08% | 0.33 | 0.004785786 |
| AFFX-M27830_3_at | 3 | 0 | 56.98% | 64.59% | 0.33 | 0.000125338 |
| 39330_s_at | 18 | 13 | 32.09% | 59.28% | 0.32 | 5.20933E−08 |
| 38513_at | 1 | 0 | 110.74% | 0.00% | 0.32 | 0.018688899 |
| 38514_at | 15 | 6 | 61.78% | 105.37% | 0.32 | 0.000540193 |
| 38249_at | 1 | 0 | 125.03% | 0.00% | 0.32 | 0.032927778 |
| 40159_r_at | 8 | 2 | 44.43% | 142.14% | 0.31 | 0.000233904 |
| 31793_at | 18 | 11 | 26.05% | 78.65% | 0.31 | 3.93917E−08 |
| 1407_g_at | 3 | 3 | 103.25% | 36.74% | 0.31 | 0.011293086 |
| 679_at | 18 | 13 | 28.78% | 99.05% | 0.29 | 4.51226E−07 |
| 31578_at | 2 | 0 | 97.66% | 25.75% | 0.29 | 0.007051512 |
| 35919_at | 18 | 11 | 46.18% | 106.62% | 0.29 | 1.81969E−05 |
| 37149_s_at | 18 | 11 | 21.71% | 107.80% | 0.29 | 6.22517E−07 |
| 38976_at | 18 | 11 | 25.29% | 76.39% | 0.28 | 3.2957E−09 |
| 40234_at | 13 | 7 | 66.74% | 42.91% | 0.28 | 0.00028357 |
| 36984_f_at | 18 | 12 | 43.61% | 62.82% | 0.27 | 1.29007E−06 |
| 35762_at | 6 | 11 | 113.70% | 33.73% | 0.27 | 0.014506959 |
| 40517_at | 17 | 11 | 104.46% | 28.39% | 0.26 | 0.00823259 |
| 33093_at | 16 | 2 | 69.35% | 48.21% | 0.26 | 0.00029945 |
| 31357_at | 2 | 0 | 103.57% | 0.00% | 0.26 | 0.007347235 |
| 40171_at | 13 | 3 | 37.94% | 47.28% | 0.26 | 1.05199E−07 |
| 32451_at | 18 | 12 | 25.90% | 120.19% | 0.25 | 2.39588E−07 |
| 33021_at | 6 | 1 | 97.55% | 35.50% | 0.25 | 0.004695929 |
| 38615_at | 18 | 9 | 34.99% | 83.46% | 0.25 | 4.07788E−08 |
| 31495_at | 18 | 2 | 32.65% | 28.87% | 0.25 | 1.18847E−08 |
| 33530_at | 18 | 11 | 24.85% | 75.22% | 0.25 | 1.54097E−10 |
| 37066_at | 18 | 9 | 43.80% | 137.20% | 0.24 | 7.29636E−06 |
| 1350_at | 15 | 4 | 100.74% | 42.91% | 0.24 | 0.005501102 |
| 37096_at | 18 | 13 | 24.93% | 112.49% | 0.24 | 3.23509E−08 |
| 37975_at | 18 | 9 | 70.09% | 147.08% | 0.24 | 0.00047768 |
| 34546_at | 18 | 12 | 23.10% | 83.64% | 0.24 | 1.25053E−10 |
| 36237_at | 9 | 5 | 108.27% | 20.82% | 0.24 | 0.008197345 |
| 38273_at | 17 | 10 | 115.58% | 35.63% | 0.23 | 0.011839271 |
| 37434_at | 1 | 4 | 110.01% | 0.00% | 0.23 | 0.008442055 |
| 31477_at | 18 | 3 | 39.08% | 54.04% | 0.23 | 9.20022E−08 |
| 34013_f_at | 4 | 1 | 109.08% | 25.75% | 0.22 | 0.007714844 |
| 32054_at | 9 | 4 | 114.79% | 0.00% | 0.22 | 0.01007966 |
| 36548_at | 1 | 0 | 110.11% | 0.00% | 0.22. | 0.007761926 |
| 33244_at | 3 | 5 | 120.82% | 35.50% | 0.21 | 0.012985421 |
| 39765_at | 16 | 11 | 102.09% | 51.93% | 0.21 | 0.004436498 |
| 33742_f_at | 6 | 1 | 122.43% | 0.00% | 0.20 | 0.01325763 |
| 34014_f_at | 4 | 0 | 118.62% | 0.00% | 0.20 | 0.01081195 |
| 732_f_at | 18 | 13 | 103.04% | 33.80% | 0.19 | 0.00411348 |
| 36464_at | 18 | 10 | 43.19% | 95.80% | 0.19 | 2.16889E−07 |
| 36372_at | 18 | 9 | 40.34% | 108.09% | 0.19 | 8.08044E−08 |
| 33914_r_at | 13 | 10 | 121.82% | 59.48% | 0.19 | 0.011614331 |
| 38908_s_at | 16 | 13 | 120.86% | 36.74% | 0.18 | 0.010807145 |
| 32620_at | 1 | 0 | 122.71% | 0.00% | 0.18 | 0.011137823 |
| 31381_at | 18 | 5 | 44.53% | 139.85% | 0.18 | 4.28083E−07 |
| 37897_s_at | 10 | 1 | 43.31% | 117.05% | 0.17 | 1.75752E−07 |
| 32579_at | 18 | 12 | 115.35% | 42.20% | 0.17 | 0.007245986 |
| 39894_f_at | 5 | 7 | 129.98% | 30.05% | 0.17 | 0.014547453 |
| 36071_at | 9 | 7 | 123.49% | 24.79% | 0.16 | 0.010474576 |
| 38879_at | 18 | 12 | 34.29% | 78.02% | 0.16 | 1.95313E−09 |
| 36710_at | 18 | 11 | 26.79% | 123.45% | 0.14 | 1.44995E−11 |
| 31859_at | 18 | 12 | 46.44% | 102.63% | 0.13 | 2.49904E−07 |
| 39318_at | 16 | 0 | 58.49% | 20.34% | 0.13 | 7.90142E−06 |
| 35418_at | 3 | 0 | 118.93% | 25.75% | 0.12 | 0.006185865 |
| 31666_f_at | 7 | 0 | 128.18% | 25.75% | 0.10 | 0.008367069 |
| 32821_at | 18 | 11 | 27.51% | 117.66% | 0.09 | 5.03219E−12 |
| 31574_i_at | 3 | 0 | 134.33% | 0.00% | 0.08 | 0.009640967 |
| 2041_i_at | 16 | 10 | 135.87% | 25.75% | 0.03 | 0.007719335 |

TABLE 9b
Examples of MDS Disease Genes

| | | | Entrez | | |
|---|---|---|---|---|---|
| Qualifier | Gene Name | Gene Title | Accession No. | Cyto Band | Unigene No. |
| 35920_at | UNK_N55205 | Human beta-type globin pseudogene | N55205 | | Hs.20205 |
| 38585_at | HBG2 | hemoglobin, gamma G | M91036 | 11p15.5 | Hs.266959, Hs.283108 |
| 40490_at | DDX21 | DEAD/H (Asp-Glu-Ala-Asp/His) box | U41387 | 10q21 | Hs.169531 |
| | | polypeptide 21 | | | |
| 36617_at | ID1 | inhibitor of DNA binding 1, dominant | X77956 | 20q11 | Hs.75424 |
| | | negative helix-loop-helix protein | | | |
| 39610_at | HOXB2 | homeo box B2 | X16665 | 17q21-q22 | Hs.2733 |
| 38012_at | FBN2 | fibrillin 2(congenital contractural | U03272 | 5q23-q31 | Hs.79432 |
| | | arachnodactyly) | | | |
| 41188_at | UNK_W28186 | ESTs, Weakly similar to GOLGI 4- | W28186 | 8q22.1 | Hs.296398 |
| | | TRANSMEMBRANE SPANNING | | | |
| | | TRANSPORTER MTP [H. sapiens] | | | |
| 1115_at | PF4 | platelet factor 4 | M25897 | 4q12-q21 | Hs.81564 |
| 37809_at | HOXA9 | homeo box A9 | U41813 | 7p15-p14 | Hs.127428 |
| 1520_s_at | EDN1 | endothelin 1 | J05008 | 2q14 | Hs.126256 |
| 32609_at | H2AFO | H2A histone family, member O | AI885852 | 1q21.3 | Hs.795 |
| 41071_at | SPINK2 | serine protease inhibitor, Kazal type, 2 | X57655 | 4q11 | Hs.98243 |
| | | (acrosin-trypsin inhibitor) | | | |
| 36618_g_at | ID1 | inhibitor of DNA binding 1, dominant | X77956 | 20q11 | Hs.75424 |
| | | negative helix-loop-helix protein | | | |
| 38487_at | KIAA0246 | KIAA0246 protein | D87433 | 3p21.31 | Hs.301989 |
| 34397_at | OA48-18 | acid-inducible phosphoprotein | AF069250 | 17, 17q21 | Hs.278670 |
| 37508_f_at | HYPA | Huntingtin-interacting protein A | AA675900 | 2q23.3 | Hs.107213 |
| AFFX- | 18SRNA5_Hs_AFFX | 18SRNA5 control sequence (H. sapiens) | M10098 | | |
| HUMRGE/ | | [AFFX] | | | |
| M10098_5_at | | | | | |
| 41562_at | BMI1 | murine leukemia viral (bmi-1) oncogene | L13689 | 10p13 | Hs.431 |
| | | homolog | | | |
| 1519_at | ETS2 | v-ets avian erythroblastosis virus E26 | J04102 | 21q22.2 | Hs.85146 |
| | | oncogene homolog 2 | | | |
| 39209_r_at | PPBP | pro-platelet basic protein (includes | M54995 | 4q12-q13 | Hs.2164 |
| | | platelet basic protein, beta- | | | |
| | | thromboglobulin, connective tissue- | | | |
| | | activating peptide III, neutrophil- | | | |
| | | activating peptide-2) | | | |
| 36749_at | CPA3 | carboxypeptidase A3 (mast cell) | M73720 | 3q21-q25 | Hs.646 |
| 34892_at | TNFRSF10B | tumor necrosis factor receptor | AF016266 | 8p22-p21 | Hs.51233 |
| | | superfamily, member 10b | | | |
| 39736_at | CDC42 | cell division cycle 42 (GTP-binding | M35543 | 1p36.1 | Hs.146409 |
| | | protein, 25 kD) | | | |
| 32663_at | RHAG | Rhesus blood group-associated | X64594 | 6p21.1-p11 | Hs.169536 |
| | | glycoprotein | | | |
| 37018_at | H1F2 | H1 histone family, member 2 | AI189287 | 6p21.3 | Hs.7644 |
| 39208_i_at | PPBP | pro-platelet basic protein (includes | M54995 | 4q12-q13 | Hs.2164 |
| | | platelet basic protein, beta- | | | |
| | | thromboglobulin, connective tissue- | | | |
| | | activating peptide III, neutrophil- | | | |
| | | activating peptide-2) | | | |
| 33986_r_at | HSPCB | heat shock 90 kD protein 1, beta | W28616 | 6p12 | Hs.74335 |
| 31522_f_at | H2BFG | H2B histone family, member G | Z80779 | 6p21.3 | Hs.182137 |
| 35576_f_at | H2BFC | H2B histone family, member C | AL009179 | 6p21.3, | Hs.137594, |
| | | | | 6p22-p21.3 | Hs.151506, |
| | | | | | Hs.154576, |
| | | | | | Hs.180779, |
| | | | | | Hs.182138, |
| | | | | | Hs.182140, |
| | | | | | Hs.352109, Hs.356901 |
| 40877_s_at | UNK_AF041080 | Homo sapiens D15F37 pseudogene, S3 | AF041080 | 15q11-q13 | Hs.286132 |
| | | allele, mRNA sequence | | | |
| 33352_at | H2BFQ | H2B histone family, member Q | X57985 | 1q21-q23 | Hs.2178 |
| 31523_f_at | H2BFH | H2B histone family, member H | Z80780 | 21q22.3, | Hs.137594, |
| | | | | 6p21.3, | Hs.151506, |
| | | | | 6p21.31, | Hs.154576, |
| | | | | 6p21.33, | Hs.180779, |
| | | | | 6p22-p21.3, | Hs.182137, |
| | | | | | Hs.182138, |
| | | | | | Hs.247817, |
| | | | | | Hs.285735, |
| | | | | | Hs.352109, |
| | | | | | Hs.356901, Hs.367748 |
| 39032_at | TSC22 | transforming growth factor beta- | AJ222700 | 13q14 | Hs.114360 |
| | | stimulated protein TSC-22 | | | |
| 39070_at | SNL | singed (Drosophila)-like (sea urchin | U03057 | 7p22 | Hs.118400 |
| | | fascin homolog like) | | | |
| 1065_at | FLT3 | fms-related tyrosine kinase 3 | U02687 | 13q12 | Hs.385 |
| 36501_at | PPM1A | protein phosphatase 1A (formerly 2C), | S87759 | 14q22.3-q23.1 | Hs.57764 |
| | | magnesium-dependent, alpha isoform | | | |
| 38097_at | UNK_AF010313 | Homo sapiens Pig8 (PIG8) mRNA, | AF010313 | 11q24 | Hs.343911 |
| | | complete cds | | | |
| 33989_f_at | TEGT | testis enhanced gene transcript | W28869 | 12q12-q13 | Hs.74637 |
| 39971_at | LYL1 | lymphoblastic leukemia derived sequence 1 | M22637 | 19p13.2 | Hs.46446 |
| 32260_at | PEA15 | phosphoprotein enriched in astrocytes 15 | X86809 | 1q21.1 | Hs.194673 |
| 33131_at | SOX4 | SRY (sex determining region Y)-box 4 | X70683 | 17p11.2, | Hs.83484 |
| | | | | 6p22.3 | |
| 35224_at | UNK_AF070569 | Homo sapiens clone 24659 mRNA | AF070569 | 17p13.3 | Hs.29206 |
| | | sequence | | | |
| 286_at | H2AFO | H2A histone family, member O | L19779 | 1q21.3 | Hs.795 |
| 37194_at | GATA2 | GATA-binding protein 2 | M68891 | 3q21, | Hs.367725 |
| | | | | 3q22.1 | |
| 37179_at | NFE2 | nuclear factor (erythroid-derived 2), 45 kD | S77763 | 12q13 | Hs.75643 |
| 1257_s_at | QSCN6 | quiescin Q6 | L42379 | 1q24 | Hs.77266 |
| 32819_at | UNK_AJ223352 | Homo sapiens mRNA for for histone | AJ223352 | 6p21.33 | Hs.247817 |
| | | H2B, clone pjG4-5-14 | | | |
| 31528_f_at | H2BFE | H2B histone family, member E | Z83738 | 6p22-p21.3 | Hs.182432 |
| 35672_at | DKFZP434N093 | DKFZP434N093 protein | AL080144 | 1q44 | Hs.33363 |
| 37185_at | PAI2 | plasminogen activator inhibitor, type II | Y00630 | 18q21.3 | Hs.75716 |
| | | (arginine-serpin) | | | |
| 34378_at | ADFP | adipose differentiation-related protein; | X97324 | 9p21.2 | Hs.3416 |
| | | adipophilin | | | |
| 37532_at | ACADM | acyl-Coenzyme A dehydrogenase, C-4 to | M91432 | 1p31 | Hs.79158 |
| | | C-12 straight chain | | | |
| 40878_f_at | UNK_AF041081 | Homo sapiens D15F37 pseudogene, S4 | AF041081 | 15q11-q13 | Hs.286132 |
| | | allele, mRNA sequence | | | |
| 41470_at | PROML1 | prominin (mouse)-like 1 | AF027208 | 4p15.33 | Hs.112360 |
| 40775_at | ITM2A | integral membrane protein 2A | AL021786 | | |
| 36347_f_at | H2BFD | H2B histone family, member D | AA873858 | 6p21.3, | Hs.154576 |
| | | | | 6p22-p21.3 | |
| 36780_at | CLU | clusterin (complement lysis inhibitor, SP- | M25915 | 8p21-p12 | Hs.75106 |
| | | 40,40, sulfated glycoprotein 2, | | | |
| | | testosterone-repressed prostate message 2, | | | |
| | | apolipoprotein J) | | | |
| 36713_at | DKFZP434C091 | DKFZP434C091 protein | AL080170 | 1q44 | Hs.51692 |
| 40698_at | CLECSF2 | C-type (calcium dependent, carbohydrate- | X96719 | 12p13-p12 | Hs.85201 |
| | | recognition domain) lectin, superfamily | | | |
| | | member 2 (activation-induced) | | | |
| 39698_at | UNK_U51712 | Cluster Incl U51712: HSU51712 Human | U51712 | 4q11-q12 | Hs.13775 |
| | | normal gingiva Homo sapiens cDNA, | | | |
| | | mRNA sequence. | | | |
| AFFX- | 18SRNAM_Hs_AFFX | 18SRNAM control sequence (H. sapiens) | M10098 | | |
| HUMRGE/ | | [AFFX] | | | |
| M10098_M_at | | | | | |
| 36711_at | MAFF | v-maf musculoaponeurotic fibrosarcoma | AL021977 | 22q13.1 | Hs.51305 |
| | | (avian)oncogene family, protein F | | | |
| 41138_at | MIC2 | antigen identified by monoclonal | M16279 | Xp22.32, | Hs.177543 |
| | | antibodies 12E7, F21 and O13 | | Yp11.3 | |
| 31665_s_at | UNK_W27675 | EST, Weakly similar to cDNA EST | W27675 | 3q25.1 | Hs.332404 |
| | | EMBL: D71941 comes from this gene | | | |
| | | [C. elegans] | | | |
| 40088_at | NRIP1 | nuclear receptor interacting protein 1 | X84373 | 21q11.2 | Hs.155017 |
| 39341_at | TRIP6 | thyroid hormone receptor interactor 6 | AJ001902 | 17p13.3, | Hs.119498 |
| | | | | 7q22 | |
| 857_at | PPM1A | protein phosphatase 1A (formerly 2C), | S87759 | 14q22.3-q23.1 | Hs.57764 |
| | | magnesium-dependent, alpha isoform | | | |
| 1842_at | UNK_S62138 | Oncogene Tls/Chop, Fusion Activated | S62138 | | |
| 34320_at | UNK_AL050224 | Homo sapiens mRNA; cDNA | AL050224 | 17q21.2 | Hs.29759 |
| | | DKFZp586L2123 (from clone | | | |
| | | DKFZp586L2123) | | | |
| 41357_at | ATP5B | ATP synthase, H+ transporting, | W27997 | 12p13-qter | Hs.25 |
| | | mitochondrial F1 complex, beta | | | |
| | | polypeptide | | | |
| 40076_at | TPD52L2 | tumor protein D52-like 2 | AF004430 | 20q13.2-q13.3 | Hs.154718 |
| 39420_at | UNK_S62138 | Cluster Incl S62138: TLS/CHOP = hybrid | S62138 | 12q13.1-q13.2 | Hs.337761 |
| | | gene {translocation breakpoint} [human, | | | |
| | | myxoid liposarcomas cells, mRNA | | | |
| | | Mutant, 1682 nt]. | | | |
| 34850_at | UBE2E3 | ubiquitin-conjugating enzyme E2E 3 | AB017644 | 2q32.1 | Hs.4890 |
| | | (homologous to yeast UBC4/5) | | | |
| 430_at | NP | nucleoside phosphorylase | X00737 | 14q13.1 | Hs.75514 |
| 37218_at | BTG3 | BTG family, member 3 | D64110 | 21q21.1 | Hs.77311 |
| 33885_at | KIAA0907 | KIAA0907 protein | AB020714 | 1q21.2 | Hs.24656 |
| 36709_at | ITGAX | integrin, alpha X (antigen CD11C (p150), | Y00093 | 16p11.2 | Hs.51077 |
| | | alpha polypeptide) | | | |
| 31888_s_at | TSSC3 | tumor suppressing subtransferable | AF001294 | 11p15.5 | Hs.154036 |
| | | candidate 3 | | | |
| 32508_at | KIAA1096 | KIAA1096 protein | AL096857 | 1q23.3 | Hs.69559 |
| 35842_at | UNK_AL049265 | Homo sapiens mRNA; cDNA | AL049265 | | Hs.71968 |
| | | DKFZp564F053 (from clone | | | |
| | | DKFZp564F053) | | | |
| 34308_at | H2AFL | H2A histone family, member L | U90551 | 6p21.3 | Hs.28777 |
| 37033_s_at | GPX1 | glutathione peroxidase 1 | X13710 | 3p21.3 | Hs.76686 |
| 40617_at | UNK_AC004381 | Homo sapiens Chromosome 16 BAC | AC004381 | 16p12.2 | Hs.268371 |
| | | clone CIT987SK-44M2 | | | |
| 32857_at | SOS1 | son of sevenless (Drosophila) homolog 1 | L13858 | 14q21 | Hs.348496 |
| 1940_at | KRAS2 | v-Ki-ras2 Kirsten rat sarcoma 2 viral | M54968 | 12p12.1 | Hs.351221 |
| | | oncogene homolog | | | |
| 38653_at | PMP22 | peripheral myelin protein 22 | D11428 | 17p12-p11.2 | Hs.103724 |
| 39315_at | ANGPT1 | angiopoietin 1 | D13628 | 8q22.3-q23 | Hs.2463 |
| 262_at | AMD1 | S-adenosylmethionine decarboxylase 1 | M21154 | 6q21-q22 | Hs.262476 |
| 40365_at | GNA15 | guanine nucleotide binding protein (G | M63904 | 19p13.3 | Hs.73797 |
| | | protein), alpha 15 (Gq class) | | | |
| 31895_at | BACH1 | BTB and CNC homology 1, basic leucine | AB002803 | 21q22.11 | Hs.154276 |
| | | zipper transcription factor 1 | | | |
| 40827_at | IARS | isoleucine-tRNA synthetase | U04953 | 9q21 | Hs.172801 |
| 40979_at | C14ORF3 | chromosome 14 open reading frame 3 | AJ243310 | 14q23.3-31 | Hs.204041 |
| 36785_at | HSPB1 | heat shock 27 kD protein 1 | Z23090 | 7p12.3 | Hs.76067 |
| 34610_at | GNB2L1 | guanine nucleotide binding protein (G | W25845 | 5q35.3 | Hs.5662 |
| | | protein), beta polypeptide 2-like 1 | | | |
| 40815_g_at | IDS | iduronate 2-sulfatase (Hunter syndrome) | L40586 | Xq28 | Hs.172458 |
| 34857_at | UNK_Z24724 | H. sapiens polyA site DNA | Z24724 | 3q29 | Hs.324507 |
| 39969_at | H4FG | H4 histone family, member G | AA255502 | 6p21.3 | Hs.46423 |
| 39389_at | CD9 | CD9 antigen (p24) | M38690 | 12p13.3 | Hs.1244 |
| 32241_at | TARDBP | TAR DNA binding protein | AL050265 | 1p36.22 | Hs.193989 |
| 40916_at | UNK_AL035494 | Human DNA sequence from clone | AL035494 | | |
| | | 635G19 on chromosome Xq22.1-22.3 | | | |
| | | Contains a LAMR1 (Laminin Receptor 1 | | | |
| | | (67 kD) (RPSA, 40S Ribosomal Protein | | | |
| | | SA, P40)) pseudogene and part of a novel | | | |
| | | protein. Contains ESTs and GSSs | | | |
| 32808_at | ITGB1 | integrin, beta 1 (fibronectin receptor, beta | X07979 | 10p11.2 | Hs.287797 |
| | | polypeptide, antigen CD29 includes | | | |
| | | MDF2, MSK12) | | | |
| 33219_at | KIAA1097 | KIAA1097 protein | AB029020 | 1p31.1 | Hs.173694 |
| 33758_f_at | PSG11 | pregnancy specific beta-1-glycoprotein 11 | U25988 | 19q13.2 | Hs.334408 |
| 38363_at | TYROBP | TYRO protein tyrosine kinase binding | W60864 | 19q13.1 | Hs.9963 |
| | | protein | | | |
| 2077_at | UNK_L40385 | Homo sapiens integrin alpha 6 (ITGA6) | L40385 | 2q31.1 | Hs.227730 |
| | | subunit gene, exons. | | | |
| 33501_r_at | IGHA1 | immunoglobulin heavy constant alpha 1 | S71043 | | |
| 38201_at | BCAT1 | branched chain aminotransferase 1, | U21551 | 12pter-q12 | Hs.157205 |
| | | cytosolic | | | |
| AFFX- | BACTIN5_Hs_AFFX | BACTIN5 control sequence (H. sapiens) | X00351 | 7p15-p12 | Hs.288061 |
| HSAC07/ | | [AFFX] | | | |
| X00351_5_at | | | | | |
| 1353_g_at | IL8RA | interleukin 8 receptor, alpha | U11870 | 2q35 | Hs.194778 |
| 33143_s_at | SLC16A3 | solute carrier family 16 (monocarboxylic | U81800 | 22q12.3-q13.2 | Hs.85838 |
| | | acid transporters), member 3 | | | |
| 41694_at | BN51T | BN51 (BHK21) temperature sensitivity | M17754 | 8q21 | Hs.1276 |
| | | complementing | | | |
| 35012_at | MNDA | myeloid cell nuclear differentiation | M81750 | 1q22 | Hs.153837 |
| | | antigen | | | |
| 38391_at | CAPG | capping protein (actin filament), gelsolin- | M94345 | 2cen-q24 | Hs.82422 |
| | | like | | | |
| 38112_g_at | CSPG2 | chondroitin sulfate proteoglycan 2 | X15998 | 5q14.3 | Hs.81800 |
| | | (versican) | | | |
| 32673_at | BTN2A1 | butyrophilin, subfamily 2, member A1 | U90543 | 6p22.1 | Hs.169963 |
| 39706_at | CPNE3 | copine III | AB014536 | 8q21.2 | Hs.14158 |
| 33500_i_at | IGHA1 | immunoglobulin heavy constant alpha 1 | S71043 | | |
| 35536_at | KIAA0604 | KIAA0604 gene product | AB011176 | | Hs.129801 |
| 41198_at | GRN | granulin | AF055008 | 17q21.32 | Hs.180577 |
| 1832_at | MCC | mutated in colorectal cancers | M62397 | 5q21-q22 | Hs.1345 |
| 35807_at | CYBA | cytochrome b-245, alpha polypeptide | M21186 | 16q24 | Hs.68877 |
| 37623_at | NR4A2 | nuclear receptor subfamily 4, group A, | X75918 | 2q22-q23 | Hs.82120 |
| | | member 2 | | | |
| 916_at | PTPRN | protein tyrosine phosphatase, receptor | L18983 | 2q35-q36.1 | Hs.89655 |
| | | type, N | | | |
| 35013_at | UNK_AF013512 | Cluster Incl AF013512: Homo sapiens | AF013512 | 20q11.23-q12 | Hs.154078 |
| | | lipopolysaccharide binding protein (LBP) | | | |
| | | exon 15, complete sequence and complete | | | |
| | | cds. | | | |
| 39245_at | UNK_U72507 | Human 40871 mRNA partial sequence | U72507 | | Hs.234216 |
| 36879_at | ECGF1 | endothelial cell growth factor 1 (platelet- | M63193 | 22q13.33 | Hs.73946 |
| | | derived) | | | |
| 37054_at | BPI | bactericidal/permeability-increasing | J04739 | 20q11.23-q12 | Hs.89535 |
| | | protein | | | |
| 31792_at | ANXA3 | annexin A3 | M20560 | 4q13-q22 | Hs.1378 |
| 1252_at | D5S346 | DNA segment, single copy probe LNS- | M73547 | 5q22-q23 | Hs.178112 |
| | | CAI/LNS-CAII (deleted in polyposis | | | |
| 37145_at | GNLY | granulysin | M85276 | 2p12-q11 | Hs.105806 |
| 36447_at | FCN1 | ficolin (collagen/fibrinogen domain- | S80990 | 9q34 | Hs.252136 |
| | | containing) 1 | | | |
| 31562_at | RHOK | rhodopsin kinase | U63973 | 13q34 | Hs.103501 |
| 37967_at | D6S49E | DNA segment on chromosome 6 (unique) | AF000424 | 6p21.3 | Hs.88411 |
| | | 49 expressed sequence | | | |
| 31586_f_at | UNK_X72475 | H. sapiens mRNA for rearranged Ig kappa | X72475 | | Hs.367983 |
| | | light chain variable region (I.114) | | | |
| 37215_at | PYGL | phosphorylase, glycogen; liver (Hers | AF046798 | 14q21-q22 | Hs.771 |
| | | disease, glycogen storage disease type VI) | | | |
| 39872_at | UNK_AL031588 | Human DNA sequence from clone | AL031588 | 22q13.2-q13.3 | Hs.122552 |
| | | 1163J1 on chromosome 22q13.2-13.33. | | | |
| | | Contains the 3′ part of a gene for a novel | | | |
| | | KIAA0279 LIKE EGF-like domain | | | |
| | | containing protein (similar to mouse | | | |
| | | Celsr1, rat MEGF2), a novel gene for a | | | |
| | | protein similar to C. elegans B0035.16 | | | |
| | | and bacterial tRNA (5- | | | |
| | | Methylaminomethyl-2-thiouridylate)- | | | |
| | | Methyltransferases, and the 3′ part of a | | | |
| | | novel gene for a protein similar to mouse | | | |
| | | B99... | | | |
| 988_at | CEACAM1 | carcinoembryonic antigen-related cell | X16354 | 19q13.2 | Hs.50964 |
| | | adhesion molecule 1 (biliary | | | |
| | | glycoprotein) | | | |
| 35094_f_at | LILRA3 | leukocyte immunoglobulin-like receptor, | AF025527 | 19q13.4 | Hs.113277 |
| | | subfamily A (without TM domain), | | | |
| | | member 3 | | | |
| 39591_s_at | FGL2 | fibrinogen-like 2 | Z36531 | 7q11.23 | Hs.2659 |
| 38194_s_at | IGKV1D-8 | immunoglobulin kappa variable 1D-8 | M63438 | 2p12 | Hs.156110 |
| 41627_at | SDF2 | stromal cell-derived factor 2 | D50645 | 17q11.2 | Hs.118684 |
| 266_s_at | CD24 | CD24 antigen (small cell lung carcinoma | L33930 | 6q21 | Hs.286124 |
| | | cluster 4 antigen) | | | |
| 34105_f_at | UNK_AI147237 | Homo sapiens isolate RP immunoglobulin | AI147237 | 14q32.33 | Hs.300697 |
| | | heavy chain FW2-JH region gene, partial | | | |
| | | cds | | | |
| 1339_s_at | UNK_X14675 | Cluster Incl X14675: Human bcr-abl | X14675 | | |
| | | mRNA 5′ fragment (clone 3c). | | | |
| 39128_r_at | PPP2R4 | protein phosphatase 2A, regulatory | X73478 | 9q34 | Hs.236963 |
| | | subunit B′ (PR 53) | | | |
| 33274_f_at | IGL@ | immunoglobulin lambda locus | M18645 | 22q11.1-q11.2 | Hs.181125 |
| 1825_at | IQGAP1 | IQ motif containing GTPase activating | L33075 | 15q26.1 | Hs.1742 |
| | | protein 1 | | | |
| 37233_at | OLR1 | oxidised low density lipoprotein (lectin- | AF079167 | 12p13.2-p12.3 | Hs.77729 |
| | | like) receptor 1 | | | |
| 36105_at | CEACAM6 | carcinoembryonic antigen-related cell | M18728 | 19q13.2 | Hs.73848 |
| | | adhesion molecule 6 (non-specific cross | | | |
| | | reacting antigen) | | | |
| 36338_at | UNK_W28504 | Cluster Incl W28504: 48e7 Human retina | W28504 | | Hs.348515 |
| | | cDNA randomly primed sublibrary Homo | | | |
| | | sapiens cDNA, mRNA sequence. | | | |
| 33273_f_at | IGL@ | immunoglobulin lambda locus | X57809 | 22q11.1-q11.2, | Hs.8997 |
| | | | | 6p21.3 | |
| 33150_at | UNK_AI126004 | ESTs, Weakly similar to cDNA EST | AI126004 | 4q13.3 | Hs.322901 |
| | | EMBL: T00542 comes from this gene | | | |
| | | [C. elegans] | | | |
| 35714_at | PDXK | pyridoxal (pyridoxine, vitamin B6) kinase | U89606 | 21q22.3 | Hs.38041 |
| 41471_at | S100A9 | S100 calcium-binding protein A9 | W72424 | 1q21 | Hs.112405 |
| | | (calgranulin B) | | | |
| 1117_at | CDA | cytidine deaminase | L27943 | 1p36.2-p35 | Hs.72924 |
| 33284_at | MPO | myeloperoxidase | M19507 | 17q23.1 | Hs.1817 |
| 38894_g_at | NCF4 | neutrophil cytosolic factor 4 (40 kD) | AL008637 | 22q13.1 | Hs.196352 |
| 31506_s_at | DEFA3 | defensin, alpha 3, neutrophil-specific | L12691 | 8p23.2-p23.1, | Hs.274463, Hs.294176 |
| | | | | 8pter-p23.3 | |
| 34350_at | RSN | restin (Reed-Steinberg cell-expressed | X64838 | 12q24.3 | Hs.31638 |
| | | intermediate filament-associated protein) | | | |
| 38895_i_at | NCF4 | neutrophil cytosolic factor 4 (40 kD) | X77094 | 22q13.1 | Hs.196352 |
| 39593_at | FGL2 | fibrinogen-like 2 | AI432401 | | Hs.351808 |
| 33963_at | AZU1 | azurocidin 1 (cationic antimicrobial | M96326 | 19p13.3 | Hs.72885 |
| | | protein 37) | | | |
| 35591_at | F11 | coagulation factor XI (plasma | M13142 | 4q35 | Hs.1430 |
| | | thromboplastin antecedent) | | | |
| 37864_s_at | IGHG3 | immunoglobulin heavy constant gamma 3 | Y14737 | 14q32.33 | Hs.300697 |
| | | (G3m marker) | | | |
| 36674_at | SCYA4 | small inducible cytokine A4 (homologous | J04130 | 17q12 | Hs.75703 |
| | | to mouse Mip-1b) | | | |
| 37121_at | NKG7 | natural killer cell group 7 sequence | S69115 | 19q13.41 | Hs.10306 |
| 32612_at | GSN | gelsolin (amyloidosis, Finnish type) | X04412 | 9q33 | Hs.290070 |
| 39413_at | OPHN1 | oligophrenin 1 | AJ001189 | Xq12 | Hs.128824 |
| 41827_f_at | UNK_AI932613 | Human rearranged immunoglobulin | AI932613 | | Hs.350074 |
| | | lambda light chain mRNA | | | |
| 41440_at | D6S2245E | Ke6 gene, mouse, human homolog of | D82061 | 6p21.3 | Hs.288354 |
| 33979_at | RNASE3 | ribonuclease, RNase A family, 3 | X55990 | 14q24-q31 | Hs.73839 |
| | | (eosinophil cationic protein) | | | |
| 38533_s_at | ITGAM | integrin, alpha M (complement | J03925 | 16p11.2 | Hs.172631 |
| | | component receptor 3, alpha; also known | | | |
| | | as CD11b (p170), macrophage antigen | | | |
| | | alpha polypeptide) | | | |
| 32275_at | SLPI | secretory leukocyte protease inhibitor | X04470 | 20q12 | Hs.251754 |
| | | (antileukoproteinase) | | | |
| 35315_at | ORM1 | orosomucoid 1 | X02544 | 9q31-q32, | Hs.572 |
| | | | | 9q32 | |
| 33757_f_at | PSG11 | pregnancy specific beta-1-glycoprotein 11 | M69245 | 19q13.2 | Hs.334408 |
| 40278_at | KIAA1080 | KIAA1080 protein | AB029003 | 16p12 | Hs.155546 |
| 36983_f_at | HP | haptoglobin | X00442 | 16q22.1 | Hs.75990 |
| 37105_at | CTSG | cathepsin G | M16117 | 14q11.2 | Hs.100764 |
| 34415_at | ACVR1B | activin A receptor, type IB | Z22536 | 12q13 | Hs.99954 |
| 41840_r_at | UNK_H08175 | Homo sapiens clone IMAGE 25997 | H08175 | | Hs.6524 |
| AFFX- | 28SRNA3_Hs_AFFX | 28SRNA3 control sequence (H. sapiens) | M27830 | | |
| M27830_3_at | | [AFFX] | | | |
| 39330_s_at | ACTN1 | actinin, alpha 1 | M95178 | 14q24 | Hs.119000 |
| 38513_at | KIAA0061 | KIAA0061 protein | D31765 | 8q22.1 | Hs.170114 |
| 38514_at | IGLL3 | immunoglobulin lambda-like polypeptide 3 | M27749 | 22q11.23 | Hs.348935 |
| 38249_at | UNK_Z97632 | Cluster Incl Z97632: Human DNA | Z97632 | Xq26.3 | Hs.9030 |
| | | sequence from PAC 196E23 on | | | |
| | | chromosome Xq26.1-27.2. Contains the | | | |
| | | TAT-SF1 (HIV-1 transcriptional | | | |
| | | elongation factor TAT cofactor TAT-SF1) | | | |
| | | gene, the BRS3 (Bombesin Receptor | | | |
| | | subtype-3 (Uterine Bombesin Receptor, | | | |
| | | BRS-3) gene, an unknown gene coding | | | |
| | | for two isoforms, a predicted CpG island, | | | |
| | | ESTs and STSs, complete sequence. | | | |
| 40159_r_at | NCF1 | neutrophil cytosolic factor 1 (47kD, | M55067 | 7q11.23 | Hs.1583 |
| | | chronic granulomatous disease, autosomal | | | |
| | | 1) | | | |
| 31793_at | DEFA1 | defensin, alpha 1, myeloid-related | AL036554 | 8p23.2-p23.1, | Hs.274463 |
| | | sequence | | 8pter-p23.3 | |
| 1407_g_at | NR2C1 | nuclear receptor subfamily 2, group C, | M21985 | 12q21.32-q21.33 | Hs.108301 |
| | | member 1 | | | |
| 679_at | CTSG | cathepsin G | J04990 | 14q11.2 | Hs.100764 |
| 31578_at | UNK_M96936 | Cluster Incl M96936: Homo sapiens | M96936 | | |
| | | cystic fibrosis transmembrane | | | |
| | | conductance regulator (CFTR) gene, | | | |
| | | exons 23, 24a, and 24. | | | |
| 35919_at | TCN1 | transcobalamin I (vitamin B12 binding | J05068 | 11q11-q12 | Hs.2012 |
| | | protein, R binder family) | | | |
| 37149_s_at | UNK_U95626 | Cluster Incl U95626: Homo sapiens ccr2b | U95626 | 3q21-q23 | Hs.105938 |
| | | (ccr2), ccr2a (ccr2), ccr5 (ccr5) and ccr6 | | | |
| | | (ccr6) genes, complete cds, and | | | |
| | | lactoferrin (lactoferrin) gene, partial cds, | | | |
| | | complete sequence. | | | |
| 38976_at | CORO1A | coronin, actin-binding protein, 1A | D44497 | 16q13 | Hs.109606 |
| 40234_at | DGCR6 | DiGeorge syndrome critical region 6 | X96484 | , 22q11.21 | Hs.336664 |
| 36984_f_at | HPR | haptoglobin-related protein | X89214 | 16q22.1 | Hs.328822 |
| 35762_at | KIAA0483 | KIAA0483 protein | AB007952 | 1q41 | Hs.64691 |
| 40517_at | KIAA0372 | KIAA0372 gene product | AB002370 | 5q21.1-q21.2 | Hs.170098 |
| 33093_at | IL18RAP | interleukin 18 receptor accessory protein | AF077346 | 2p24.3-p24.1 | Hs.158315 |
| 31357_at | UNK_W26214 | Cluster Incl W26214: 22d11 Human | W26214 | | |
| | | retina cDNA randomly primed sublibrary | | | |
| | | Homo sapiens cDNA, mRNA sequence. | | | |
| 40171_at | FRAT2 | GSK-3 binding protein FRAT2 | AF062739 | 10q23-q24.1 | Hs.140720 |
| 32451_at | MS4A3 | membrane-spanning 4-domains, | L35848 | 11q12-q13.1 | Hs.99960 |
| | | subfamily A, member 3 (hematopoietic | | | |
| | | cell-specific) | | | |
| 33021_at | UNK_AF035314 | Homo sapiens clone 23651 mRNA | AF035314 | | Hs.134526 |
| | | sequence | | | |
| 38615_at | GW112 | differentially expressed in hematopoietic | AF097021 | 13q14.2 | Hs.273321 |
| | | lineages | | | |
| 31495_at | SCYC2 | small inducible cytokine subfamily C, | D63789 | 1q23, | Hs.174228, Hs.3195 |
| | | member 2 | | 1q23-q25 | |
| 33530_at | CEACAM8 | carcinoembryonic antigen-related cell | M33326 | 19q13.2 | Hs.41 |
| | | adhesion molecule 8 | | | |
| 37066_at | PRTN3 | proteinase 3 (serine proteinase, | X55668 | 19p13.3 | Hs.928 |
| | | neutrophil, Wegener granulomatosis | | | |
| | | autoantigen) | | | |
| 1350_at | CYP4F2 | cytochrome P450, subfamily IVF, | U02388 | 19pter-p13.11 | Hs.101 |
| | | polypeptide 2 | | | |
| 37096_at | ELA2 | elastase 2, neutrophil | M34379 | 19p13.3 | Hs.99863 |
| 37975_at | CYBB | cytochrome b-245, beta polypeptide | X04011 | Xp21.1 | Hs.88974 |
| | | (chronic granulomatous disease) | | | |
| 34546_at | DEFA4 | defensin, alpha 4, corticostatin | AI250799 | 8p23 | Hs.2582 |
| 36237_at | SLC22A6 | solute carrier family 22 (organic anion | AB009698 | 11q13.1-q13.2 | Hs.23965 |
| | | transporter), member 6 | | | |
| 38273_at | ATPASEP | ATPase type IV, phospholipid | AJ006268 | 18q23 | Hs.91471 |
| | | transporting (P-type)(putative) | | | |
| 37434_at | UNK_W28907 | Cluster Incl W28907: 53e12 Human | W28907 | 16p11.2 | Hs.111429 |
| | | retina cDNA randomly primed sublibrary | | | |
| | | Homo sapiens cDNA, mRNA sequence. | | | |
| 31477_at | TFF3 | trefoil factor 3 (intestinal) | L08044 | 21q22.3 | Hs.352107 |
| 34013_f_at | POU1F1 | POU domain, class 1, transcription factor | D12892 | 3p11 | Hs.89394 |
| | | 1 (Pit1, growth hormone factor 1) | | | |
| 32054_at | CCNT2 | cyclin T2 | AF048732 | 2q14.3 | Hs.155478 |
| 36548_at | KIAA0895 | KIAA0895 protein | AB020702 | 7p15.3 | Hs.6224 |
| 33244_at | CHN2 | chimerin (chimaerin) 2 | U07223 | 7p15.3 | Hs.286055 |
| 39765_at | KIAA0320 | KIAA0320 protein | AB002318 | 15q15-q21 | Hs.150443 |
| 33742_f_at | UNK_W27838 | ESTs, Highly similar to CGI-11 protein | W27838 | 8p22-q22.3 | Hs.19575 |
| | | [H. sapiens] | | | |
| 34014_f_at | POU1F1 | POU domain, class 1, transcription factor | D10216 | 3p11 | Hs.89394 |
| | | 1 (Pit1, growth hormone factor 1) | | | |
| 732_f_at | MUC3 | mucin 3, intestinal | M55406 | | |
| 36464_at | SGP28 | specific granule protein (28 kDa); | X94323 | 6p12.2 | Hs.54431 |
| | | cysteine-rich secretory protein-3 | | | |
| 36372_at | HK3 | hexokinase 3 (white cell) | U51333 | 5q35.2 | Hs.159237 |
| 33914_r_at | FECH | ferrochelatase (protoporphyria) | D00726 | 18q21.3 | Hs.26 |
| 38908_s_at | UNK_AL096744 | Homo sapiens mRNA; cDNA | AL096744 | 6q21 | Hs.115521 |
| | | DKFZp566H033 (from clone | | | |
| | | DKFZp566H033) | | | |
| 32620_at | FETUB | fetuin B | AB017551 | 3q27 | Hs.81073 |
| 31381_at | PGLYRP | peptidoglycan recognition protein | AF076483 | 19q13.2-q13.3 | Hs.137583 |
| 37897_s_at | TFF3 | trefoil factor 3 (intestinal) | AI985964 | 21q22.3 | Hs.82961 |
| 32579_at | SMARCA4 | SWI/SNF related, matrix associated, actin | U29175 | 19p13.2 | Hs.78202 |
| | | dependent regulator of chromatin, | | | |
| | | subfamily a, member 4 | | | |
| 39894_f_at | BRD1 | bromodomain-containing 1 | Z98885 | 22q13.33 | Hs.127950 |
| 36071_at | UNK_AF070633 | Homo sapiens clone 24672 mRNA | AF070633 | | Hs.5010 |
| | | sequence | | | |
| 38879_at | S100A12 | S100 calcium-binding protein A12 | D83664 | 1q21 | Hs.19413 |
| | | (calgranulin C) | | | |
| 36710_at | CAMP | cathelicidin antimicrobial peptide | Z38026 | 3p21.3 | Hs.51120 |
| 31859_at | MMP9 | matrix metalloproteinase 9 (gelatinase B, | J05070 | 20q11.2-q13.1 | Hs.151738 |
| | | 92 kD gelatinase, 92 kD type IV | | | |
| | | collagenase) | | | |
| 39318_at | TCL1A | T-cell leukemia/lymphoma 1A | X82240 | 14q32.1 | Hs.2484 |
| 35418_at | UNK_J04178 | Human abnormal beta-hexosaminidase | J04178 | | Hs.166299 |
| | | alpha chain (HEXA) mRNA, partial cds | | | |
| 31666_f_at | KIAA0168 | KIAA0168 gene product | W28731 | 20pter-p12.1 | Hs.80905 |
| 32821_at | LCN2 | lipocalin 2 (oncogene 24p3) | AI762213 | 9q34 | Hs.204238 |
| 31574_i_at | UNK_M14087 | Human HL14 gene encoding beta- | M14087 | | |
| | | galactoside-binding lectin, 3′ end, clone 2 | | | |
| 2041_i_at | ABL1 | v-abl Abelson murine leukemia viral | M14752 | 9q34.1 | Hs.146355 |
| | | oncogene homolog 1 | | | |

TABLE 10a
Genes that Are Differentially Expressed in Bone Marrow Leukocytes
of AML Patients Compared to Bone Marrow Leukocytes of MDS Patients

| | No. of Present | | | | | | |
|---|---|---|---|---|---|---|---|
| | (Disease-Free | No. of Present | No. of Present | COV | COV | | P value (unequal) |
| Qualifier | n = 18) | (MDS n = 13) | (AML n = 31) | (MDS) | (AML) | AML/MDS | (AML vs MDS) |
| 34660_at | 18 | 10 | 29 | 54.01% | 105.61% | 5.84 | 0.000144386 |
| 38514_at | 15 | 6 | 26 | 105.37% | 120.77% | 5.70 | 0.000779173 |
| 34583_at | 0 | 7 | 31 | 69.48% | 110.90% | 3.68 | 0.001199784 |
| 37754_at | 0 | 3 | 18 | 115.39% | 142.43% | 3.63 | 0.011026039 |
| 1065_at | 11 | 9 | 31 | 90.81% | 97.87% | 3.18 | 0.000991198 |
| 38112_g_at | 18 | 10 | 26 | 113.41% | 152.55% | 3.16 | 0.024540324 |
| 35869_at | 17 | 8 | 24 | 91.11% | 93.84% | 3.13 | 0.000775029 |
| 39421_at | 5 | 9 | 29 | 103.36% | 66.21% | 3.02 | 7.85452E−05 |
| 31441_at | 18 | 7 | 26 | 105.41% | 94.21% | 3.02 | 0.001363431 |
| 32096_at | 0 | 2 | 22 | 32.55% | 68.90% | 2.99 | 9.17711E−06 |
| 31682_s_at | 16 | 6 | 21 | 128.12% | 156.72% | 2.95 | 0.037202183 |
| 41468_at | 18 | 12 | 30 | 84.04% | 128.80% | 2.84 | 0.012052005 |
| 36908_at | 1 | 7 | 21 | 46.98% | 110.36% | 2.82 | 0.003273591 |
| 36881_at | 6 | 9 | 28 | 63.80% | 60.09% | 2.73 | 9.4524E−06 |
| 32941_at | 1 | 1 | 24 | 61.72% | 82.86% | 2.70 | 0.000382164 |
| 39591_s_at | 14 | 3 | 23 | 82.29% | 112.66% | 2.64 | 0.007494998 |
| 33777_at | 4 | 4 | 27 | 110.34% | 67.35% | 2.64 | 0.000716653 |
| 829_s_at | 18 | 9 | 30 | 66.31% | 51.58% | 2.60 | 4.66512E−06 |
| 39710_at | 18 | 12 | 31 | 75.35% | 53.92% | 2.51 | 3.16598E−05 |
| 34862_at | 14 | 9 | 30 | 60.62% | 66.67% | 2.50 | 8.04417E−05 |
| 39593_at | 17 | 5 | 24 | 133.17% | 126.86% | 2.45 | 0.035689733 |
| 39023_at | 12 | 8 | 30 | 75.77% | 65.61% | 2.43 | 0.000237592 |
| 943_at | 2 | 5 | 30 | 105.70% | 64.35% | 2.43 | 0.001282565 |
| 39693_at | 10 | 6 | 27 | 77.89% | 44.84% | 2.40 | 3.3839E−05 |
| 37692_at | 18 | 13 | 31 | 37.04% | 53.96% | 2.39 | 2.6376E−06 |
| 39936_at | 0 | 0 | 13 | 0.00% | 101.86% | 2.39 | 0.003441962 |
| 32755_at | 0 | 6 | 26 | 64.72% | 72.62% | 2.38 | 0.00040754 |
| 37242_at | 6 | 4 | 24 | 25.75% | 77.72% | 2.37 | 0.000301362 |
| 1196_at | 9 | 5 | 29 | 45.94% | 36.99% | 2.31 | 7.92494E−08 |
| 33412_at | 18 | 12 | 31 | 101.15% | 67.43% | 2.31 | 0.002202294 |
| 38111_at | 18 | 11 | 26 | 112.71% | 136.12% | 2.30 | 0.049415576 |
| 41332_at | 7 | 5 | 23 | 45.61% | 47.34% | 2.29 | 1.66686E−06 |
| 35523_at | 0 | 3 | 12 | 32.55% | 137.20% | 2.29 | 0.030920214 |
| 1486_at | 0 | 0 | 2 | 0.00% | 112.84% | 2.29 | 0.009301972 |
| 40789_at | 13 | 12 | 29 | 58.72% | 53.21% | 2.28 | 2.79762E−05 |
| 38717_at | 16 | 12 | 31 | 53.01% | 46.64% | 2.28 | 4.22538E−06 |
| 40607_at | 18 | 11 | 29 | 72.62% | 84.10% | 2.28 | 0.002572096 |
| 32668_at | 9 | 7 | 30 | 28.39% | 73.10% | 2.27 | 0.000231537 |
| 40517_at | 17 | 11 | 31 | 28.39% | 112.77% | 2.27 | 0.010476436 |
| 1750_at | 4 | 3 | 28 | 98.72% | 55.44% | 2.26 | 0.001304584 |
| 36955_at | 1 | 0 | 15 | 79.87% | 54.56% | 2.25 | 0.000321281 |
| 907_at | 11 | 7 | 30 | 37.04% | 64.86% | 2.24 | 8.16727E−05 |
| 41184_s_at | 11 | 4 | 26 | 55.58% | 50.74% | 2.23 | 1.97258E−05 |
| 41654_at | 13 | 13 | 31 | 63.36% | 61.26% | 2.20 | 0.000242084 |
| 36958_at | 5 | 4 | 24 | 98.77% | 71.35% | 2.18 | 0.004702152 |
| 34961_at | 6 | 4 | 25 | 57.01% | 94.75% | 2.18 | 0.005703652 |
| 36215_at | 10 | 8 | 31 | 47.24% | 73.80% | 2.16 | 0.000669701 |
| 35255_at | 8 | 11 | 31 | 39.30% | 34.77% | 2.16 | 5.14431E−08 |
| 38220_at | 15 | 11 | 31 | 38.11% | 65.81% | 2.13 | 0.000178291 |
| 478_g_at | 9 | 10 | 31 | 49.05% | 52.24% | 2.13 | 2.94378E−05 |
| 1752_at | 0 | 2 | 13 | 36.74% | 112.73% | 2.12 | 0.015930736 |
| 1751_g_at | 14 | 10 | 29 | 64.41% | 51.39% | 2.11 | 0.000156536 |
| 2025_s_at | 18 | 13 | 31 | 72.85% | 43.44% | 2.10 | 0.000219645 |
| 40514_at | 15 | 9 | 30 | 74.54% | 41.49% | 2.10 | 0.000251048 |
| 33396_at | 18 | 13 | 31 | 41.83% | 42.37% | 2.08 | 2.05862E−06 |
| 36465_at | 8 | 8 | 30 | 50.67% | 55.20% | 2.08 | 8.85843E−05 |
| 39175_at | 1 | 10 | 30 | 59.26% | 54.10% | 2.07 | 0.000188386 |
| 34651_at | 9 | 6 | 29 | 51.75% | 41.89% | 2.07 | 1.24348E−05 |
| 40274_at | 1 | 1 | 7 | 83.17% | 37.78% | 2.06 | 0.00073264 |
| 33132_at | 3 | 6 | 21 | 70.53% | 83.17% | 2.04 | 0.006459359 |
| 37716_at | 2 | 2 | 21 | 62.88% | 103.64% | 2.03 | 0.017474825 |
| 1826_at | 0 | 0 | 6 | 0.00% | 115.39% | 2.03 | 0.020294144 |
| 41163_at | 3 | 7 | 27 | 75.69% | 56.86% | 2.03 | 0.001369303 |
| 38780_at | 18 | 11 | 31 | 55.98% | 40.32% | 2.01 | 3.92427E−05 |
| 37742_at | 15 | 11 | 30 | 42.08% | 39.40% | 2.00 | 2.93088E−06 |
| 39695_at | 18 | 13 | 31 | 63.88% | 43.26% | 0.50 | 0.015968764 |
| 189_s_at | 18 | 12 | 26 | 78.05% | 86.03% | 0.50 | 0.045325806 |
| 36591_at | 18 | 12 | 23 | 45.40% | 66.57% | 0.50 | 0.002088984 |
| 40091_at | 18 | 13 | 31 | 69.19% | 67.25% | 0.50 | 0.0251031 |
| 37192_at | 16 | 10 | 20 | 73.54% | 107.72% | 0.50 | 0.038809111 |
| 37508_f_at | 5 | 12 | 25 | 54.29% | 48.50% | 0.49 | 0.005986356 |
| 38672_at | 16 | 13 | 29 | 38.75% | 40.67% | 0.49 | 0.000466268 |
| 595_at | 18 | 13 | 31 | 69.36% | 71.91% | 0.49 | 0.024721542 |
| 988_at | 18 | 11 | 19 | 66.84% | 88.68% | 0.49 | 0.0224487 |
| 38879_at | 18 | 12 | 25 | 78.02% | 131.77% | 0.48 | 0.048416929 |
| 1270_at | 5 | 10 | 15 | 73.97% | 51.55% | 0.48 | 0.028022671 |
| 33813_at | 17 | 13 | 27 | 71.61% | 65.87% | 0.48 | 0.024491367 |
| 38508_s_at | 0 | 3 | 3 | 60.23% | 46.69% | 0.48 | 0.009358692 |
| 31793_at | 18 | 11 | 29 | 78.65% | 93.55% | 0.48 | 0.038896092 |
| 35966_at | 18 | 11 | 23 | 71.23% | 87.60% | 0.47 | 0.024494425 |
| 37022_at | 0 | 1 | 1 | 68.34% | 56.27% | 0.47 | 0.017741088 |
| 35918_at | 0 | 1 | 0 | 74.49% | 106.85% | 0.47 | 0.031777745 |
| 37405_at | 17 | 12 | 25 | 80.65% | 102.85% | 0.47 | 0.042999764 |
| 35672_at | 7 | 10 | 16 | 57.69% | 47.98% | 0.47 | 0.006513708 |
| 37285_at | 18 | 13 | 29 | 80.13% | 104.31% | 0.47 | 0.041326534 |
| 106_at | 15 | 11 | 13 | 76.73% | 89.63% | 0.47 | 0.032103848 |
| 35372_r_at | 18 | 13 | 31 | 68.06% | 64.99% | 0.46 | 0.016257736 |
| 34832_s_at | 17 | 13 | 22 | 55.51% | 35.90% | 0.46 | 0.004593746 |
| 37024_at | 18 | 13 | 31 | 54.07% | 44.79% | 0.46 | 0.003808253 |
| 40617_at | 14 | 13 | 30 | 47.11% | 31.96% | 0.46 | 0.001395047 |
| 40647_at | 18 | 12 | 28 | 79.35% | 94.78% | 0.46 | 0.035333446 |
| 1257_s_at | 11 | 11 | 28 | 60.11% | 81.89% | 0.46 | 0.008111764 |
| 32606_at | 18 | 11 | 20 | 63.57% | 46.42% | 0.45 | 0.00953403 |
| 39436_at | 18 | 13 | 30 | 63.27% | 81.91% | 0.45 | 0.009742823 |
| 307_at | 18 | 11 | 23 | 56.85% | 71.70% | 0.45 | 0.004782498 |
| 40769_r_at | 4 | 4 | 2 | 38.85% | 40.26% | 0.45 | 0.000211588 |
| 266_s_at | 18 | 12 | 29 | 61.15% | 90.85% | 0.44 | 0.008056514 |
| 40446_at | 18 | 13 | 31 | 44.68% | 34.31% | 0.44 | 0.000704454 |
| 37701_at | 18 | 13 | 30 | 64.25% | 67.61% | 0.44 | 0.009267628 |
| 37200_at | 16 | 13 | 25 | 79.24% | 115.77% | 0.44 | 0.031508083 |
| 31888_s_at | 8 | 9 | 14 | 85.62% | 109.66% | 0.44 | 0.041656711 |
| 35601_at | 10 | 8 | 8 | 66.02% | 82.52% | 0.43 | 0.009959894 |
| 36713_at | 17 | 12 | 24 | 64.31% | 107.70% | 0.42 | 0.009155286 |
| 32607_at | 18 | 13 | 31 | 75.16% | 72.53% | 0.42 | 0.017390821 |
| 39969_at | 9 | 10 | 17 | 65.92% | 61.21% | 0.41 | 0.007818399 |
| 35256_at | 5 | 4 | 21 | 95.34% | 94.09% | 0.41 | 0.04921945 |
| 40202_at | 18 | 13 | 28 | 73.36% | 78.79% | 0.41 | 0.014426687 |
| 40888_f_at | 18 | 13 | 27 | 48.91% | 99.29% | 0.41 | 0.001073335 |
| 33080_s_at | 14 | 10 | 21 | 64.27% | 60.24% | 0.41 | 0.006397054 |
| 38615_at | 18 | 9 | 12 | 83.46% | 105.92% | 0.40 | 0.027533758 |
| 34319_at | 18 | 13 | 29 | 74.51% | 91.40% | 0.40 | 0.015241249 |
| 38585_at | 18 | 11 | 30 | 89.71% | 102.22% | 0.40 | 0.036102175 |
| 39908_at | 16 | 9 | 28 | 61.51% | 85.20% | 0.40 | 0.004441005 |
| 32434_at | 18 | 13 | 25 | 66.53% | 98.06% | 0.39 | 0.007297027 |
| 38740_at | 18 | 13 | 26 | 84.94% | 47.34% | 0.39 | 0.02390029 |
| 31410_at | 8 | 4 | 3 | 76.91% | 39.75% | 0.38 | 0.013889529 |
| 35785_at | 18 | 13 | 31 | 79.64% | 52.50% | 0.37 | 0.015532383 |
| 34627_at | 1 | 1 | 0 | 100.39% | 92.48% | 0.37 | 0.046901998 |
| 36709_at | 14 | 13 | 25 | 70.92% | 54.38% | 0.37 | 0.008038515 |
| 36979_at | 18 | 13 | 31 | 51.62% | 61.20% | 0.37 | 0.000860098 |
| 31792_at | 18 | 13 | 24 | 76.88% | 99.81% | 0.37 | 0.013348641 |
| 33304_at | 16 | 10 | 13 | 73.50% | 84.12% | 0.37 | 0.009784856 |
| 34435_at | 17 | 12 | 17 | 80.35% | 38.40% | 0.36 | 0.013878626 |
| 32529_at | 18 | 13 | 25 | 65.96% | 80.53% | 0.34 | 0.003603237 |
| 37351_at | 18 | 9 | 18 | 108.42% | 90.27% | 0.33 | 0.048368083 |
| 37149_s_at | 18 | 11 | 27 | 107.80% | 146.84% | 0.33 | 0.049620516 |
| 681_at | 18 | 11 | 25 | 83.85% | 129.48% | 0.32 | 0.014844334 |
| 936_s_at | 1 | 2 | 1 | 80.03% | 76.61% | 0.32 | 0.010611416 |
| 38012_at | 0 | 1 | 3 | 89.31% | 86.34% | 0.32 | 0.019096181 |
| AFFX- | 7 | 11 | 13 | 61.99% | 99.69% | 0.32 | 0.002060589 |
| HUMRGE/M10098_5_at | | | | | | | |
| 1369_s_at | 17 | 13 | 30 | 106.91% | 92.27% | 0.32 | 0.042429136 |
| 2002_s_at | 18 | 13 | 31 | 71.59% | 62.78% | 0.32 | 0.004937967 |
| AFFX- | 8 | 12 | 17 | 82.11% | 159.11% | 0.32 | 0.013096935 |
| HUMRGE/M10098_M_at | | | | | | | |
| 35379_at | 2 | 0 | 1 | 57.96% | 73.91% | 0.31 | 0.00107944 |
| 34498_at | 18 | 12 | 25 | 83.51% | 88.80% | 0.30 | 0.011505282 |
| 1962_at | 18 | 11 | 23 | 66.68% | 90.59% | 0.30 | 0.002680156 |
| 1115_at | 15 | 10 | 16 | 110.52% | 164.09% | 0.29 | 0.04243399 |
| 35920_at | 0 | 9 | 12 | 104.95% | 122.47% | 0.28 | 0.029872483 |
| 39209_r_at | 13 | 10 | 16 | 91.15% | 130.54% | 0.27 | 0.014467252 |
| 39208_i_at | 17 | 12 | 23 | 109.89% | 138.31% | 0.26 | 0.032941521 |
| 40215_at | 13 | 11 | 12 | 78.46% | 57.77% | 0.22 | 0.00377906 |
| 33849_at | 18 | 13 | 30 | 76.02% | 70.56% | 0.21 | 0.002751892 |

TABLE 10b
Genes that Are Differentially Expressed in Bone Marrow Leukocytes of AML Patients Compared to Bone Marrow Leukocytes of MDS Patients

| Qualifier | Gene Name | Gene Title | Entrez No. | Cyto Band | Unigene No. |
|---|---|---|---|---|---|
| 34660_at | RNASE6 | ribonuclease, RNase A family, k6 | AI142565 | 14q11.1 | Hs.23262 |
| 38514_at | IGLL3 | immunoglobulin lambda-like polypeptide 3 | M27749 | 22q11.23 | Hs.348935 |
| 34583_at | FLT3 | fms-related tyrosine kinase 3 | U02687 | 13q12 | Hs.385 |
| 37754_at | LGALS3BP | lectin, galactoside-binding, soluble, 3 binding | L13210 | 17q25 | Hs.79339 |
| | | protein (galectin 6 binding protein) | | | |
| 1065_at | FLT3 | fms-related tyrosine kinase 3 | U02687 | 13q12 | Hs.385 |
| 38112_g_at | CSPG2 | chondroitin sulfate proteoglycan 2 (versican) | X15998 | 5q14.3 | Hs.81800 |
| 35869_at | MD-1 | MD-1, RP105-associated | AB020499 | 6p24.1 | Hs.184018 |
| 39421_at | RUNX1 | runt-related transcription factor 1 (acute myeloid | D43969 | 21q22.3 | Hs.129914 |
| | | leukemia 1; aml1 oncogene) | | | |
| 31441_at | UNK_X55989 | Human ECRP gene for eosinophil cationic related | X55989 | | |
| | | protein | | | |
| 32096_at | LYL1 | lymphoblastic leukemia derived sequence 1 | AC005546 | 19p13.13 | Hs.158947 |
| 31682_s_at | CSPG2 | chondroitin sulfate proteoglycan 2 (versican) | D32039 | 5q14.3 | Hs.81800 |
| 41468_at | TRG@ | T cell receptor gamma locus | M30894 | 7p15-p14 | Hs.112259 |
| 36908_at | MRC1 | mannose receptor, C type 1 | M93221 | 10p13 | Hs.75182 |
| 36881_at | ETFB | electron-transfer-flavoprotein, beta polypeptide | X71129 | 19q13.3 | Hs.74047 |
| 32941_at | ICSBP1 | interferon consensus sequence binding protein 1 | M91196 | 16q24.1 | Hs.14453 |
| 39591_s_at | FGL2 | fibrinogen-like 2 | Z36531 | 7q11.23 | Hs.2659 |
| 33777_at | TBXAS1 | thromboxane A synthase 1 (platelet, cytochrome | D34625 | 7q34-q35 | Hs.2001 |
| | | P450, subfamily V) | | | |
| 829_s_at | GSTP1 | glutathione S-transferase pi | U21689 | 11q13 | Hs.226795 |
| 39710_at | P311 | P311 protein | U30521 | 5q21.3 | Hs.142827 |
| 34862_at | UNK_AA005018 | ESTs, Highly similar to CGI-49 protein | AA005018 | 1q44 | Hs.238126 |
| | | [H. sapiens] | | | |
| 39593_at | FGL2 | fibrinogen-like 2 | AI432401 | | Hs.351808 |
| 39023_at | IDH1 | isocitrate dehydrogenase 1 (NADP+), soluble | AF020038 | 2q33.3 | Hs.11223 |
| 943_at | RUNX1 | runt-related transcription factor 1 (acute myeloid | D43968 | 21q22.3 | Hs.129914 |
| | | leukemia 1; aml1 oncogene) | | | |
| 39693_at | UNK_N53547 | Homo sapiens clone 25036 mRNA sequence | N53547 | 11q13.1 | Hs.13662 |
| 37692_at | DBI | diazepam binding inhibitor (GABA receptor | AI557240 | 2q12-q21 | Hs.78888 |
| | | modulator, acyl-Coenzyme A binding protein) | | | |
| 39936_at | CCR2 | chemokine (C-C motif) receptor 2 | U95626 | 3p21 | Hs.395 |
| 32755_at | ACTA2 | actin, alpha 2, smooth muscle, aorta | X13839 | 10q23.3 | Hs.195851 |
| 37242_at | UNK_U79260 | Human clone 23745 mRNA, complete cds | U79260 | 16q12.2 | Hs.284741 |
| 1196_at | CHC1 | chromosome condensation 1 | D00591 | 1p36.1 | Hs.84746 |
| 33412_at | LGALS1 | lectin, galactoside-binding, soluble, 1 (galectin 1) | AI535946 | 22q13.1 | Hs.227751 |
| 38111_at | CSPG2 | chondroitin sulfate proteoglycan 2 (versican) | X15998 | 5q14.3 | Hs.81800 |
| 41332_at | POLR2E | polymerase (RNA) II (DNA directed) polypeptide | D38251 | 19p13.3 | Hs.24301 |
| | | E (25 kD) | | | |
| 35523_at | PGDS | prostaglandin D2 synthase, hematopoietic | AF150241 | 4q22.1 | Hs.128433 |
| 1486_at | POLR2J | polymerase (RNA) II (DNA directed) polypeptide | L37127 | 7q22-q31.1 | Hs.80475 |
| | | J (13.3 kD) | | | |
| 40789_at | AK2 | adenylate kinase 2 | U54645 | 1p34 | Hs.171811 |
| 38717_at | DKFZP586A0522 | DKFZP586A0522 protein | AL050159 | 12q11 | Hs.288771 |
| 40607_at | DPYSL2 | dihydropyrimidinase-like 2 | U97105 | 8p22-p21 | Hs.173381 |
| 32668_at | SSBP2 | single-stranded-DNA-binding protein | AL080076 | 5q14.1 | Hs.169833 |
| 40517_at | KIAA0372 | KIAA0372 gene product | AB002370 | 5q21.1-q21.2 | Hs.170098 |
| 1750_at | FARSL | phenylalanine-tRNA synthetase-like | AD000092 | 19p13.2 | Hs.23111 |
| 36955_at | GP36B | endoplasmic reticulum glycoprotein | U10362 | 5q35.3 | Hs.75864 |
| 907_at | ADA | adenosine deaminase | M13792 | 20q12-q13.11 | Hs.1217 |
| 41184_s_at | UNK_X87344 | H. sapiens DMA, DMB, HLA-Z1, IPP2, LMP2, | X87344 | 6p21.3 | Hs.180062 |
| | | TAP1, LMP7, TAP2, DOB, DQB2 and RING8, 9, | | | |
| | | 13 and 14 genes | | | |
| 41654_at | ADA | adenosine deaminase | X02994 | 20q12-q13.11 | Hs.1217 |
| 36958_at | ZYX | zyxin | X95735 | 13q12, 7q32 | Hs.75873 |
| 34961_at | TACTILE | T cell activation, increased late expression | M88282 | 3q13.2 | Hs.142023 |
| 36215_at | PRKACB | protein kinase, cAMP-dependent, catalytic, beta | M34181 | 1p36.1 | Hs.87773 |
| 35255_at | RANBP7 | RAN binding protein 7 | AF098799 | 11p15.3 | Hs.5151 |
| 38220_at | DPYD | dihydropyrimidine dehydrogenase | U20938 | 1p22 | Hs.1602 |
| 478_g_at | IRF5 | interferon regulatory factor 5 | U51127 | 7q32 | Hs.334450 |
| 1752_at | CALR | calreticulin | AD000092 | 19p13.3-p13.2 | Hs.16488 |
| 1751_g_at | FARSL | phenylalanine-tRNA synthetase-like | AD000092 | 19p13.2 | Hs.23111 |
| 2025_s_at | APEX | APEX nuclease (multifunctional DNA repair | M80261 | 14q11.2-q12 | Hs.73722 |
| | | enzyme) | | | |
| 40514_at | LOC51614 | hypothetical 43.2 Kd protein | AF091085 | 20pter-q12 | Hs.169992 |
| 33396_at | GSTP1 | glutathione S-transferase pi | U12472 | 11q13 | Hs.226795 |
| 36465_at | IRF5 | interferon regulatory factor 5 | U51127 | 7q32 | Hs.334450 |
| 39175_at | PFKP | phosphofructokinase, platelet | D25328 | 10p15.3-p15.2 | Hs.99910 |
| 34651_at | COMT | catechol-O-methyltransferase | M58525 | 22q11.21 | Hs.240013 |
| 40274_at | DBP | D site of albumin promoter (albumin D-box) | U48213 | 19q13.3 | Hs.155402 |
| | | binding protein | | | |
| 33132_at | HSU37012 | cleavage and polyadenylation specificity factor | U37012 | 8q24.23 | Hs.83727 |
| 37716_at | MOX2 | antigen identified by monoclonal antibody MRC | X05323 | 3q12-q13 | Hs.79015 |
| | | OX-2 | | | |
| 1826_at | ARHB | ras homolog gene family, member B | M12174 | 2pter-p12 | Hs.204354 |
| 41163_at | P24B | integral type I protein | AL109672 | 15q24-q25 | Hs.179516 |
| 38780_at | AKR1A1 | aldo-keto reductase family 1, member A1 | J04794 | 1p33-p32 | Hs.89529 |
| | | (aldehyde reductase) | | | |
| 37742_at | GLB1 | galactosidase, beta 1 | M34423 | 3p21.33 | Hs.79222 |
| 39695_at | DAF | decay accelerating factor for complement (CD55, | M31516 | 1q32 | Hs.1369 |
| | | Cromer blood group system) | | | |
| 189_s_at | PLAUR | plasminogen activator, urokinase receptor | U09937 | 19q13 | Hs.179657 |
| 36591_at | TUBA1 | tubulin, alpha 1 (testis specific) | X06956 | 2q36.2 | Hs.75318 |
| 40091_at | BCL6 | B-cell CLL/lymphoma 6 (zinc finger protein 51) | U00115 | 3q27 | Hs.155024 |
| 37192_at | EPB49 | erythrocyte membrane protein band 4.9 (dematin) | U28389 | 8p21.1 | Hs.274122 |
| 37508_f_at | HYPA | Huntingtin-interacting protein A | AA675900 | 2q23.3 | Hs.107213 |
| 38672_at | PPP1R10 | protein phosphatase 1, regulatory subunit 10 | Y13247 | 6p21.3 | Hs.106019 |
| 595_at | TNFAIP3 | tumor necrosis factor, alpha-induced protein 3 | M59465 | 6q23.1-q25.3 | Hs.211600 |
| 988_at | CEACAM1 | carcinoembryonic antigen-related cell adhesion | X16354 | 19q13.2 | Hs.50964 |
| | | molecule 1 (biliary glycoprotein) | | | |
| 38879_at | S100A12 | S100 calcium-binding protein A12 (calgranulin C) | D83664 | 1q21 | Hs.19413 |
| 1270_at | RAP1GA1 | RAP1, GTPase activating protein 1 | M64788 | 1p36.1-p35 | Hs.75151 |
| 33813_at | TNFRSF1B | tumor necrosis factor receptor superfamily, | AI813532 | 1p36.3-p36.2 | Hs.256278 |
| | | member 1B | | | |
| 38508_s_at | TNXA | tenascin XA | U89337 | 6p21.3 | Hs.169886 |
| 31793_at | DEFA1 | defensin, alpha 1, myeloid-related sequence | AL036554 | 8p23.2-p23.1, | Hs.274463 |
| | | | | 8pter-p23.3 | |
| 35966_at | QPCT | glutaminyl-peptide cyclotransferase (glutaminyl | X71125 | 2p22.3 | Hs.79033 |
| | | cyclase) | | | |
| 37022_at | PRELP | proline arginine-rich end leucine-rich repeat | U41344 | 1q32 | Hs.76494 |
| | | protein | | | |
| 35918_at | DLEC1 | deleted in lung and esophageal cancer 1 | AB020522 | 3p22-p21.3 | Hs.200188 |
| 37405_at | SELENBP1 | selenium binding protein 1 | U29091 | 1q21-q22 | Hs.334841 |
| 35672_at | DKFZP434N093 | DKFZP434N093 protein | AL080144 | 1q44 | Hs.33363 |
| 37285_at | ALAS2 | aminolevulinate, delta-, synthase 2 | X60364 | Xp11.21 | Hs.323383 |
| | | (sideroblastic/hypochromic anemia) | | | |
| 106_at | RUNX3 | runt-related transcription factor 3 | Z35278 | 1p36 | Hs.170019 |
| 35372_r_at | IL8 | interleukin 8 | M17017 | 4q13-q21 | Hs.624 |
| 34832_s_at | KIAA0763 | KIAA0763 gene product | AB018306 | 3p25.1 | Hs.4764 |
| 37024_at | PIG7 | LPS-induced TNF-alpha factor | AF010312 | 16p13.3-p12 | Hs.76507 |
| 40617_at | UNK_AC004381 | Homo sapiens Chromosome 16 BAC clone | AC004381 | 16p12.2 | Hs.268371 |
| | | CIT987SK-44M2 | | | |
| 40647_at | XK | Kell blood group precursor (McLeod phenotype) | Z32684 | Xp21.1 | Hs.78919 |
| 1257_s_at | QSCN6 | quiescin Q6 | L42379 | 1q24 | Hs.77266 |
| 32606_at | BASP1 | brain acid-soluble protein 1 | AA135683 | 5p15.1-p14 | Hs.79516 |
| 39436_at | BNIP3L | BCL2/adenovirus E1B 19 kD-interacting protein 3- | AF079221 | 8p21 | Hs.132955 |
| | | like | | | |
| 307_at | ALOX5 | arachidonate 5-lipoxygenase | J03600 | 10q11.2 | Hs.89499 |
| 40769_r_at | NUP214 | nucleoporin 214 kD (CAIN) | D14689 | 9q34.1 | Hs.170285 |
| 266_s_at | CD24 | CD24 antigen (small cell lung carcinoma cluster 4 | L33930 | 6q21 | Hs.286124 |
| | | antigen) | | | |
| 40446_at | PHF1 | PHD finger protein 1 | AL021366 | 6p21.3 | Hs.166204 |
| 37701_at | RGS2 | regulator of G-protein signalling 2, 24 kD | L13463 | 1q31 | Hs.78944 |
| 37200_at | FCGR3A | Fc fragment of IgG, low affinity IIIa, receptor for | J04162 | 1q23 | Hs.176663 |
| | | (CD 16) | | | |
| 31888_s_at | TSSC3 | tumor suppressing subtransferable candidate 3 | AF001294 | 11p15.5 | Hs.154036 |
| 35601_at | UNK_L00022 | Human Ig active epsilon1 5′UT, V-D-J region | L00022 | | |
| | | subgroup VH-I, gene | | | |
| 36713_at | DKFZP434C091 | DKFZP434C091 protein | AL080170 | 1q44 | Hs.51692 |
| 32607_at | BASP1 | brain acid-soluble protein 1 | AF039656 | 5p15.1-p14 | Hs.79516 |
| 39969_at | H4FG | H4 histone family, member G | AA255502 | 6p21.3 | Hs.46423 |
| 35256_at | UNK_AL096737 | Homo sapiens mRNA; cDNA DKFZp434F152 | AL096737 | 2p23 | Hs.5167 |
| | | (from clone DKFZp434F152) | | | |
| 40202_at | BTEB1 | basic transcription element binding protein 1 | D31716 | 9q13 | Hs.150557 |
| 40888_f_at | EEF1A1 | eukaryotic translation elongation factor 1 alpha 1 | W28170 | 6q14.1 | Hs.181165 |
| 33080_s_at | KIAA0474 | KIAA0474 gene product | AB007943 | 1p36.1-p35 | Hs.75151 |
| 38615_at | GW112 | differentially expressed in hematopoietic lineages | AF097021 | 13q14.2 | Hs.273321 |
| 34319_at | S100P | S100 calcium-binding protein P | AA131149 | 4p16 | Hs.2962 |
| 38585_at | HBG2 | hemoglobin, gamma G | M91036 | 11p15.5 | Hs.266959, |
| | | | | | Hs.283108 |
| 39908_at | PAF65A | PCAF associated factor 65 alpha | AF069735 | 11q13.1 | Hs.131846 |
| 32434_at | MACS | myristoylated alanine-rich protein kinase C | D10522 | 6q22.2 | Hs.75607 |
| | | substrate (MARCKS, 80K-L) | | | |
| 38740_at | BRF1 | butyrate response factor 1 (EGF-response factor 1) | X79067 | 14q22-q24 | Hs.85155 |
| 31410_at | TACI | transmembrane activator and CAML interactor | AF023614 | 17p11.2 | Hs.158341 |
| 35785_at | UNK_W28281 | ESTs, Moderately similar to MM46 [H. sapiens] | W28281 | 12p13.1 | Hs.336429 |
| 34627_at | KRTHA5 | keratin, hair, acidic, 5 | X90763 | 17q12-q21 | Hs.73082 |
| 36709_at | ITGAX | integrin, alpha X (antigen CD11C (p150), alpha | Y00093 | 16p11.2 | Hs.51077 |
| | | polypeptide) | | | |
| 36979_at | SLC2A3 | solute carrier family 2 (facilitated glucose | M20681 | 12p13.3 | Hs.7594 |
| | | transporter), member 3 | | | |
| 31792_at | ANXA3 | annexin A3 | M20560 | 4q13-q22 | Hs.1378 |
| 33304_at | ISG20 | interferon stimulated gene (20 kD) | U88964 | 15q26 | Hs.183487 |
| 34435_at | AQP9 | aquaporin 9 | AB008775 | 15q22.1-22.2 | Hs.104624 |
| 32529_at | P63 | transmembrane protein (63 kD), endoplasmic | X69910 | 12q23.3 | Hs.74368 |
| | | reticulum/Golgi intermediate compartment | | | |
| 37351_at | UP | undine phosphorylase | X90858 | 7 | Hs.77573 |
| 37149_s_at | UNK_U95626 | Cluster Incl U95626: Homo sapiens ccr2b (ccr2), | U95626 | 3q21-q23 | Hs.105938 |
| | | ccr2a (ccr2), ccr5 (ccr5) and ccr6 (ccr6) genes, | | | |
| | | complete cds, and lactoferrin (lactoferrin) gene, | | | |
| | | partial cds, complete sequence. | | | |
| 681_at | MMP8 | matrix metalloproteinase 8 (neutrophil collagenase) | J05556 | 11q22.3 | Hs.73862 |
| 936_s_at | PPP1R2 | protein phosphatase 1, regulatory (inhibitor) | U68111 | | |
| | | subunit 2 | | | |
| 38012_at | FBN2 | fibrillin 2(congenital contractural arachnodactyly) | U03272 | 5q23-q31 | Hs.79432 |
| AFFX- | 18SRNA5_Hs_AFFX | 18SRNA5 control sequence (H. sapiens) [AFFX] | M10098 | | |
| HUMRGE/ | | | | | |
| M10098_5_at | | | | | |
| 1369_s_at | IL8 | interleukin 8 | M28130 | 4q13-q21 | Hs.624 |
| 2002_s_at | BCL2A1 | BCL2-related protein A1 | U27467 | 15q24.3 | Hs.227817 |
| AFFX- | 18SRNAM_Hs_AFFX | 18SRNAM control sequence (H. sapiens) [AFFX] | M10098 | | |
| HUMRGE/ | | | | | |
| M10098_M_at | | | | | |
| 35379_at | COL9A1 | collagen, type IX, alpha 1 | X54412 | 6q12-q14 | Hs.154850 |
| 34498_at | VNN2 | Vanin 2 | D89974 | 6q23-q24 | Hs.121102 |
| 1962_at | ARG1 | arginase, liver | M14502 | 6q23 | Hs.332405 |
| 1115_at | PF4 | platelet factor 4 | M25897 | 4q12-q21 | Hs.81564 |
| 35920_at | UNK_N55205 | Human beta-type globin pseudogene | N55205 | | Hs.20205 |
| 39209_r_at | PPBP | pro-platelet basic protein (includes platelet basic | M54995 | 4q12-q13 | Hs.2164 |
| | | protein, beta-thromboglobulin, connective tissue- | | | |
| | | activating peptide III, neutrophil-activating | | | |
| | | peptide-2) | | | |
| 39208_i_at | PPBP | pro-platelet basic protein (includes platelet basic | M54995 | 4q12-q13 | Hs.2164 |
| | | protein, beta-thromboglobulin, connective tissue- | | | |
| | | activating peptide III, neutrophil-activating | | | |
| | | peptide-2) | | | |
| 40215_at | UGCG | UDP-glucose ceramide glucosyltransferase | D50840 | 9q31 | Hs.152601 |
| 33849_at | PBEF | pre-B-cell colony-enhancing factor | U02020 | 7q11.23 | Hs.239138 |

1. A method comprising comparing an expression profile of at least one gene in a bone marrow sample of a patient of interest to a reference expression profile of said at least one gene, wherein each of said at least one gene is differentially expressed in bone marrow mononuclear cells (BMMCs) of patients who have a blood disease as compared to BMMCs of disease-free humans.

2-20. (canceled)
